SEQUENCE LISTING (56 sequences)

SEQ ID NO: 1 - DNA, 141001 bp, Homo sapiens
  source 1..141001: mol_type = genomic DNA
aatttataaaggaaaaaggtttaattgactcacagttcagcatgtctggggaagtgttaggaaacttacaatcatggcagaagagaaagcaaaccatcctttctcacatggtgacaggaagagcaaagcggggtaagccccttacaaaaccaccagatctcatgagaactcactatcacgagaacaccatggaggtaactgcccccatgattcaattacctcccaccaggtccctcccacgacatgtggggattatgcgaactccaactcaagatgagatttgggtggggacacagccaaaccatatcagaagcttaaccttctttggagcatgattattcagttgaacctaagttcagtagtcacccagttatgctgtcttcagctactattttccatatgtttctcaaacatctgatatatcacactggctagtgcactttcttccaccagcataccatctcaatttaccactttaacaattggactgccactttgtgtcagggactatctgtgctccaactactacaagtgataaggtcctcactgacagccagggagcaagtgatccagctctaaaactcaccttatcatctgctttcctagaccactcctaacaaccaactattctgggttgagttctccaagaggcagagagttcaggatacagaatgttgttttgtttttgttgttgttgctgttgttgtttgtgtgtgtgtttgggcttttttgagacggagtctcactctgttgcccaggtagaagtgcagtggcatgatctcagctccctgcaacctccacctcctgggtttaagtgattcccctgcctccacctcctgagtagctgggactacaagtgtgcgccaccacacccagctaatttttgtgtttttagtagaaatggggttttaccatgttggctaggctgctcccaaactcctgacctccagtgatccacctacctctgcctcccaaagtgctgggattacaggcgtgagccaccacacccagcccagaatgtttattagaatgcacaattaataccagaggcagtggggaaggaaggactgagcagaggaggaagttgagttgtgattcaacccaacaactgcctggctggcatggggagctctggagttaaatagggccatcagactttcccagtgtggggccaacatgactgggtctttatacccccacctctgtcagtcactcaacgtggtctccctgcaacaaggtgactcttgcagccgagacaatccctgaagggacagagggctgaagcctgtctgccaacagcactcccagtggctggaacaagtccttccctataggggaatctgggcggcacacctccatctccatgtccatcacatacgatatcacagacatttaaatattttgataactgtacataagagtttcctttataatcttatagatcttattttatgcatttgaaaatattcttctgagacagggcttttatcatattgccatagggtgccacgatataaaaaaggttaaatactctctgattcagaagtatccaatgatgacttctctctcatgcatttaattgaaaatctggtttttctccttctctgctagttctctacctctctccccacctcccacatcatagcctattcacatatgtctgaatctcatgatagacaagttcaggttcttttcccaggttctttttaccacatccccccacccccacataaaaagtatatatggcacagcctaggttccacccaaatcctttctcctcttcttcctgggcccacaactctcctacatacattggtataccttgcgcttagggatggccatgtgactaagttctaacagtggaacatgatcagatgccacttccagcctctaagacagccagtgtgtttcctccataagctccttctcttcctcccaactggagactctaaatgatgaccctgcctcaagcaagcaaacaacaagtccctcaggggtggtgtaggctgcaaatggaaggagcttgagtcccaaaccttccacggagaaggctggctaccaacctggatcactcacccaagactgctcgaagagttggtttgaaccattgtgttttggggtctatttattacaacagtttagcttgctttgtgaatagatttagtggcagagcctccaaattctatagatacattgatctcagtcctaaccgcatctggaacaccattaaataaaggaattgcaaacccagagaaggtaatgaatttgtctaaggtcatacaagatggctaggatcaggacccaactctccagttttctttcttctctgctattctgccttctgtgatcctacataagtgggcatgattgtataacatatgcggccatgagatttctctttcagcaagagaaagggacaggaagaaagagagggaatgcattttcttggcctgaattagtgtgagccattagttacctacattgactaaattatctggaatgaacattcaactctacatcacatatagttaaaatgacagatctgcttaagattgtttctagcatacgttatttcaatttaggcaaatgtgaccattcagtgtgaggggaccatactgtcattaggtccctgtcagttctcaattatactgttatcttagagggggaaaaatgtgaaatttgaatgtagacgagtgttgatttgactgctacagtttattttacgtatagaaataaaataatgtgtagcaaaagcattattacaaagatgataatgaaataactagtatttataatagtataatagtatagtatttataatagtatgatagtttaatgactatttgtcagatgttgtgtaagaaactttatacacacacacacacacacctcatttaattcctgtatcaatcaggatacaggacgctgtggtaacaactcctcaaatctcggtggcttgcacaacaaatgcttatttcttttttttttttgacaccaagtcttgctctgtaacaggctggagtgcaatggtgcaatctcggctcactgcagcctctgcctcctgggttcaagcgattctcctgcctcagtctctcgagtagctgggaacacaggcacgcgccaccacatctggctaatttttgtgattttagtagagatgggatttcaccatgttgctcaggctggccttgaactcctgacctcaagcgatccacccacctcagcctcccaaagtgctgggattacaggcatgagccactgcgcccagccccaaatgtttatttcttgctcatgtgacatgtacttcctcgagtttttccttcctgagatctaagctgaaggaacagctctctggagccacgccattctggtggcggaaaggaagagtaaaagtggtagaaccttgcaatgctcttgaagcgcctatttggaatgtctacatcatgtaaatggtaatggacaagtatgtataatccccacaccaaaaaaaggggacactattggggacaataaccacatttcaatgctgcaagacggatattgactgcacccccttcccactttcagaaagaagaagagtaattttgctgaactccttctagagactggaaatgtcccttccagttggggtgattagggaaggctttggtaaaatttgagctagagtttgaaggttaggtagactactggtgggtgaagaaagaacaaggacctttgtaggcaaaggaaaacctcagaattacagaggtggaaaaagagttctagtcaagccacttcagctggctacagagtaggtgggaaagaaaatgggaggacaagggctcagatgatggggggttggggcattggggggacacttgaaagctaaactaaggggttgaacttaatttaggaggcagttagaagcttttacatatttttgagcaagagagtgacataattaaaatgatctgggccaggtgtggtggctcacacctgtaatcccagcactttgggaggctgaggagcttgggtcacctgaggtcaggagatcgagaccagcctggccaacatggtgaaatcccgtcctactaaaaatacaaaaattagccgggagtggtggcatatgcctgtaatcccagtagctgggaggctgagacaggaaaatcgcttgaacccgggaaacaggttgcagtgagccgagatcgtgccactgcactccagcctgggcaacagagcgagactccatctcaaaaaaacaaaacaaacacacacaaaaaaccaaaaataaataaataaaatgatcacttctgaatactgatctaactaggggttgcagggtgggctgatatagggagaaactggagagcaaggagatcactaaggtccctacatgtccagaaccaagatagaggtcttgaactaggatggtggcagttagaacaacaacaacaaaaagtcaattccaggctgagtgcagtggctcatgcctgtaatcccaacgctttgggaggctgaggtgggagttagaaagcagcctgggcaacactgcaagacctcctctctaaaaaaaaaaaaaaaaaaaagttagccaggtgtggtggtgcccacctgtagtcccagcaactcagaaggctgaggtgggaagattgcttgagccccaggagttcaagcttgccgtgagctacgattgtgccactgcactccagcctgagcaagaccttgtctccaaaaaaaggtcaattccactgacttttctaaggtgtacaccatcaaggggcagctccatctccaggccattggctcatgagacattctgtagtcagaaggctagggcagattgctttgagcaagcccccatggtggttctcactcctacttctttgggtatatgcccctctgtttaaaaataaagttaatatgcatttaaaaaaaaaaaggagaaaaaggtcagttccagaaactgtgtgaataaagcattttacttgctttttctattaatctataacatatgttgattttttaaaaagaatataagagctatgcaaattggagcttcaagacaacttcccatctccctaggaggagatggctgccctaaacccccctacatagaaatcatcccactgcttgggcttaaacttgatgttggggaaatgaaaaatccaagctaaggccgaagcctggggcctgggcgaccagcagaatgaggaccactggtcagtttcaggctgaggtgcgtcttccaggggacaatctctagctggcccttaaacattcagacttcaagctctatttacagcataaaggtgtttcaaaagacgtgatacaaataactgcaaatgctctgcgatgtgttaagcactgtttgaaattcgtctaatttaagatttttttttctgacgtaacggttagattcacgtttctttttttttaagtacagttctactgtattgtaactgagttagcttgctttaagccgatttgttaaggaaaggattcaccttggtcagtaacaaaaaaggtgggaaaaaagcaaggagaaaggaagcagcctgggggaaagagaccttagccaggggggcggtttcgggactacgaagggtcggggcggacggactcgagggccggccacgtggaaggccgctcaggacttctgtaggagaggacaccgccccaggctgactgaaagtaaagggcagcggacccagcggcggagccactggccttgccccgaccccgcatggcccgaaggaggacacccacccccgcaacgacacaaagactccaactacaggaggtggagaaagcgcgtgcgccacggaacgcgcgtgcgcgctgcggtcagcgccgcggcctgaggcgtagcgggagggggaccgcgaaagggcagcgccgagaggaacgagccgggagacgccggacggccgagcggcagggcgctcgcgcgcgcccactagtggccggaggagaaggctcccgcggaggccgcgctgcccgccccctcccctggggaggctcgcgttcccgctgctcgcgcctgcgccgcccgccggcctcaggaacgcgccctcttcgccggcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtaagcgccgcggctccgaaatctgcctcgccgtccgcctctgtgcacccctgcgccgccgcccctcgccctccctctccgcagactggggcttcgtgcgccgggcatcggtcggggccaccgcagggcccctccctgcctcccctgctcgggggctggggccagggcggcctggaaagggacctgagcaagggatgcacgcacgcgtgagtgcgcgcgtgtgtgtgtgctggagggtcttcaccaccagattcgcgcagaccccaggtggaggctgtgccggcagggtggggcgcggcggcggtgacttgggggagggggctgcccttcactctcgactgcagccttttgccgcaatgggcgtgtgtgtgtgtgtgtgtgtgtgtgtgtgtgtgtgtgtgtgtggaggggtccgataacgacccccgaaaccgaatctgaaatccgctgtccctgccgctgttcgccatcagctctaagaaagacgtggatcgggttctagaaaagatgactccctgcacgcccctccctgcacctcccgagcagtgattccgacagggccttcactgcccctgattttaggcgggggccggccccctccccttttcctccttcagaaacccgtaggggacatttgggggctgggagaaatcgaggagatggggaggggtccacgcgctgtcactttagttgcccttccccctgcgcacgcctggcacagagacgcgagcagcgccgtgcctgagaacagtgcgcggatcccactgtgcacgctcgcaaaggcagggttcacctggcctggcgatgtggacggactcggcggccgctggtccccgttcgcgggcacgcacagccgcagccacgcacggatgggcgcggggctgcaggtgcatctcggggcggatttctttctcagcgctcggagcgcagggcgcccggcgtgtgcgctccctgccggaggcgcggggctggcgcgcagggctcgcccctcactgcggcagtgggtgtggaccctggtgggcgaggaagggggaggataggctgtgcctcctcccactcccgcccccagcccccctttttttccccctcggaacgcgaggtgccatcttttttcggcgtgtcacgtctttacggtgccatgccaaaccgggtggccgggcttcataggacagggcggggcctggcattaaagggagggggacaatcagcgctgaaatcttggcgttttgctgctgcgggcgtgagcactgggggcgttcgcccagcaccttcttcgggggctctttgctttgtctgtagaggttacgtgatctgcgctcccagccctggtttctggcttttattctgagggtgttcagtcaacctcccccctacgcccatgcgcctctctttcctttttcgctcctcatttccgagcccattgttggatctcgaggcttgctgggttcgatgaactcgagtcaaccccccgacccccggcacgcatggaacgggcgtgaccgcgcgcagcctcgtctcggagtctgccggcgccgggaagcttctgaagggatgggattcgagtctccgtgcgcgctgcgggcggcggcagagggatctcgcccctccctacaccccaagtgtcctgagggccacgccacaccaggttgcccagcgagggacgctggctacccatccggggatgggtggggagccctggcggggcctctccggctttacgccctgttgcttcgcctggccggagaatgtgaggaaggggcataaggttactggtgcttcggccacacccatctttctgagcccactggactgggcgcagaggggggattgccatggaaaccacaggtgtccggagaggggatcttggggctggcctcaccccttccctgcggagattggggaccctggggtagggggagccgcgcccagtcggcctcctggaggacacgggaggaagccccgaacccccgcgcctgaggctgtttctgattggcccctggaggccgcagacacgcagataggcggccctgggtgtatttttattaatattatgtccgtactgattaatattatttatcttaaataaatttcacccgtgtccaagttcaccgcgcccccaaaaccgagtctggggcggcagggggaactcctggccaacgaatccatgcctcgccctcctgtgatgaacctggtacgcacggttttctggttaattctatcgctgaaaactggtgcggggggcgcacttctgagacggaagagcatctaggagctgaatcctccacgcgggtcgcccaggttgatctgaatttctggggaatggcttggctgcccgcccgggaccaggccgaccctccttgacggtggcgtagagggctggagcctgggtactgcgaggctcctcgcatggctgggcccgccgcgaggggttgcagagcggctcagggatcgattcaagcatcgtctctcctccctcgcccccagacagagctgggcgcggggttccccttccagatggagcgagggtctcggggtggccccggaaaaggggagcccgcggccacggctacgtattgccatctcgcgagcagagatgtcacctcctgcctttggaggaaagggagcccggtggggatgagcgcatttagcccaatgctgggaacaaagcgcactccgcgcttctgcgatttcgctccattttgaaatgtgttggcgctttggtggggccgctgcggtgggcaaggccgggggcgctgttaatggaggaacctcagggggacggtccttcgtaggaaactctatcctggctctgcgcgcgctttaaggaaatggcttccctccaggacctcgagggatgcagcttttgcgcggatgacggtggggtgctgaaccagccggtgcgcctctggaaatgtctgggcacggatcctggggccatcgacgactcctccccattcccagcaggcgggagctcttacattccgagcgagtgacccctctcaccctctggcgctcacacacctgtaactccaaacctccgtctcagaatggtccaggctggaagggatgatgggggctccgacagcgactgcctagctcacccctctgcgtgctcaggctccaggctcagcaggaccaatttgagttctatctgatccccctcggccccttaactgacccatcctacaggagacagggaaatgtctttcctaccgcggttgattctggggtgtcattttgtgttttgtgatggctgcttatatttactgtataagcattgtatttactgtataagcattgtattataattactgtataagctgcttatatttactgtataagcatctccaaatcctccctctacgtaaacaaattaatggataaacagataagtgtatcccctgcccccacccctgctacgcaggtccggagtgactcttgaagctcatacattccttggccaagtttgcttctctaacagatgtttatatagcaataacctggcttggctcttgggttcacctttggacgatttggggaaggggcttgttggctttgctgggttttggatgagtgacagtccatgactgttcctgctggaagggcgtgacttttaagtggtttctaatatcaggcattgctcctccgacaggaacaaaagaaatggatactgcccataaattgttagaaaacttagaatcgctttgattgaggaaaggttagatttattccggttggaaaaagtggcctttctattaaacgtgccctttgaccctcatgcccttggaggtcggtgccagcctggagatgggataagattgtggttttccttctgcctttttaacatctgttgttacagtccatttgttgaaaatttaaagaaactgttttattccactttccctcagcatttatgtgtgtggtttcagtagctctgtggctatatgtacgaacacgtgttatttttccaattggacatgtgataattttccaactggaccttgccttctattgatgtatttatttagcatcttccttactccctccttgaaaaagaatcactcaaaaacaaataaaaacagccgtaggggcctaatacagtgctagacatacaagaggtattcggtccataccaaatggattttatccatgaaggataaatggggaaatacagtgggaagcaggtgggaaactgcgtttgactctgctctttcctccaccaccactttcctcatcaccgtgttcagagacccccaaagccccctcacactcccagaaacacccccctggccactcctaacttgccatgcccaggagttaggtgcttccactagtgacatggagctggcgtttggggggcacctcagcaggtgacgggaagagaagaccccagcctcaccagctgggctgcagcagggagaggagtcctcatgttccagcagggactctcagctgttttcctgtaaaaccatggttctcaactgggggccactgagatgtctagagagatgtttttgttttcacaactcggggagggtgctactgacatcttgtgggtagaggccaggaatgctgttaaacatcctacaaggaaggcacaggacagtctcctacatcaaaatatgacccagtcccaatgtcaccactgctggggttgacactggcactgctatcttaattacattcattgagtgtcttttaggaggccctattctaagtgcttgctaagattatctcatttaatcctcacaacacttccgctatgtagcaggtgctgttattatctccgtgatggggaaactgaagcacagagagggttagtaacttgctaaaggtcacagagccagtgggtggtggagctggttgcctgacactagttccctcccctctcagccacatgtgggtttacttggccattgtggactagtctgggaacccagatatgatctataacattgacccagtagaatattgattccaaaaccactgtctcacaaatgaatttttacaagagtctgtaatcggagcatgacccagaataaggttagggagatgtggagttaaagctctcaatttcttatctggccccgacacagagagcaaggcatttcactctacattggtgctctgtttataaaacaaagagcaaatatctcttcctaaggtccttaaacctcttcccccaatccagggtttctggactgctctgccatatgacggggcagctggtttgattgacccagggaaggctggaaatcaagactgggggatcaagacgtagattcagtgtggccaaggtcaagtctctgaggtttagggacatcagatccccagcttaggttctgtacctcggcaaggtgaaagcgttggcgcccactgatgaggcctgctctgagattgtgggtgtgggttgagttgggtgggcataggcaagtcctcttgtaagaatcttttggcaaagatgggcctgggaggcttttctcacttcctggggcccaggctttgcaataagtattccattatactgtggtaccttggggctacctgagaatcctctgtctcgcccctgttgccttgccaaagagtttgctgtccaagaattcctttcctgtctccaggtgccatgctcctgccacctctgccaggttccctgcctgcccagatggctcccaactgagtgtgaggaggaatttgagacaggttttgagctttctgggttctccagttaggaaactttctgtaagcatgcagatagaatgggcttcagcaaaatacaaactcgaacaacttccatgtatagtcccttaattttctttgcttttttcatatttcatcaggctccatgctgagcccaatcagggacccgatagaaatccaaacaccatgtcagcgagtccccaagaaatgcattttgtgccaaggctattcaaggaaggtttgggagcagctcaagggcagacactgttaccctcccccaggtccccagtgcagggcagtgttctgcatgtggaggcagtttggcctaatggttaaggaggtaggctctgatcgggcctcctgggcacaaatcccagctccctgctcactgtgagacctaagccatattgtttagctgcttggagagttttttgtcatccacaacttggagtatgatggtacctgtctcacgggttgccatggggttcacacaagctaacccggtactcactagggccaagcacatagtaactgctcagtaaatggcatcatcggcggtgtcctgtggatgagtgcttgtgattggctgaatgaccagaggggtctaaagatcctggtgatggaatcagttgtacagataaattgttacactgagtagggatcaagataggaaaagtcggcaactacccagctcccctgcaccaaactgggcagaagtggatcctctgaaaattgcacacacccatgtttaaatgtacacacagaactcttgccacaggcaagcggagatttgtcatctgctgtccctgcctcatcttcttcctgaaatccactccatgccaggaataaactgcatgctctccaccagcccaaactgacctgccttcccgccagccatcccgggcagggtgacctggcttagtacatcgggttcagagatctttccagtttactcgttgaataaaaagtgagggctgatcgagaaagtaatggcagtcagggaaggcgaaggaggtaaagaagagattttacaaatgaagtaattcaacagagtgctgacattggtaaactggcaaacagatttcagggtggttggttgagagtagagtagaaaaggattaaataaagcaaacttgtggtgtactgaatcttaggaattccatgtatccaataagtatagtcatttatgaattaataaattcggcctaagaagccttcttatcgcttaaatcaagactaagtaacaatatatcagttttaaaaagtcattatatcagaaaatcatttaaatgatacacatagatttccaagattttactttaaccgaaactatataaatgtgaatttgttcacccatcttttgacacagggctcaggtcttctcttggtgtctggatcagccagttgaaatttcttgtctgttttgcctatgccacattaataatgcactgtctgggtcctccgatttcagtttggattttgggtttacattgtggagtcatctgaatgcagaatccttcagggattttacttttttttttttttttcatggtctttaccatcccatttgatagtaaatattactcacctttatgaagtctttccaaaacattcaactaaattttcttaaaatcattgaatgatttgaagagcttattcctcagcacttttactccatcagcttgcaccttattttttaatctttttttgagacggagtctcgctctatcgcccaggcttaagtgcaatggcgcgatcttggctcactgcgacctccacctcctgggttcaagcaattccgcctcagcctccgccgtagccgggactacaggtacacaccataatgctcggctgatttttgtatttttgtagggatggggtatcgccatgttggccaggctggtcccgaacttctgacccaagtgatccacccacctcggcctcccaaagtgctgggattacaggtgtgagccaccgcgcccggccagcttgcaccttatttaggatatgtgattattatagcaagtctggtgtacatacaagattttgaatgggcacagatgacctttagtaagtgcttggctgtgataagaggcagtcctgactgcagatcaggctgtgtggaccccagccttgcatgtttacagaccttcatgtcttattcttacagggtatcagaagaacacctactggggaaacttataaattagtaaaaggtgggcattctccccgcccatcttctgtctgtctgccaggactagcacagcactttgaagtcattcacatagaatcccaacttaagagggtaaaatcctcctcaacagactgaaaataagtttaaattccctttgctatattaactcccctgaggaaagagtcttagatcaatgtccaacactaaaaacagttttaaatcagcaagtgagaattaaatctgaagcaattgataataatgtttcattcattcctctcctttggccccgtccaccctactgctaaatccaggcatcaaagagaagagggacataattatctctagtcccagctgctggttttccttccagcctatggcccagttttctgttttactgagaaggctggtgatgttatcttgggatctaagtctgcagtttcaccacaaaaagtccagggatgcactttcatgcttgtgtcctcctccctgggatagcaaggatattagaagacccctggctctgtaattgcttgtcatgtgctctacagacgccacagaatgccaagaacgaagtgctgggaaggacaaattcatggaaccgtgggacggtgctcctcccccagcgtaaaggacagctcctcctcctgaattggagccagcgttctaaatcatgtgtcaacagagttgtcctggatcggatccagttctgccattgatttgcaggtcatttcagtggtacctgtttccagttgttcttaattgaacagtggcaccaaactattgtcttgcctcatccccctcccatggcctgtcccccaaaaagagacttcttgggtaattaatcagggcaacatcaggcagtctgggcgcggtggctcacgcctgtaatcccagcactttgggaggccgaggcgggcagatcatgaggttaggagattgagaccatcctggctttgtgaaaccccgtctctactaaaaatacaaaaaattagccgggcgtggtggcgggcgcctgtagtcccagctactcgagaggctgaggcaggggaatggcgtgaacccgggaggtggaggttgcagtgagccgagatcgcaccactgcactctagcctgggcgacagagctagacttcttctcaaaaaaaaaaaaaaaaaggaatctctttggttttatatatattttttttatatatataatatatattaaaatataatatatatatttatataatataatatataaatatattatatattatatattttatatattatatattatatatattatatattatatatttatatatttatatattatatatatttatatattatatatttatatatattatatatttatatataatatatattatatattatatattatatattatatattatatatttatatatattatatattatatatattatatattatatatttatatattatatatttatatatattatatattatatattatatatttatatattatatatttatatattatatatatttatatatattatatattatatattatatatgtatatattatatatgttatatattatatatatttatatatataatatattgtatatattatatatctaatatattatatatattatatatattatatattataatatatattatatattatatattatatatatttttatatatataatatgtataatatataatatatataaaaacatatataatatatattatatattatatatatattatatatattatatatattaaatatattttatatatattatatatatatacacatatatatatataaatgaggccaggctcggtggctcacacttgtaatcccagcactgtgggaggatcacttgaagccaggagtctgagactagcctgggcaacaaaacaagatcctgtctctacaaaaggaaactgtaaaaattagctgggcatgatggcatgtgtctgtagccctagctacttgggaggccgaagcaggaggatcgcttgagcccaggagttcaaggctacagtgagctatgattgtcccatagcactccagcctgggtaacacagcaaggccctgtctctaaactttttttttttaattctatttatatttacatgtatttaaatgtgaatattcactacctatttgttgcatgcctgcattttttatactgggcttgccaaaaacccgaacagctttctactttgacaatgtatcagaatttaaatcagcaatatgttaataagccaagcaaaggttatatatgcaaataaaactgttgtctataacctcctgttacactggggcacagcaaaagtcatggtgtagtcgcatgtgaacctgtccctttcatagctgctcattgccaggaaacatcaggaatagccatttggaagagtcatcagccctcccaccatccgttttctgtcttgtcttttccctatgagcaggggaaattccacgctggccccaatccccagtgcagcggctcagcctctgcctctgctgctggtccccatgaggccagcttagaaacggaggattttgcagaacatccctaaatccgcttgaataatgaagtgatcattcataaactcacctgaaccttattaaaacctatttaatatttttcctggataatcctatagggataacttgcctcctgggcttctctccaccgggttcagttcttcctttagtggtgaagttcctcccttcttagcatctcaactgtgcctgagaaaaggccagtggcggctgcactctgttccctgtggagtgttaataaagactgaataaattgaaataaatccctttcaatgtcattaagtgctataaataatcatgaaccaatgttcgatggctgatgagaaatgcaagaaaaaatttttaatcagtaggattcataagttgacaatctgggccaagttaaaaaaaataaaaataaaaagacttttaaaaagatcttatcgtttgttaccagtaagactgaattccagaagcaagctactccctcatttgtgggcccctgttatcactggctgcttagggttgccaagccctgaattcatttgtcaactaagagatttttggccaagattaagatttcccatgcctccatatttccatctgagaaatggagattatactgtcttccccctcagaatggatgataatgtggtctctcttctgttcgcatagtcatagaactgaaataaaacaacttaagagaattcctttgagcttctcagaagtgctgcagggctgggggatgcctcccaggagccgcagtcaggtgctgatctgaagtctttggtgggctgactttagcctgacctgaaatagtatagctgctgccacctggctcccttagcgtcagtcagacggtgcagctggttcctaggggtgagggctgagccagcagggtccgtgcccaggagggatgcatgggtggccacagcccagcctgcactgatcttgtctgtccccttctttggaaggaaggagccccaaaccagggtgcaagacagtgggtgggggtgccttgagcatgacctcaagtgatttccagcccctgccagtgctgacttctctggggaagggctgggacttccttctgggctcaagtcacgacccttggatggaatttcctgggagcttttctgttttttctggagttttcagttttttcctaaccagacagggacttggtacagaatctcatattctaattatgcctaggagcagcctctccccaccactcacagtgtttagcatgtgacaggaatcgattaaggcatgagtgattaaattaaagccaggcattgacttggatggtgtaatattctgacatctgtttggtgtcaaaggcacggggcaggcgcgttaattgaactgcttgcacctggcatttgaattgagccagagcggggctaaagtcagtttgccttcaccctgtaaatggagggtttctccggagcgtggatggtgggaggtatttcagggtgtatgcataacccccaccctgacaatggcccatctcttctccagcgtggccaggtttgagtgccagtcctgggtgtccagtggccccatagccttgcgttttagtaaaatgctgcccccattaccacctggtctgtgcacttcggtcactggaatttgccatcttccagtcccgaatgtggcaagccatggagccttaagctcttctccctccacatcctggaacagacccgccagtttcttccaggcattgcctcagtttgcccctctgtttccagtcacactctcaccagcgataaaatgattttagaccttatcatctcaccctcggatccttatggaaacaataatgagttgttccctgtttcaattccaaaattcatatccaatccgttttgcatgccattgccaaattcctcccagagcaaccccgtcacctgccctggccctctccaagtgtggtcctgccatgggcatcgcctgctaagccaagctggcctcgagctgcctgcccgggtccccacaccttggctcacctccctgcccagtcccgcctcctgccagcctgccctgtggctccttcatagatgccgtgctctttctgccccttgctcacccatggcagccttgcccctctctccctgccccaccccctatttaaattgacctgaccttcctcagtgtccatcttccccgaagctttccccagccttggcactcaaggtccagaggctacgcgtttcctctcacctgtggcagcgccgtgctccccagtgcctcacagtttccttcttgcccccgcttcctgtgtaggactcatctgcccacaggttgcacgtcctgtgagggcaaggactgtgtcttatgtgactttccttctccagtcacagagctgggcacatagatagctcaaaaccctctttattaacacagttggatgttgagaaatcaaacaggccaatgtcaaatgagctctccttatttaaatcaagtcagttctccacctcctagcactcagttccagtactctatatacatggaaataataaaaaacacatttcctttgaaacattctataatcgttcctttgccctacttcagaccaacttaacgcactccccattggtccaaatgagttttgctatacgaagatgctgataataatagcagcagtggattattctgctaaaaccattgcctcgttaatcctcagtcccgaggtggggattattatcctcattttgcagagaagcaaactgagactcagagatttcacagctggggagggagccagctcatccctctgtccaggcccaagctctctcccgcttgccttcctgcctctgcaacctcagagcatcccccatctggttctactgcctgtgctagtcgtgcaggagccaaaagacacgtctttagtgctaaggactggagaagccatgccctccagcctctgtgaatgggtcatatgtaacatgagcctggagaaattatttgaaaccaaaggcaagcctctaaaccaggctgctgcttcatggcgccggtgacggcagaaccaaatttagtgctgtgggcaggtccacacttatcaaatagagaagctcatttttcttccggctcacatcaagcatgaaaaatgttcacacataccccccacacacacatgctttccggaggggtccatgtggctagaggctggaagatgtggatgagaggagcctggcaggtaagcccagggaagatgacattcagcttcccagacagcatctacagggagaaatttaattaaaagtggggcggtttccctgagcaaggcagacaaagtcagccctctactgttaagaaaaagggtcacagtgagaggggaggtgaggagactgagtctgtattttctagtctgttgggctacactacctgatcccccttcctcaaaaatccactttactttccccatgtctacaccaatgtggttcacactctgggaccaggaaaagggggagtgatggggaacagagaagggaggagctcacacagctgaggctggggttatgcatatcgaattacttagaatttgcaacctcacagggtactttcatggcgttgaaatacacttcccacagccaccctccctctaactaaaagcaagagtcatttctcagttctggtcttgcctcccacgttctcctccacatttaagaaaatccaccagctacaaagtgaagataccatatgtgatatcccaccctagtttctgttttatcagggtttggagcaggtggagcaggcagagggatcatttcagcctataaattgtattaagggtgagtactgagtcattcttcaagaaaagttttagaagcatccaaaactgaagggtggagccacctggagacagtatcatcagtcctggccccgagcatggcctgcataggcccccatggatcccagcgggagctgcagagtgcgggcaccttggcacacagccctgagtgcaaaattaggagctgggcagagggcatctctctgtcgccattgggcagcccagggcacactggtcatagccttagaccacgaacaccctgtgcccgggggacagatgcaaccagtgtgccctgggctgcccaatggcaacagagagatcgacacctggaccccatgtcacggggactccactactaaggctcctaagactgccaccttccagtgggataagccctgcctcctactgggcccacaatgtgcagagaacacttgggactacctggctttctggatacacaaatattgatccaatctggactaattagaaggtcagtcccaataacaaatcgaagtcagctgggcgtgatggctcactcctataatcccagcactttgggaggctgaggtgggcagatcatttgaagccagaagttcaagaccagcctgggcaacatagcaaaaccctgtctctactaaaaatacaaataattaggctgggtgtggtggctcatgcctgtaatcccaacagtttgggaggctgaggcaggtggtcacctgaggtcaggagtttgagaccagcctggccaacagggtgaaaccccgtgtctactaaaaacataaaaattagccaagcatgatggcatgtgcctataatcctggctactagggaggctgagacaggagagaatcgcttgaatccaggaggtggttgcagtgagctgagatggtgccactgcactccagcctggttgacagagcaagactctgtctcaaaaaaaaaaaaaaaaaaaaaaaagccatgcctggtggagcactacgtgtaatctcagctatttgggaggctgaggcacgagaatcacttgaacctgggaggcagtggttgcagtgagctgagatcgcgccactgcactccagcctgggcgacagagtgagtgagactccatttcaaaaaaataataaatctgagtcactttaatattgttatttggatgtcaacctctaggtgtttgagacaggagagtgatatgggggcactggaaacacacaggcacggggtgtcctcacacttgggtagcccacacgatgtgatttcagggtgctgggaggtccccccactccccaaattactaacaagtggatagtactttacagtttatatgatctcatttgattcttaacatgagcctgtgagtgaaaaattccttcccctcttctacagattaggacgttgagattcagggaggttcagagggattcagggaagtcaagtggcacctggagtcccgtggctaatttgaggccggtaggggattcgaacccaggatttgtgcttcttatgcctgggcttctgctccctggggcatggtcttccccctagctttcccattcactgctttagcctaggggtcctaccctttattaaactgccagtgcctcactgcttttctcccccaaagacaaaaaaaaagtgtttttgcttttgttttgtttttcatgggcagagacctggaatttcagcttgagaatttgtgccatatgataaataaatcaacagatggctttttccttaaaaaaaaaaaaaaaaaaaactaagatgtatttgcagtgaggcataatttgtaccaaaaagtgctcaccacactgtagtcatgggggcaggaggcagccgcgggtgaagggagaaatcttggagtccaggcagcccccttctgggctgaactggggagctgggggtgctgccagccctgccaggttctcctaggaggcggcagctcatatggctgtgggaggaggcagagggagcctcatatgcacccacatttccagggatctagaagacagaaggaggaaaaccaccatcatgttaaagcagacagttaggtaacacatcctgtaatacaagttattttttccacatctaaaggctaaaaatagttgttagaatttaaagataattggtaaatgagtttctatccttctagtttcacatcaaatggaatcatgctgccttcacatcactagtgcccgttatttgtgtttaatttccacaatgttgtctaattccactctttgggcttccccagggatccagcctccctcactcgcccatcgcagggagatgctttattcatctttgtgtcttctgtgccgggcatagcgcatggcacagaataagcactcagtaattgattcacgagtgaataaatggatgagtgggtgagttcaatattgactacaaaaaccctaaggccacactggtgagtggctgcgcctgtagtcccagctgctggggaatctgaggcaggaggatctcttgagcccaggagtttgaaactagcctgggcgatatagcgagaacctgtctcaaatgacaaaaacagggccaggtgcagtggctcacgcctggaatcccagcactttaggaggccaagatgggaggatcacttgaggccaggagtccgagaccagcctgggcaacatagggagaccctgtctctacaaaaaattttttaaaaattagctgggcatggcggtgtgcgcttgtagtcccagctactcaggaggctgaggcaggaggatcacttgagcccaggaaattgaggctgcagcgagccatgatggcaccactgcactgcagcctgggcgtcagaacgagacctgctctcaaaaaaacaaacaaacaacaaaaaaaaaggctttcttaaagagacttgagaacagaaaggggaacagatacataacttatatatttatttgttcatctttccaccttcctggagggtggaggggaacaggtctgtatttggagttttgaatgctaaaagtgggaatacatgtactgtttgccatgatctgttcaaaagttaagccaaatgccttagattctcctgaaaactggaatgccactgtaaactataagccccacttcaaagataaaagatcttgatgaacagggctgggtctgtggactgggcctctccccaccacacaaggaagggtggtgccagttgaaggaaaatcacttaaatccttgctgtctcctaataaggtgtggtcccaggtagggctgtcagaattagcaaattaaaacacagggcatctgtgaaaattagaatttcagataacaacaaataattggcataggctgcataatgtccctcaaagatatcaggtcctaatctccagaacctgtaaatgtgatcttatttggaaaaggggtctttgtagatgtggttaaattaaggattttgagatggggggattatcctgtattatctaggtaggtcctaaatgcagtcacactcatccttgtaagaggaaggaagagagagatggaaaacacagaagagaagacaatgtggtgatggaggcagagattggagtgaggtggccacaagccaaggactgctggcagctaccagcagccagaaaagtccaggaaccaattctctcttggagctccagagggagtgtggccctgctgacaccttagcttcaacctagtgatcctgattttggactttggccttcagaagtgtgagggaatgaatatctgttgttttaagccaccaagtttatggtcatttcctacagcagccacaggaatcaaaaacagtaagtatgtcccatgcaatgtttgtgacacacaccaaaaatattacttgttgttcacctgaaattcaaatttaactgggtctcctgtattttatttggccaacctagttcccaggcccaaagaaagaggcttttgaaatttgcaagaaagctggttggagctgtcagaaagtggactttgtaaacacagtaccaccgaaccaatttgaactgtactacctctagacaaaagagagggcagtcagacagttgttcgtgatttcttctttcaacagtcatttgagcacttactacaaaacagaagctatgtgtaagggtggaggcgttagctgttaatcaggacctccaggctaagtttctgtattagtccgttttcacgctgctgataaagacatacccgagactggggaatttacaaaagaaagaggtttaattggacttacagttccaagtggctggggaagcctcacaatcatggcagaaggcaaggaggagcaagccacatcttacatggatggcagcagacagacagggagagagagcttgtgcaggggaactcctctttttaaaaccatcagatctcgttagacttattcactatcaagagaacagcacagaaaagacctgcccccatgattcagttacttcccaccagatccctcccacaacatgtgggaattcaagatgagatttgttaccatatcagttaccaacccttccagataaatcacgtgaaatatcgccattaacagagtgagctcaggtggttcttcagtgcatttctgatacctgaaccttccctgggaatttcacagaccatcaggctctccaccctttgatagcaggatagcagggcccaggttctgcaggaggagatgttaccacaggcctgaaagggagggaggggcagatgctacaggaagatgctggctctggattcgctggaggagctttcaagggaagtagatacacactgtctccatcatttcatgtccatcacactctaaaatgctttggacaagaagcaaatgttaaagacaaatgtggcccattttcctgtacaaagagggctgctcccatgccaggctattggcactggtgggcatgaggcttctctgctgccctggccggggggttctctcactcaccattggctctctgacacctggagagaccaccacccttgggctttcatgatgctcacagaatccacactgttggagctttaaggagcctggatcaactggaacaggcagggagtactaggacagcccagcattgccccaaaatatccaggcctgataaaagagaaaaacaggtagctcacaggaaaaggataaaaaaaggaggagggatttaacatgaaaaggtgcttgatctccctcataataaaaagactgctgattccatccaggcaagtgacagaaaaaaaaaaattaatttaaaaagactgctgataaaaccacagcgagacactgctgctcagggatctgagggtgtgggcagccaggctgccacgcatcatgggtcggagaggaagaccacacccctggagcagagggcggctgatctgtcagatgccctttgacagcacctcagcttccaagaattaaccctttctatgtgagcagaggcatccatggggggacacactggtgaatcatctgttatgtagaagtctggaaaacatcaggatggaactggtgaaataagtgtggcctctgacggaatggagcggtccgtctgcactgctgcgggtgcccctcagatcctgtgggtcagtgagaaaagcagtgaggaacaaggcaggtactgtgtactgtcctctgcgtgcaaggaaggccagcgcatgcaacagagtccacacagacatagcctaactctggaaggaagaatgagaatgcagtttcagtggtggcctctggtggggagaaactgggtgaagggagatgtcatttccatttctctactattaattttgtattaccatgcttaaatgttactttttacctttttttttttttttgagacagggtctctctctgttgcccaggcaggagtgcagtggtacaatcatggttcactgcagcctgaacctcccaggctcaagcaatcctcccacctcagcctcctgagtagctgggactataggcacgcataccaccgtgcccagctattttttttaatcaagatggagtttttctatgttgcccaggctggtctcaagctcctggactcaagcaatcctcctgcctcagcctcccaaagggctgagattaaaacgtgagtcaccctgcccagccaattgctttttaaaaaagattaaatgcatgtatacgctcaggcatcagcacacttggaaaggatgaaaatatccggaagaagggttcttttaaaaggctcctcaagtgatgctggcaggcatgacgaatgtccctggtcacaaaagctctgatctggcctaaccctgtcatgttagagactggagtgcgtgtgtgtgcgcgcaaagtgtggggggatgggggtgagtgtgtgtggtgtgtaagcatgagtgtgtatgtgtgtggtgtgggggtgtgtgctgtgtgagcgtgtgtgagtctgtgtgtgtagtgtgtgtgtgaagtatgtggtgtgtatgtgtgacgtgaggtgtgtgtggtgtgtgagttgtgtatggtgtgtgcatgagcatgtgtgtgggcatgtgatgtgtgtgtggtgtgtaagcatgtgtgagtgtgtatgtttgagcatgtgtggtgtgttgtgatatgtgtgtggtgtgtgagcatgtgtgtgtgatgtgtctgtgtgtggtgtgtgtgagcatgtgtgttgtgtgtgtggtgcatgtgtgtggcgtgtgagcgtgtgtgtgcattgtgtctgtgagcatgtgtgagtgtgtgtgtgttcagcatatataaggcatgtaactgaacacagcactttagagggctctcctggagtcagagggggtgggtaggaggagaagggaggtgggctagtgtgctgaagtatctactccttgtcatagtctgtgacaacccagactagcccatgagccaccctgttccctgcatttccaatgagacctcggtggacatgttccctgaggtgaggctgactgatgtcatttgacgatcttgatgccaaatccttttatatcaaaaacaaccagaacactctcttttctcttagtgctttcacccagatgaccacatttcatcctcccagccactctgggccaggtggcactgctggtttgaaagggaggtctcccctggagtaacttccgtgggcggattcacaccctgcccacagtcctgtcccagtcagcccaccatggtggtctccggttcctccagaattcccgcttttcagctcatccccacattcccggagggactgagagcgcagccccagggccctgctctttgggggccgtctctacacccagagaagcagcaaggcattcctaggtttctctttcagatgcagaacttcagtgttcagagatgttcccactggtcctgagagggctcagttcagctttaatgactgcgctgttgcgtgtgctctgcagagggcgggtggcccagcgtggctgactgcagttttcctgacgtggagcccgagcctgccccgctgtttattaattaaggatcactctgcttgcagaaccctgaactccccagaactgtgaggtgggagaaccccgagaggccacctggccccacttcccacctgctgcccaaaccccctctctgccttcctgacagtcaccccaactcccagtgatccccatcaaccatctgacaaggggactgagagggaagagaaaggaggggcccaaagaggaaggtaaaactgtcgggaacagcccccaaatgtgtgacagccttcagtggagttgcccactttcccttttctcctccctgcaggacctcccttctccccagtcctccccaacttctgaggttacattgagaaaagtctgcagagaggtgccagcatcacaaggtgttaaggaccacgagtttggcattttaacagatgccagagccacttgagaaatgtggtaactaagcccagagaggtacagttaacctccccagagtcacacagcaggttcatggcaaagctggactagcacaggtgtccttcccctgcagatccccttctgtgccccacatcacctccctccagtgtctgggccacctggagatgggccctcagactcacccggccagaggtgccatctcatgggagaggtctggccaggaagcatcgatatttgagatcccaagaaatgaagacttggcctgtcagatgacagacttcggtcatgggaacacgtgatctgttttacacatgcgtcccctcagcagcagctttccagaacattcccactttcttctgtagtgagaagaactctttccctgcagcctcctgcccaactcctccttcagtgtctttgcttcagtgtctttgataaaccattctgctttgcagagtgcgagctctgccttgcagggttcgcatctgcctgtgctgagtaaccaacgctaaggtcgagtggtcggtcacctctcataagagctagggttgtctcatgctgatgactaggacttgccctcaaggagaaaaataaatcaaaacaaaagcaaaaacagcaaacatgcatctcttaaagaaggctctgagtccaggtaaatttccttccactgaagcagccaggctgaattcgaattatctttgcccctgcttaaaaactaatgcaaattttcctagagaatatccactaattcctggagggggcatgggcattcctgatgcccatgagaggaccatttgctcttccctcagtatgctaaataacagaagcgacatttgttgctggaaagtatcagtgaagttaataaggtttttcttgcccagggtgagggaacagttcccaatgacaaatgctgtatgggaaggggctgtagaactgccagcccctttggtccatccgtaaagtgaactctgtggatcctggaggattccagcgtctttttttttttttctttttttttaagacagagccttgctgtcacccaggctggagtgcagtggcacgatctcagttcactgcaacctccgcctcccgggttcaagcgattctcatgtctcggcctcccgagcagcaagactacaggtgcgcaccaccatgcccgactaatttttgtattattagtagagacgggggtttcactctgttggccaggctggtctcaaactcctgacctcaggtgatccacccgcctcagcctcccaaagtgctgggattacaggcatgagccaccatgcccagccagcatctttcatttttctgtctgctttggccctttcctctctcactgtcttccttttccatttccaaagtcagtccatctcactattagcacaaaaactgctagagcgcttgtcattggtcatctctccctgcacctggctggtctgttcttggccactgaagcgtttcccccagctgttgctttaatcattttattgttattatgccttacttaagaaatggatatgagatgcatttacctgtctcttcctgccactctgcagagccagtaagatgtggtggaaagggcccaggctttggaggagggctggctggggttggatcttggctgccccctactagctgtgtgaccttgggtaagtagctggacctctctgagcctggttcggaatcatagcacctctctttcagggctgctgtaaggaatagcagtggtgtgtataaagcagagcgcacagccagcaactggcccctagccacactgctgagcacctactgtgataagctgccattgtggtgtgtgaagcaaaggggaaacatgcctgctgtagtgagcttcctgtagggcaggttgtagaaccagaggtgggttccaaggttacaaagggactcttagtgtattagtctgttctcacattactataaagacctacctgagactggatcatttataaagaaaagaggtttaattggctcacattggctgggtgcggtggctcacgcctgtaatcccagcattttgggaggccaaggccggcggatcacttgaggtcaggaatttgagaccagcctggccaacatggtgaaaccctgtctcttctaaaataaaatacaaaaattagctggccatggtggtgtgcgcctggaatcccagctactcaggaggctgaggtggaagaattgcttgagcccgggaggtggaggttgcagtgagccaagatcgccccactgcactctagcctgggcagcagactgagactctgtctcaataaaaaaaaaaaaaaagaaaagaaaaagaattgcaagaaataaattattgtttatgagctatatggtctgtggtaccttgttgtgggactgggagtcttggcgtctccctgaccctgcctgttgctgcagcaccgctcagccctgcctgctccctacctgcctcccctcggcctctcctgcctccaccgggcccctggtgcctcctctagagacagtcctcctgggaccgattgtgttctcacttacacgaggcatccaggactacagataaccagaggaaggggcgccccccccgcctgccctcctccctggcatcctcacgctgcagaggtcagagcctcatcccagccccttacctgcccctactctgtggagaaccgtggtcagttcgccaggccggatccacgaacggccttgtggaagatggtgagctcacacccagagctggctccgatgaccctgtctcctttacatgtttctaccttcccctccctaccttcccccactgctgggcgcagagtggaggcagatgaggtttaaagctcagaagggcttaaacgggttggggcgcagtggctcatgcctgtaatcccggcactttgggaggccaaggcagaggatcacttgagcccaggagttcgagaccaacctgagcaacatagtgagaccgcgtctctacaaaaaataaaataaataaaattagctttgcagggtggcatgcacctgcagtccctgctactcagaaggctgaggtgggaggatcgcttgtgcccaggagtttgaggctgcagtgagctatgctggcaccacagcactccagcctgagtaacagaatgagatcctgtctcaaaacaaacaaacaaacaaacaaaagaaggcttaaagggggctccaggtgggcttggcagcacaaagctatgaagttctatcttagacacaagttctgttactgggcctttgcaggctggcctgggtacctggctgccatagacagggaaccttccagatgagctgcaggcgtggagcacaggagccagggtgctcttcctgggctctgtccacaggcagaacgtacacagtctttgtacacgtccggcggctctggtgcctatttttgtttgtgtttttcttttgtttggggggatggatttggtttcccccgagccctctgtcctcctgtcacctggctggtgctcggcaatgttgaccagctgcctggctggagttggcagtggctaaggctgtgacagctaacatgttcctgagtcctctcatttcttcaccataatgccctgttgagtttgcagatactgtctctgtttttatctcccggggaaactgaggctcagagtggctaggccaccttcccatggtccctcagctcatgagggccacacagggcattgcggtggccttctcctcagccttgaccctccggccccagcattgctgcctcaaggggtctcctctgctgagccgtgcaccttctgcctggcagctccaactctgtggctgtgttcagtggctcagcactgccccttgaccctccctggccttctgcggatgccagactggagcactctgacaaggtctggggtggttgtatgggtcctgtgacctctatacacctcccagtgcctgggaatcctgcagatacaccctccttagccgtccctaaccatagaggacatttctgaggtccccgagagagtggggcacccctgcaggatccaactgctgggcccaggaaggatagcagcagcatgaggggttccattagccacaaactcacggcatggaaccttcacccacctcgcccctcatctgctgtttagcacctggcacgccgtgtatacttactgattattacattttaatggcaaattatagtggcaaacgtatgcatctttgcacaattgttgtacagcatgatgaacaagtcattaatagtaaagaataaatgtgaaagtgagaaaaatctgactgccaaagtttttactccttccttccctccccagacttttaaatgaaagtttagggataatcccttagttgtcctgctagtaggacttgcaattaaaagaattgggccaagaacacttctacgcttctccttttaggtttgggtgtaaattcggggtatttctcactgatgaaagcctggtgcagggcagaccgtgggaagctttcatttccggaatggaccatcaacatcccttggagaagaattctcttctccagacccagacctggtgtcctggcacccattgggcaagtgggtcctagaagacaaacctggtcagagcctggaggctgcttagcattccccacgcacattagcagctcggagagctcaggaagccgcagcccctccttgcctcaccagcctggatcaggacagcatcccctggaagacacacagggcctggcctctgattacccagcctggagggaaagctcaatcgagcatcatgtcacccggtgcccccatgcagggtggcactggtgagacccccaagccaatgataccacctcacaggagtgcaggcccattgtggccagatcatcttgacttttcaagataaatcagaaatcgtatttccatgagatatccctatttgcaagtgatggtgactaaattagaagtttttgaatattgtaacatgttcgtaggctgtttgtctggtttaaactctatctggaggaattcaagctagacttcaggaataacttcttgaggcaaggattttgagaccttagggaaagaaggacgtcttgggggtattctgactgttgtcctcctggaagggaagaacagagaactagaagactgcccttagcgaagttcaaagcacctaagcccgggaccctcagcaagtgttcttgagtcacagattctccctgaggcgcctctttctggctccatagaatggctgattctgtaactcggtgagtttgctttttttttttcctccatcacccaggctggagtgcagtgaagctggagtgccgtggagcgatcactgcaacctctgtctcccaggttcaagcaattctccttcctcagcctcccaagtagctgggattacaagcatgcagcaccacacctggctaatttttgtgtttttaatagagacggcccgaagtgctaggattacaggcatgagccaccgcggccagccataactctgtgactcttgttacaaaggccttatattttgctctttgagggtggttttggtttgatgcctgttggttgccatcttttaactagggatgttttatcaaaatgcccagccaaagtgtccaaacaaattataccttaaagtttgaaaatgtctggcacttctaattcaatgcctgttgtgccaggcactgggctgctgaggaactgagtcccgtccctgcaggctagctagagaacacacacacacacacacacacacacacacacagagtggtcttacaagtcagttttatattctacctatatgcaataaaggtattattatgttgaggtgccttgatataaaaatttttcttaaaggagaggatgcctaaaacaggcattacctgaaacctcctctctccagcattggttgtcttctgtcatgactcagggttttcactgagaatgggatggaaatgtggtctaaagatagggccaatgttgggactggatcccctctgggaagtcagaccaggctagggcaggtccttgaagccatcaggaaaagcctctggagccagaaacaaaacaaaaaaaaaatggtgttaactaaactcagtctcaaatcctgaataggactcaagtcaagcaaaataattaaaggagttagcaaagggcaagtcagagagaccgagcaacaccaatgtcttccgggagccctgtggcgagtgacagagcctggactctggagtagaactcatcttgtgtcttcttctgccactcgttagctgggtgaccttgagccaagccccttaacctcttggaccctatgttcttatctctaagtaggggctggtaatatcttcccctttgaggaatgccctctaaggggtgttgtgaagattcggtaaggtggcaggggtaggactcctggccagaaacaggcacataataaatgctaagtctctccttctctccacctgctggatgctgtagatactaaggatttcgatgtgaatgagacaaaacccctgccttccaggagcctttgagaatcagagaactagacccatttccagaacaaggggatgcagggtctggataaagttttggggatcaatagagcagagggctcccagaggatcccatagggttgactcctaactcaagggcatgagacaacccccaggaagggcaccctggaaggggtccggctgtccctgatttacttgtgggcactgggggaatgcccggagccatccagccctcagggctctgtgtgattctgggttcctcccataaaagataatcagattctttcacgttaatgtctttctccacctcattgcacatcatgcagctattcattgactcagcaagtatcagctttgcatgcgaccttggcctacccactttagcttttagtaatagctcccttcttgaataatacaaccagtggggaaacagaacctaactcttacctctgggaggcttatttgctttgagaacatatgtcctgcagttttgttcatatggcagtgaagtttcgtgcacacactctagagccaggcagcctgggttcaaagcgcagctctgccaggtcctaactgcatgaatttgggcaagtcgctcaacctctccatgcctgagtttcctcatctgtaagattggagcaatggtaatacctgctttttagggttgagaagagaattaaatgaattaagatgggtaaagtgcttagagtggagctttgcaagtagtaagtgctatgtaagtgttcgatttaaaatgaaagacccttaaatacattctttgttcatttcacaagcccttcatttcacaaccttacatttcacaaccaagctctgtctcccctggaatccagccataactctgctcacaagtgtgagacaggccccagcagagctgcacgaagaggagagaaggcagccccccagactcccaaccccctgtccaagatggcaaaaccagaacacagcctctgtaccaccccagcaggtattcagaatctgcaatctccaaagcccacttcaattgtaaatgtagagccacgtgcgctttaagtcacctgtcactctggaggctcttttgctcagttcctcaccattagcagggatgacagggagtgcaggagtgcggtcgactcccagatattggagagcgctgggctagctgcccattctcccggcctccactcctctttgctgtccagccatcacttgctctttgaaggcaaacaaaacagaaaacagtgccaaaagtatgggaagaaagccagcttctcccctggggtgcctgtgatgccatgcccaccctccctgaccacgcagcccctgtggaccctcagggccccaagcccccatttccatcacatgcgtacacccatgtgtgtccatagccgcccatctcagtcaataaggctgctcctgcccacttggaatagtggtgacaaccaggagtggcttatgggaactatcccaatggcctgacagcatgtccgctgcaaaccgctgaggtaggacactgccctcatgtctagctgatcagcaagaggcgcagttgctttcttaggtaacattgctgctgtgtcctggccattgctggggggtggcacttaatctacaccagaattttccctcctgtatcttccaagctgcttggatcttggtgctgaattaggttggactttgtcttgtggggaagggaggactatagaccctcaacgtaagcaatggtcagactattctaagaaaactcgccgaattaaagcatgaggtaaatttagttctgacttctgtccaccccactgccactgtccccttttatcccatgatcccttgcttttcttttcctcctctctccctatctcttgtgtttgacgcatgataggaattcagaaatatatgtttgtggatttgtttattcacgtagcaaaccatttcttgagtgcctaccatgggccaggtagaatgggcggccccgggctgcagtggtttcttcagcccctctccagggtttacactgtgcaagacggtttgtgatgggtcctcccatcgaggaccacactcttctttctctgtgccccttggtcctcagtctctgaccccacttcaaaggcagcattcactcagggaagctcccatacaatgctagtcagagtaaaagtttggacaaattgccaggaagcagcttgtcagtatgcataaacagcctttaaaatattactactctttgacccagaatttcacttctaggaatctgtcctaaggaagtagtcacatgcaaaagatttatgtaccaagatgttcatcaaagtgttgttttataacaggaagtctcagaagctggataaatatccaacctctggaaatggttagatagaatagtatgtagccattagaaaattatgtctatggggtttaaaatgtcatgggaaaacacttctgacataaaagagcatgagaactgtatatttagcataatcttaactatgttttagaatgcacaggaaaaaaatgtacaaacatattcatagtgatgtctctggtggtaggattatgatcagtaagtacttctgtctcttcatattttcctgtatttgataatacatgcatatgttgtttttaaaataagaaaaattttaagtttaaaattggagctgaaaagtgtttttaggtcaggcgaggtggctcacacctgtaatagcaccactttgggaggctgaggcagtcagatcacttgagcccaggagttcgagaccagcctggccaacatggtgaaaccccatctctactaaaaataaaaaaattagccatgtgtggtggcacacatctgtaatcccagctacttgggaggctgaggcatgagaattgcttgaacccaggaggtggaggttgcagtgagccaagatcgtgccactgcactctagtctgggcaacagagtaagactctatgtcaaagaaaaaaaaaaaagaaaagcctttttaaacagtagcagacataactatataatccttactaagctgtcggtcaaatttttatttatatatttattttattcatttattatttttagacagggtctcactctgttgcccaggctggagtacagtggcgtgatcatggctctcttcaaacttgacctcccgggctcaagtgatcctcccatcttagcctcccaagtagatgggaccacaggtgcataccaccacacctggctaattttttttattttttatttttagagatggtgtttactatgttgcccaggctagtctcaaactcctgggctcaagctatcctcccacctcggcctcccgaagtgctggggttaccagcatgagccactgtacccagccctcaaatttttaaaaatctataagagacattattggacaattagagaaattcacatatggacttataatagtatcagagtgtgtggtgtgatggttctggagggaatggactttttctttggagacaggcttttctatgcccacccttttatcttgctaacttatcatcatccaggttccagcagaaacattacttcccccaggaaatttcttaagggtgcagtatcatgatgtctgcagcaaattctcaaatagctcaggaaaaaagtacgtgtgtggtatgagtgtgtgtatgtatgtgtgtatatatatacacatatatacacatatatatacatatatgtgtatatatatacatatatgtgtatatatatacacacacatacacatatatatacacacacacatacatacatgtatttttatataattatatatgcagagagtgcaaatgttgccaagttaaagattggtgagtctaggtgaagggaatatggtatttattgtattatttgtgcaacttttcttaagtttgaaaattttcaaaacaaaaaattggaggaagaaggcatgccagtctaccccaagccctccattggaatgctgaaaatctaaacaatgtgatttggcaatttcatttcttttctgttgtgggccagtagtccttagatgttggggaagggggtagtcgctgaggtgtggttgacttaggatggaagaagcagaagtcaagactcccagggtcaaagtggtttgctctgctgacccaagtgtgggaggcccagagtcagcgtttcaggtgtgctaattcagcatggttctattcacggccaaagtccaccctgggcacctctctggcagcaatcttgggtgactctactaaggccaggcctccatgaccctatgtctggatcccatatctccacctctcccactgtctcaggaacggtgcttagctttttcttttccctctcctgtcttctttgccagcatgtagaaagtttaaataattcccctctttacaacaaaacaaaacatacccccttcagtcaaccaccctagctctcttctccttttcccagccagatttttttaaaagcatcctaggccaggcgcggtgactcacgcctgtaattccagcactttgggaggccaaggtgggtggatcacaaggtcaggagatcgagaccatcctggctaacatggtgaaaccccatctctactaaaaatacaaaaaagtagccgggagtggtggcaggtgcctgtagtcccagctactcgggaggctgaggcaggagaatggcgtgaacctggtaggcggaggttgcagtgagccgagatggcgccactgcactccagcctgggtgacagagtgagactccgtctcaggaaaaaaaaaaaaaaaaaaaaaaaaagcatcctcagcactttggcaactccatctcctcccaacatgtccctgttactggaatccagccaggactcagccccgatctttctactctaaccagttgtctcagttaacaaggacaggtttatgctgcagtgacaaacaagatcccaaattcttgtggcttcacacatctggcaccacctcatcttccagccttaggagtcatcttttagttccttgaaaactctttacagttttctgttggggccttgtcatatactattcccctggaatgttctttcctatcccctccctttcaccttgctaacttgtgcccatccttcaggtctcagcagaaacatcacttccttggggaagttttctccaacacccacactacacaggtgtcccatctacactcctatgactttgtggtacttgtctcacttcattttccactgccttccccacaaggcacctgcacaagggcaaggaccgtaccactgtacctatgtcactcattgctgtggtcacctgcactctggctgcctaccttaactacacattagaatcacctgaggagcttttaaagccacaatgcaagactccaccctaggccaattggatccaaatccctggggtagggccagacatcagtggagttatatatacatatatatattttgtttgtttgtttgtttgttttttgagacagagttttgctctgtcacccaggctggagtgcagtggcgcgatcttggctcactgcaagctccgcctctcgggttcacaccattctcctgcctcagcctcctgagtggctggaactacaagtgctcgccaccacgcccagctaatttttttgtgtttttagtagagatggggtttcaccgtgttagccaggatggtctcgatctcctgacctcatgatctgcctgcctcatcagcctcccagagtgctgggattacaggcatgagccactgcacccggccatcagtggatatatttttaaagcactgcagagaattctgttgcatcagcttgagaaccactgatctgccttgtgcttcacatttaaaacttttttttaatgaataaataaaccccaaaaaattaatctccctaagcctccctagaagataggatggtaaggatattttcctaggtaaaaatatgttaatttcatatttcatgaaatttcatgtttcatttcaatcaagctctgtcatacaccttacatggggcaagcccagtgcctgggcagggtgtaattatactcattacacaggcaaggaaaagtcacattaggtgatggagcacaaataggcagttaatggtttcagggctagttaggatatgtttgtctttcaattgcaagtaatagaagcccaaagaaattggttatttatataatataattgattggttcccaaatttgaaaaattcaggaatagacccagcttaggtacagctggatccagtcactcaaacaatgtcacaaagaaccctttgacaggaatgtatcctgtgttgactctactttgctctgagtagtctttccccaggtgatgataaaaatggtcatcatcgccaggcttgtgtcctgtttagtaggaatatacaagaagagctcagtaaatgctggccccaccactaagcaaaaacaaaacttttgttgttgttattgttgttttaaataacagcttagacctttcttctttccttgttattctctttcatctgtaatccagttttctacttctgaagtatagaatgttctgatgatttattcttcattacccacaacttgcacatgtttatttaaaaatgccaggattgcctggccgttgtgtgctgttaacctttgtttgctgttagtggatccctgaagttcaggctcccaggggagcagataatgggtatccagttcctgcaatatccaccctctggcaagccaagttccttcctgggtaaggttttgcctacctgcattcctagggaagtttctgggcctgaccaccaagccagctctgagaaggggtgcataagccccaccatgctttggctctgtccctatagaatattttatgttgttactgaaaactaaaggaagatgggtgcggtggctcatgcctgtaatcccagcactttgggaggccaagacagattgatcactcgatgccaggagttcaagaccagcctggccaacatggtgaaaccttgtctctacaaaaacaaaacaaaacaaaaattagccgggtatggtggcatgcacctgtggtaccagctactcaagaggctgaggcacaagaatctcttgaacctgggaggtagaggttgcagtgagccgagatcgcactactgcattccagcctgggtgacagagcaagattctgtctccaaaaaaaaaaaaaaaaagaaaaggaaagctaaaggagagagactaaaatgatatcaggttcctggagaacaaacagacatgattttgcttcatggcaggacagccggaagaagtgggattatatcctcacattacaaataagaaaactgagactcagaatggttaagtcacttgtcccaggccacacagccagtaaattacagaaacagaatttgaacccaaatcttccagctccaaagcttgtgttcttttcactacctcctgcttaattttttaatttctaagattagacccttcatctatccatgacacctgcctgtcatcccctgaaaaaaggtgaacgccgttcagaaatttttctagcctgagctcactcccagttcacttatttttgctttgtcatggctgcccagtccccacttgtagaccaggaataggtcatggctgcggggactacacgctgtcgctgctgcaagggccggcctctgtttccggggctgagtgggggccagacctgccaggagcaccatcttctgtgggtcctgcctggatgtcacatcccggccccaagaagtcactgcaaaccttcgtattattgagcttcacatcctagaatttgctgtcactgtggctgctgcatgaagttgtcctgagagaaacgggcattgtcattaacagggaaattgatggtctgggggaaaagtcatcctcattctcttgcagatctatgggtgattgagactggctgatgttgaaggggtttctcagccatcgtgtgccatgttatggaacagtggtgtagccagccatttgacacccagcgctgacctttgtttaacaacctcacctatatatgacaaaatgattgtcagaaataatcgtgtaatgaaatgactgtaataatggccagaaaagaaacgcagatagtaaaatgtttctcttgttgaactctgtacatataattgcaccaggatttttttcaaataaaaagtaaatattatactacaaaaaagggaaaaagcacaagcatttattaaatagctttctatatctttctgagttttgatcctttgattgcagactgatgtaatattttatgtaaatcattgcttggttactaagtgaactttaagaaaagtgagacgtctgcagaagttgcccataatttagcagctactgtattgtaccattgatgtacggctttattttcttgattaattatttaaacaatataattcacaattttaaaataataaatttccacttaaaatggtatttaaactcagcaaaatatatcatctatgagtaaaatttgtatttaccaagcaaaaatattacagtttgtggttcacatgctgtctcactgttttaaattttaaatacaaaaactccaagtaggctgggtgtggtggctcacacctgtaatcccagtactttgggaggctgaggcaggcatatcgcttgagttcaggagttcaagatttgcctgggcaacatagtgagatcctgtctctactgaaaacaattagctgggtgtggtggcacatgcctgcggtcccagctactcaggaggctgagataggaggatcacttgaaccctgggggacagaggttgcagtgaggcaagattgcaccactgcactccagcctgggtgacagattgagaccctgtctcaaaaaaagaaaaaaaaaaaagaaacacaaaaactccaggtggtcgcacagaatgacaggactgaagtaacttagctccaatttctgtcttcataatcactgtcctaccattgtctgtgcttagaatctacttgcttaatgcaggaacatgtgttctcacagagatggaaaatgcaaatggcgccagaagcaagctggaaattctgaaccattaagaatttactctctgccaggcacggtggctcacgcctgtaatcccaggactttgggaggctgaggcaggcagatcatctgaggtcaggagttcaagaccagcctggccaacatggtgaaacttcatctctacaaaaatacaaaaattagccaggcatgatggtgggtgcctgtaatcccagctactcgggaggctgaggcaggagaatcgcttgcacctgagaggtggaggttgcagtgagccgagatctatctgcaccattgcacttcagcctgggagacagagtaagactccatctcaaaaaaaaaaaaaaaaaaaaagaacttactctcaaaataaatacgtgtggctgactccacatatggtagggccaactgtataactagaagttctccaaataacttctgtggagaaaaaaaagtttattaaaggttaacttttttaaagtgctaactagaaccttactaacactgagatcgcaccaattgtttataacttagacagggccgggtgcagtggctcatgcctataatcccaacactttgggaggccgaggcaggtggatcacttgatgtcaggagttcgagaccagcctaaccaacatgatgaaaccccatctctactaaaaatacaaaaattagccaggcacggtggtacacgcctgtaatcccagctactggggagggtgaggcaggagaatctcttgaacccaggaggcggagattgcagtgggccaagatcgcaccattgcactctagccccagcaacaagagtgaaactctgtttcaaacaaacaaacaaaaaaaaaaacctcttggaccaggaaaatattttttaagggaggagtattttatcactggcattgtttaggattgcaggcacatgatgctaatgaaaagcagactaactattagttggttttattactgtttttgaactctctctctcccttttttttttttttgagacagagtctctctctctgtcacccaggctggaatgcagtgactgcagtctcagctcactacatcctctgcctcctcagttcaagtgattctcgtgcctcagcctcccgagtagctgggattacagggcaccacaccaggctaagtttttgtatttttagtagaggcagggtttcaccatgttgcccaggctggtctcaaactcctggcctcaagcgatctgcccatcttgacctcccaaagtgttgggattacaggcgtgagccaccgtgcctagccctgtttttgaactctctagagacagtccagccccttattacttgtcctgaggcagctgctcccttcacctggccccccgcattgtgttccggacccttgtcctggtggtgctaaagaatatctctgtcgatcctttggggactggggaaactgaggcccagtgccacgcgatgccatttgttcagggaagattaggtcatctgctaggtccccagtcacttgaccttcttcccagacaggaagaagctgctctgggtctctcagtgctccacgtgtctttgcacattgaaatgttttctgattttttttttttttttttgctgttacatttacttttaaaaaataacaagcaataaaatgttacatttgagaaggttgaaatgagaattgatttgagttaaattctagcagatttttcttagaagaatgatatcatcatctccagctacctgcaattgatctactctgaattaagaaagagacttccatttgttgtttatattttgcactcttgatgtgtttctttaaattatggtcatgggccaggtgtaggagctcacacctgtaatcccagcaccttgggactctgaggagggaggatcactggaggccaggagttcaagacctcgtctgtacagtaaattttaaaaattagccaggcatggtagcattcacctgtagtcttagctacttgggaggctgagatgggaggattgcttgagccagaactttgaggctacagtgagttattttcacgccactgccctctagcctggctgacagagcaagacctgcctcaaaaaaataagtaaaaaataaattaaatttcaatcattagcagtcattaggatatttaaatacagtatgttgaatcaaagttacgcatgtgtgtatttttttttccagagagttgtttatcatgtgggttttaatttaactttaaaaaaatgttggctggacagttgcccaaatggtatcatcagccatttggttgagaacgtatgtcctgcgggctcctctgtcactggagttttgctagctgacagccactggctagttagagactgcagtcagcacagatgcaggcgtggacttgcgcacgtaaccatgtcaatgcaaagccatcacttcttaaaaattctgaaccctgctgtctgagatggtggtgcagcggatagaactctgctctaagaggcagtagctaattccatgtcttctttgcccttgactagctgagtgactttgcacatggggcttgcctctctgttgccttgtctgcaaagtggaatcatcttttccttgctagacagaaggtggaccctggacctatggcctttttgagtttcccccccgcttcttagaaggacctctgatcctactgagtttaatacccacgggttaataattgggaaaagcaaaggaagcgcttctgtttaggtaattatatgcatgtttttgtctttttctggctggaaagatatccaagccactgggaaggtccgtggctacccagggtagccctctctggggagggctgctatatccaagagcccctcatgagaatttgaaaatcgaccatggtagggcctgctgacttttgacagctaatggtgtgctgagaattgtccctccaaagatgcctttccattccctcgggagagtctgggcagcccctactgggggctgggatgctggctcttccctcagcctccaccccaactgctctcttccctcctcccctccccagccccctaatttctctcacaaggctttgttctgcagcaacctttcctaatgcagtcctggcctcttcgcagcttcattacataaccttccgtggactcctggtccaaggatcaccccagaaagccagtcagaggtaggcacgcagctggggtccatttacttaccttccccaccccctcggaactcagaggtggtgcaggaatttggactccaagaattaacagctccaccaccatcaccagagccaaaactcaggatgcatgtgcttcatctgctgcttatttccagctgagagccagtggtgccatggttccttagggagccggtcccctgatgccggctcctggccccaaatctctctgatccgggctcttccagaatgtcttgtctccaccatcgcctttgaccaatggtgtccctttgcctggtaatgtcccctttgcctgatgatggccctgtcactcctctctttagcacagaggaggctgtttcatcccttcaagcctgccctcccttcaagtcttagctcaagttcaccttctccgcagagccttctccaatcttcttgactacgtctcctctcagctccagcaacctctgtctctggcactgattccttacttagctaagagaatcacagacacttggggctcaggacaatctgctttctctcttcttacccatggccttggactgtgtgtacctctttgtctccactcccaaacccaacccccagagggcagagagcatgttgtctgtccctttgctcagcatgaagccatgcgtgtggtagatcggcagagttccataacttgtgttgaccgaggggtcactttgctctgaaattacccctgtgtccttcagtatttgcacagatagcttcctggccagaccgaatatatccaagggcatggcccacctctgctcctgtttccaggtccctggtgggggttagttcatgccttcctcataatctgcccactggcctggtcctcaaggtcttcccaactgctcagccagagttgagaaaatgggtcgctccatcctgtttgtgtcgttctctccttcctggcccactctcctgcccacaggtatccaggggctgcctgtagcattagaggacatacatgcacatgcgtgggcatgggacactcacgtagcctccaagcacagcatcaataatgcattctgtgctttatagcatggaaagctgctctaaactttattacacagtggacatgtctgaagcagctcccaaatccacccctgagtgtgttggaattggcaagcctatcacttgggagtctagtttttttgttcgttaataatagatgcttcctgtggccccagcttggcaattttgatttaaagtgatcttaactgaagagactaatggacgggtctgaatttgtgccttttaagcacaaagtattgctcttaattaactggattctatcctttgagcaggcagaggccttcccccaagggcgtcattaacgatccacatctggacatcttccaaagccttcttctgtttcaggccaaccgcaggtgtgttcctgaacacccaggaggctatgagagccacatatgcctcccaaatacacacagtgtgcatgcccagggacatagagcagtgtgcaaagtcccattccatctctctccacctgggagaggatggctcttctgtctgattcatggctcaaagtggtaaaggagctccccactccccgtcccacgcctactcagagtctgcaaatatgtatgcgatatgagagctcgtcagttagctgtcttcagtgtggcgcacatttgaggagtctgactcccctccagcacaggccaatgtgcactgctctcctatctttgtacccccactgttgcactgtgcagaggttggagccatagaagtaccagagctgtgaaaggagaggccccctctcacctctgccctggtctccatccccactttctctaggaagctagtaggtgctgacaggggagagaagggaggggaggggtccagaaacagtggctcatgcctgcaatcctagcactttgggaggctgaggcaggaggatcatttgaggtcaggagtttgagaccagcctgggcaatgtagcaagaccctatctctacaaaaagaaaaaatgtaattagctgggtgtggtggtgggcacctgtagtcctagctacttgggaggatgaggtgggaggattgcttgagcccaagagtttgaggttacagtaagctgtgattgcaccactgcactccagcctgggcaacagagctgagaccctatctcaaaaaaagaaaaaaaaaaagaaaggagagagagagaaagaaaagaaaagaaaaaaaaaaaagaagggaagggaaagcccagaagagtgtggggagaggaggcggccgtcattctggggccctcagtgtgcacaaccagataacacatgctctgtgggcttttgtaccattttgcttgagcataaagaaaggaaggctgcccctaaatagaaagcactctggaggcaaacaaatctgactccaatcctggccctgccactttcccagctgaggacttagacaagcaccctagcctcttggacattctcagagccatctgctgcaagtgggtgctgccatacccaccttactgggcaggcttgggggaccaagggtggtaaatggctcagtctttcatgatgcggccacacagcaggtgcgccatccaggtccatttctttccttcctttcccccaaatcaagttgtcattaaagtactagtccacattaatgaaatcaactgtattaattttctatttgctgctataataaatcatcagaaatttagtggcttaaaccaacacaaatgtattaccttacagttctggaggccagaagccctccataggtgtcactgggctgaaatcaaggttttggcaaggttgcggtcctttctggagggtccaggggagaatccattttcttcctttttccagcttctaaaggtttcatgcattccttggctcatgatcttctatagctatagtcagaaaaattttccatcaatcatcttcaaagccagcaatggcaggatgagtcctcacatcaccttgctctgacaccagttctctgcctccctcttccacatgtcaggaccctcatgattactttgggctcactctgataatctgggatgatctctctattttagagtcagctgactgggaaccttaattccatctacaaccccaattcctctttgccatgtacagtgacatattcacaggttctggggattaggacgagcctgtctctgaaaggctactttacatgaaaattcatttttttaattaagatttttttttcctcttgagacaaggtctcactctatggttcaggctggagtgcagtggtatgatcacagctcactgcagcctcgacgtctctgggctcaggtgatcctcccacctcagcttccctagtagctggaactacaggggtgagcccccatgcccagctaatttttttttttttttttttttgagacagagtctcactcagtcacccaggctggtgtgcagtggtgcaatctcagctcacagcaacctccgcctcctgggttcaagtgattcttgtgcctcagcctcccaaggagctgggactacaggtgtgcaccaccacgcccgactaatttttgtatttttagtaaagatggggtttcaccatgttggccaggctggtctcaaactcctgatctcaagtgatccaccaacctcagcctctcaaagtgctgggattacaggtgtaagccaacatgcccggccccagctaatttttaaatattttttttgtagagatggggttttaccattttgtctaggctggtcttgaactcctgggctcaagcaaacctcccaccttggtctcccaaagtgctgggattacagcatgagccactgcactcggccttaagagaagatttaataattaatactttacaacaagatctggaagaggtgggatgagtaactaaatgaggatacaagtaacccgggtcatatttgctaatacccttggtcacattgaacttgatatcttatcagattttcctaatcagctcctttagcagcagtgttgcagcatcttatctcattttgttttttgtttttttgcctagcacatgcctgtaaatcactggattgaggtgtttagatgtttgttgtcctttggatgcttcttataaatccatatttcatggctccctggaaagtgctatgcaaatgataagctgcaaggatggaaaggaaattgcagtgctcctgaattgtaaatgggcttttacgaggaggtttctaattactcgctctttctcttgaactgaggagttgaagtgtaggtggcagatccataacagataatcatgtgtgtgatgtgacttcagcctgagcgtcgaggaccaagtcacagagcaggaacagccactctccagtgtccttggggctacgtctgaggagaacctgggatttcatatatgacctgcactggctggggggctctcttgacgtaacgtgttccctctgagcatgttacagattctgacattcttatgttccttctgtggagagacatgtacttagtgacctaactcactttagcatatttttgctcatcgtttgtgtagcttaaaggaatcagataattaccccctccccactactttcggaagcacaaatgcaatgccctagaattgtactggggactcaaaaagaaaagagagtagtaaaatctattaaaggggacaaagacagcctatatactacaagctttctatttttatggcagagaatgccattttctaagtaaacagagaactgcatttgacctgcaatatcaaatgcatggatttgatgctttggaaagcaactgttttctgcgttaatctgggtgtcttccgtgaaatgtcctcctgcctttggcttaaacactagctttgtctacagccattccatcctgaacctgcccaatcttgtctgaatcctggtttcaccactgacaagctgtgtgtccttgggcaagttacttcacctgtctgtgcttcagagtcctcatctgtgagttggggaatctggacagaatctaccccatagggcgtagtgaggatgtgttgaattatcccaagtggctacacagagtaagcactcaaatgatgtcatcgttgtcatgattgctgttaccagagcctagagttcattctgatactcgagtctgtggcccatccagcccaggtaaggaatagttggaggagttgggcatgttcagcttgaagaggagacgacaggggatatgggatagttgaatctgtgaagggccccctgggatgaagaactggcatgttctgtgtggctccagggcactgagcaggacccatttgccaaagtctcagggacacagtttctagctatagacagaaaaattttctgtcactcagaggatgaaaatagaatgagcccccttaagaggtaatgagctccctgtcattggaaggattccagaagagctaggtaaccactttaggtgctatcaaggggcttttttctttaaagtcctttccaaaagcttctgagattgcataaacaataggaagccatcttggtgctttaacacaaactctccccagtgatgagggttgagccaaagccagattggcaagcagagaggagacttgtgtacaaggagttcctcgagtcaattgctttttccttgttctagccagccagagggctcctgttggaaaacaggagaccggagaggctgaggcctgaccaaaccagcttctgcaggccagctgggaggccacaactcctacctacgggaaaactgaagggcatctctatttttagattagcaaaagaaaataaatttaagtttgagtctcctttgcaacttttaaaagacatctttattgagatgatcattcacattctataaaattcccccactttgagttacaattcagtggttttagtcttccttgatgattttgatggtcttttcttaaggctcttggaagacccagaagcctctcagacacaggtgggtgtggagggcgtagcacagaggcagacttctcatttcctgggtctcccctttaatgactctcagagacccctccttccccctgcccctggcttctaccccaggggtgtagagttttgccattttccaagcagaacttcatttcctcttctgtgtctacactctttgtgcttctttcttgccagctttttctcctttgcccgcccttccttccttccttccctccctccccccttccctccttccctctttccctccttccccccttccacccttccccccttccccccttccctccttccttccttccttcctgcctgccttccttccttccttccttccttccttccttccttccttccttccttcctggtatgtgactaatttctgtttcaggacataaatgttgtccaggctgttctttggtctttctgttggataatggacatttggcattgagagaggctgctttttctgaaatcatgttcttggggcccagaacctaggtgtgtgcttctgactttgttttcttcctgatccaaattctgatatgtccatttaaattgatctagacccacagggcactgtgggacagatcctcagtggaacatgactctgtaacgagagcattttgttttgtcaaaatgagaacatattattgcctttcatctgattgtaaacataatacatgtttataaaacagtataatgagacaaaaatgtagacactaataagggaaaatctccctaattgtatttctcttcacagagaaagcccctgttgggcatatatactctagtttgtttatttgtttgactacacatatatgtattcttttcttatgtataaaaattctgaacatgcacatttctgcaactactgttttcacttgatgatgcatggacctctctagagtgtacgtttcttcttccttacaaagcagttggcttcgcccagggtgcaccaggacacggttttggctctgtccccagggtgtcacgggaccaggggatgatctcacagggtctgccatctgccctgcctggccggaggctgcatcgagagggccaaggggcaccacgtgtcgtgggtactgtcaaacaagagccttcagagccttccacagtctttcttttgcttcccagcattgcttccccgctggtggactctgaatctagaactagctccaggcgcctctccaaattcagacgggagctggggcactattataatgcaaatctaggcaaagccctcccaataccaggatccagaatggggtggggccctttgccctgaaaagctgtttagtttgaaaatacaaacaggagacagaaaagtttggctaaattaatggataaagttttaacgatggtaaccatagtagggttcatcgacagccagcgatggttctgaacacttgacatgtattaactcacctaatccccacattttacagacaatgcaaaggaggctctgggaggttgagtgacttgccccaaagtcgcacagctcctaagtgaaggattcggagtggactccaggcagcctggtctgactccctgcactgcgctgtgcttatctctggccccaatgccgccatgcagaagtgtctgggggcactttgtctctgtcagacagaattcggagatgtgtatgcttgccctggtatggcacttctctttttttgagacagaatctcactctgtcaccctggctggagtgcagtggcatgatctcagctcactgcaacctccgcctcccaggttcaagcaattcttgtgcctcagcctcccaagtagctgggattatagatgtgcaccatcgtgcctagctaaatttttgtacttttagtaaagatgttgttttgctgtgttggccaagctgatctcgaacttttggcctcaagtgatctgcctacctcagcctcccaaagtgctgggattacaggcatgagccaccatgcctggcagtgtggcacttcttacgtgtgttcagcggacactgtttatcttctgtccctccaagacggtgctgagctcaggtcgttcattactggcagacaactgctgatttccaacagaattgccatcctcttctcccctgcgactttcagagtgtgacctcagactcaaaaattagaagtgaaaacatcttaaaaactatcaccttttcttcctaatcctcctctcccctccctgtcttccttgttgtccccatctaatgaactatcatggcaaaaagagcccatttctggtcattttctgtggcctttcaaactcccacctaccccactgctcctgggtgcattacccgaaagctgagacttcagtgcagaaagtgccaggccctctgtccccccagatcgccttccttgtcttccctgtgcttgcctgtcacattgtgtgggttccagcgctggaaggaatgaggaacagattctctggttctccttttgaagtttaccttcgctccaccacttctgagaccttcccggaagttgccccttgtttctctcctctccagggctgccccagagctgcctctcacctcttcctgctgtcaccccaccaccatcagggcagaagttgggacaaagcctctcctactggctcctgcttttctcccttaggtccagcctcctcttctccatcttcaggagtctccttctccactcacacgtcatgacttcagcacctcgcatcagtccagaatatgactgcttgttcaagtgccacctttctcatgcatttttttctagtgacaatcacagccaccctgtggggcaggagtgtcatcatccccatgtttcaaatgaagaattgcagttcagagagggcaagtgactggcccagcctcaacagctagccagtggaccccaccagggcttctgactccagtccgggttccctttccacccaaatccatggagggagctgagccgagaacaggtgtccttcaggaagacgtgaagccaaagcctccacctccaaactcaggggcccagggagtccaggcacccatccactcacaaggctggatatggtgcattccaggagaggggttgggggcgagtggcctctctgtgtacccgtggggatagatgcgcaagtggcatcgccacatcgtgagtcctggcttcatgggtgagctccaggtccaacgagaagccaagcagggggcccttcaagctcagctttgggcccgggtcggggtacagggtagagcgggcctccccagcccctgccatgaggccaaggcagtgcatcgttcgcagcgtacattcagaaaccaaagcctaggagctggttatcattccggtttacagctgatggaagagcaggtgcttccgagaacccacagtgctctttggccagtgacccaagggtgcctctgagaggcctcgcagcacccggaggtgctgctgaggcaacgccctgactgtaagaaggaccattcatcctcagagagtggccgtgatgctgctgcgacagtcccaccatccctcccgactctcactcccaacagacttcccactgtaaagctgaactctccagcaaatcacctctcgccagactctctcctcactctctctgggtccactagaggttcctcagcctctctttgccttggttttcccagctgtaaaatggagcaaagagggcctatgtacccacaaaggtgtggttggagcgactcctcctacattagggcctcgagtggggcttcatgattggttggtggaggtctccaaacccacccagtgccaccgaaggctgagactgcagatgcaatgccacaggtgtccttcctcagcctgggcagctgaacatcatgtgtaaaacggggataataagataataacagccccttgcacctatgtggctgtgaggattaaacaagataaatgtgtaacagtgcctggctatagaaatatttactcttgttattaagggaagaatatgtgtggctaaaaagggatcgaagatgtaaaagccaatccctccccctctagcatatttaagggtaatgttgagttggtttgtggaccatttgctgcctgttagagctggaaggtagggaccccctctcaacagcgatgctacaaattatacccattggaggtcaaccaaaagacaaagcttattggctggacatggtggctcacacctgtaatcctagcactttgggaggccaaggcaggcggatcacttgagatcaggagttcgagaccagcctggccaacatggtgaaaccccatccctactaaaaatacaaaaattagctgggcgtggtggtgcacacctgtaatcccagctactcaggaggctgaggcaggagaatcactagaacccaggaggtgaaggttgcagtgagccgagatcgcaccactgtactcaaaccgaggcaacagagggagacgcaatctcaaaaaaaaagaaaaaaagacaaagcttgttaataccagcatattgttaagggaataaagtaggctgcagaacaactggtgtaatatggtgccatgtagggaaaattacatgtgtgcataggagaggggtctgcaaggttgtgccctaagatgttagagtggttcctttgcttttctcttttataattttgtatttgacttttaaataaggaccataaatcacttttataaaatacattctctccagcccctactactcctttaaagaataagagtggtttgcccaagaaagacagttttttttgctctggttttcttgattctgacatcagaggaaactgcttctcatccacttggggctctgggttcaggggattcatttcaggcagattaaagtggtgaccaggggcattcgtggacacagggagggacaggagcaccatcagtttgtctcacacaaccactgtcatcctcactgaaggctgttgcctgatcaaaaacagtattgggccaggcacggtggctcacacctgtaataccaccactttgggaggctgaggtgagtggatcacttgaggtcaggagttcgagatcaacctggccaacatggtgaaaccttgtctctactaaaagttcaaaaattagccaggcgtggtgggtgcctgtagtcccagctacttgggaggctgaggcaggagaattgcttgaacccgagaggtagaggttgcagtgagccgagatggcaccaccacactccagcctgggcgaccgagggggactctgtcttaaaaaaaaaaaaaaaaaatatatatatatatatatatatatgtcaaaaatggggtagtttttagatctatagtagttctaaaaacaaaggccatccaagcatgacagatttacaagcactattggctattccagtagttacaatggaggagagaagcttttagttaaaacaaacaaacaacacaacaaacccagaaaccttaggtcaaaaccaaaattgtcctctcagacacaatctgggaattttctcatgacagtgggcattagccaactgacatcagcagcaaccatccgtgtgcacacagtggcaccacctcctcccaaaaagcagccttcatctatgccctcatacaatcgttgattattctctttggattgaggcccggaattatttaagtttcttcttgccagcatgagtctttcctttctgtatgctccttatcttctctctttaatttggcagttctgcttgaaatctgggtctttcattagtagtagttcaatttggttccagaacattctgtggtgtgatgcaatgtgaccagagctcacacttcagagctcttcaagggccagtcttactgagcacctcccagtggctgcctgtgtgctgggcgccacttgtggtgggcaggagagaggaggggacacaaaaggagacacagctccttcttagaagctcaaagttggggaccagctgccacagaagagtatgtttagcatctgagacaccaagatccagcgtcacaagggtgtttattaagcctcctcatctctttctttttctttttttttttttttttcctcaggcagtcttactctgtcacccaggctggagtgcagtggcatgatctcggctcactgcatgcaaccaccacctcccgggtttaagcaattctcctgcctcagcctccccagtagctgggattacaggtgcccaccaccacacccagctaatttttgtgtttttagtagagacagggtttcaccatgttggtcaggctggtctcgaactcctgacctcagatgattcacccacctcggcctcccagtgtgctgggattacaggtgtgagccaccgcgcctggccttgctgttgattcatctatagtatgtttgacttgatgacctccagttaccttagacagaggttctcatctaagctccaactttccatttcctttgtcctcgtctttccccttaacccctccacatttctctcaaaatcaccccacttctaaaaaatactgtttatttttcttttaaatttcaaattatctatactcattgaaataaatcaaaatagcatggaataagcgaaaaaaatggatcccacccttccccactcccattccctagggctaaccatagttaaccatttaatgactaggtttttttgttgttgttattttttatttatttattttgagacagagtcttactctgtcacccaggctggagtgcagtggtgtgatctcggctcactgcaacctctgcctcccaggttcaagcattctcctgcctctgcctcctgagtagctgggattacaggtgcctgccaccacacctggctaatttttgtacttttggtagagacagggtttctcaatgttagccaggctggtctcgaactcctggcctcaagtgatctgcccaccttggccttccaaaatactgggattaaggtatgagccaccgcacccagccctcctgggctcttttcctttagttgcactcgctccccgctcctggagtagagggatttccgagagactgtgggctccagccttcacctaggcccaggactaggatgcctgccctaacatttatctttataccttaaagcaaaacagctggaccataagcattcaagaacaaactgtgaataaggagaaagttctcccaggaaacaagagctttagttctgttgggccagcccttatattccttagctgttaccagtcactgcttgatttaatctcggctatcacttggcctgacaggtctgctgctggtgccaggatgtctgggttttgaagcctggctccattacatacttcctgtgtgaccttgggcaacttactcaacctgtctgttcctcagtttccccagctgtattatgtcagcataatagtttgttgtgtgaattaaatgaggtaataactggaaatgcttcaaacatggttcctatcatgagaaatcctgctttccgcctaaatgtgctggaaaattcctggtggtgcagaacaggagaccagagcaaaggaaagacagggtgcagaagccaaaaattaccttggagaacaaagcgcatgttaaggttatttttggattctaggtttatctctgcttggtcttcagttacctgcaagagatccatttaggggatttttgtttgtttttaacgatagctttattgagatataattcatatgccataaaagtcactcttttaaaatgtttccggtatattcacaaggctgtgcagccttccctgtccttgattccagtctgagtttttaactgaagggataaggaggaccacgctttccccagaccagaaccgcgggccagggggcgattctgctgagtcaccgcgggcgcctggtgcgcggcggcggagcccgggaccttccttggctgccccctagcgagggccgcagcgcagcctgagacacccgccggggccgctccacggccgtcggatttagactggaagctcggtccaggtccccagcttgatgcgcccgcggtgtaggagaccagcccgactcgggcttcccctgagcccctggactcttgactccagcagggcctgggtaatgaacgtcagctcccctttcccaaaggggttgctctgttgggaaggcacccgtttgatacagtagcatagagatgggttttagcatcaaaatatcagaattcaagccttgctctctgcttactagctgtgtgaccctaaaaaggtttctgaacgtctctgagcttcagtttcctcatcattccttctcacggggtggttgtgagcattacagagatcctctctgtgaagcccctgtgagtggctcatcctgagggctgaaataaacatgttattaataatccaaaactggcaagggatgttgactggtccccctcccttgcccaaggagctttctagaacctgagttatcattaccaaactgtactgccttgagtaagaaagttagaaggaatgggaaggatggtggcaggtggaggaaggcggattggtcatcacctccttgcagcaagaaacagccccagatcgtgggaaacctacagacctgctagacagactaggagcaaaagctggggctttaagaatccccagggaggttctcctgagagagtagccagttggattttgtaagcagagatttgtttggggaggaggtgacaacgtagggagcagaggggcaaagctgtcgggaatcctgccttgagggcagggatgtgtgttggggggagttgggtcactggggctcggtggccttgggcaagtttctacctctcaggtcctttacccacctagggtcgccatcctgcccacctcacaggttacagtgagcctggatgcactgtcatgggcaggtgcccaggaaaatggcagacatgttccaaacagcacgcagcattccccagtgatgcccagggtcaccttggaggtgggcgagatgcctggggtttctcgtccaccccacaacacctcaggggacagccaaagctgtcccttcaggtaagctgcacagaagatgtgaactctgctgcaaagactctattctttgggagcaaaagggacccagggtctcacctgcacatccctgtccctgagggcctaggggttcttggaggccccagccttggcaaaatgaggaagaaggtgaaggttgtctgggcccctgccaggctccttcctcggccacgcactccccttcctgcacacacacccttctccctccaccccatctccattgttgtcagaaaagtcacaataaaaaggtccatattgtctagttcccatacttttaatttttaaaattttatttatttatttatttatgtattttttgagacagagtcttaacccaggctggagttcagtggcatgatctaggctcactgcaacctctccctcctgggttcaagtgattctcatgcctcagcctcccgagtagctgagattacagatatgtgccactatgcccagctaatttttgtatttttagtagagacggggtttcaccatgttggccaggctggtctcgaactcctggcctcaagtgatctgcctgcctgagcctccggaagtgctgggatttcaggtgtgagccaccgcactcggctccacacttttcacttattaaaagactgtggtgtccatcaatggatgaatgaataaaccaatgtggactatccctcccattacccaaggaatgaagcacggagccgtgccaagatctggattcacagtgaaagaagccagtcaccaaaagccacgtgctgtgtgacttcccttatacgaaatatccagaagagatacatccatggtgacagaaagtagatgagcagctggggactggcgaaggggagaagggggagcagctgtctatgaggtccagcctttcttctgggtttggtgagaatgttttggaactagatagaggtgatagttgtacaacattgtgaatgtactaaatgccactgaatcattcattttaaatcgttctttacgttgcatgaattttaagtcaatcaaaaacagttgtttgaaaagagaaaagcctatgggtagcggcagcagtgattggatttatgattcgattccatggctcatccctcccctgcctcaccccctcgccctccgacgtcttcttcttttactctgaactgttatctttgttctcatctctctctctctctctcaaccctgcagacacttttccctttctttgtctgcccccaccctccagatttccgtgtctccagtgtctccctacgaggcatgaattgagactgggagggtgtgattctgaagaaggcaccaacagtgactcagctagccccttcccccaccccgccccccgggcctcaatttagctaaaaaaccacagggacggactcaggaggcaatacctttccaagggtccctaaaaaatgtcccattttagtgtccaggtttcactcaactttagtgcctcccctaaaatgtgttccttacctcccaccccactgcatctaagtcactgcctgagaaaacaggattgaggaaaggagaaaggaagagagagagagaggaggagagagagagagagggaggaaggctgatggatttagaaaagaagaaaacaagtggtctgaggaaaacagccttggtgtgtttattttcctgtctgtgtatcgcttctcggccttttggctaagatcaagtgtattttcctgtctgtgtgtctcgcttagattacagggatctgtgggtgatgacacgtctggtccaggctgcgtagtcacctcaagggcatgcttattgatgtgtttttcaattcactatctttgcatgggagtcccaggccaagaggcacagctgcgccatttgtctgttggtttagatatcctttatccagttcttccagagaaatcatcctgcccttctggaggaggtgggcagcaggggtcagagatgggagggaaaggaaggagccaggtccttggctaggatgccagggtcccctgcctctcacctggcctgggctggaggcctcctgctgtcctgtcactgatcactaccccgccccagcctcctgagttagaagacacaggctaaagtagagtatttcttcattgaaaaacccatacaaaataaaggttcataaaaaataaaaatttagactgggtgctgtggctcacacctgtgatcccagcactttgggaggccaaggcaggtggatcgcttgagccctggggttcatgaccagcctgggcaacatagtgaaaccccatctctacaaaaaatacaaaaaattagccaggcatggtggtgcatacctgtggtcccagcttctcagcctatggacccacatagaatacaatgtcagcataagaagggagccctggggtcaccaaatggtttgggcggcaaagaacctgaaggttgagagaagtggcttggttacccagctgttggatgtgagacctggccactgcttcttccataccctagacctgcaccctgacatctcaagtaaaaagttgggggatgttttatggtccaggatgaaggaagggcagtgaggggcagcggagcatcactttgcatttctgtctgcctcttactggctgtgtgacctggggcaggtaacttcccagactcctgggaatcataacacctatgatgatgatgatgatgatgatgatgatgacacctacctcaaggattgccctgaagggtcacagagatgcctgcaaggcacctgcatggagcaagcgccccttctctggcaggtgctgggtgagcactacctgctgccaggccctggggctatggcactgcgtgaccctgcaagtcctacctggcgaagctgtcgttcttgtgctcagtcagtgttggttgtaagactgagaagagtcacttcattttgctctccagggacatctttctgggtcctattttctgcctatgtcaagtagcgcctcaaggatgctcctgaaaatgggcttgtctttcttaacatggcaggtaggtcccaaagcattagcatggggcagctgacctagcccagccaatgcagtgcagtgactcttgcaaccgagtctaatcagaaggtccatgaacctacgagcatttcctgtcccaggatcagggtggaggctgagcctccctgcttagagattcttcccatgcattccacttttttccccaaaagaaaatattgacccttgagaggcacacagtttatttattttgcatagtaaatagtagcctgtattttaaggatgagttgatttctgcatcagcccctgtaggtcatcagccttctattggtgcatctgactctctctagccctgcagggatggtggagggggaggggaaggagggatctttattggaaaccaggacagtgagactcattgccctgtcatctgctctgtggtgctgaatgaggcagcccaacagagaaataccctgagcgagcatccccagcctccaaaacagtggcgcattgccctgagtcctgggaatgacctttgattctcctgctcctgacttggaacccatggaaacctctagaagcagctgaggaaaacccaacatgaaaagcagaactccacactgagaatataggaggtgatcggaacatacaatgattcttgctaagaccgattcacagtttttcttttttttcgatcgaagaaatactggagaagcctaaagaaggagtctaaaaactctggcacgtgggccaaaactgtccttgagctaagaatgattttcacatttttaagtggttgaaaaatgaaataaaataagatgatgttttgtgacacatgaaagctatgggaaattcaaattctaatatctataaatagtgttttatcagaacacagtcatgctcatttatttatgctcgatggctgctttcccgctacaattacgttgagcagttacaacagagaccacgtggcccacaaagccttacaatatttactatctggccctttccagaaaaaaatgtgccgactcttgaccttaacctcagcaatttgggaggccgaggcaggcggatcgcttgagctctggagttcatgaccagcctgggcaacatagtaagactccatctctacaaaaaatacaaaacattagccaggcatggtggtgcacacctgtggtcctagccactcgggagactgaggtgggaggatcgcctgagcccaggaagtcgaggctgcagtgagctgtgatggcaccactgcacctcagcctgggcgacagagcaagaccttgtctccaaataaataaataatgcaaagtaaaataaataaaaccatataaaaaggaatcaatttaaaattataatgaaagctggccgggcatggtggctcacgcctgtaatcccagcactttgggaggctgaggtgggtggatcacgaggccaggagatcgagaccatcttggctaacacggtgaaaccccgtctctactaaaaatacaaaaaaaaaattagccgggcacagtggcgggcgcctgtagtcccagctactcgggaggctgaggcaggagaatgtcttgaacccgggaggtggagcttgcagtgagccgagatcgtgccacttgcagtccagcctgggcgaaagagcgagactccgtctcaaaaacaaaaacaaaaacaaaaacaaaaaaaaattataatgaaagccaaggggcatagtagaacaaattttctagagctcattaagtcaaatgagtcaccagttagtaaaacgcagtcacggggaagagagggcaggattctttgaagcagcggctctcctaaaaacaacccacccttgtccagctgccttccctcctgagggtgttccctttgactgtgtgacccccatcccctatttcccaaccgtccaagcccacctctagcataatacgagcttttaatccctctccctgaccccaacccgattttgaagcccagtctagtattttctcaaatacacttcttggctccattccttcctttccatcacctctgccttttcactgcatgcttggaccactgcagtcagctccctatgaacagttgctctctacccatccaatcggccccgcctgctgctgccaaattcaccgagggcacctctgtggtgctgcctgtggacaaagtccaagccagccacctcacccacctacaggtgagtggggagcagccagcgtgtccagtggtttaccccatcgccacagacttggtgatgtgtcgatgtgcagagaaggggtgttggcagccacaacacaagcaaccccgccccatgtgagatctaagatgggcgtgctgggagccacctctgagaatccaacagaaggcagaggggagaacggctcacacggcacaaacactccttccttttttttttttctttttcctttttgaaaggagtctcactctattgcccaggcaggagtgcagtggtgcaatctcagctcactgcaacctccgcctcctaggttcaagcgattctccagcctcagcttcccaagtagctgggattacaggtacactccaccatgcccggctaatttttgtgtttttagtagagacggggtttccctatgttggccaggctggtcttgagctcctgacctcaggtgatctgcctgccttggcctcccaaagtgctgggattacaggtgtgagccatggggcctagcctccttccatttaaatgtatgcctaatttgcccattgagaacggctgagacgcattttaagtggccagggtctacttagagttagtgctcatgaccaggcccaggtcaagcctggctggccagatggtgcctttgacctgctctgtctctgtgcaaaggaatgagctgaaggatgggggtgcagtgtgtgggcagtgggctggggctggcaggactcagtgactaagggaagagaactttcctcactaccagcctgtcttttcagggcaccgcggggggctttgggacttggtgatgaacacagcacagagagctgtccagcatgcgggtccctggcttctcacacttcccaggctccttcagaggctctctccaaagggagctgctctctctagaacccatgaatttggaatataggcaaccactgcattggggaccactgacctcaaacatagagaccagagcaaatggggctcatcacgtgaaactcatctggaactctagcaggttcttttatatatatatatatatatatatatatatatatatatatatatatatatatattttttattattatactttaagttctagggtacatgtgcacaacatgcaggtttgttacatatgtatacatgtgccatgttggtgtgctgcacccattaattcatcatttacattaggtatatctcctaatgctatccctccccactccccccaccccacaacaggccccagtgtgtgatgttccccttcctgtgtccaagtgttctcattgttcaattcccacctacgagtgagaacatgctgtgtttggtttttttgtccttgcgatagtttgctgagaatgatggtttccagcttcatccatgtccctacaaaggacatgaactcatcattttttatggctgcatagtattccatggtgtatatgtgccacattttcttaatccagtctatcattgttggacatttgggttggttccaagtctttgctattgtgaatagtgccgcaataaacatacgtgtgcatgtgtctttataacagcatgatttatattcctttggttatatacccagtaatgagatggctgggtcaaatggtatttctagttctagatccctgaggaatcgccacactgtcttccacaatggttgaactagtttacagtcctaccaacagtgtaaaagtgttcctatttctccacatcctctccagcagctgttgtttcctgactttttaatgatcgccattctaactggtgtgagatgttatctcatggtggttttgatttgcatttctctgatggccagtgatgatgagcattttttcacgtgtctgttggcgaactctagcagcttcttttcacaagttcatggagagaggtttcccactgagggaatcacatctgtctgatcaaaagaggcttgggaaatggctctcctgttcattccctgaaaacctctgatggaaccactgccactgtggcagccccagcactggcaccccagccatgattggtgccccagccacatctctgctgtgagccccagagccctggttaattaatcatccacgtgttgatggggagaggcccattcacaaaagcgacataaagcccagggagacgtggccgtggcaagaagggtgtgggactacattccgcccccaactgagagattcagaaaccagaaaaaaatggaaaaacatactgtgctcttgggtgggaaaactaaatatcatgaagggagcaatttttatagttttggcctataatacaattccagccgaaatcccagtggaactttgagaatttgcaggaaaaaaaaaaatgtctaaagtacatctggaagacaaacttacaagaaggtcaaataattttgaaaaagaaaatgatatctaagcccacctagagaataagacttgagatccaaagctaaatcaggaggctctagcaaaattgacagataagcaggacagagtgcatggtgcattcacctggggaagagggcagattggtctacaaataggcctgggtccactgactttagctgttatatttggggagaaacttttcaacctcactccatcttaaacctaaaaatattccagatgaattaataaatataaaaaattagaccactaaaaatgtagaagaaaatggatgatctttctataccatagagcaatggaataaatcacaaaggaaaacagatttgactatataaaacttaaaccctgcccatcaaaaaccatcagaaaccaaaataaaaggcaaccaactggagaagatagttgccacaaatatgatcaagggttaatgttattcataaattaagagcccacacaagtcattagaataagcactgagacctgaacagacaagcaaaaagaatgagagtgggtcggcgcggcggctcatgcctgtaatcccagcactttggaaggctgaagcaggcggatcacttgatcccaggagttccaacaccagcctgagcaacatggtgaaaccctgcctctacaaaagtcataaatattagccgggtgtgatggcacacgcctgtagtcccagctactcaggaggctgaggtgggtggatcacttgagcccgggaggtagagtctgcagtgagccaagatcacaccgctgcactccagctggagcaacagagtgagaccctgacttaaaagaaaaaaaaaaaaaaagaggagaaaaatgctgatctcactagtaattaaaacatcaggccaggcgcagtggctcacacctttaatcccagcactctgggaggctgaggcaggcagatcacttgagatcaggagttctagaccagcttggccaacatggtgaaatcccgtctctacaaaaaatacaaaaattcgccaagcgtggtggcacatgcctgtgatcccagctactcgggaggctgagacaggagaattgcttgaacacgggaggcagaggttgcagtaagctgagatcgtaccattccagtccagcctgggctacagagcgagactctgtcccagaaaaaattaaaacatcacatatttaaacaactctaggatatcatttaaaaaaacattaatagactgttttttagagcacttttaggttcacagtgaaactgagtggaaggtacagagacttcccgtatgttccctgccctccacgtacagcctcccccactgccaacgtcctgcaccagagtggtacacttgttacaaccaatgaatcctcattaacatatcattatcacccaagttcatagtttacattagtaaaacatcatctttcatctataagcacaaaaattttttggcatttatttaggtgtatgattaactcagtgttgacaagactcacacttcatacccacttgcactgcatctgagaagcaattggtgtctacagccgctacaccctcaacaagcccgatcttgtttgaaaagcaattggtgatgcttctcaaaattctatggacaaagtcagccgggcatggtggctcatgcctgtaatccctaaactttgggaggccgaggcaggcagatcacctgaggtctggtgaaaccctgtctctactaaaaatgcaaaaattacccaggcatggtggctggggcctgtaatcccagctactcgggaggctgaggcaggagaatcgcttgaagcaaggaggcggaggtttcagtgagccaagattgcaccactgcactccagcctgggtgacaagagtgaaactccatctaaaaaaaaaaaattatggacaaagtttttcaaaaagatatttaatgcaactttatttgtaatattggaacatctgaggccatttcagtgctaactattaggggatggttaggaaaatatggtacatatgtggaaaggaacatttggtagttagtgcccctgatgtttacaaaggcttttagtgaccaacaaatgctcatgctataatcttatgtgaaaaaagcaagtagcataattgcaactatatttttaatgcatagaataaaaggctagaaggaaatatcacagatccttgacatacattcccaaacctttgtaaatccgcggattcatgaaaacagacacatttgcacaagtgcctgatcttttctgttatacattcattagaagtcaagccctggtgccacaaagtatctgccttttcaaatgtgatcagaatgttctcttttgcttcaaggccatttttcacgaagcagtggcatttttgcctcttcatcagagtcaccgtgtgccctggaggactgagaacagcagagccgttttaggatgggacagggcagccaggaggattgggctcactccctactgagtgcctcactcccgtacagcccccatagaggaagaggggttcaaatttattcctcagccagatggcatgtgccgcctgtcctggaatttcacatcacttatgatggaccaaaattccaaaagctgaatccatgattgtcaaagtctggtatggcaggatgtcaacagtaatcgtttctgggcagagggatgattttctcttcccatcttgctttgtataaatacattttctataataaggttgtattacttttctcatcaagaaatagcaaagtactgttttactcaaaatatgaatagagccaggcatggtggcagcttatgcctgtaatcccaacactttgagaggcggatatgggaggatcactttagcccaggagtttgagaccagcctgggcaacatagtgagacccccgtccccactcccccaaagaaaacccacaaagcatttatcctggattattcacaggggccaaaaaaaaaaaaaaaaattcaggcctcctatagccatgagctacgaatatgaaaatatgcaaatgtgtaagaaaagccagcacatccgatttttacttttactttcacacctctgtccaccatgttccaagagaagaaacttggtcattgaaaggaatagatcaaatccaaagaacaaaaccactgtgctcattaaacttcttagtgttcacaaagctttagctgcaggttgaatggggcaacccgaattggctggctcacctgggctgcagggagcagagatcgcgacactgcactccagcctgggcaacaaagcgagactctatctcaaaaaaaaaaaagttcataaattcaaagttatgaattatttttaaaataataataatttacaataaagatgaggacaaagtgtgagtaaatggtggtttctatccagctctgttgagctgaagtggcatctccctgctggggcttttggggaagaagggtgtgtgttgctcttcagatcccaagcctcatgcccctactgggccctgtggggtgcttctcagcccaccaggagagccaccgttggaacacacacgtgggggacctggtgggtgccggtgtggtgaatgggggccacagcctgactccaggaagccagcaaactcggagctggaggagtcaggacacccccgatgagtcaagagttggttttgctgccagttgacatctgattgaaccatctcttcacttctccgtgcctcactttccttaccagacaggctctgctgatgctgtccctctcctgttcagtcgtgccctcaccgttaaagagaaagagcaaactgctgggcagcagcattgatttttttaatgaagtggaaagagagctgggaataacaagtcgggcccacctcacctgcctcacctggtgggtttatttgttttgtttttttttttttgttttgagacagagtttcaccctgtcacccaggctggagtgcagtggtgtaatctcagctcactgcaacctccacctgccaggttcaattgattctcctgcctcagcctccccagtagctgggattacaggcacctgccacatgcctggctaattattgtatttttagtagagatggggttttaccatgttggccaggctggtctcgatcccctgacctcaggtgatccacccacctcggcctcccaaagtgctgagatcacaggcgtgagccaccatgcctggccgtcacctggtggtgttgaatatgaactgctgcggtgttggtaaattaagcaagcagatagatgtaaataacgcttgggcaggaatatggagcacgggatgaggatgggcggccaactgttagagagggtagcagggaggctgagatctgcctgccatgaactgggaggagaggctcctctctctcttcacccccactctgccccccaacactcctcagaacttatcctctcctcttctttccccaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaaggttagtggacagccatgcacagcaggcccagatcactgcaagccaaggggtggcgggaacagtttgcatccagaattgcaaagaaattttaaatacattattgtcttagactgtcagtaaagtaaagcctcattaatttgagtgggccaagataactcaagcagtgagataatggccagacacggtggctcacgcctgtaatcccagcactttggaaggcccaggcaggaggatcccttgaggccaggaatttgagaccggcctgggcaacatagcaagaccccgtctctaaaataatttaaaaattagccaggtgttgtggtgcatgtctatagtcctagctactcaggatgctgaggcagaaggatcacttgagcccaggagttcaaggttgcagtaagctgtgattataaaactgcactccagcctgagcaacagagcaagaccctgtcaaaaaaaaaagaaaagaaaaaagaaagaaagaaatttaccttgagttacccacatgagtgaatgtagggacagagattttagggccttaacaatctctcaaatacagggtactttttgaggcattagccacacctgttagcttataaatcagtggtattgattagcatgtaaaatatgtgactttaaacattgctttttatctcttacttagatcaggcctgagtggcctctctttagcaagagttggttagccctgggattcttactgtagccacattaataaacaacatcgacttctaaacattctataataccatcttttggccaaattgacttcgcctcttcctctctctttccaaatgaaatgtgtttcatttcactgtcagaccacatggttggggaccccacagagcacacagccctccctctgccttcccatgctggcccttcacccactgctggagtgccaggttggtccaagggttggaccaagttgtctgaggttgtctcaaggttggtcgaggctgtctccgcgctgggttgtgctacaaggagcccttctttccatgggtgtggctggcagtgagtgctcacagcaacagcccacagtgcagcccgagggcaggatggactcagtccctgcctccatacccatttctaaggaggcaaaatggcaaacactctacttttctcttttaatgctaaaaataagaaaacaccttgcagcccagggtatgggtagtgcatggaagccgtggagttgtgaggtgggaagtgacctctgctggatatgtctattcaggaagattgctggagtgggtggggtctctgggaggtcccctgagtgtgggaagctgggaccaccagctttctcgcacagggagtggccatcccagcttggagaggttccaggactggttgggaggcacgtttcagatttctatctgttgaatcagcgaagatattggattatgaggaatttgggaattaggaaagtgggtgcaggtgggttgggggtaggtgaaggaagacatgggcgtattgggggagcaggggctgctcagaggtgttccagaagctctgggtgaggaggtgagagggaccggggaatgcagctcggcccagcctccctgcctgaggtcagccatcacgtggtgatggcaagatggaaatgtgctttctgactgctccagccagtgctgccagattcagctccccagggagggcacctgagaggctccaagccaggagatctgttttctcctttgttttgtttttttttgttttgttttgttttattatactttaagttctagggtacatgtgcacaacgtgcaggtttgttacatatgtatacatgtgccatgttggtgtgctgcacccatcaacttgtcatttacattaggtatatctcctaatgctatccctcccccctccccccaccccctgttttctcctttgaatccttcttagaggccgggcgcggtggctcacgcctgtaatcccagcactttgggaggctgcggcaggaggattgcttgagcccaggagttccagaccagcctgggcaacatagtgagacctcgtctctacagataataattttaaaaattatccgggcatagtggcatgcacctatagtcccagctactcaagaggcagaggcaggaggatcacttgagcccaggaggcggaggttgccgtgagccaagatcccaccactgcactccagcctgggcgacagagacccccatgtcaaataataataataataaataaatccttctcagtcccttcctcactgtgtccccctccactgaatttttccacctcctctcccacttcccccactcccgctttccctctccttctctccccactccatctttttctttctctgctgtttctcgtccctccctcctctccatcccacaacactgcctaccctgtccctgccccaccctggtgctcaggatgtgtgaagtgaggggtggtagcccccaagacctcaaccccgaaggttagcctgttgaaaccactttctcccagctgcccccctggcagttggtgctgctgggggaaactgggattgggggccagattttgcctcttttcctgacaaagagagatgaagagttctctcaccaggtgcctgggactggggtgtgggtgtcccagcctatcccagcgcatctgttctgcatcatgattaatagtgctgctttcagccgggcgcggtggctcacacctgtaatcccagcactttgggaggctaaggtgggcagatcacaaggtcaggagttcgagaccagcctggccaacatggtgaaacctcgtctctactaaaaatacaaaaattaaccaggtgtggtggtgggtgcctgtagtcccagctacttgggaggctgaggcaggagaatcacttgaatctgggaagcagaggttgcagtgagccaagatcgtgccactgcactccagcctgggtgacagagtgagactccgtcctaaaaaaaaaggagttttgctctgtcgcccaggctggagtgtagtggcgccatctcggctcaccgcaacctgcgcctcccgggtgcaagcgattctcctgcctcagcctcccaagtagctaggattacaggcgcctaccaccacgcctggccagttcttgtatttttagaagagacggggtttcaccctgttggccaggctcgtctgggactcctgacctcaggtaatccgcccacctcagcctcccaaagtgctgggattgcaggcatgagccaccgtgcccagtcaactccttctcaaaaaaaaaaaaatagtgctgctttctctttcaagtgtcctgatttgggtgatagtaaatgccactctacttataagggatctacctcagaatgctaattgggacatttttgtagcactctactgttggcagcaggtgatgctcacaacagcccgtgagggtggatgacgtccgcttcacagatgacaaaggagcctcatgctcagaccgtgggctgccagagcaggtccatggctgcagccccacatggaccatatttcccccttgtcactctttccaccaagctcccttggaacttcagttattaagctctcttgggtggaatccaagttagaatcacaacatgtgcctcatatggattgtgccagtgaaaaatgacattctatttagaggcagggcagcctggcttagagtcagtttaaaatatgtattatgctgcaacaaatgtaccatgatcctgtaagatgttcacaacaagggaactggatgtggggtatactgtctgtactaacttcacaagttttctgtaaatctaaaactgttccaaaataacaagttcgtttaaaattaactccaggagaccaggtacggtagctaatgcctataatcccagcacttcggaaggctgaggcaggtggattgcttgagcccaggagtttgagacaagcctgggcaacatggtgaaatcctgtctctaaaaaaaatcacaaaaattagccaggtgtggtggcgcattcctgtagtcccagctacttgcggggctgaggtgggagaatcatctgagcccaggagtttgaggctgcagtgagctgtgattgtaccactgcactccaacctgggcaacagagcaagaccctgtctcaaaaaacaaaaatgaaataaagtccaggaaagaagtaggttttaccactcttattttctgaagagaaaactaaatttaatgtgtaaagtgaggacaagttcaccaagttagtgtttgagttgcctaaaatatgtttgctaaaactattcaaagctttcacataaaacatgatcagaagttctatgccaaaacatatgtgtgtgtatatatatatgcactatatatactgtatataaaaatgcaaaatctaaattgccaaccttttagaaattgctctgaaaggaaagcatttcaagataatttgcttacccaaagaatatactttccaagaaagcaagtaatacttaaggtgttcataatcctcatcaaattaattcttgctactgaaagcttacaaggagctgttttgatgtcgggtgtgacaggtttgacttggcagaaggtgtcactttactaacaacattttaaataagtgacagaagacaagaaactacacgttaaatgccagaacaaagagtgtctaagtggatgctaagagttgaaatatggctggatacctgcccaagagagctgaaaagtagatgaaagttggttacctataaactagtgcaccctaatgaattaaaaggtgttgatgagttaacttgttatgccttccagataagacatgcaaatggggcttcttcctccttcactacttccaagggatttaacaaggagaccaatgcaaatgataaggactgtagggctcaagctggggacagattggggaaagggggaccatcatgcccatatagatgtccctgtgccctggcagtcaaggctgctgaaaaataacaaaacccagaagtctgcgtgatgctgcctctccatttgtccaaagccttcttgcggcagtttgcaggcttttgcaaaagctccaggaccaaggagctatgttcatgctggaagcttgttcaggattagctgttctttgtgggatgggtgcagccagggccaggtgtccagggacagtgttttaacaaagggcatgaggtgtctgatctcacagtggaactccacttgcctttttttcatcttctcattctgcttcatgcacagaaccagccccatcctgaaactgactctaaattactcccgccccaggtggagtgcctttctcggagttcaacagagccttcctgtcgcccaagggacaactccactgaatgcccaagccacacccaaaacctaacaagtaaaaaccaaattctgtgctcccccatcctgggccattcctggtttctctactgctgttggtgataccaccatcagcttgtccatcatgaccctggccagttcctcccacaaccctccacagcacccagggacctcacctccattccatccgacacagatctcctcaccacaaaccttggttttgcaacagcagccatgagacctttacaccctccgcccttcatcctgtcccccactgaggccccagagccattccttaaagcagcgcgccacaaactataacccacaagccaattctggtacccagcctgttttgcacagccagtgaactgacaatgatcttttcatacagccagaaaaacaaaacaaaacaaaaaacaacaaaaaaaaaccccaccattctgagcatgtgacttccatgttcaagatgtctcatgttcagaaaggcccctggaaaaggaggaaggggagctgggcacaaagggagaccctctcagctgagctcctcccatccagacattttcctggacttcctatccaatgacttcccttagcttcttatcagccacccctgtctgcccaggaggctggaagatgtggccttttaactgggcacagctctgtcctctatcatatcagggctctgttcccaaggagggtagagagaatggacaccaggtggaccctcagcagtctgtgccacagagggagtgtttgcaatttccagactaaaagtccccatgtgcttgacggggtatgtgactacaacgtgatgcttgacttttcctcatatgaccagagccactttgtccatctggtacaatgtcagctatctgctaggggccctccaggattcccagtcaattccatatctgcatcaccaccattggcactaaataaaataaaatactcaagttcctgctggtgagcatgagcagtgctacactgggcccttcaaccaaggtgacatgataatgactgaaaataatcactgccacttattggggacgtctcatctgccaggcatggtacaaagtgctttaaataagcattcaacaatttcatgctgacagaagccctgtgagccagtggagctactactatgcccattatacaggggagaaaactgaggcagagagaggttaggtaattcgctcagcctcacacaaccaataggtggtggagccaggatttgggccccatctgcctgactctctagaggctctatcttccagtcttccagagttgagtctaagccatgaataggacaattagacagcagaggaaacccattcagccaccatgtgcatgaagagtaaggaatttctgtcatacagaggggagtgaattcactgagctgagagctgaggaaccattgatctgatggctgagacaccactgggaagactggagaggcttttctgggcatgcagtgccaggcacaggaggagctgagggaagatgactaagaggtactggcaaagaattcagaaattctgatggaagctttacatgttaccatcacatccatccatctatccacccatccatccacccatatcttcctccctccacccaatcatgcatacatccagtcatctatacaccacccacccacccatccatccatccatccatcccttcatccatcccatcatccatccaattatacatacatccaatcatatatctgtacataatccattcttccctcggttcatccatccatccattcatccatccatccacccatcccttccttcatccttcctatcatccatccaatcatatatctgtacataatccattcttccctcggttcatccatccatccattcatccatccatccacccatcccttccttcatccttcctatcatccatccaatcatacatatatccaatcatacatctgcacatcaccagctcatccatctatccatttatccatccatccttccttccatccatcattcatccatcatacatacatctaaccatacatctctacatcattcattcttccatcgattcatccaattatccatcattccttcctccatccatcccattatccatttgatcatacatatatcatctatacatcatccattcatccatccatccatccatccacccatatcttcatccaatcaatcatacatacatcgaatcatctacacatcacccatccatccatccatccattcatctatccacccatccatccatccatccatccattcatctatccacccatccatccatccatccatccatccatccatgtaaccatccagtcatatatccaattacacatccatccagttatacattcatacatgcatctaatcattcaattatacatacacacatccatataattctacatccaattatacctccatccaattacacattcatacacccacctaataaattattaattcatatatccatccatataattatacatcaattatacatccatctaatcattcagtaattcacccaccatccagtcatctatccaataatacattcatccaatcatccatccatccatccacccattcatccatccatccgtccgtccacccatcatggtatgagccatgatttaccacgatggtcccctgtggacagcccaggtggggcagaactgaagggaagcccagggctgcccccataaacatttgcctcctttacatggatgagaactagatccacatgtataaatcctcatgatttgaaggtgcttttaccaacattcactcatgggattctcccaggagctctaggaggaggcaggtagagttgaggtcatctcacgcattttacagatgaggaaacggaggccctgagaggcaggtccaaggccacctgaccagaaagaagtggaactgggacttgaacccagccatcttgccccttggtcccatgctctctagcctgtaactcctgcttcctggtggggcatctccaggaggaccctatcggctggccatgggcctgccctggagtcttttgctctgtgtggccatccttcctccctcaggagagtgtgtgctcccagagcacaggctgtatcttctgagcattttgtcccttcccagtacctagcactcagctctgtatacattgggctctcaagaattctcaaccttccagagtgtaaggccttgacctgctcagccctggatactgcatgatgcattgataagcccataaaataaccagggcagattgactcccagtggccaaagtgccacagggaagggacaattcagcccttctaggaggaggaggaggtagttttctcatttctattaaggcaacaaaagctgccttactaaggacattcttggtggagggcgtgactgtcaaccactgtgatcatttgggcctctcttgcccaggcttcccattctgaaaggacagttttattgtaggtacacatggctgccatttcaaatgtaactcacagcttgtccatcagtccttggaggtctttctatgaaaggagcttggtggcgtccaaacaccacccaatgtccacttagaagtaagcaccgtgtctgccctgagctgactccttttccaaggaaggggttggatcgctgagtgtttttccaggtgtctacttgttgttaattaatagcaatgacaaagcagaaggttcatgcgtagctcggctttctggtatttgctgcccgttgaccaatggaagataaacctttgcctcaggtggcaccactagctggttaagaggcactttgtcctttcacccaggagcaaacgcacatcacctgtgtcctcatctgatggccctggtgtggggcacagtcgtgttggcagggagggaggtggggttggtcccctttgtgggtttgttgcgaggccgtgttccagctgtttccacagggagcgattttcagctccacaggacactgctccccagttcctcctgagaacaaaagggggcgctggggagaggccaccgttctgagggctcactgtatgtgttccagaatctcccctgcagacccccactgaggacggatctgaggaaccgggctctgaaacctctgatgctaagagcactccaacagcggaaggtgggccccccttcagacgccccctccatgcctccagcctgtgcttagccgtgctttgagcctccctcctggctgcatctgctgctccccctggctgagagatgtgctcactccttcggtgctttgcaggacagcgtggtgggagctgagccttgcgtcgatgccttgcttgctggtgctgagtgtgggcaccttcatcccgtgtgtgctctggaggcagccacccttggacagtcccgcgcacagctccacaaagccccgctccatacgattgtcctcccacacccccttcaaaagccccctcctctctctttcttcaggggccagtaggtcccagagcagccatttggctgagggaaggggcaggtcagtggacatctgatcttggtttagtatccttcattttgggggctctgggtgtggcctgggcctctggactttggccacggtgtttgttccagcccttctcctaacctgtcctttccagacactcggcatctaggttattagcacctcgcatactttctgacatgctcctcagtcctgattttgaccatcttctcttgcttcccatctgtgtcagtcaagactgcatttggctgtaagaaacagaaaccccaactaactgtggcatttacatgaagaggtttacttttctcacataatcagatgtctagacttggccagcacctcaagggtcattgatgctctcctgtctttattttctgtcatctttagtggttggattgttgcctcatggttacaaagtggctgctgcacttccaggcatcacatctgcctttgaagcaggaacaagttgcaaagtaaagtggccaaaagggccctgaaactaaatgtgtccccttaggaaagcaggagttttcttgcaagtggcaatcttctgcttatgtctcattggccagagctgggtcttacggccaccccttgctgcgagcaaggctgggacattgagcattttgccgtccaacctctttagcagaataaaccaagggggaagaacgttaatagtggcttttgagtcactagttggcagtatctgcccctctatctttccatcctccccatggagtttcaaggttcctttctcagtacttcttcaggctctgcacgttcatttggatcttgtgtcttggggtgaaaaactggcccaagtgtctccccaagcatccacctttggattaatttggaaaatggctgtcaagtgcccgcctcttgcttggtataatgctacagctttagaggacgcagcaggcatgggccttgccgctgaggttcttagcctcatgagaatatccagatcagattctcttggctccttcttagagccagtgatgcaagacacttcctgctcatcttgtcgggacggttttacaagttgcctgccatcctgagaaagtctacaaaacgatgccagacctcatgccagcttcccaagccttgactctcagtgctccctcaacaggattctggaagaatctcccaaacaagtcgcaatgccctctggaccctgtgcaggcatgagactcaagagcattggctcccacccctggtggagggaacactgctggggctgggatcttgcctggttgctccgcctgcacccaagacaaccataattaaaatgtccttcattgaacttggaaagccttcaaagctgacaactccttatgtgtacccggaaaggcctgggagtgtgccagggcattgctcgggagggacgctgatttggaagcatttacctgatgagagactgacagcagctcctggtagccgagctttccctcctgcctctgctgtgaaggtggacccatccaacagtcaaatgcctgactctggacaggagcggacctatttattgccatgcaagggactctgcacttttgaattgtgggtcatgggcttggatttaggggttagagctgggagaagtcttggaagtcacctagagatgacactgccattttgcagatgaggaaaccgtccaatcaaaatggaccaaggacttgcccaaagcctcacagcaaaaccataggcccccgcactaaccccagagtccctgtgctgtcttaaggatcatatagttgtaagcaatcatctggttttcagtatttcttcttttaaaatgcctggggccatgcccagcagtctgtttcactgcagcgtttacacagggctgccgggctttcctggtggatgagctgggcggttcatgagccagaaccactcagcagcatgtcagtgtgcttcctggggagctggtagcaggggctccgggccctacttcagggctgctttctggcatatggctgatcccctcctcactcctcctccctgcattgctcctgcgcaagaagcaaaggtgaggggctgggtatggctcgtcctggcccctctaaggtggatctcggtggtttctagatgtgacagcacccttagtggatgagggagctcccggcaagcaggctgccgcgcagccccacacggagatcccagaaggaaccacaggtgagggtaagccccagagacccccaggcagtcaaggccctgctgggtgccccagctgacctgtgacagaagtgagggagctttgcgtgtttatcctcctgtggggcaggaacatgggtggattctggctcctgggaatcttgggttgtgagtagctcgatgccttggtgctcagttacctccctggctgcctgccagcctctcagagcatttagggccttctggacttctagatgctcctcatcttgcctcagtcagcgcgtcagttccagagacttctctgcagggttttctggggcaggtggtggcagacccgtgccttcttgacacctgaggtcagtccaccctcctgctcagactgcccagcacagggtcacctcccaaggggtggaccccaagatcacctgagcgcacagagggtgcagatgactggaccacaccttttggtgatcttaatgaggtggtcccagaggagctcagacatgcaatctagcatccagttctgggactctgtctccttttcaaacgtattcatgtagaacaggcatgacgagaatgccttgtcaacatgggtgatggggaatcaatcagacagggcgccgggctcaaggctgcagtcacccaagagtggctcagcccaccaggccctaggaaacgcctgcacagcctggagctcctggagtcatttccttcatgtcttcttcactgcacttacgtaaagatgccagccattggtttggtgatttggagggtgcccagttgcccaacaagaaatgcagaagaggcctagccaggatttcaccagcagtggagagtagagaagatgtggccagaaaagagtttcctttccctcctaaagatggtactccctgcagctactggggaagcctgcagcattctctagggctctgtgtgttgagagcagccccaccctggccccttctgagtgcatttctgctttgtgacttgatccgtgaagtcccctgagatgggcagaggggatgtcctcgaagctggggcagagcctcatccttgaacgtgaaggacgtttgaagactgtggcatgatcacaggatgagatcacagggaacttgagtttctctcctcctctcccttcacagttatttcactgagggaaatccctcccctgcccagaatgaaaactctagccaactcttgacttttccatcactccaaagtagttgaaagtacattagtctccacagtggcaaaacagtgtgcaaaagctaaataattagaacagccagtcccatgtgacagtcaaagcttctaactccattcaaagttgcagccattcccctcgagggctggcagggaggggaggggtaagagaaacaggaaggttcttactgagttggtcctggtgtgagctgcgtcacactccctgcagaggtttcaaggagactctctctctctctgtctccatggggaccttatttgaattcttctactcttaccccagcctgccatctccagctatcctcccctgaagagcccttctgctgcgctggattctggtggccatgtcatctcctcggccccgtgggagtctgaagatctggctgcagcctcacctctgaggtcctgctagttgccacctcttaaacatgatctgaggctcccatgcactctgacctgtgcccacatggggcccacgggaaacacgctggcaagcaaactgtgggtgtgcagacggttctcagggctgcagcacctgtcctttgctctgcccccaaagcaaggccagcccatcttccatcctctagtgttccttggtggggccctgaccacagtccaccaggtccctaaccagaggggacacacaccaggtgtcctcaatgtattgccttgaaacagttgtgctgggactgtgatggggggtggccatgtagccacccccaccacccccaagccactctctccaaggaaatcctcctaaagatccctttacatcctccatgtggtggggaggttctagagttgggtgcatgtgtcttcagctactgacaatgcagaccttagttggcacctcgctctggcctatcctgtttgctgttcttggcgctccagtgaaactccccatgggccatccagttggggtgcagtgtggccacccccttgcaggttcctgccttgctggagagcacagggccctcctggctcttgtaaaacactccccatggtacagagaggccagcagtgatgtgaggcccaacctccctccatggtgttcccaagcagctccctttctggggtcaaggggtggcaaagacagtgcagcgtccaatttctgactcaagccgggcctggctatcgcagctctgcactgtgtgtgacagcaaggcaactcacccagtgccgtggcagtgaccgtgtccgaggaagcctcctcacaccctctgtctcaaggactctggcatttagctggacttgctgtagctctgagcctttctgccattgccatcaccttgtcagaaactcaggccgaatctgcactcagagttgtgcccaggcagttgagccaacacttgctcagcgatattgtcacatgacaaggcactgtcaccactgggcatcgtgggtagcgcagtgtcggctggatggacccggagggtgtctgtgtcatgctagtgctagtgatgggagccccgtgagcccattgcccgccctcccatgccctcagcagctgcctggggacagccaatggcctgggtgtttctgaggctaccacatggcttccaggaaactcgagaacctttctctcccttgcctacactcttcacacaggcctgtgctggccagcggtggggatccggcattcctatcttaggtgcagagagtgactgactcattgcaggcctgggagataagactgatggcccagccagcaagatgtatggatttctcagaggcagtggcctctgtcattgtcctcaggaaatgctggtgattctggtggcctgaggtcaatgcatgtcaacgtggccaacttgccttataaactttttttctggacaattgcgtgcactgtcctgtaacagtgtcctgttgtttatgatgcagaaataggtgtttttaaagcctattgattttggtactattaatgtggtcaggaactttctcagtctttcttgtttggggtgagctgtggcttcctaaacaggaacccaagacacccccaaaagctgctcaccagcactgccagcctccctcttaccaagtagcacccgttcaggacattctgcgaaaggcatttgcccagaagttgggaggaaggaaatgtaacattttggggcacctaccatatgccaggcaccaggctaaacgtgttcacacaaattctcttactaaccctcaccatccttctacaagacaaactagtatcttcatcttggggttcaagatgaggaaatggaggctcagagaggttgaatgaatgccggtgcctggatatgaaccccatctgcctgactccgcaacccaggcaaagtctttccttgaacttcccagcagccactgcttagacacagcctccacaaccatggctcagcagcaaattgcttctctgacctcactcagcctgtgtgtccttgttgagtgaggcattcaggaccctggtcccaaagtggagaaagtctttcctactaggtcatagctacacctgcatgtgggtgctgtgccttttgtttagtgaacttttatcaccagcatcctcagcaatgacatttgcagagaagccagagctgaggcaccttggtattcttgggatgtgactttcctgaatgtttaagggaaaatgcccgaaggtacagagagcttggtttctagtaaacaataactgtcttgcttttaccccccttcatttgctgacacatacaccagctgaagaagcaggcattggagacacccccagcctggaagacgaagctgctggtcacgtgacccaaggtcagtgaactggaattgcctgccatgacttgggggttggggggagggacatggggtgggctctgccctgaaaagatcatttggacctgagctctaattcacaagtccaggagattttagggagttggttcttatcaaaggttggctactcagatatagaaagagccctagtggtttttttctaataccatttctgggtaattcctaaggcatttagtgttctgaaagatgctagccttgtccagcctgggagttgagaatgaatgtctaacagaaactctaggccgggcctggtggctcacgcctctaatcccagcactatgggagacccaggtgggcagatcacctgaggtcaggagtttgagaccagcctggccaacatgtgaaatcctgtctcactacaaataaaaaaattagccgggtgtggtggtaggtgcctataatcccagctactcaggaggctgaggcaggacaatcgctcgaacccaggaggtggacgttgcagtgagccgagatcgcatcattgcactccagcctgggcaacaaaagcaaaactccgtctcaaaaaaaaaaaagaaactcaaatatgtgtgacaggcgattctcactgcaggctgccctgtggctgatccaggagcaaggccttaaccatgtcatccccaagcgattgcttgtaaactttcttctgtgcagccttcaacccttattatgattttcttctcaggaaccaaactgctgtattcaagaaaggcagctttgtgtaatcatttatcataaatatcttaagaaaaatcctagagattcctaattttaggaaatgggagacctatggtactgatataatgtgggctgggcttgttttctgtcatttgctagataaatgaacttgagagcctactgtaaaatgtggaagcttctagattgcagaagggctggaaagacactgttcttttctcccgagtgatgggatctgtccagtatttagagctgcctctgaggccatctgattctaggagactctgcctcgttgaggatattttgaggcctaactacacattcctgcccccagagaggtcacagcctatagcaggctgatgtttctcatgtcacatggcacagaaaggcacattttcgttctcaggctaacaaagagcttcaaaaactattagaagggacagtggctataagagaagaacctcagtcaatgtgtgaaattaactaggaacctggctcctgtttcttttaggtcatgtttttcagcttaggtaaaactagaggctttgataaagcatgacctctagaaatcattgcttttcataaatggaagtgggtttgagttttttctactgattgttagtgcaggtgatgtctacatgcccccagaacatattccatgcaacaaaaaaagcccaggtcaccgtctttgctgggaacttgacttttgtgctcactgaattttaagctttctgacagcagcctggaatcatggagggataaagtacctattagtaagatggaaaaaggtgtttcaggttggagctgcagtctgttgagagtaagctatgggaaggcctgtatacgaggggtggacttttcttctgtaagtgtccagagaccaggcctcctgaagagggcatgggggcttaacttacctggactactgtgtttacaatactcatttatcttgaactcctcctaacccctgagaattgctacatttagtatttgctgagtacttcctagcatcctagggaatcaatagaacattctcccaaccaggctgggtgcggtggctcatgtctgtaatcccagcactttgggaggccaaggtaggcagatcccttgaggccaggagtgcaagactagcctggctgacatggtgaaaccccgtctttactaaaaatacaaaagttagccaggcatggtggtacacacctgtaatcccagctacatgggaggagtaggaggcaggagaattgcttgaacctgggaggtggaggttgctgtgagccgagatcatgccactgcactccagcctgggcgacagagtgagtgagactctgtttaaaaaaaaaaaaaaaaagaacattctcctaacctggcttcttcctccaggggtgtaattaatcatgtcagtttcctcattgatacacacacacacacactacaatcctgtatccattacttttcaaggtacatttactatttacgtttggggtccttgtctcttttttaatagtgtttcttaaagtcttgtattatatcagagtacagtaacatcccagtcaagagcactctagtaagctctaggaggaaagcgacttccggaaggcagtggagacctgtcctgttggggcagcataggggcagcccctgcctctggtcagttctggcgctcaggctcagggttgcctctgggctgttcttcccagagactgacaaagggctcccataaggcacctgcagagcctgtgagaagctgaagtcaatgttttcctgacaccagttgatctgtgcaggatccattgatttaaccacctgctgtgtggcatgcactgtggtcgatgccaggaacaggaattggaggggcccatgagcatggccagtatcacaggctggaggtgctgctgcgctctgaccgggcctcttggggatgagcccatgtcaaccaccttgcctccgatggggtcgggcccacaggttacctttgtgtgtccatgaccacaccttcctccccgacctcatccaaatctctttcttttccaagcccctgaatccttcagggctgcaggttttgtttaaagcagagctggtgagttgcataggttgttgcattgggactagatggggtgttcaaagagttgggagttaaaaaacataaagggtatttattaggagaaccaaggagtgtaattctcctgttcttaatatgcggccaggttaatgaatgtcacgtgaatgaaccagaaaaaaatgaagtgtgcccttgatcagctgggttggtgtgcagcaagctgtgtgaccaggggacagcagtggtcctgagggccgtcactgtctgccgtgcagagcccttcctcccacgggggcctacctcacctgtgccaagggcttgtctgtggtcagtgacctggatagatctgaatggggcttctttttcgaggagtcttatggcaggtctctcagtaaagactccattcttgatgatcacacattttggattttccaaatctgtcagagaatgggcttgaggcggggtttgtgggcactagtttcactggtttcatttaccaaaaaggggagcagaagtcaagtatggtggctcatccctgtaatcccagaggcaagagaattgcttgagcccaggagttcgagaccagcctgagcaacataaggagaccccgtctccacaaaaatgaaaaataacattttagtcagacgtggtggcatgcatctgtggtcccagctgcttgggagggtgagatgggagggttgtttgagccctggagttaaagttgcaatgagctgtgattgcaccactgcactctagcctgggtgacagaacgagaccctgtctcaaaaaaaaaaaaaaagaaagaaagaaaggaaaaaaaaaactcatgcctgtaatcccagcactttggggaccggggtgggcagatcacgaggtcaggagatcaagactatcctggccaacatggtgaaaccccgtttctactaaaaatacaaaaattagccaggtgtggtggcacgtgcctgtaatcccagttactcgggaggctgaggcaggagaatcgcttgaaccagggagtcagaggttgcagtgagctgagatcgtgccactgtactccagcctgggcgacagagtgagactctgtctcaaaccaaaaaaaaggggtggggggcgggggcaggagaacagtgagaggtagggagaggaaaggggattctcgctacacccaaaccagataccatctagaggctagaatctttgggaggctcaaattccctagaaagcaggagaagcttctgtagccctcccgctttcccagtagattaagcccagggcggctccagatgtgtgacatgctctgtgcccaaccagagcccatcataggcagaggaataacacccacaccagaagggccctcggaggtcaccacgtccaagaaccctctttacagatgaggaaactgaggcccagagaggggagagccacctagcgagctggtggcggctagaccaggagagctgtcattccaagcaagcaaaggcaacgagacgagcccagagctgtgctcccatctctttgttagggggcctgggatgccctctcagtgtcattttgtccaggatgatgctccctctcttaagcgattaatgcgcccttgctaaccttttgctatcgctgcctcttcaaaccagaggagttgagagttccgggccggcagaggaaggcgcctgaaaggcccctggccaatgagattagcgcccacgtccagcctggaccctgcggagaggcctctggggtctctgggccgtgcctcggggagaaagagccagaagctcccgtcccgctgaccgcgagccttcctcagcaccgtcccgtttgcccagcgcctcctccaacaggaggccctcaggagccctccctggagtggggacaaaaaggcggggactgggccgagaagggtccggcctttccgaagcccgccaccactgcgtatctccacacagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaaggtctgtgtcttgagcttcttcgctccttccctggggacctcccaggcctcccaggctgcgggcactgccactgagcttccaggcctcccgactcctgctgcttctgacgttcctaggacgccactaaatcgacacctgggtgcagctgctccactccctcggcctcctcccgtgctcaggctgtggccgcacgcgcccctcacgcttgcccgccactctgcatgtcaccagcacccccgctccgtgctacccaccttgtttgactctctggccacttgatttgtccacaacggcccatcagcccacaggaggtttggtgggtgccttccaccgacaggatgacgggtgccctcatggtgtctagaactctccaaccctcccatgtaggcataagcagccccactttgcagatgaggaaacggaggctcagagaagtacagtaacttgccgaaggccaatgagtagtaagtgacagagccaggtttgggatccaggtaggttgtctctgaaagacacgcctgtcctgcatcccacaacgcctcccaggaggtgctggagtgtggacgcctaacacagagatgtgcagggcacacacagcaggtgacacacacagcatccagaggtggcccagagctcatgctgtgcctttggcccagtgccctgcccccacccactctgccttgtggcaggaagacaaggagcagacacaagatctccctggtccacatgccaccacctccctctgcagaggacaaggggatcctcatgctggcattggagggggttgagcagggcccaccttgagccctcaggagcacgaccacagcagccctgcagggagggattggtgggaggagagtcccaagtatcagggagaggagagttggtgtcccacaggagacctcagagccacaaggcgagcttgttcataaatttgggacccttagcatttcacagttatttgcagagcccagaaatggatgttactgaagctcacagttgcaagcatctgttaaatttttattagattttacttttagggaaaactttgaaatgctataaagaagcctgtgtttaaaagttaagacagaggctgggggcgatggctcacgcctgtaatctcagcactttgggaggccaaggcaggtggatcatttgaggttaggagttcgagaccagcctggccaacatggtgagaccctgtctctactaaaattacaaaaaattagctgggcgtggtggcgggcacctgtagtcccagctactggggaggctgaagcaggataagtgcttgaacccaggaggcagaggttacagtgagccaagatcacaccactgtaccctaagcctgggcgacagagtgagactctgtctcaaaaaataaaataaaataaagttaagagagaaaaaaatatatcctatatcctttgttaaattccaaaacagtaggggacaaataactgacttgacaggttactacaatatttcctgaaatgatgttttcttgaatactggcctactagaggttcataggtgtgtttggattaaaaaagagttccatggcccagtgactgggggaaaaaaataaaagactaaagtaagttaaacaggcttttctgctgcaggacttgtcagagcctttaatgtactaatggccattgtgaccctctgagaaggtcacagagtgggtttcccaaacttacttgattctacctgctaacatttcctggaggaagtttgggaaatgccgatttagcagattcttttgttgtgccgtggatggtgctggttgatgtgggcaaaacaaagaacacgtgagtcagatccgcctggggctcttactaaagtgcaggttcccaggtgccactttaggcttacagacccagttgtggggtaagcctgggagtcttttagcaggtgattctgccacatagtatagttggaaaacctctgggcatactcattgctggtccctctagaaatccaggtgacaatagccaatgagaagctccaagagacccagttgtccatggggtagagggaatgtgatattgaaaccaaagaagaaaatctatgatcagttttcagcagtgactgtcaagagaaggagaagggtgagttagcgctgatgctggctgacaggtcagcgggttggtttcaccaaggagtgtgatgaaggctgatgttgtctgtgggaatgtatgatggtaactggtttgtagctaatttggggaagcagtgagaattcgtgccctttgaagaccagtaagtggcaagaaacccaccaggcctggctcagggctgggctgggcttggctcgtctcagagcagctggggctggtggccaaagccaccattagtgaggggcaggccctgggggtacaaccagcaactaggggacaaagacaaccctgccagcctctcctattctggaggcgtgtgaccagaaatggagatgggttggtcagcataagatggccaggaaggtggaaatcaggactgctggcaatctagccacatgggcaggggagccgggtggttccaggcagtttccaaggccaagagggtgagcaggcacctcacagggaatcagggccaagcctggctgcagtgtggagacaatgcacccacccccatccttggatcttgcaggaggctgggtcctcactgagctaccaacatccatggccctgaggcttttaaaacacccatccatggagtggggctggtcccagtggggtgaggctgaccctggcagaaacagggcaggagcctgtgggttagggagactgcaccttccttagatagcctccatgccatcatgtccccgtgacagtttctgctgcgtcccctctgcatggtcccaccctcggccagcctgctgccccctcttgccaggttgcgctaatcagtgaccccagtgtgctgtgttgatactaacaatgcgaggcctagcagattcaagggaaaagagaaccaactgggtttccaccagacccaactaaacaaacatggacctatcccagagaaatccagcttcaccacagctggctttctgtgaacagtgaaaatggagtgtgacaagcattcttattttatattttatcagctcgcatggtcagtaaaagcaaagacgggactggaagcgatgacaaaaaagccaaggtaagctgacgatgccacggagctctgcagctggtcaagtttacagagaagctgtgctttatgtctgattcattctcatatataatgtggggagtatttgtcactaaagtacagctgtcatttaaagtgctttgtattttggggcaggcttttaaaaagtccagcatttattagttttgatacttaccccagggaagagcagttggcaggttcatgaagtcatgctcctaattccagctttcttagtgtactttcagtgagaccctgacagtaaatgaaggtgtgtttgaaaaccaaacccaggacagtaaatgaaggtgtgtttgaaaaccagccctaggacagtaaatgaagccatcttctcactgcataaactgcacccagatcttcgcccatccttctcagtatttcacttcacccattgtttactgtctcaatgactggggaaatgtctggggaaatgctcccgtaattgcacagtggcgtttttcctggaaaatcccaccatggctctagataagacctatttttcttaaaggtatctaaaatttccagcataaattctgtctgaaacacctgaattttaatcagtactggagcccggagggcatctccagttgccacatagctctgagcattcagtggtgtgttgagggctgctcccggaagtgcctgcagagtcagggctccccagcctcatctagtgaggcagtggaagggcctgtggggatttggagagctggcctgggtctctgaagtgatagtgacagctgcttgtcaatcacggtgcacatttagtgctgggggcagggggcagggaataccagcctcatgcatgcatgcattcatttgttccttccttcattcattcattcagtacacatgggtacaacatccctgccctggagttgcccagagtctagggaggggaaagatctattaccctgggcctcggccagctggggagtgctgctggtggagaggggccgtgtgcagcgagggaaggaggagtcgtcaatacccccaccccagctttgctttcttgtcatcagccccagggccccagcctgtgtccctcctctcccattgctacttcatctcctgggtcctccttaccaagcctgaccacacagagggccttggccgcttccatggggaattggaaagcaataagatagcatcccctagaagcccagtgaagtctgggacaggacccttctctgagctctgacttgctcttggaaacacttcgaggcttagcctccccactttgtttcccgagagtgtgacctgttcccctccaaacacccccttctcctccagggccatgcccacccgtcaaaatcccccacgggcaggacgaactgtgggtgtcagtcaccatctatcctgcatcctggttccagggccccccccagccccgcctccatagggacaggcgtgcagacacccgtccctggctgcttcctcttgtggaatgggttcaaaagtaagcagtgttgtttacactgacaaactgaaaaaaaaagaaaaagagataacattggaggcttggcacagtggctcatgcctgtaatcccagcactttgggaggctaaggtgggaggatgtccccagcccaagagttctagaccagcctgggcaacatagcaagaccccatctcaaaaaaaaaatttaattggccaggcagaggtgggaggatcacttgaacccaaagggtggaggctgcagtgagccgtgatggcaccactgcactccagccagggcaacagagggagaccctgtctctaaaacaaacaaacaaacaaacaaacaaaagagttaacattggccagattaggattcaccagatagtgttaatattagtttgatttgagactttaatcagaaagcacatgtgtggtgggggtgggtgtaacctaagtcaggtagaatctttccaacttggggggggcacactcctgattgtagccatatgagtctgtcagtgtggtggaagaggccatgggttaatgggcaggtaaaaaagcaccttgcctggaattgagtagaaagtaaggcccttcagaccccgtgacacacttggggacattttcttgagtaacatcctaagattcatgtaccttgatgatctccatcaacttactcatgtgaagcacctttaaaccagtcgtctccaaattcaggggcacagtaacatccaacaggctggagaaagaacgtactagaacttccattcctttttcatgtcctcttctaaaagctttgtcagggccaggcgcggtggctcacgcctgtaatcccagcactttgggaggccgagacgggtggatcacgaggtcaggagatcgagaccatcctggctaacacagtgaaaccccatctctactaaaaatacaaaaaaacgagccgggcgtggtggtgggcgcctgtagtcccagctactcgggaggctgaggcaggagaatggcgtgaacccaggaggcagagcttgcagtgagccgagattgcaccactgcagtccagcctgggcgacagagcgagactccgtctcaaaaaagaaaaagaaaaagaaaaagaactgtgattggggaggacggtcactttcctgttcttactgatcagaagggatattaagggtacctgattcaaacagcctggagatcactgctttcaaccattacctgccttatttatttttagttactgtccttttttcagtttgtttccctcctccatgtgctgacttttattttgattttatttatgtttatgtttaagacatccacacgttcctctgctaaaaccttgaaaaataggccttgccttagccccaaacaccccactcctggtagctcagaccctctgatccaaccctccagccctgctgtgtgcccagagccaccttcctctcctaaatacgtctcttctgtcacttcccgaactggcagttctggagcaaaggagatgaaactcaaggtaaggaaaccacctttgaaaagaaccaggctgctctgctgtggtttgcaaatgtggggtttgtttatttgttttttagcctcaaagacctttcttcaaatgagttctggcatagaagcaccgtgtaaaatagttagaattctgggcaaaggggaaaagagagctgggggccatccctctcagcaccccacaggctctcatagcagcagctcctaagacacctggtgggaccttggtttcgaaatcgctactctaaggctgggcacggtggctcacacctgtaatcccagctctttaggaggccgaggagggtggatcacctgagatcaggagttcgagaccagcctggctaacatggcaaaaccctgtctctactaaaaatacaaaaattagccgggcgtggtggtatgcgtggtggtaatcgcagctactcgggaggctgaggcacaaggattgcttgaaccccagaggcagaggttgtagttagctccagcttgggcgacagagcaagaccctgtcgcaaaaattgtttaaaaaacaaacccaaaattgctactctcattgggttcctttgcccattcctgattttggcaagagaaatgcttccagattgccctgatctgggtaggacagcatcacgccatagcaacactgccccgtgagctcactgccccctcaactagcttgtggtccttggttaatgtcagtttcttttttgagtttgtgttatgtctaagggtcatctgctgggtaacggaacccagggactgccctagtccctagactgtgccatgcccgactctgccagctttgtcagtgatgctggtgctcgcctcctcgggtgctcgcctggtctgagcacacccaaggagttcttgaggccttagggttgtttgcgagagaatgaaagaacacgacctagctctctttagcatccttggtcaggttcaacactgcccccaggggcctctggtggagccaaccaccatcagccaaataaatccataattagagtcagaaaatggatgtctgcatatgtgtagtgcactaatgtcctgccgatgattgacatggagtggagagtgacctgatcattgctgtgagctctgctggccttggcacaactcatgctgataactaatgcacacagttcctctgggaggaaatgtcctcagggaacttggagtttgggtggggatgtgggtttgtgtgcccagcaagcccttgtggttgtagcagacactagtggcatctaggaggcaaagggtcaccccagtcttagccacgttttgagtcaaggtggcggagtggggctggtgttgactcttggtggcagtaacttttcccaatggtgaaaaacccctctatcatgtttcatttacagggggctgatggtaaaacgaagatcgccacaccgcggggagcagcccctccaggccagaagggccaggccaacgccaccaggattccagcaaaaaccccgcccgctccaaagacaccacccagctctggtaagaagaacgttctcttgaatcttagaggaagctgaagctctcagaggtacagccttcattttaggaggccttaggccactgagaatgaataacccctggcagctggtcagcagcttgcagtttactaagcactggagtcttcattgccttctcagtccttttgatttctgaggcaaatgttgaatccctacctttttttttttttttcttttgagacagagtttcgcttttgttatccaggccggagtgcagtggtgtgatctcagctcactgcatcctccacctcccaggttcaagcgattctcctacctcagcctccctagtagctgggattacaggcacctgccactatgcccggctaattttttgtatttttagtagagacagggtttcaccatgttggccaggctggtctcgaacgcctgacctcaggtgatccacctgcctcggcctcccaaagtgctgggattacaggcatgagccaccactcccagcctgaatcctcactttttatcaatgaagaaattgaggctgattctgcagcatgataaaaaaaaatacagaaaaaggaaaaaaaagaaagaaatcgagcctctgagagtttgcttgactgagtctaaccagctcattttaaacccgaggaaaatgcagtcacatgactactaagtggcagctctcggagcctctctggccccaagtccagggttccatagaggcagccccagcatggcatgttttcagtccccaaatgagactctggagacaaatgtctctggagacagagcagcagcctggataagtcacaatgggtgacgtcactcagggctcaacccctgggcagcttaacttgctagggacgttaggagtctgctgcaaaacctgagggtcttagctgagcagtcacaggctgggcccgttgccctgggctcctgtgagtaaaacccagtcaattttgagtacccagtaaggcatccattgagttattttgcagccaggagtgctattaagaacagtcgcggctgggcgtggtggctcatgcctgtaatcccagcactttgggaggccaaggtgggcggatcacctgaggtcaggagttcgagaccagcttggccaacatggcaaaaccccgtctctaataaaaatacaaaataattagctgggcgtggtggcgggcgcctgtaatcccagcttctcaggagggtgaggaaggagaatcacttgaacccaggaggcagaggttgcagtgagctgagatcgcaccattgcactccagcctggatgacaaaagtgagattccttctcaaaaaaaaaaaaaaaaaaacagtcgtcctctttggggattagggacagcctgcctgcctgcccgagcacttctctcttccattgccccagtgaagtattccaggcccctgggtttagactctgcaccatgtaggggtgtctgacctgcacttgctccttggtggcacgggcagcctatggcacttgctgcgggctgtgaccaaagcctggcctggatcttggatcttggtgactctgcttctccctggcctgagggagctgcccagagcctgcccaccacctgctgcgtgtctttgcggtggcatttctcgcacacatgccgtgcagtggcacccccaaggatggccattcactaaggcccattgtttttgtcttttcgcttcgtgttttctggcctggtgtttttctcatatacatgtgatccagggataattcccagaattttgacaggattttaagtagcgtttggatcctgctgtttttttttcacttaacatcgggccagttgactcacactctgttttttgttgttgtttttttgagacggagtctcactgtgtcacccaggctgaagtgcagtggcacaatcttggcatactgcaacctctgcttcccaaattcaagcagttttcctgcctcagcctcctgagtagctgggactacaggcacaggccaccacgccctgctaatttttgtatttttagtaaagacagggtttcaccattttggccagcctagtctcgaactcctgacctcaagtgatccgcccacctcggcctcccaaagtgctgggattacaggggactcacactttgtaacaacctgaaacaacgtgatgcatttccctttgggtcttacctgctcttcggtggctgcctgcaggtggagagaccctcccccttgggcccctcgaccttgtttcagaatggggcccctgctgggccagctgtgggtgcctgccacgtgaaggactcattaaggccctgtttaagcctgatgataataaggctttcgtggatttttctctttaagcgactaagcaagtccagagaagaccaccccctgcagggcccagatctgagagaggtactcgggagcctacttcgctgggagcagcctccctttgcgtgtgtggccattcactggcttgtgtttctagagccgggaggacccttttctgcaatgcagggttcacacagggttcgcagcctgaagatggagcagtccgaattctcttccctgtgcagtttgcgcagctgtgtttgtctgatgggctttctaatcctgtgtgctctccttgacttcagggacaatggcattacaggcatgagccaccatgcctggctgtctccctatgtttcagatgaagacataggcttaaggaggtcaggtgacttgcccacgaccactctgtaaataagaggcatgaaaagtatttggagccaccaccaccaagcccactggtcaccctgggtctctgaagtcagggaggcaggaggatgggaggtctgaggaggcagagaggctgagcctggaggccctggaggccgaggccccatctgttgtttccttatgtggaaaataagaggcttcgtttgtcctattgccacagagcgtactacttcaggaacatccaagacatggaaatccgcagggcacggtggctcacgtctataatcccggcactttgggaggttgaggtgggagaatcgcttgaggccagaagttcaagaccagcctgagcaacatagtcagaccccgtctctataaaaaacattatttttaaaaaagacatggaagtcaaattctaaaaactggtgctggctgggtgcggtggctcatgcctataatcccagcactttgggaggccgaggcgggtggatcacctgaggtcaggagttcaagaccagcctggccaacatggtaaaacctctactaaagaaatctttactgaaaatacaaaaatccagtctctactaaaataagtctctactaaaaatacaaaaattagccaggcgtggtgctgcacacctgtaatatcagctactcgggaggctgaggcaggagactcgcttgatcccatgcagcggaggttgcagtgagccgagatcacgccattgcactccagcctgggcatcagaataagactccgtctcaaaaaaaaaaccacaaaaaaacaaaacaacaacaaaagaaaactagtgcttattcgtcactggccaagctgcccattggctacatgggtgcttcaaagagctgcccttctccaggtctggccagcaggtatgtgttacagcaaatgcctggggcagcggcaggggcattgctgcgggaagcttctggacttgcaggaaagctaagttctcagactgcaggggagctaagcacacctcggcacagggtgaggcctgcggttctcagacttcagtctttgtggagcttgagaaaaatgaggctttgcaggtcccacccctagagattctgctctatccactcttgaaggggatcgagaaatttgcattttgcaactcccactttcctccttgaaagctccggagattctgacgcagggttccgtgggccacactttggaaaatacagacccatgagatagaataccagactgttgaagtgtaacgggggcctgggaagtgcagtaacagaagcaagtttgagggtaaaggacacccagaggagggagggacagcatctgcatggagaggagaagagaccccccagcagcttccagggtgttggaagggtgcgctagtaactgctatgcatggcaggtggggaactgtacgtcagggcacagcagcatgaagcggtatggctcgtgtggacagctagggacaggcaggcgtggagcaggcatcctgttctgaaggccaaatcccacagaggagccagggtgctggcaggagccctgaactagccgaacagctgaacagctgaacattcaccctgtggggaaagggtcagaagcgtccaggcttgagggcacagctgggtctcgtcactgcatcacccttatttaggataaaggccctgaagaattgtattagaggttggcaaagcatatctaccacctcctggagccacgctggccgcagggattataattatttccattttcaaattaaggcctctgagctcagagaggggaagttacttgtctgaggccacacagcttgttggagcccatctcttgacccaaagactgtggagccgagttggccacctctctgggagcgggtattggatggtggttgatggttttccattgctttcctgggaaaggggtgtctctgtccctaagcaaaaaggcagggaggaagagatgcttccccagggcagccgtctgctgtagctgcgcttccaacctggcttccacctgcctaacccagtggtgagcctgggaatggacccacgggacaggcagcccccagggccttttctgaccccacccactcgagtcctggcttcactcccttccttccttcccaggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgagagtggctggctgcgcgtggaggtgtggggggctgcgcctggaggggtagggctgtgcctggaagggtagggctgcgcctggaggtgcgcggttgagcgtggagtcgtgggactgtgcatggaggtgtggggctccccgcacctgagcacccccgcataacaccccagtcccctctggaccctcttcaaggaagttcagttctttattgggctctccactacactgtgagtgccctcctcaggcgagagaacgttctggctcttctcttgccccttcagcccctgttaatcggacagagatggcagggctgtgtctccacggccggaggctctcatagtcagggcacccacagcggttccccacctgccttctgggcagaatacactgccacccataggtcagcatctccactcgtgggccatctgcttaggttgggttcctctggattctggggagattgggggttctgttttgatcagctgattcttctgggagcaagtgggtgctcgcgagctctccagcttcctaaaggtggagaagcacagacttcgggggcctggcctggatccctttccccattcctgtccctgtgcccctcgtctgggtgcgttagggctgacatacaaagcaccacagtgaaagaacagcagtatgcctcctcactagccaggtgtgggcgggtgggtttcttccaaggcctctctgtggccgtgggtagccacctctgtcctgcaccgctgcagtcttccctctgtgtgtgctcctggtagctctgcgcatgctcatcttcttataagaacaccatggcagctgggcgtagtggctcacgcctataatcccagcactttgggaggctgaggcaggcagatcacgaggtcaggagttcgagaccaacctgaccaacagggtgaaacctcgtctctactaaaaatacaaaaatacctgggcgtggtggtggtgcgcgcctataatcccagctactcaggaggctgaggcaggagaatcgcttgaacccaggaggcagaggttgcagtgagccgagatagtgccactgcactccagtttgagcaacagagcgagactctgtctcaaaacaaaataaaacaaaccaaaaaaacccaccatggcttagggcccagcctgatgacctcatttttcacttagtcacctctctaaaggccctgtctccaaatagagtcacattctaaggtacgggggtgttggggaggggggttagggcttcaacatgtgaatttgcggggaccacaattcagcccaggaccccgctcccgccacccagcactggggagctggggaagggtgaagaggaggctgggggtgagaaggaccacagctcactctgaggctgcagatgtgctgggccttctgggcactgggcctcggggagctagggggctttctggaaccctgggcctgcgtgtcagcttgcctcccccacgcaggcgctctccacaccattgaagttcttatcacttgggtctgagcctggggcatttggacggagggtggccaccagtgcacatgggcaccttgcctcaaaccctgccacctccccccacccaggatcccccctgcccccgaacaagcttgtgagtgcagtgtcacatcccatcgggatggaaatggacggtcgggttaaaagggacgcatgtgtagaccctgcctctgtgcatcaggcctcttttgagagtccctgcgtgccaggcggtgcacagaggtggagaagactcggctgtgccccagagcacctcctctcatcgaggaaaggacagacagtggctcccctgtggctgtggggacaagggcagagctccctggaacacaggagggagggaaggaagagaacatctcagaatctccctcctgatggcaaacgatccgggttaaattaaggtccggccttttcctgctcaggcatgtggagcttgtagtggaagaggctctctggaccctcatccaccacagtggcctggttagagaccttggggaaataactcacaggtgacccagggcctctgtcctgtaccgcagctgagggaaactgtcctgcgcttccactggggacaatgcgctccctcgtctccagactttccagtcctcattcggttctcgaaagtcgcctccagaagccccatcttgggaccaccgtgactttcattctccagggtgcctggccttggtgctgcccaagaccccagaggggccctcactggcctttcctgccttttctcccattgcccacccatgcacccccatcctgctccagcacccagactgccatccaggatctcctcaagtcacataacaagcagcacccacaaggtgctcccttccccctagcctgaatctgctgctccccgtctggggttccccgcccatgcacctctgggggcccctgggttctgccataccctgccctgtgtcccatggtggggaatgtccttctctccttatctcttcccttcccttaaatccaagttcagttgccatctcctccaggaagtcttcctggattcccctctctcttcttaaagcccctgtaaactctgaccacactgagcatgtgtctgctgctccctagtctgggccatgagtgagggtggaggccaagtctcatgcatttttgcagcccccacaagactgtgcaggtggccggccctcattgaatgcggggttaatttaactcagcctctgtgtgagtggatgattcaggttgccagagacagaaccctcagcttagcatgggaagtagcttccctgttgaccctgagttcatctgaggttggcttggaaggtgtgggcaccatttggcccagttcttacagctctgaagagagcagcaggaatggggctgagcagggaagacaactttccattgaaggcccctttcagggccagaactgtccctcccaccctgcagctgccctgcctctgcccatgaggggtgagagtcaggcgacctcatgccaagtgtagaaaggggcagatgggagccccaggttatgacgtcaccatgctgggtggaggcagcacgtccaaatctactaaagggttaaaggagaaagggtgacttgacttttcttgagatattttgggggacgaagtgtggaaaagtggcagaggacacagtcacagcctcccttaaatgccaggaaagcctagaaaaattgtctgaaactaaacctcagccataacaaagaccaacacatgaatctccaggaaaaaagaaaaagaaaaatgtcatacagggtccatgcacaagagcctttaaaatgacccgctgaagggtgtcaggcctcctcctcctggactggcctgaaggctccacgagcttttgctgagacctttgggtccctgtggcctcatgtagtacccagtatgcagtaagtgctcaataaatgtttggctacaaaagaggcaaagctggcggagtctgaagaatccctcaaccgtgccggaacagatgctaacaccaaagggaaaagagcaggagccaagtcacgtttgggaacctgcagaggctgaaaactgccgcagattgctgcaaatcattgggggaaaaacggaaaacgtctgttttcccctttgtgcttttctctgttttcttctttgtgcttttctctgttttcaggatttgctacagtgaacatagattgctttggggccccaaatggaattattttgaaaggaaaatgcagataatcaggtggccgcactggagcaccagctgggtaggggtagagattgcaggcaaggaggaggagctgggtggggtgccaggcaggaagagcccgtaggccccgccgatcttgtgggagtcgtgggtggcagtgttccctccagactgtaaaagggagcacctggcgggaagagggaattcttttaaacatcattccagtgcccgagcctcctggacctgttgtcatcttgaggtgggcctcccctgggtgactctagtgtgcagcctggctgagactcagtggccctgggttcttactgctgacacctaccctcaacctcaaccactgcggcctcctgtgcaccctgatccagtggctcattttccactttcagtcccagctctatccctatttgcagtttccaagtgcctggtcctcagtcagctcagacccagccaggccagcccctggttcccacatcccctttgccaagctcatccccgccctgtttggcctgcgggagtgggagtgtgtccagacacagagacaaaggaccagcttttaaaacattttgttggggccaggtgtggtggctcacacctaatcccaacacctggggaggccaaggcagaaggatcacttgagtccaggagttcaagaccagcctgggcaacatagggagaccctgtctctacaattttttttttaattagctgggcctgttggcactctcctgtagttccagctactctagaggctgaggtgggaggactgcttgagcctgggaggtcagggctgcaatgagccatgttcacaccactgaacgccagcctgggcgagaccctgtatcaaaaaagtaaagtaaaatgaatcctgtacgttatattaaggtgccccaaattgtacttagaaggatttcatagttttaaatacttttgttatttaaaaaattaaatgactgcagcatataaattaggttcttaatggaggggaaaaagagtacaagaaaagaaataagaatctagaaacaaagataagagcagaaataaaccagaaaacacaaccttgcactcctaacttaaaaaaaaaaatgaagaaaacacaaccagtaaaacaacatataacagcattaagagctggctcctggctgggcgcggtggcgcatgcctgtaatcccaacactttgggaggccgatgctggaggatcacttgagaccaggagttcaaggttgcagtgagctatgatcataccactacaccctagcctgggcaacacagtgagactgagactctattaaaaaaaaaatgctggttccttccttatttcattcctttattcattcattcagacaacatttatggggcacttctgagcaccaggctctgtgctaagagcttttgcccccagggtccaggccaggggacaggggcaggtgagcagagaaacagggccagtcacagcagcaggaggaatgtaggatggagagcttggccaggcaaggacatgcagggggagcagcctgcacaagtcagcaagccagagaagacaggcagacccttgtttgggacctgttcagtggcctttgaaaggacagcccccacccggagtgctgggtgcaggagctgaaggaggatagtggaacactgcaacgtggagctcttcagagcaaaagcaaaataaacaactggaggcagctggggcagcagagggtgtgtgttcagcactaaggggtgtgaagcttgagcgctaggagagttcacactggcagaagagaggttggggcagctgcaagcctctggacatcgcccgacaggacagagggtggtggacggtggccctgaagagaggctcagttcagctggcagtggccgtgggagtgctgaagcaggcaggctgtcggcatctgctggggacggttaagcaggggtgagggcccagcctcagcagcccttcttggggggtcgctgggaaacatagaggagaactgaagaagcagggagtcccagggtccatgcagggcgagagagaagttgctcatgtggggcccaggctgcaggatcaggagaactggggaccctgtgactgccagcggggagaagggggtgtgcaggatcatgcccagggaagggcccaggggcccaagcatgggggggcctggttggctctgagaagatggagctaaagtcactttctcggaggatgtccaggccaatagttgggatgtgaagacgtgaagcagcacagagcctggaagcccaggatggacagaaacctacctgagcagtggggctttgaaagccttggggcggggggtgcaatattcaagatggccacaagatggcaatagaatgctgtaactttcttggttctgggccgcagcctgggtggctgcttccttccctgtgtgtattgatttgtttctcttttttgagacagagtcttgctgggttgcccaggctggagtgcagtggtgcgatcatagctcactgcagccttgaagtcctgagctcaagagatccttccacctcagcctcctgagtagttgggaccacaggcttgcaccacagtgcccaactaatttcttatattttttgtagagatggggtttcactgtgtcgcccaggatggtcttgaactcctgggctcaagtgatcctcctgcctcagcctcgcaaattgctgggattacaggtgtgagccaccatgcccgaccttctctttttaagggcgtgtgtgtgtgtgtgtgtgtgtgggcgcactctcgtcttcaccttcccccagccttgctctgtctctacccagtcacctctgcccatctctccgatctgtttctctctccttttacccctctttcctccctcctcatacaccactgaccattatagagaactgagtattctaaaaatacattttatttatttattttgagacagagtctcactctgtcacccaggctggagtgcagtggtgcaatctcggctcactgcaacctccgcctcccaggttgaagcaactctcctgcctcagcctccctagtagctgggattacaagcacacaccaccatgcctagcaaatttttatatttttagtagaggaggggtgtcaccatgtttgccaagctggtctcaaactcctggcctcaggtgatctgcctaccttggtctcccaaagtgctgggattacaggtgtgagccaccacgcctgcccttaaaaatacattatatttaatagcaaagccccagttgtcactttaaaaagcatctatgtagaacatttatgtggaataaatacagtgaatttgtacgtggaatcgtttgcctctcctcaatcagggccagggatgcaggtgagcttgggctgagatgtcagaccccacagtaagtggggggcagagccaggctgggaccctcctctaggacagctctgtaactctgagaccctccaggcatcttttcctgtacctcagtgcttctgaaaaatctgtgtgaatcaaatcattttaaaggagcttgggttcatcactgtttaaaggacagtgtaaataattctgaaggtgactctaccctgttatttgatctcttctttggccagctgacttaacaggacatagacaggttttcctgtgtcagttcctaagctgatcaccttggacttgaagaggaggcttgtgtgggcatccagtgcccaccccgggttaaactcccagcagagtattgcactgggcttgctgagcctggtgaggcaaagcacagcacagcgagcaccaggcagtgctggagacaggccaagtctgggccagcctgggagccaactgtgaggcacggacggggctgtggggctgtggggctgcaggcttggggccagggagggagggctgggctctttggaacagccttgagagaactgaacccaaacaaaaccagatcaaggtctagtgagagcttagggctgctttgggtgctccaggaaattgattaaaccaagtggacacacacccccagccccacctcaccacagcctctccttcagggtcaaactctgaccacagacatttctcccctgactaggagttccctggatcaaaattgggagcttgcaacacatcgttctctcccttgatggtttttgtcagtgtctatccagagctgaagtgtaatatatatgttactgtagctgagaaattaaatttcaggattctgatttcataatgacaaccattcctcttttctctcccttctgtaaatctaagattctataaacggtgttgacttaatgtgacaattggcagtagttcaggtctgctttgtaaatacccttgtgtctattgtaaaatctcacaaaggcttgttgccttttttgtggggttagaacaagaaaaagccacatggaaaaaaaatttcttttttgtttttttgtttgcttgtttttttgagacagagtttcactctgtcgcccaggctggagtgcagtggtgcgatctccgcccactgcaagctccacctcccgggttcatgctattctcctgtctcagcctcccaagtagctgggactgcaggtgcccgccaccacacctggctaatttttttgtatttttagtagagacggggtttcaccgtgttagccaggatggtctcaatctcctgacctcgtcatctgcctgcctcggcctcccaaagtgctgagattacaggcgtgagccaccgtgcccggccagaaaaaaacatttctaagtatgtggcagatactgaattattgcttaatgtcctttgattcatttgtttaatttctttaatggattagtacagaaaacaaagttctcttccttgaaaaactggtaagttttctttgtcagataaggagagttaaataacccatgacatttccctttttgcctcggcttccaggaagctcaaagttaaatgtaatgatcactcttgtaattatcagtgttgatgcccttcccttcttctaatgttactctttacattttcctgctttattattgtgtgtgttttctaattctaagctgttcccactcctttctgaaagcaggcaaatcttctaagccttatccactgaaaagttatgaataaaaaatgatcgtcaagcctacaggtgctgaggctactccagaggctgaggccagaggaccacttgagcccaggaatttgagacctgggctgggcagcatagcaagactctatctccattaaaactatttttttttatttaaaaaataatccgcaaagaaggagtttatgtgggattccttaaaatcggagggtggcatgaattgattcaaagacttgtgcagagggcgacagtgactccttgagaagcagtgtgagaaagcctgtcccacctccttccgcagctccagcctgggctgaggcactgtcacagtgtctccttgctggcaggagagaatttcaacattcaccaaaaagtagtattgtttttattaggtttatgaggctgtagccttgaggacagcccaggacaactttgttgtcacatagatagcctgtggctacaaactctgagatctagattcttctgtggctgcttctgacctgagaaagttgcggaacctcagcgagcctcacatggcctccttgtccttaacgtggggacggtgggcaagaaaggtgatgtggcactagagatttatccatctctaaaggaggagtggattgtacattgaaacaccagagaaggaattacaaaggaagaatttgagtatctaaaaatgtaggtcaggcgctcctgtgttgattgcagggctattcacaatagccaagatttggaagcaacccaagtgtccatcaacagacaaatggataaagaaaatgtggtgcatatacacaatggaatactattcagccatgaaaaagaatgagaatctgtcatttgaaacaacatggatggaactggaggacattatgttaagtgaaataagccagacagaaggacagacttcacatgttctcacacatttgtgggagctaaaaattaaactcatggagatagagagtagaaggatggttaccagaggctgaggagggtggaggggagcagggagaaagtagggatggttaatgggtacaaaaacgtagttagcatgcatagatctagtattggatagcacagcagggtgacgacagccaacagtaatttatagtacatttaaaaacaactaaaagagtgtaattggactggctaacatggtgaaaccccgtctctactaaaaatacaaaaattagctgggcatggtggctcacgcctgtaatcccagcactttgggaggccgaggcgggccgatcacgaggtcaggagatcgagaccatcctagctaacatggtgaaaccccgtctctactacaaatacaaaaaaaagaaaaaattagccgggcatggtggtgggcgcctgtagtcccagctactcgggaggctgaggcaggagaatggcgtgaacccgggaggcggagcttgcagtgagccgagatcgcgccactgcactccagcctgggcgacaaggcaagattctatctcaaaaaaataaaaataaaataaaataaaataataaaataaaataaaataaaataaaataaataaaataaaataaaatgtataattggaatgtttataacacaagaaatgataaatgcttgaggtgatagataccccattcaccgtgatgtgattattgcacaatgtatgtctgtatctaaatatctcatgtaccccacaagtatatacacctactatgtacccatataaatttaaaattaaaaaattataaaacaaaaataaataagtaaattaaaatgtaggctggacaccgtggttcacgcctgtaatcccagtgctttgtgaggctgaggtgagagaatcacttgagcccaggagtttgagaccggcctgggtgacatagcgagaccccatcatcacaaagaatttttaaaaattagctgggcgtggtagcacataccggtagttccagctacttgggagaccgaggcaggaggattgcttgagcccaggagtttaaggctgcagtgagctacgatggcgccactgcattccagcctgggtgacagagtgagagcttgtctctattttaaaaataataaaaagaataaataaaaataaattaaaatgtaaatatgtgcatgttagaaaaaatacacccatcagcaaaaagggggtaaaggagcgatttcagtcataattggagagatgcagaataagccagcaatgcagtttcttttattttggtcaaaaaaaataagcaaaacaatgttgtaaacacccagtgctggcagcaatgtggtgaggctggctctctcaccagggctcacagggaaaactcatgcaacccttttagaaagccatgtggagagttgtaccgagaggttttagaatatttataactttgacccagaaattctattctaggactctgtgttatgaaaataacccatcatatggaaaaagctcctttcagaaagaggttcatgggaggctgtttgtattttttttttctttgcatcaaatccagctcctgcaggactgtttgtattattgaagtacaaagtggaatcaatacaaatgttggatagcaggggaacaatattcacaaaatggaatgggacatagtattaaacatagtgcttctgatgaccgtagaccatagacaatgcttaggatatgatatcacttcttttgttgttttttgtattttgagacgaagtctcattctgtcacccaggctggagttcagtggcgccatctcagctcactgcaacctccatctcccgggttcaagctattctccttcctcaacctcccgagtagctgggttgcgcaccaccatgcctggctaacttttgtatttttagtacagacggggtttcaccacgttggccaggctgctcttgaactcctgacgtcaggtgatccaccagccttgacctcccaaagtgctaggattacaggagccactgtacccagcctaggatatgatatcacttcttagagcaagatacaaaattgcatgtgcacaataattctaccaagtataggtatacaggggtagttatatataaatgagacttcaaggaaatacaacaaaatgcaatcgtgattgtgttagggtggtaagaaaacggtttttgctttgatgagctctgttttttaaaatcgttatattttctaataaaaatacatagtcttttgaaggaacataaaagattatgaagaaatgagttagatattgattcctattgaagattcagacaagtaaaattaaggggaaaaaaaacgggatgaaccagaagtcaggctggagttccaaccccagatccgacagcccaggctgatggggcctccagggcagtggtttccacccagcattctcaaaagagccactgaggtctcagtgccattttcaagatttcggaagcggcctgggcacggctggtccttcactgggatcaccacttggcaattatttacacctgagacgaataaaaaccagagtgctgagattacaggcatggtggcttacgcttgtaatcggctttgggaagccgaggtgggctgattgcttgagcccaggagtttcaaactatcctggacaacatagcatgacctcgtctctacaaaaaatacaaaaaatttgccaggtgtggtggcatgtgcctgtggtcccagctacttgggaggctgaagtaggagaatcccctgagccctgggaagtcgaggctgcactgagccgtgatggtgtcactgcactccagcctgggtgacaaagtgagaccctatctcacaaagaaaaaaaacaaaacaaaaaacccaaagcacactgtttccactgtttccagagttcctgagaggaaaggtcaccgggtgaggaagacgttctcactgatctggcagagaaaatgtccagtttttccaactccctaaaccatggttttctatttcatagttcttaggcaaattggtaaaaatcatttctcatcaaaacgctgatattttcacacctccctggtgtctgcagaaagaaccttccagaaatgcagtcgtgggagacccatccaggccacccctgcttatggaagagctgagaaaaagccccacgggagcatttgctcagcttccgttacgcacctagtggcattgtgggtgggagagggctggtgggtggatggaaggagaaggcacagcccccccttgcagggacagagccctcgtacagaagggacaccccacatttgtcttccccacaaagcggcctgtgtcctgcctacggggtcagggcttctcaaacctggctgtgtgtcagaatcaccaggggaacttttcaaaactagagagactgaagccagactcctagattctaattctaggtcagggctaggggctgagattgtaaaaatccacaggtgattctgatgcccggcaggcttgagaacagccgcagggagttctctgggaatgtgccggtgggtctagccaggtgtgagtggagatgccggggaacttcctattactcactcgtcagtgtggccgaacacatttttcacttgacctcaggctggtgaacgctcccctctggggttcaggcctcacgatgccatccttttgtgaagtgaggacctgcaatcccagcttcgtaaagcccgctggaaatcactcacacttctgggatgccttcagagcagccctctatcccttcagctcccctgggatgtgactcaacctcccgtcactccccagactgcctctgccaagtccgaaagtggaggcatccttgcgagcaagtaggcgggtccagggtggcgcatgtcactcatcgaaagtggaggcgtccttgcgagcaagcaggcgggtccagggtggcgtgtcactcatccttttttctggctaccaaaggtgcagataattaataagaagctggatcttagcaacgtccagtccaagtgtggctcaaaggataatatcaaacacgtcccgggaggcggcagtgtgagtaccttcacacgtcccatgcgccgtgctgtggcttgaattattaggaagtggtgtgagtgcgtacacttgcgagacactgcatagaataaatccttcttgggctctcaggatctggctgcgacctctgggtgaatgtagcccggctccccacattcccccacacggtccactgttcccagaagccccttcctcatattctaggagggggtgtcccagcatttctgggtcccccagcctgcgcaggctgtgtggacagaatagggcagatgacggaccctctctccggaccctgcctgggaagctgagaatacccatcaaagtctccttccactcatgcccagccctgtccccaggagccccatagcccattggaagttgggctgaaggtggtggcacctgagactgggctgccgcctcctcccccgacacctgggcaggttgacgttgagtggctccactgtggacaggtgacccgtttgttctgatgagcggacaccaaggtcttactgtcctgctcagctgctgctcctacacgttcaaggcaggagccgattcctaagcctccagcttatgcttagcctgcgccaccctctggcagagactccagatgcaaagagccaaaccaaagtgcgacaggtccctctgcccagcgttgaggtgtggcagagaaatgctgcttttggcccttttagatttggctgcctcttgccaggagtggtggctcgtgcctgtaattccagcactttgggagactaaggcgggaggttcgcttgagcccaggagttcaagaccagcctgggcaacaatgagacccctgtgtctacaaaaagaattaaaattagccaggtgtggtggcacgcacctgtagtcccagctacttgggaggctgaggtgggaggattgcctgagtccgggaggcggaagttgcaaggagccatgatcgcgccactgcacttcaacctaggcaacagagtgagactttgtctcaaaaaacaatcatataataattttaaaataaatagatttggcttcctctaaatgtccccggggactccgtgcatcttctgtggagtgtctccgtgagattcgggactcagatcctcaagtgcaactgacccacccgataagctgaggcttcatcatcccctggccggtctatgtcgactgggcacccgaggctcctctcccaccagctctcttggtcagctgaaagcaaactgttaacaccctggggagctggacgtatgagacccttggggtgggaggcgttgatttttgagagcaatcacctggccctggctggcagtaccgggacactgctgtggctccggggtgggctgtctccagaaaatgcctggcctgaggcagccacccgcatccagcccagagggtttattcttgcaatgtgctgctgcttcctgccctgagcacctggatcccggcttctgccctgaggccccttgagtcccacaggtagcaagcgcttgccctgcggctgctgcatggggctaactaacgcttcctcaccagtgtctgctaagtgtctcctctgtctcccacgccctgctctcctgtccccccagtttgtctgctgtgaggggacagaagaggtgtgtgccgcccccacccctgcccgggcccttgttcctgggattgctgttttcagctgtttgagctttgatcctggttctctggcttcctcaaagtgagctcggccagaggaggaaggccatgtgctttctggttgaagtcaagtctggtgccctggtggaggctgtgctgctgaggcggagctggggagagagtgcacacgggctgcgtggccaacccctctgggtagctgatgcccaaagacgctgcagtgcccaggacatctgggacctccctggggcccgcccgtgtgtcccgcgctgtgttcatctgcgggctagcctgtgacccgcgctgtgctcgtctgcgggctagcctgtgtcccgcgctctgcttgtctgcggtctagcctgtgacctggcagagagccaccagatgtcccgggctgagcactgccctctgagcaccttcacaggaagcccttctcctggtgagaagagatgccagcccctggcatctgggggcactggatccctggcctgagccctagcctctccccagcctgggggccccttcccagcaggctggccctgctccttctctacctgggacccttctgcctcctggctggaccctggaagctctgcagggcctgctgtccccctccctgccctccaggtatcctgaccaccggccctggctcccactgccatccactcctctcctttctggccgttccctggtccctgtcccagcccccctccccctctcacgagttacctcacccaggccagagggaagagggaaggaggccctggtcataccagcacgtcctcccacctccctcggccctggtccaccccctcagtgctggcctcagagcacagctctctccaagccaggccgcgcgccatccatcctccctgtcccccaacgtccttgccacagatcatgtccgccctgacacacatgggtctcagccatctctgccccagttaactccccatccataaagagcacatgccagctgacaccaaaataattcgggatggttccagtttagacctaagtggaaggagaaaccaccacctgccctgcaccttgttttttggtgaccttgataaaccatcttcagccatgaagccagctgtctcccaggaagctccagggcggtgcttcctcgggagctgactgataggtgggaggtggctgcccccttgcaccctcaggtgaccccacacaaggccactgctggaggccctggggactccaggaatgtcaatcagtgacctgccccccaggccccacacagccatggctgcatagaggcctgcctccaagggacctgtctgtctgccactgtggagtccctacagcgtgccccccacaggggagctggttctttgactgagatcagctggcagctcagggtcatcattcccagagggagcggtgccctggaggccacaggcctcctcatgtgtgtctgcgtccgctcgagcttactgagacactaaatctgttggtttctgctgtgccacctacccaccctgttggtgttgctttgttcctattgctaaagacaggaatgtccaggacactgagtgtgcaggtgcctgctggttctcacgtccgagctgctgaactccgctgggtcctgcttactgatggtctttgctctagtgctttccagggtccgtggaagcttttcctggaataaagcccacgcatcgaccctcacagcgcctcccctctttgaggcccagcagataccccactcctgcctttccagcaagatttttcagatgctgtgcatactcatcatattgatcacttttttcttcatgcctgattgtgatctgtcaatttcatgtcaggaaagggagtgacatttttacacttaagcgtttgctgagcaaatgtctgggtcttgcacaatgacaatgggtccctgtttttcccagaggctcttttgttctgcagggattgaagacactccagtcccacagtccccagctcccctggggcagggttggcagaatttcgacaacacatttttccaccctgactaggatgtgctcctcatggcagctgggaaccactgtccaataagggcctgggcttacacagctgcttctcattgagttacacccttaataaaataatcccattttatcctttttgtctctctgtcttcctctctctctgcctttcctcttctctctcctcctctctcatctccaggtgcaaatagtctacaaaccagttgacctgagcaaggtgacctccaagtgtggctcattaggcaacatccatcataaaccaggtagccctgtggaaggtgagggttgggacgggagggtgcagggggtggaggagtcctggtgaggctggaactgctccagacttcagaaggggctggaaaggatattttaggtagacctacatcaaggaaagtgttgagtgtgaaacttgcgggagcccaggaggcgtggtggctccagctcgctcctgcccaggccatgctgcccaagacaaggtgaggcgggagtgaagtgaaataaggcaggcacagaaagaaagcacatattctcggccgggcgctgtggctcacgcctgtaattccagcactttgggaggccaaggtgggtggatcatgaggtcaggagattgagaccatcctggctaacacagtgaaaccccgtctctactaaaaatacaaaaaattagccgggcgtggtggtgggcgcctgtagtcccagctactccggaggctgaggcaggaaaatggcgtgaacccggaaggcggagcttgcagtgagcggagtgagcagagatcgcgccactgcactccagcctgggcgacagagcgagactccgtctcaaaaaaaaaaagcacatgttctcgcttctttgtgggatccaggagatagagaatagaaggatggttaccagaggctgggaagggtagtgaggggatggtggggggatggtcaatgggtacaaaaaaaatagaataagacctagtatttgatagtgcaacagggtgactatagtcaataataatttaattgtacatttaaaaataactaaaagatagccgggtgcagtggcttacgtctgtaatcccagtactttgggaggctgaggtgggcgtttgagaccagcctggccaacatggtgaaaccccatctctactaaaaatacaaaaattagccaggcatggtggcgggcgcctgtaatcccagctactcgggaggctgaggcaggagaatcacttgaacctgggaggcagaggttgcagtgagccgagatcttgccactgcactccagcctgggtgacagtgaaactccgtctcaaaaataaaaataaaaatacagctgggcacggtggctcacgcctgtaatcccagcactttgggaggccgaggcgagcggatcacaaggtcaggagatatagaccatcctggctaacacggtgaaacccggtctctactaaaaatacaaaaaattagccaggcgtggtggcaggtgcctatagtcccagctactcacaaggctgaggcaggagaatggcatgaacctgggaggcggagcttgcagtgagccgagattgtgccactgcactccagcctgggcgagagagtgagactccgtctcaaaacaaaaacaaaaacaaaaacaaaaacaaacacacaacaaaaacctaaaagaatataaatggattgtttgtaacacaaaggacaaatgtttgaggggatggataccccattttccatgatgtgattattatacattgtgtgtctgtatcaaaacatctcatgagccccataaatatatacacctaactatgtacccacaaaaattaaaaaaatatattttttaaggtgaagagggaggcgagatgctggccttaacccctaacccgttgttctccctgcaagctgtccacagggcctctcagactcgaggttcagctatatggatgcatgagcttggtccccagccaacatgggagacacttcaccatcggcagcagctacagcacaggaaccctgggtcactgccatgtcccctctgtgactttgtttaaacagaaaatgatgctctgggccggctgtggtggcccacacctataatcccagcaccttgggaggcgggggtgggcagattgcctgaggtcaggagttggagatcagcctggccgacatggcgaaaccccatgtctactaaaaatacaaaaactagccaggcatggtggcacatgcctgtaatcccagctacttgggaggctgaagcaggagaatcacttgaacccaggaggcagaggctgagtgagccaagatcgtgccaatgcactccagcttgggtgagggagtgagactccgtctcaaaaaaaaaaaaaaagaaagaaaaagaaaagaaagtgatcctactggaaccatgcttactcccctccccacctcacactgtgtagaaattagtgctgtcggccaggcgcggtggctcatgcctgtaatcgcagcactttgggaggccaaggcaggcggatcacgaggtcaggagatcaagaccatcctggctaacacagtgaaaccctgtctctactaaaaatacaaaaaattagccgggcatggtggcaggcacctgtagtcccaactacttgggaggctgaggcaggagaatggcatgaacctgggaggcggagcttgcagtgagccaagatcgcgccactgcataccagcctaggtgacagagtgagactcagcaaaaaaagaaagaaagaaagaaagaaatcagtgctgtctatacttctttctgcagtgatggaaatattctgtatctgtgctgtccagtatagtagccactagctacatgtggcacttgaaacatggctggtacagttgaggaagagtggctgccatatcggacgacacagctatagattctgtcaccccaccccgagagtccagagcggggacttctgccttaggccctattcagggctgatttttacttgaacccttactgtgggaagagaaggccatgagaagttcagtctagaatgtgactccttattttctggctcccttggacactttgtgggatttagtctccctgtggaaagtattccacaagtggtgccaccaccccagctgtgagagcagctgggagctgcttttgtcatctttccctggaaagtcctgtgggctgtctcttcctcatgccttgtcccatgcttgggcatggtgtcaagcgtcaggagggagaaagggtccttatttatttatttagagagggacccttcttctgttcccaggctggagtgcagtggtgcgatctcggctcactgcaacctccgcctcctgggttcaagtgattctcctgcctcagcctcctgagtagctgagattacaggcacatgccaacatgcctggctaattttttttttttttttttttttttttttttgagatggagttgtactctcattgcccaggctggaatgtaatggcacaatctcggctcactgcaacctccacctcctggattcaagcaattctcctgtctcagcttcccaagtagctgggattacaggtgcccgccaccatgctcaactaatttttgtattttttttttagtagagacgaggtttcaccatgttggtcagactggtctcaaactcctgacctcaggtgatccacctgcctcggcctcccaaagtgctaggattacaggcatgagccaccacgcccggcctgaaagggttcttatttagtgtgcattttgacattcaatttaattccaaggtcttgtggggtcatggtttacaggatgttgatatagaaaagacttcacttaatgggccgggcgcagtggctcatgcctgtaatcccagcactttgggaggccgaggcaggcagatcaggaggtcaggagattgagaccatcctggctaacacagtgaaaccccatctctactgaaaatacaaaaaattagctgggcgtggtggcaggcacctgtagtcccagccactcggttggctgaggcaggagaatggcatgaacccgggaggcggagcttgcagtgagcagagaccatgccactgcactccagcctgggcgacagagcaagactctgtctcaagaaaaaaaaaaaaaaaacagactttacttactggaagccaaccaatgtatatttagagtaatttttcctgggctgagctgtcatttacttttgcagtatctcaagaagaagagtttacagtgtaaatatttgatgcacactttgattatatagatgaagcaaactattttcaagagctttgcaaggacttacttgtatccaaacaccattctaaaggagtcttacctacttctaaaggctggtctctacttggaaccacttgcttggccctggttcaagtcctgctgcaaacctggaagtcctgtcattgtcttcttccctccagagcagtggcacccaatctaatttttgctgtgccccagcagcccctggcactttgccctgtagactgcagacctcatgtaatgtatgttaagtccacagaaccacagaagatgatggcaagatgctcttgtgtgtgttgtgttctaggaggtggccaggtggaagtaaaatctgagaagcttgacttcaaggacagagtccagtcgaagattgggtccctggacaatatcacccacgtccctggcggaggaaataaaaaggtaaagggggtagggtgggttggatgctgcccttgggtatatgggcattaatcaagttgagtggacaaaggctggtccagttcccagaggaggaaaacagaggcttctgtgttgactggctggatgtgggccctcagcagcatccagtgggtctccactgcctgtctcaatcacctggagctttagcacgtttcacacctgggccccaacctggagaggctgaccaatgggtctcaggggcagctcggttgctggagtttttgtttttatttatttttatgtatttaaggcagggtctctgtattagtccattctcacactgctaataaagacatacccaagactgggtaatttataaaggaaagaggtttaatggactcacagttccacatggctggggaggcctcaaaatcatggcggaaggcaaaggagaagcaaaggcatttcttacatggcgacaggcaagagagcgtgtgcaggggaactcccatttataaaaccatcagacctcatgagatttattcactatcatgagaacagcatgggaaagacccgcccccatgattcagttacctcccactgggtccctcccatgacacatggaattatgggagctacaattcaagatgagatttgggtggggacacagccaaaccatatcagtctccctctgtcatccaggctggagtgcactggcatgatctcggctcactgcagcctctacctccctgggtcaggtgatcttcccacctcagcctcccaggtagctggaactacaggtacctgccactatgcctggctaaatattttgtatttcctgtggagacgaggttttgccacgttgcccaggctggtcttgaactcctgaggtcaagcaatatgcccacctcggcctcccaaggtgctgggattacaggtgtgagccacagtgctcggcctaagtcactgcagtttttaaagctcccaggtgattcttcagtgcagtcaaaagtgagaactggctgggtgcggtggctcatgcctgtaatcccagcaccttgggaggcgaaggtgggcagatggcttgaggtcaggagttcaagaccagcctggccaacatggtaaaaccccatctctactaaaaatacaaaagttagctgggtgtggtggtgcgtgcctgtaatcccagctacttgggaggctgaggcatgagaattgcttgaacccaggggacagaggttgtagtgagccgagatcgtgccactgcactccagcctgggcaacagagtgagattccatctcacaaaaaaaaaaaaaaaagcgagaaccactgtcctaggccctgatgtttgcaggcaactaaaaaaggaagtggacatccccagtcagctgtggcgcaccaagaacaagtcatgggaacataacctaattttctaaatgggttactaggcacttagagcaaaacaatgatgccgaaatcctgatttcagcaaagcctctgcctgcctgtcttggaagtatccacatgaggctgctggggccttggtgtccccagcagtttctagtctctaggtcttgctgtgggtgtctgtgcagtgagggtgtgtgtggcgctgggtgagctctgtctaggcctggcacaggatgcggtctggtagctgctgcttctcttctgcagaagcgcagccaagcaccctctggggtttcaggcccacacccagcctgaagttctgggagtggctcactttccaaccttcagggtctcccagcagctgactggggagtggtggagggaaaagggattgtattagtccgttttcacgccgctgatgaagacatacccgatactgggcagtctaaaagatagaggtctgatggactcacagttccacgtgactggggaggcctgacaatcatggtggaaggtgaaaggcttgtctcacacggtggcagacaagagaaaagagcttgtgcaggggaactcccctttataaaaccatcagatctcgggagacttattcactatcatgagaacagcacgggaaagaccctcctctatgattcaattacctcccaccaggtccctcccacaacatgtaggaattgtgggaactacaattcaagatgacatttgggtggggacacagccaaaccatatcagggcgtcccagaaagggtatagggtctgagacccaagtcagcatgagaaagtatgcttctcatggtggcccagttgggtggaagtggcagccgggccgtctttccaccaggccactcaagtagcagctgagagacccctgccctggccagtccccgccctcccctcttgccactgcctctggttctgaacagatgggcaccctcatcttgtatttgtgattaatgtctaacaatgtagttttgtgagaagggtttgctgatacagccttgctgcagatgctgcgaactgtggcctggggcagaccttacctccagacacgccctgaggcaggggagggcactggcccgtagctggccgagagctctcgggttgcgcgacagggatacttttcagcggctgggtcgctatccaaagtgagaaaacgaggagggaccaggaggctgtccgcctcaagagatgtgggggccaggtccagttatctggggaagcagtaagcttctctgctgtttctaaccccaggcctcccctggtctaaggcagggcctcccagcctcggggcactttaaagatatctgggcctggccccatccccacagtctgactgagtgggtctggatagggcctgagcattggtgatttcctgggtgaaaggaggcccctcacagtctctggaagcttctctgtgttaggaaaagctctgggcttgactctgctttgaaagtcaagatccgcaaatcctctcagcctcagtttctccttcagcaagatgaaatggaaatgctgtacctacgtcccggggtggttgtgagacccaaaaaagacaatgttctggaaggttcctggtgcgttgcagtcctctaagaacctgagttagagccacgctgagtctcagcttcttggctccttctgtttcaaactcgtccatgtgatagctcaggaagggtaggcagggccctgccccctactcagaaaacaccatcctggtcctggggatccccgcagcattagtcccctgttttcccagtgtattgagaaaaattgctaacaagcagtggggcacaccaccagcctcctgggttcctttcagtttggggatttttggacattcccaggaatgtcttaaaaaacacttcaaaaaacattaacataaatatttttatcaaagcctgtattaaatggtctttcaagaaaatacagtaacaggtcaggcatggtggctcatgcctgtaaccccagcactttgggaggccaaggcaggcagatcacctgaaatcaggagttcaagaccaacctggccaacacagccaaatcccatctctacaaaaaatacaaaaattagctgggtgtggtggcacacacctgtagtcccagctacttgggaggccgaggcaggagaattgcttgatcccggaggcggaggttgcagtgagctgagatcgtgccactgcactccagcgtgggtgacaaggtgaatctttgtctcaaaaaaaaaaaaaaaaaaaagataaaatacagtatacagtaatagagaacaatccttttttcaaagtagtgaccccaaatgaacaaaatatgcatctagcttaaatgcgaacctggttttctctacgcccattcaagcccctgcaataggggcccttcaccccgcatccatggactcctaaaattatatggaaaatggctgtgtgtgagtgtggatggacatgtgcacacatatttttggctttaccagatgctcaaagagcctaggacccaaaaagggctgagaatgaccgtgtcggccacttcagggtcatcaggaattgctgtgcactgctcacttctccagtgaacactttctgcttctgtgtttcctggtatcctttgggactcctggctaggtcatgtgtttctctactttcaaaagggcttcagccaggcacgatggcatgagcctgtagtcccagttgctctggaggttaaggtgggaagattgcttgagcccaggaatttgaggccagcctgggcaagtagataggtagatgattgatagatagatagatagataaatagatggatagataagtcgctagacagtcatccatccacccatccacacataaaaaggcctttgtcatgtcatgttttgtggcccacctgccagtgttgcccacagttgctgcccctccaaactcatcagtcactggcaaacaggaggaatgtgtggctcatgtctgggcatcagtggctgtgggagacatccttgatcttctccagcttctccttccacattttcctttgcaatctggcaatatctattaaaataaaatgtgcatgccttttgacctaagagcttcacttctaggacccacttacacgtgtgtgacatgatgttcatacgggtttatttatctgaggttgttcatacacaccattgcctgtaatcactaaaggcgggagcagcctacacatccatccacagaggagtagatgccttttggtacatccgtggcgacggaatactaagcagcctgtgtatctatacactcacacgtgtttgtttatgtgtggaatatctctggagggtacacaagaaacttaaaatgatcactgtctctggggagggtacctgggtgcctgggaggcaggtcagggaaggagtgggcacaggtattaccaattggaagacaataaaaacaacagctcctggccaggcgcagtggctcacgcctgtaatggcagcactctgagaggctgaggcgggcagattgcttgcgtccaggagttcaagaccagcctgggcaacatagcaaaaccccgtttctattaaaaatacaaaaaattagccaggtgtggtggcatgcacctgtaatcccagctactcgggaggctgaggtgggagaatcacctgagcctgggaggtcaaggctgcagtgaggtgagattgtgccaccgcactctagcctgggcgatagagcaagaccctgtctcaaaaacaaacaaaaaacagtccctggcactctgggccaggcctggcagggcagttggcagggctggtctttctctggcacttcatctcaccctccctcccttcctcttcttgcagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaataaggtctccattcatggattccaaggacaagaaagtcatatagaatgtctattttttaagttctttcccacgcacccttagataatttagctcagaacaggaaatgatagtattaataaaagctggacatcaggattaacagctctctctggggccctgaaggtgagagttctcagacttgctcatttgcagttgcttctttgtgatgctggcaaaccatcctagtcccattcaaagggcaatacaaagccttgtggctgacctcacgatgcagcactcagtttgcaagaccggcaccagtgtatgcaaacctgagaaggttggggatgaggatatgggatctttcatccctggaaatttagtccagaggcctggggctggagcagaacaccaagccaatcagcttaatgaatggcttagattcctgctaggtttgcagagctgccttctttcctttggtaccttattatagattgaggagtatttctgctaaaccaagatagggataaccagatagcatcttcatagcaatgccacaaaggaaaacaaaaacaaaacagtaatccatcatattattccttagtaactatgccaaggtcatgatactgaatccttagattgtttcaaaatactacttttctttgctcttcctgatgtgtttgccaccgcaggcagatgtttaagtaaaacagattttaactgcagctacaaaagcagcaacaggccagcaaaagagaagtgctatctcagagagcatggctttcagagccacaagagacagcctcactggctgtttcagcttgactgccatgcaaagaagagagcagagggagaaccagccccacccacttattcatcttgtacaaaaaaaaagcacctaccagcctaggctacatagtgagacactatctccacaaaaaacccacgaaaactagctgggtatggtggcacatgcctacagtcccagctactggtaaggctgtggtgggaggatctcttgaggccaggaaggagatccaggctgcagtgagccaagattgcaccactgcactccagtctggacaatcgagcaagatcccatctcaaacaataaaaaaaaaaagcgtgtaacctcctcagaagaaagatgttataatctcaggcagca

SEQ ID NO: 2 - DNA, 133953 bp, Homo sapiens
  source 1..133953: mol_type = unassigned DNA
ggacggccgagcggcagggcgctcgcgcgcgcccactagtggccggaggagaaggctcccgcggaggccgcgctgcccgccccctcccctggggaggctcgcgttcccgctgctcgcgcctgcgccgcccgccggcctcaggaacgcgccctcttcgccggcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtaagcgccgcggctccgaaatctgcctcgccgtccgcctctgtgcacccctgcgccgccgcccctcgccctccctctccgcagactggggcttcgtgcgccgggcatcggtcggggccaccgcagggcccctccctgcctcccctgctcgggggctggggccagggcggcctggaaagggacctgagcaagggatgcacgcacgcgtgagtgcgcgcgtgtgtgtgtgctggagggtcttcaccaccagattcgcgcagaccccaggtggaggctgtgccggcagggtggggcgcggcggcggtgacttgggggagggggctgcccttcactctcgactgcagccttttgccgcaatgggcgtgtgtgtgtgtgtgtgtgtgtgtgtgtgtgtgtgtgtgtgtggaggggtccgataacgacccccgaaaccgaatctgaaatccgctgtccctgccgctgttcgccatcagctctaagaaagacgtggatcgggttctagaaaagatgactccctgcacgcccctccctgcacctcccgagcagtgattccgacagggccttcactgcccctgattttaggcgggggccggccccctccccttttcctccttcagaaacccgtaggggacatttgggggctgggagaaatcgaggagatggggaggggtccacgcgctgtcactttagttgcccttccccctgcgcacgcctggcacagagacgcgagcagcgccgtgcctgagaacagtgcgcggatcccactgtgcacgctcgcaaaggcagggttcacctggcctggcgatgtggacggactcggcggccgctggtccccgttcgcgggcacgcacagccgcagccacgcacggatgggcgcggggctgcaggtgcatctcggggcggatttctttctcagcgctcggagcgcagggcgcccggcgtgtgcgctccctgccggaggcgcggggctggcgcgcagggctcgcccctcactgcggcagtgggtgtggaccctggtgggcgaggaagggggaggataggctgtgcctcctcccactcccgcccccagcccccctttttttccccctcggaacgcgaggtgccatcttttttcggcgtgtcacgtctttacggtgccatgccaaaccgggtggccgggcttcataggacagggcggggcctggcattaaagggagggggacaatcagcgctgaaatcttggcgttttgctgctgcgggcgtgagcactgggggcgttcgcccagcaccttcttcgggggctctttgctttgtctgtagaggttacgtgatctgcgctcccagccctggtttctggcttttattctgagggtgttcagtcaacctcccccctacgcccatgcgcctctctttcctttttcgctcctcatttccgagcccattgttggatctcgaggcttgctgggttcgatgaactcgagtcaaccccccgacccccggcacgcatggaacgggcgtgaccgcgcgcagcctcgtctcggagtctgccggcgccgggaagcttctgaagggatgggattcgagtctccgtgcgcgctgcgggcggcggcagagggatctcgcccctccctacaccccaagtgtcctgagggccacgccacaccaggttgcccagcgagggacgctggctacccatccggggatgggtggggagccctggcggggcctctccggctttacgccctgttgcttcgcctggccggagaatgtgaggaaggggcataaggttactggtgcttcggccacacccatctttctgagcccactggactgggcgcagaggggggattgccatggaaaccacaggtgtccggagaggggatcttggggctggcctcaccccttccctgcggagattggggaccctggggtagggggagccgcgcccagtcggcctcctggaggacacgggaggaagccccgaacccccgcgcctgaggctgtttctgattggcccctggaggccgcagacacgcagataggcggccctgggtgtatttttattaatattatgtccgtactgattaatattatttatcttaaataaatttcacccgtgtccaagttcaccgcgcccccaaaaccgagtctggggcggcagggggaactcctggccaacgaatccatgcctcgccctcctgtgatgaacctggtacgcacggttttctggttaattctatcgctgaaaactggtgcggggggcgcacttctgagacggaagagcatctaggagctgaatcctccacgcgggtcgcccaggttgatctgaatttctggggaatggcttggctgcccgcccgggaccaggccgaccctccttgacggtggcgtagagggctggagcctgggtactgcgaggctcctcgcatggctgggcccgccgcgaggggttgcagagcggctcagggatcgattcaagcatcgtctctcctccctcgcccccagacagagctgggcgcggggttccccttccagatggagcgagggtctcggggtggccccggaaaaggggagcccgcggccacggctacgtattgccatctcgcgagcagagatgtcacctcctgcctttggaggaaagggagcccggtggggatgagcgcatttagcccaatgctgggaacaaagcgcactccgcgcttctgcgatttcgctccattttgaaatgtgttggcgctttggtggggccgctgcggtgggcaaggccgggggcgctgttaatggaggaacctcagggggacggtccttcgtaggaaactctatcctggctctgcgcgcgctttaaggaaatggcttccctccaggacctcgagggatgcagcttttgcgcggatgacggtggggtgctgaaccagccggtgcgcctctggaaatgtctgggcacggatcctggggccatcgacgactcctccccattcccagcaggcgggagctcttacattccgagcgagtgacccctctcaccctctggcgctcacacacctgtaactccaaacctccgtctcagaatggtccaggctggaagggatgatgggggctccgacagcgactgcctagctcacccctctgcgtgctcaggctccaggctcagcaggaccaatttgagttctatctgatccccctcggccccttaactgacccatcctacaggagacagggaaatgtctttcctaccgcggttgattctggggtgtcattttgtgttttgtgatggctgcttatatttactgtataagcattgtatttactgtataagcattgtattataattactgtataagctgcttatatttactgtataagcatctccaaatcctccctctacgtaaacaaattaatggataaacagataagtgtatcccctgcccccacccctgctacgcaggtccggagtgactcttgaagctcatacattccttggccaagtttgcttctctaacagatgtttatatagcaataacctggcttggctcttgggttcacctttggacgatttggggaaggggcttgttggctttgctgggttttggatgagtgacagtccatgactgttcctgctggaagggcgtgacttttaagtggtttctaatatcaggcattgctcctccgacaggaacaaaagaaatggatactgcccataaattgttagaaaacttagaatcgctttgattgaggaaaggttagatttattccggttggaaaaagtggcctttctattaaacgtgccctttgaccctcatgcccttggaggtcggtgccagcctggagatgggataagattgtggttttccttctgcctttttaacatctgttgttacagtccatttgttgaaaatttaaagaaactgttttattccactttccctcagcatttatgtgtgtggtttcagtagctctgtggctatatgtacgaacacgtgttatttttccaattggacatgtgataattttccaactggaccttgccttctattgatgtatttatttagcatcttccttactccctccttgaaaaagaatcactcaaaaacaaataaaaacagccgtaggggcctaatacagtgctagacatacaagaggtattcggtccataccaaatggattttatccatgaaggataaatggggaaatacagtgggaagcaggtgggaaactgcgtttgactctgctctttcctccaccaccactttcctcatcaccgtgttcagagacccccaaagccccctcacactcccagaaacacccccctggccactcctaacttgccatgcccaggagttaggtgcttccactagtgacatggagctggcgtttggggggcacctcagcaggtgacgggaagagaagaccccagcctcaccagctgggctgcagcagggagaggagtcctcatgttccagcagggactctcagctgttttcctgtaaaaccatggttctcaactgggggccactgagatgtctagagagatgtttttgttttcacaactcggggagggtgctactgacatcttgtgggtagaggccaggaatgctgttaaacatcctacaaggaaggcacaggacagtctcctacatcaaaatatgacccagtcccaatgtcaccactgctggggttgacactggcactgctatcttaattacattcattgagtgtcttttaggaggccctattctaagtgcttgctaagattatctcatttaatcctcacaacacttccgctatgtagcaggtgctgttattatctccgtgatggggaaactgaagcacagagagggttagtaacttgctaaaggtcacagagccagtgggtggtggagctggttgcctgacactagttccctcccctctcagccacatgtgggtttacttggccattgtggactagtctgggaacccagatatgatctataacattgacccagtagaatattgattccaaaaccactgtctcacaaatgaatttttacaagagtctgtaatcggagcatgacccagaataaggttagggagatgtggagttaaagctctcaatttcttatctggccccgacacagagagcaaggcatttcactctacattggtgctctgtttataaaacaaagagcaaatatctcttcctaaggtccttaaacctcttcccccaatccagggtttctggactgctctgccatatgacggggcagctggtttgattgacccagggaaggctggaaatcaagactgggggatcaagacgtagattcagtgtggccaaggtcaagtctctgaggtttagggacatcagatccccagcttaggttctgtacctcggcaaggtgaaagcgttggcgcccactgatgaggcctgctctgagattgtgggtgtgggttgagttgggtgggcataggcaagtcctcttgtaagaatcttttggcaaagatgggcctgggaggcttttctcacttcctggggcccaggctttgcaataagtattccattatactgtggtaccttggggctacctgagaatcctctgtctcgcccctgttgccttgccaaagagtttgctgtccaagaattcctttcctgtctccaggtgccatgctcctgccacctctgccaggttccctgcctgcccagatggctcccaactgagtgtgaggaggaatttgagacaggttttgagctttctgggttctccagttaggaaactttctgtaagcatgcagatagaatgggcttcagcaaaatacaaactcgaacaacttccatgtatagtcccttaattttctttgcttttttcatatttcatcaggctccatgctgagcccaatcagggacccgatagaaatccaaacaccatgtcagcgagtccccaagaaatgcattttgtgccaaggctattcaaggaaggtttgggagcagctcaagggcagacactgttaccctcccccaggtccccagtgcagggcagtgttctgcatgtggaggcagtttggcctaatggttaaggaggtaggctctgatcgggcctcctgggcacaaatcccagctccctgctcactgtgagacctaagccatattgtttagctgcttggagagttttttgtcatccacaacttggagtatgatggtacctgtctcacgggttgccatggggttcacacaagctaacccggtactcactagggccaagcacatagtaactgctcagtaaatggcatcatcggcggtgtcctgtggatgagtgcttgtgattggctgaatgaccagaggggtctaaagatcctggtgatggaatcagttgtacagataaattgttacactgagtagggatcaagataggaaaagtcggcaactacccagctcccctgcaccaaactgggcagaagtggatcctctgaaaattgcacacacccatgtttaaatgtacacacagaactcttgccacaggcaagcggagatttgtcatctgctgtccctgcctcatcttcttcctgaaatccactccatgccaggaataaactgcatgctctccaccagcccaaactgacctgccttcccgccagccatcccgggcagggtgacctggcttagtacatcgggttcagagatctttccagtttactcgttgaataaaaagtgagggctgatcgagaaagtaatggcagtcagggaaggcgaaggaggtaaagaagagattttacaaatgaagtaattcaacagagtgctgacattggtaaactggcaaacagatttcagggtggttggttgagagtagagtagaaaaggattaaataaagcaaacttgtggtgtactgaatcttaggaattccatgtatccaataagtatagtcatttatgaattaataaattcggcctaagaagccttcttatcgcttaaatcaagactaagtaacaatatatcagttttaaaaagtcattatatcagaaaatcatttaaatgatacacatagatttccaagattttactttaaccgaaactatataaatgtgaatttgttcacccatcttttgacacagggctcaggtcttctcttggtgtctggatcagccagttgaaatttcttgtctgttttgcctatgccacattaataatgcactgtctgggtcctccgatttcagtttggattttgggtttacattgtggagtcatctgaatgcagaatccttcagggattttacttttttttttttttttcatggtctttaccatcccatttgatagtaaatattactcacctttatgaagtctttccaaaacattcaactaaattttcttaaaatcattgaatgatttgaagagcttattcctcagcacttttactccatcagcttgcaccttattttttaatctttttttgagacggagtctcgctctatcgcccaggcttaagtgcaatggcgcgatcttggctcactgcgacctccacctcctgggttcaagcaattccgcctcagcctccgccgtagccgggactacaggtacacaccataatgctcggctgatttttgtatttttgtagggatggggtatcgccatgttggccaggctggtcccgaacttctgacccaagtgatccacccacctcggcctcccaaagtgctgggattacaggtgtgagccaccgcgcccggccagcttgcaccttatttaggatatgtgattattatagcaagtctggtgtacatacaagattttgaatgggcacagatgacctttagtaagtgcttggctgtgataagaggcagtcctgactgcagatcaggctgtgtggaccccagccttgcatgtttacagaccttcatgtcttattcttacagggtatcagaagaacacctactggggaaacttataaattagtaaaaggtgggcattctccccgcccatcttctgtctgtctgccaggactagcacagcactttgaagtcattcacatagaatcccaacttaagagggtaaaatcctcctcaacagactgaaaataagtttaaattccctttgctatattaactcccctgaggaaagagtcttagatcaatgtccaacactaaaaacagttttaaatcagcaagtgagaattaaatctgaagcaattgataataatgtttcattcattcctctcctttggccccgtccaccctactgctaaatccaggcatcaaagagaagagggacataattatctctagtcccagctgctggttttccttccagcctatggcccagttttctgttttactgagaaggctggtgatgttatcttgggatctaagtctgcagtttcaccacaaaaagtccagggatgcactttcatgcttgtgtcctcctccctgggatagcaaggatattagaagacccctggctctgtaattgcttgtcatgtgctctacagacgccacagaatgccaagaacgaagtgctgggaaggacaaattcatggaaccgtgggacggtgctcctcccccagcgtaaaggacagctcctcctcctgaattggagccagcgttctaaatcatgtgtcaacagagttgtcctggatcggatccagttctgccattgatttgcaggtcatttcagtggtacctgtttccagttgttcttaattgaacagtggcaccaaactattgtcttgcctcatccccctcccatggcctgtcccccaaaaagagacttcttgggtaattaatcagggcaacatcaggcagtctgggcgcggtggctcacgcctgtaatcccagcactttgggaggccgaggcgggcagatcatgaggttaggagattgagaccatcctggctttgtgaaaccccgtctctactaaaaatacaaaaaattagccgggcgtggtggcgggcgcctgtagtcccagctactcgagaggctgaggcaggggaatggcgtgaacccgggaggtggaggttgcagtgagccgagatcgcaccactgcactctagcctgggcgacagagctagacttcttctcaaaaaaaaaaaaaaaaaggaatctctttggttttatatatattttttttatatatataatatatattaaaatataatatatatatttatataatataatatataaatatattatatattatatattttatatattatatattatatatattatatattatatatttatatatttatatattatatatatttatatattatatatttatatatattatatatttatatataatatatattatatattatatattatatattatatattatatatttatatatattatatattatatatattatatattatatatttatatattatatatttatatatattatatattatatattatatatttatatattatatatttatatattatatatatttatatatattatatattatatattatatatgtatatattatatatgttatatattatatatatttatatatataatatattgtatatattatatatctaatatattatatatattatatatattatatattataatatatattatatattatatattatatatatttttatatatataatatgtataatatataatatatataaaaacatatataatatatattatatattatatatatattatatatattatatatattaaatatattttatatatattatatatatatacacatatatatatataaatgaggccaggctcggtggctcacacttgtaatcccagcactgtgggaggatcacttgaagccaggagtctgagactagcctgggcaacaaaacaagatcctgtctctacaaaaggaaactgtaaaaattagctgggcatgatggcatgtgtctgtagccctagctacttgggaggccgaagcaggaggatcgcttgagcccaggagttcaaggctacagtgagctatgattgtcccatagcactccagcctgggtaacacagcaaggccctgtctctaaactttttttttttaattctatttatatttacatgtatttaaatgtgaatattcactacctatttgttgcatgcctgcattttttatactgggcttgccaaaaacccgaacagctttctactttgacaatgtatcagaatttaaatcagcaatatgttaataagccaagcaaaggttatatatgcaaataaaactgttgtctataacctcctgttacactggggcacagcaaaagtcatggtgtagtcgcatgtgaacctgtccctttcatagctgctcattgccaggaaacatcaggaatagccatttggaagagtcatcagccctcccaccatccgttttctgtcttgtcttttccctatgagcaggggaaattccacgctggccccaatccccagtgcagcggctcagcctctgcctctgctgctggtccccatgaggccagcttagaaacggaggattttgcagaacatccctaaatccgcttgaataatgaagtgatcattcataaactcacctgaaccttattaaaacctatttaatatttttcctggataatcctatagggataacttgcctcctgggcttctctccaccgggttcagttcttcctttagtggtgaagttcctcccttcttagcatctcaactgtgcctgagaaaaggccagtggcggctgcactctgttccctgtggagtgttaataaagactgaataaattgaaataaatccctttcaatgtcattaagtgctataaataatcatgaaccaatgttcgatggctgatgagaaatgcaagaaaaaatttttaatcagtaggattcataagttgacaatctgggccaagttaaaaaaaataaaaataaaaagacttttaaaaagatcttatcgtttgttaccagtaagactgaattccagaagcaagctactccctcatttgtgggcccctgttatcactggctgcttagggttgccaagccctgaattcatttgtcaactaagagatttttggccaagattaagatttcccatgcctccatatttccatctgagaaatggagattatactgtcttccccctcagaatggatgataatgtggtctctcttctgttcgcatagtcatagaactgaaataaaacaacttaagagaattcctttgagcttctcagaagtgctgcagggctgggggatgcctcccaggagccgcagtcaggtgctgatctgaagtctttggtgggctgactttagcctgacctgaaatagtatagctgctgccacctggctcccttagcgtcagtcagacggtgcagctggttcctaggggtgagggctgagccagcagggtccgtgcccaggagggatgcatgggtggccacagcccagcctgcactgatcttgtctgtccccttctttggaaggaaggagccccaaaccagggtgcaagacagtgggtgggggtgccttgagcatgacctcaagtgatttccagcccctgccagtgctgacttctctggggaagggctgggacttccttctgggctcaagtcacgacccttggatggaatttcctgggagcttttctgttttttctggagttttcagttttttcctaaccagacagggacttggtacagaatctcatattctaattatgcctaggagcagcctctccccaccactcacagtgtttagcatgtgacaggaatcgattaaggcatgagtgattaaattaaagccaggcattgacttggatggtgtaatattctgacatctgtttggtgtcaaaggcacggggcaggcgcgttaattgaactgcttgcacctggcatttgaattgagccagagcggggctaaagtcagtttgccttcaccctgtaaatggagggtttctccggagcgtggatggtgggaggtatttcagggtgtatgcataacccccaccctgacaatggcccatctcttctccagcgtggccaggtttgagtgccagtcctgggtgtccagtggccccatagccttgcgttttagtaaaatgctgcccccattaccacctggtctgtgcacttcggtcactggaatttgccatcttccagtcccgaatgtggcaagccatggagccttaagctcttctccctccacatcctggaacagacccgccagtttcttccaggcattgcctcagtttgcccctctgtttccagtcacactctcaccagcgataaaatgattttagaccttatcatctcaccctcggatccttatggaaacaataatgagttgttccctgtttcaattccaaaattcatatccaatccgttttgcatgccattgccaaattcctcccagagcaaccccgtcacctgccctggccctctccaagtgtggtcctgccatgggcatcgcctgctaagccaagctggcctcgagctgcctgcccgggtccccacaccttggctcacctccctgcccagtcccgcctcctgccagcctgccctgtggctccttcatagatgccgtgctctttctgccccttgctcacccatggcagccttgcccctctctccctgccccaccccctatttaaattgacctgaccttcctcagtgtccatcttccccgaagctttccccagccttggcactcaaggtccagaggctacgcgtttcctctcacctgtggcagcgccgtgctccccagtgcctcacagtttccttcttgcccccgcttcctgtgtaggactcatctgcccacaggttgcacgtcctgtgagggcaaggactgtgtcttatgtgactttccttctccagtcacagagctgggcacatagatagctcaaaaccctctttattaacacagttggatgttgagaaatcaaacaggccaatgtcaaatgagctctccttatttaaatcaagtcagttctccacctcctagcactcagttccagtactctatatacatggaaataataaaaaacacatttcctttgaaacattctataatcgttcctttgccctacttcagaccaacttaacgcactccccattggtccaaatgagttttgctatacgaagatgctgataataatagcagcagtggattattctgctaaaaccattgcctcgttaatcctcagtcccgaggtggggattattatcctcattttgcagagaagcaaactgagactcagagatttcacagctggggagggagccagctcatccctctgtccaggcccaagctctctcccgcttgccttcctgcctctgcaacctcagagcatcccccatctggttctactgcctgtgctagtcgtgcaggagccaaaagacacgtctttagtgctaaggactggagaagccatgccctccagcctctgtgaatgggtcatatgtaacatgagcctggagaaattatttgaaaccaaaggcaagcctctaaaccaggctgctgcttcatggcgccggtgacggcagaaccaaatttagtgctgtgggcaggtccacacttatcaaatagagaagctcatttttcttccggctcacatcaagcatgaaaaatgttcacacataccccccacacacacatgctttccggaggggtccatgtggctagaggctggaagatgtggatgagaggagcctggcaggtaagcccagggaagatgacattcagcttcccagacagcatctacagggagaaatttaattaaaagtggggcggtttccctgagcaaggcagacaaagtcagccctctactgttaagaaaaagggtcacagtgagaggggaggtgaggagactgagtctgtattttctagtctgttgggctacactacctgatcccccttcctcaaaaatccactttactttccccatgtctacaccaatgtggttcacactctgggaccaggaaaagggggagtgatggggaacagagaagggaggagctcacacagctgaggctggggttatgcatatcgaattacttagaatttgcaacctcacagggtactttcatggcgttgaaatacacttcccacagccaccctccctctaactaaaagcaagagtcatttctcagttctggtcttgcctcccacgttctcctccacatttaagaaaatccaccagctacaaagtgaagataccatatgtgatatcccaccctagtttctgttttatcagggtttggagcaggtggagcaggcagagggatcatttcagcctataaattgtattaagggtgagtactgagtcattcttcaagaaaagttttagaagcatccaaaactgaagggtggagccacctggagacagtatcatcagtcctggccccgagcatggcctgcataggcccccatggatcccagcgggagctgcagagtgcgggcaccttggcacacagccctgagtgcaaaattaggagctgggcagagggcatctctctgtcgccattgggcagcccagggcacactggtcatagccttagaccacgaacaccctgtgcccgggggacagatgcaaccagtgtgccctgggctgcccaatggcaacagagagatcgacacctggaccccatgtcacggggactccactactaaggctcctaagactgccaccttccagtgggataagccctgcctcctactgggcccacaatgtgcagagaacacttgggactacctggctttctggatacacaaatattgatccaatctggactaattagaaggtcagtcccaataacaaatcgaagtcagctgggcgtgatggctcactcctataatcccagcactttgggaggctgaggtgggcagatcatttgaagccagaagttcaagaccagcctgggcaacatagcaaaaccctgtctctactaaaaatacaaataattaggctgggtgtggtggctcatgcctgtaatcccaacagtttgggaggctgaggcaggtggtcacctgaggtcaggagtttgagaccagcctggccaacagggtgaaaccccgtgtctactaaaaacataaaaattagccaagcatgatggcatgtgcctataatcctggctactagggaggctgagacaggagagaatcgcttgaatccaggaggtggttgcagtgagctgagatggtgccactgcactccagcctggttgacagagcaagactctgtctcaaaaaaaaaaaaaaaaaaaaaaaagccatgcctggtggagcactacgtgtaatctcagctatttgggaggctgaggcacgagaatcacttgaacctgggaggcagtggttgcagtgagctgagatcgcgccactgcactccagcctgggcgacagagtgagtgagactccatttcaaaaaaataataaatctgagtcactttaatattgttatttggatgtcaacctctaggtgtttgagacaggagagtgatatgggggcactggaaacacacaggcacggggtgtcctcacacttgggtagcccacacgatgtgatttcagggtgctgggaggtccccccactccccaaattactaacaagtggatagtactttacagtttatatgatctcatttgattcttaacatgagcctgtgagtgaaaaattccttcccctcttctacagattaggacgttgagattcagggaggttcagagggattcagggaagtcaagtggcacctggagtcccgtggctaatttgaggccggtaggggattcgaacccaggatttgtgcttcttatgcctgggcttctgctccctggggcatggtcttccccctagctttcccattcactgctttagcctaggggtcctaccctttattaaactgccagtgcctcactgcttttctcccccaaagacaaaaaaaaagtgtttttgcttttgttttgtttttcatgggcagagacctggaatttcagcttgagaatttgtgccatatgataaataaatcaacagatggctttttccttaaaaaaaaaaaaaaaaaaaactaagatgtatttgcagtgaggcataatttgtaccaaaaagtgctcaccacactgtagtcatgggggcaggaggcagccgcgggtgaagggagaaatcttggagtccaggcagcccccttctgggctgaactggggagctgggggtgctgccagccctgccaggttctcctaggaggcggcagctcatatggctgtgggaggaggcagagggagcctcatatgcacccacatttccagggatctagaagacagaaggaggaaaaccaccatcatgttaaagcagacagttaggtaacacatcctgtaatacaagttattttttccacatctaaaggctaaaaatagttgttagaatttaaagataattggtaaatgagtttctatccttctagtttcacatcaaatggaatcatgctgccttcacatcactagtgcccgttatttgtgtttaatttccacaatgttgtctaattccactctttgggcttccccagggatccagcctccctcactcgcccatcgcagggagatgctttattcatctttgtgtcttctgtgccgggcatagcgcatggcacagaataagcactcagtaattgattcacgagtgaataaatggatgagtgggtgagttcaatattgactacaaaaaccctaaggccacactggtgagtggctgcgcctgtagtcccagctgctggggaatctgaggcaggaggatctcttgagcccaggagtttgaaactagcctgggcgatatagcgagaacctgtctcaaatgacaaaaacagggccaggtgcagtggctcacgcctggaatcccagcactttaggaggccaagatgggaggatcacttgaggccaggagtccgagaccagcctgggcaacatagggagaccctgtctctacaaaaaattttttaaaaattagctgggcatggcggtgtgcgcttgtagtcccagctactcaggaggctgaggcaggaggatcacttgagcccaggaaattgaggctgcagcgagccatgatggcaccactgcactgcagcctgggcgtcagaacgagacctgctctcaaaaaaacaaacaaacaacaaaaaaaaaggctttcttaaagagacttgagaacagaaaggggaacagatacataacttatatatttatttgttcatctttccaccttcctggagggtggaggggaacaggtctgtatttggagttttgaatgctaaaagtgggaatacatgtactgtttgccatgatctgttcaaaagttaagccaaatgccttagattctcctgaaaactggaatgccactgtaaactataagccccacttcaaagataaaagatcttgatgaacagggctgggtctgtggactgggcctctccccaccacacaaggaagggtggtgccagttgaaggaaaatcacttaaatccttgctgtctcctaataaggtgtggtcccaggtagggctgtcagaattagcaaattaaaacacagggcatctgtgaaaattagaatttcagataacaacaaataattggcataggctgcataatgtccctcaaagatatcaggtcctaatctccagaacctgtaaatgtgatcttatttggaaaaggggtctttgtagatgtggttaaattaaggattttgagatggggggattatcctgtattatctaggtaggtcctaaatgcagtcacactcatccttgtaagaggaaggaagagagagatggaaaacacagaagagaagacaatgtggtgatggaggcagagattggagtgaggtggccacaagccaaggactgctggcagctaccagcagccagaaaagtccaggaaccaattctctcttggagctccagagggagtgtggccctgctgacaccttagcttcaacctagtgatcctgattttggactttggccttcagaagtgtgagggaatgaatatctgttgttttaagccaccaagtttatggtcatttcctacagcagccacaggaatcaaaaacagtaagtatgtcccatgcaatgtttgtgacacacaccaaaaatattacttgttgttcacctgaaattcaaatttaactgggtctcctgtattttatttggccaacctagttcccaggcccaaagaaagaggcttttgaaatttgcaagaaagctggttggagctgtcagaaagtggactttgtaaacacagtaccaccgaaccaatttgaactgtactacctctagacaaaagagagggcagtcagacagttgttcgtgatttcttctttcaacagtcatttgagcacttactacaaaacagaagctatgtgtaagggtggaggcgttagctgttaatcaggacctccaggctaagtttctgtattagtccgttttcacgctgctgataaagacatacccgagactggggaatttacaaaagaaagaggtttaattggacttacagttccaagtggctggggaagcctcacaatcatggcagaaggcaaggaggagcaagccacatcttacatggatggcagcagacagacagggagagagagcttgtgcaggggaactcctctttttaaaaccatcagatctcgttagacttattcactatcaagagaacagcacagaaaagacctgcccccatgattcagttacttcccaccagatccctcccacaacatgtgggaattcaagatgagatttgttaccatatcagttaccaacccttccagataaatcacgtgaaatatcgccattaacagagtgagctcaggtggttcttcagtgcatttctgatacctgaaccttccctgggaatttcacagaccatcaggctctccaccctttgatagcaggatagcagggcccaggttctgcaggaggagatgttaccacaggcctgaaagggagggaggggcagatgctacaggaagatgctggctctggattcgctggaggagctttcaagggaagtagatacacactgtctccatcatttcatgtccatcacactctaaaatgctttggacaagaagcaaatgttaaagacaaatgtggcccattttcctgtacaaagagggctgctcccatgccaggctattggcactggtgggcatgaggcttctctgctgccctggccggggggttctctcactcaccattggctctctgacacctggagagaccaccacccttgggctttcatgatgctcacagaatccacactgttggagctttaaggagcctggatcaactggaacaggcagggagtactaggacagcccagcattgccccaaaatatccaggcctgataaaagagaaaaacaggtagctcacaggaaaaggataaaaaaaggaggagggatttaacatgaaaaggtgcttgatctccctcataataaaaagactgctgattccatccaggcaagtgacagaaaaaaaaaaattaatttaaaaagactgctgataaaaccacagcgagacactgctgctcagggatctgagggtgtgggcagccaggctgccacgcatcatgggtcggagaggaagaccacacccctggagcagagggcggctgatctgtcagatgccctttgacagcacctcagcttccaagaattaaccctttctatgtgagcagaggcatccatggggggacacactggtgaatcatctgttatgtagaagtctggaaaacatcaggatggaactggtgaaataagtgtggcctctgacggaatggagcggtccgtctgcactgctgcgggtgcccctcagatcctgtgggtcagtgagaaaagcagtgaggaacaaggcaggtactgtgtactgtcctctgcgtgcaaggaaggccagcgcatgcaacagagtccacacagacatagcctaactctggaaggaagaatgagaatgcagtttcagtggtggcctctggtggggagaaactgggtgaagggagatgtcatttccatttctctactattaattttgtattaccatgcttaaatgttactttttacctttttttttttttttgagacagggtctctctctgttgcccaggcaggagtgcagtggtacaatcatggttcactgcagcctgaacctcccaggctcaagcaatcctcccacctcagcctcctgagtagctgggactataggcacgcataccaccgtgcccagctattttttttaatcaagatggagtttttctatgttgcccaggctggtctcaagctcctggactcaagcaatcctcctgcctcagcctcccaaagggctgagattaaaacgtgagtcaccctgcccagccaattgctttttaaaaaagattaaatgcatgtatacgctcaggcatcagcacacttggaaaggatgaaaatatccggaagaagggttcttttaaaaggctcctcaagtgatgctggcaggcatgacgaatgtccctggtcacaaaagctctgatctggcctaaccctgtcatgttagagactggagtgcgtgtgtgtgcgcgcaaagtgtggggggatgggggtgagtgtgtgtggtgtgtaagcatgagtgtgtatgtgtgtggtgtgggggtgtgtgctgtgtgagcgtgtgtgagtctgtgtgtgtagtgtgtgtgtgaagtatgtggtgtgtatgtgtgacgtgaggtgtgtgtggtgtgtgagttgtgtatggtgtgtgcatgagcatgtgtgtgggcatgtgatgtgtgtgtggtgtgtaagcatgtgtgagtgtgtatgtttgagcatgtgtggtgtgttgtgatatgtgtgtggtgtgtgagcatgtgtgtgtgatgtgtctgtgtgtggtgtgtgtgagcatgtgtgttgtgtgtgtggtgcatgtgtgtggcgtgtgagcgtgtgtgtgcattgtgtctgtgagcatgtgtgagtgtgtgtgtgttcagcatatataaggcatgtaactgaacacagcactttagagggctctcctggagtcagagggggtgggtaggaggagaagggaggtgggctagtgtgctgaagtatctactccttgtcatagtctgtgacaacccagactagcccatgagccaccctgttccctgcatttccaatgagacctcggtggacatgttccctgaggtgaggctgactgatgtcatttgacgatcttgatgccaaatccttttatatcaaaaacaaccagaacactctcttttctcttagtgctttcacccagatgaccacatttcatcctcccagccactctgggccaggtggcactgctggtttgaaagggaggtctcccctggagtaacttccgtgggcggattcacaccctgcccacagtcctgtcccagtcagcccaccatggtggtctccggttcctccagaattcccgcttttcagctcatccccacattcccggagggactgagagcgcagccccagggccctgctctttgggggccgtctctacacccagagaagcagcaaggcattcctaggtttctctttcagatgcagaacttcagtgttcagagatgttcccactggtcctgagagggctcagttcagctttaatgactgcgctgttgcgtgtgctctgcagagggcgggtggcccagcgtggctgactgcagttttcctgacgtggagcccgagcctgccccgctgtttattaattaaggatcactctgcttgcagaaccctgaactccccagaactgtgaggtgggagaaccccgagaggccacctggccccacttcccacctgctgcccaaaccccctctctgccttcctgacagtcaccccaactcccagtgatccccatcaaccatctgacaaggggactgagagggaagagaaaggaggggcccaaagaggaaggtaaaactgtcgggaacagcccccaaatgtgtgacagccttcagtggagttgcccactttcccttttctcctccctgcaggacctcccttctccccagtcctccccaacttctgaggttacattgagaaaagtctgcagagaggtgccagcatcacaaggtgttaaggaccacgagtttggcattttaacagatgccagagccacttgagaaatgtggtaactaagcccagagaggtacagttaacctccccagagtcacacagcaggttcatggcaaagctggactagcacaggtgtccttcccctgcagatccccttctgtgccccacatcacctccctccagtgtctgggccacctggagatgggccctcagactcacccggccagaggtgccatctcatgggagaggtctggccaggaagcatcgatatttgagatcccaagaaatgaagacttggcctgtcagatgacagacttcggtcatgggaacacgtgatctgttttacacatgcgtcccctcagcagcagctttccagaacattcccactttcttctgtagtgagaagaactctttccctgcagcctcctgcccaactcctccttcagtgtctttgcttcagtgtctttgataaaccattctgctttgcagagtgcgagctctgccttgcagggttcgcatctgcctgtgctgagtaaccaacgctaaggtcgagtggtcggtcacctctcataagagctagggttgtctcatgctgatgactaggacttgccctcaaggagaaaaataaatcaaaacaaaagcaaaaacagcaaacatgcatctcttaaagaaggctctgagtccaggtaaatttccttccactgaagcagccaggctgaattcgaattatctttgcccctgcttaaaaactaatgcaaattttcctagagaatatccactaattcctggagggggcatgggcattcctgatgcccatgagaggaccatttgctcttccctcagtatgctaaataacagaagcgacatttgttgctggaaagtatcagtgaagttaataaggtttttcttgcccagggtgagggaacagttcccaatgacaaatgctgtatgggaaggggctgtagaactgccagcccctttggtccatccgtaaagtgaactctgtggatcctggaggattccagcgtctttttttttttttctttttttttaagacagagccttgctgtcacccaggctggagtgcagtggcacgatctcagttcactgcaacctccgcctcccgggttcaagcgattctcatgtctcggcctcccgagcagcaagactacaggtgcgcaccaccatgcccgactaatttttgtattattagtagagacgggggtttcactctgttggccaggctggtctcaaactcctgacctcaggtgatccacccgcctcagcctcccaaagtgctgggattacaggcatgagccaccatgcccagccagcatctttcatttttctgtctgctttggccctttcctctctcactgtcttccttttccatttccaaagtcagtccatctcactattagcacaaaaactgctagagcgcttgtcattggtcatctctccctgcacctggctggtctgttcttggccactgaagcgtttcccccagctgttgctttaatcattttattgttattatgccttacttaagaaatggatatgagatgcatttacctgtctcttcctgccactctgcagagccagtaagatgtggtggaaagggcccaggctttggaggagggctggctggggttggatcttggctgccccctactagctgtgtgaccttgggtaagtagctggacctctctgagcctggttcggaatcatagcacctctctttcagggctgctgtaaggaatagcagtggtgtgtataaagcagagcgcacagccagcaactggcccctagccacactgctgagcacctactgtgataagctgccattgtggtgtgtgaagcaaaggggaaacatgcctgctgtagtgagcttcctgtagggcaggttgtagaaccagaggtgggttccaaggttacaaagggactcttagtgtattagtctgttctcacattactataaagacctacctgagactggatcatttataaagaaaagaggtttaattggctcacattggctgggtgcggtggctcacgcctgtaatcccagcattttgggaggccaaggccggcggatcacttgaggtcaggaatttgagaccagcctggccaacatggtgaaaccctgtctcttctaaaataaaatacaaaaattagctggccatggtggtgtgcgcctggaatcccagctactcaggaggctgaggtggaagaattgcttgagcccgggaggtggaggttgcagtgagccaagatcgccccactgcactctagcctgggcagcagactgagactctgtctcaataaaaaaaaaaaaaaagaaaagaaaaagaattgcaagaaataaattattgtttatgagctatatggtctgtggtaccttgttgtgggactgggagtcttggcgtctccctgaccctgcctgttgctgcagcaccgctcagccctgcctgctccctacctgcctcccctcggcctctcctgcctccaccgggcccctggtgcctcctctagagacagtcctcctgggaccgattgtgttctcacttacacgaggcatccaggactacagataaccagaggaaggggcgccccccccgcctgccctcctccctggcatcctcacgctgcagaggtcagagcctcatcccagccccttacctgcccctactctgtggagaaccgtggtcagttcgccaggccggatccacgaacggccttgtggaagatggtgagctcacacccagagctggctccgatgaccctgtctcctttacatgtttctaccttcccctccctaccttcccccactgctgggcgcagagtggaggcagatgaggtttaaagctcagaagggcttaaacgggttggggcgcagtggctcatgcctgtaatcccggcactttgggaggccaaggcagaggatcacttgagcccaggagttcgagaccaacctgagcaacatagtgagaccgcgtctctacaaaaaataaaataaataaaattagctttgcagggtggcatgcacctgcagtccctgctactcagaaggctgaggtgggaggatcgcttgtgcccaggagtttgaggctgcagtgagctatgctggcaccacagcactccagcctgagtaacagaatgagatcctgtctcaaaacaaacaaacaaacaaacaaaagaaggcttaaagggggctccaggtgggcttggcagcacaaagctatgaagttctatcttagacacaagttctgttactgggcctttgcaggctggcctgggtacctggctgccatagacagggaaccttccagatgagctgcaggcgtggagcacaggagccagggtgctcttcctgggctctgtccacaggcagaacgtacacagtctttgtacacgtccggcggctctggtgcctatttttgtttgtgtttttcttttgtttggggggatggatttggtttcccccgagccctctgtcctcctgtcacctggctggtgctcggcaatgttgaccagctgcctggctggagttggcagtggctaaggctgtgacagctaacatgttcctgagtcctctcatttcttcaccataatgccctgttgagtttgcagatactgtctctgtttttatctcccggggaaactgaggctcagagtggctaggccaccttcccatggtccctcagctcatgagggccacacagggcattgcggtggccttctcctcagccttgaccctccggccccagcattgctgcctcaaggggtctcctctgctgagccgtgcaccttctgcctggcagctccaactctgtggctgtgttcagtggctcagcactgccccttgaccctccctggccttctgcggatgccagactggagcactctgacaaggtctggggtggttgtatgggtcctgtgacctctatacacctcccagtgcctgggaatcctgcagatacaccctccttagccgtccctaaccatagaggacatttctgaggtccccgagagagtggggcacccctgcaggatccaactgctgggcccaggaaggatagcagcagcatgaggggttccattagccacaaactcacggcatggaaccttcacccacctcgcccctcatctgctgtttagcacctggcacgccgtgtatacttactgattattacattttaatggcaaattatagtggcaaacgtatgcatctttgcacaattgttgtacagcatgatgaacaagtcattaatagtaaagaataaatgtgaaagtgagaaaaatctgactgccaaagtttttactccttccttccctccccagacttttaaatgaaagtttagggataatcccttagttgtcctgctagtaggacttgcaattaaaagaattgggccaagaacacttctacgcttctccttttaggtttgggtgtaaattcggggtatttctcactgatgaaagcctggtgcagggcagaccgtgggaagctttcatttccggaatggaccatcaacatcccttggagaagaattctcttctccagacccagacctggtgtcctggcacccattgggcaagtgggtcctagaagacaaacctggtcagagcctggaggctgcttagcattccccacgcacattagcagctcggagagctcaggaagccgcagcccctccttgcctcaccagcctggatcaggacagcatcccctggaagacacacagggcctggcctctgattacccagcctggagggaaagctcaatcgagcatcatgtcacccggtgcccccatgcagggtggcactggtgagacccccaagccaatgataccacctcacaggagtgcaggcccattgtggccagatcatcttgacttttcaagataaatcagaaatcgtatttccatgagatatccctatttgcaagtgatggtgactaaattagaagtttttgaatattgtaacatgttcgtaggctgtttgtctggtttaaactctatctggaggaattcaagctagacttcaggaataacttcttgaggcaaggattttgagaccttagggaaagaaggacgtcttgggggtattctgactgttgtcctcctggaagggaagaacagagaactagaagactgcccttagcgaagttcaaagcacctaagcccgggaccctcagcaagtgttcttgagtcacagattctccctgaggcgcctctttctggctccatagaatggctgattctgtaactcggtgagtttgctttttttttttcctccatcacccaggctggagtgcagtgaagctggagtgccgtggagcgatcactgcaacctctgtctcccaggttcaagcaattctccttcctcagcctcccaagtagctgggattacaagcatgcagcaccacacctggctaatttttgtgtttttaatagagacggcccgaagtgctaggattacaggcatgagccaccgcggccagccataactctgtgactcttgttacaaaggccttatattttgctctttgagggtggttttggtttgatgcctgttggttgccatcttttaactagggatgttttatcaaaatgcccagccaaagtgtccaaacaaattataccttaaagtttgaaaatgtctggcacttctaattcaatgcctgttgtgccaggcactgggctgctgaggaactgagtcccgtccctgcaggctagctagagaacacacacacacacacacacacacacacacacagagtggtcttacaagtcagttttatattctacctatatgcaataaaggtattattatgttgaggtgccttgatataaaaatttttcttaaaggagaggatgcctaaaacaggcattacctgaaacctcctctctccagcattggttgtcttctgtcatgactcagggttttcactgagaatgggatggaaatgtggtctaaagatagggccaatgttgggactggatcccctctgggaagtcagaccaggctagggcaggtccttgaagccatcaggaaaagcctctggagccagaaacaaaacaaaaaaaaaatggtgttaactaaactcagtctcaaatcctgaataggactcaagtcaagcaaaataattaaaggagttagcaaagggcaagtcagagagaccgagcaacaccaatgtcttccgggagccctgtggcgagtgacagagcctggactctggagtagaactcatcttgtgtcttcttctgccactcgttagctgggtgaccttgagccaagccccttaacctcttggaccctatgttcttatctctaagtaggggctggtaatatcttcccctttgaggaatgccctctaaggggtgttgtgaagattcggtaaggtggcaggggtaggactcctggccagaaacaggcacataataaatgctaagtctctccttctctccacctgctggatgctgtagatactaaggatttcgatgtgaatgagacaaaacccctgccttccaggagcctttgagaatcagagaactagacccatttccagaacaaggggatgcagggtctggataaagttttggggatcaatagagcagagggctcccagaggatcccatagggttgactcctaactcaagggcatgagacaacccccaggaagggcaccctggaaggggtccggctgtccctgatttacttgtgggcactgggggaatgcccggagccatccagccctcagggctctgtgtgattctgggttcctcccataaaagataatcagattctttcacgttaatgtctttctccacctcattgcacatcatgcagctattcattgactcagcaagtatcagctttgcatgcgaccttggcctacccactttagcttttagtaatagctcccttcttgaataatacaaccagtggggaaacagaacctaactcttacctctgggaggcttatttgctttgagaacatatgtcctgcagttttgttcatatggcagtgaagtttcgtgcacacactctagagccaggcagcctgggttcaaagcgcagctctgccaggtcctaactgcatgaatttgggcaagtcgctcaacctctccatgcctgagtttcctcatctgtaagattggagcaatggtaatacctgctttttagggttgagaagagaattaaatgaattaagatgggtaaagtgcttagagtggagctttgcaagtagtaagtgctatgtaagtgttcgatttaaaatgaaagacccttaaatacattctttgttcatttcacaagcccttcatttcacaaccttacatttcacaaccaagctctgtctcccctggaatccagccataactctgctcacaagtgtgagacaggccccagcagagctgcacgaagaggagagaaggcagccccccagactcccaaccccctgtccaagatggcaaaaccagaacacagcctctgtaccaccccagcaggtattcagaatctgcaatctccaaagcccacttcaattgtaaatgtagagccacgtgcgctttaagtcacctgtcactctggaggctcttttgctcagttcctcaccattagcagggatgacagggagtgcaggagtgcggtcgactcccagatattggagagcgctgggctagctgcccattctcccggcctccactcctctttgctgtccagccatcacttgctctttgaaggcaaacaaaacagaaaacagtgccaaaagtatgggaagaaagccagcttctcccctggggtgcctgtgatgccatgcccaccctccctgaccacgcagcccctgtggaccctcagggccccaagcccccatttccatcacatgcgtacacccatgtgtgtccatagccgcccatctcagtcaataaggctgctcctgcccacttggaatagtggtgacaaccaggagtggcttatgggaactatcccaatggcctgacagcatgtccgctgcaaaccgctgaggtaggacactgccctcatgtctagctgatcagcaagaggcgcagttgctttcttaggtaacattgctgctgtgtcctggccattgctggggggtggcacttaatctacaccagaattttccctcctgtatcttccaagctgcttggatcttggtgctgaattaggttggactttgtcttgtggggaagggaggactatagaccctcaacgtaagcaatggtcagactattctaagaaaactcgccgaattaaagcatgaggtaaatttagttctgacttctgtccaccccactgccactgtccccttttatcccatgatcccttgcttttcttttcctcctctctccctatctcttgtgtttgacgcatgataggaattcagaaatatatgtttgtggatttgtttattcacgtagcaaaccatttcttgagtgcctaccatgggccaggtagaatgggcggccccgggctgcagtggtttcttcagcccctctccagggtttacactgtgcaagacggtttgtgatgggtcctcccatcgaggaccacactcttctttctctgtgccccttggtcctcagtctctgaccccacttcaaaggcagcattcactcagggaagctcccatacaatgctagtcagagtaaaagtttggacaaattgccaggaagcagcttgtcagtatgcataaacagcctttaaaatattactactctttgacccagaatttcacttctaggaatctgtcctaaggaagtagtcacatgcaaaagatttatgtaccaagatgttcatcaaagtgttgttttataacaggaagtctcagaagctggataaatatccaacctctggaaatggttagatagaatagtatgtagccattagaaaattatgtctatggggtttaaaatgtcatgggaaaacacttctgacataaaagagcatgagaactgtatatttagcataatcttaactatgttttagaatgcacaggaaaaaaatgtacaaacatattcatagtgatgtctctggtggtaggattatgatcagtaagtacttctgtctcttcatattttcctgtatttgataatacatgcatatgttgtttttaaaataagaaaaattttaagtttaaaattggagctgaaaagtgtttttaggtcaggcgaggtggctcacacctgtaatagcaccactttgggaggctgaggcagtcagatcacttgagcccaggagttcgagaccagcctggccaacatggtgaaaccccatctctactaaaaataaaaaaattagccatgtgtggtggcacacatctgtaatcccagctacttgggaggctgaggcatgagaattgcttgaacccaggaggtggaggttgcagtgagccaagatcgtgccactgcactctagtctgggcaacagagtaagactctatgtcaaagaaaaaaaaaaaagaaaagcctttttaaacagtagcagacataactatataatccttactaagctgtcggtcaaatttttatttatatatttattttattcatttattatttttagacagggtctcactctgttgcccaggctggagtacagtggcgtgatcatggctctcttcaaacttgacctcccgggctcaagtgatcctcccatcttagcctcccaagtagatgggaccacaggtgcataccaccacacctggctaattttttttattttttatttttagagatggtgtttactatgttgcccaggctagtctcaaactcctgggctcaagctatcctcccacctcggcctcccgaagtgctggggttaccagcatgagccactgtacccagccctcaaatttttaaaaatctataagagacattattggacaattagagaaattcacatatggacttataatagtatcagagtgtgtggtgtgatggttctggagggaatggactttttctttggagacaggcttttctatgcccacccttttatcttgctaacttatcatcatccaggttccagcagaaacattacttcccccaggaaatttcttaagggtgcagtatcatgatgtctgcagcaaattctcaaatagctcaggaaaaaagtacgtgtgtggtatgagtgtgtgtatgtatgtgtgtatatatatacacatatatacacatatatatacatatatgtgtatatatatacatatatgtgtatatatatacacacacatacacatatatatacacacacacatacatacatgtatttttatataattatatatgcagagagtgcaaatgttgccaagttaaagattggtgagtctaggtgaagggaatatggtatttattgtattatttgtgcaacttttcttaagtttgaaaattttcaaaacaaaaaattggaggaagaaggcatgccagtctaccccaagccctccattggaatgctgaaaatctaaacaatgtgatttggcaatttcatttcttttctgttgtgggccagtagtccttagatgttggggaagggggtagtcgctgaggtgtggttgacttaggatggaagaagcagaagtcaagactcccagggtcaaagtggtttgctctgctgacccaagtgtgggaggcccagagtcagcgtttcaggtgtgctaattcagcatggttctattcacggccaaagtccaccctgggcacctctctggcagcaatcttgggtgactctactaaggccaggcctccatgaccctatgtctggatcccatatctccacctctcccactgtctcaggaacggtgcttagctttttcttttccctctcctgtcttctttgccagcatgtagaaagtttaaataattcccctctttacaacaaaacaaaacatacccccttcagtcaaccaccctagctctcttctccttttcccagccagatttttttaaaagcatcctaggccaggcgcggtgactcacgcctgtaattccagcactttgggaggccaaggtgggtggatcacaaggtcaggagatcgagaccatcctggctaacatggtgaaaccccatctctactaaaaatacaaaaaagtagccgggagtggtggcaggtgcctgtagtcccagctactcgggaggctgaggcaggagaatggcgtgaacctggtaggcggaggttgcagtgagccgagatggcgccactgcactccagcctgggtgacagagtgagactccgtctcaggaaaaaaaaaaaaaaaaaaaaaaaaagcatcctcagcactttggcaactccatctcctcccaacatgtccctgttactggaatccagccaggactcagccccgatctttctactctaaccagttgtctcagttaacaaggacaggtttatgctgcagtgacaaacaagatcccaaattcttgtggcttcacacatctggcaccacctcatcttccagccttaggagtcatcttttagttccttgaaaactctttacagttttctgttggggccttgtcatatactattcccctggaatgttctttcctatcccctccctttcaccttgctaacttgtgcccatccttcaggtctcagcagaaacatcacttccttggggaagttttctccaacacccacactacacaggtgtcccatctacactcctatgactttgtggtacttgtctcacttcattttccactgccttccccacaaggcacctgcacaagggcaaggaccgtaccactgtacctatgtcactcattgctgtggtcacctgcactctggctgcctaccttaactacacattagaatcacctgaggagcttttaaagccacaatgcaagactccaccctaggccaattggatccaaatccctggggtagggccagacatcagtggagttatatatacatatatatattttgtttgtttgtttgtttgttttttgagacagagttttgctctgtcacccaggctggagtgcagtggcgcgatcttggctcactgcaagctccgcctctcgggttcacaccattctcctgcctcagcctcctgagtggctggaactacaagtgctcgccaccacgcccagctaatttttttgtgtttttagtagagatggggtttcaccgtgttagccaggatggtctcgatctcctgacctcatgatctgcctgcctcatcagcctcccagagtgctgggattacaggcatgagccactgcacccggccatcagtggatatatttttaaagcactgcagagaattctgttgcatcagcttgagaaccactgatctgccttgtgcttcacatttaaaacttttttttaatgaataaataaaccccaaaaaattaatctccctaagcctccctagaagataggatggtaaggatattttcctaggtaaaaatatgttaatttcatatttcatgaaatttcatgtttcatttcaatcaagctctgtcatacaccttacatggggcaagcccagtgcctgggcagggtgtaattatactcattacacaggcaaggaaaagtcacattaggtgatggagcacaaataggcagttaatggtttcagggctagttaggatatgtttgtctttcaattgcaagtaatagaagcccaaagaaattggttatttatataatataattgattggttcccaaatttgaaaaattcaggaatagacccagcttaggtacagctggatccagtcactcaaacaatgtcacaaagaaccctttgacaggaatgtatcctgtgttgactctactttgctctgagtagtctttccccaggtgatgataaaaatggtcatcatcgccaggcttgtgtcctgtttagtaggaatatacaagaagagctcagtaaatgctggccccaccactaagcaaaaacaaaacttttgttgttgttattgttgttttaaataacagcttagacctttcttctttccttgttattctctttcatctgtaatccagttttctacttctgaagtatagaatgttctgatgatttattcttcattacccacaacttgcacatgtttatttaaaaatgccaggattgcctggccgttgtgtgctgttaacctttgtttgctgttagtggatccctgaagttcaggctcccaggggagcagataatgggtatccagttcctgcaatatccaccctctggcaagccaagttccttcctgggtaaggttttgcctacctgcattcctagggaagtttctgggcctgaccaccaagccagctctgagaaggggtgcataagccccaccatgctttggctctgtccctatagaatattttatgttgttactgaaaactaaaggaagatgggtgcggtggctcatgcctgtaatcccagcactttgggaggccaagacagattgatcactcgatgccaggagttcaagaccagcctggccaacatggtgaaaccttgtctctacaaaaacaaaacaaaacaaaaattagccgggtatggtggcatgcacctgtggtaccagctactcaagaggctgaggcacaagaatctcttgaacctgggaggtagaggttgcagtgagccgagatcgcactactgcattccagcctgggtgacagagcaagattctgtctccaaaaaaaaaaaaaaaaagaaaaggaaagctaaaggagagagactaaaatgatatcaggttcctggagaacaaacagacatgattttgcttcatggcaggacagccggaagaagtgggattatatcctcacattacaaataagaaaactgagactcagaatggttaagtcacttgtcccaggccacacagccagtaaattacagaaacagaatttgaacccaaatcttccagctccaaagcttgtgttcttttcactacctcctgcttaattttttaatttctaagattagacccttcatctatccatgacacctgcctgtcatcccctgaaaaaaggtgaacgccgttcagaaatttttctagcctgagctcactcccagttcacttatttttgctttgtcatggctgcccagtccccacttgtagaccaggaataggtcatggctgcggggactacacgctgtcgctgctgcaagggccggcctctgtttccggggctgagtgggggccagacctgccaggagcaccatcttctgtgggtcctgcctggatgtcacatcccggccccaagaagtcactgcaaaccttcgtattattgagcttcacatcctagaatttgctgtcactgtggctgctgcatgaagttgtcctgagagaaacgggcattgtcattaacagggaaattgatggtctgggggaaaagtcatcctcattctcttgcagatctatgggtgattgagactggctgatgttgaaggggtttctcagccatcgtgtgccatgttatggaacagtggtgtagccagccatttgacacccagcgctgacctttgtttaacaacctcacctatatatgacaaaatgattgtcagaaataatcgtgtaatgaaatgactgtaataatggccagaaaagaaacgcagatagtaaaatgtttctcttgttgaactctgtacatataattgcaccaggatttttttcaaataaaaagtaaatattatactacaaaaaagggaaaaagcacaagcatttattaaatagctttctatatctttctgagttttgatcctttgattgcagactgatgtaatattttatgtaaatcattgcttggttactaagtgaactttaagaaaagtgagacgtctgcagaagttgcccataatttagcagctactgtattgtaccattgatgtacggctttattttcttgattaattatttaaacaatataattcacaattttaaaataataaatttccacttaaaatggtatttaaactcagcaaaatatatcatctatgagtaaaatttgtatttaccaagcaaaaatattacagtttgtggttcacatgctgtctcactgttttaaattttaaatacaaaaactccaagtaggctgggtgtggtggctcacacctgtaatcccagtactttgggaggctgaggcaggcatatcgcttgagttcaggagttcaagatttgcctgggcaacatagtgagatcctgtctctactgaaaacaattagctgggtgtggtggcacatgcctgcggtcccagctactcaggaggctgagataggaggatcacttgaaccctgggggacagaggttgcagtgaggcaagattgcaccactgcactccagcctgggtgacagattgagaccctgtctcaaaaaaagaaaaaaaaaaaagaaacacaaaaactccaggtggtcgcacagaatgacaggactgaagtaacttagctccaatttctgtcttcataatcactgtcctaccattgtctgtgcttagaatctacttgcttaatgcaggaacatgtgttctcacagagatggaaaatgcaaatggcgccagaagcaagctggaaattctgaaccattaagaatttactctctgccaggcacggtggctcacgcctgtaatcccaggactttgggaggctgaggcaggcagatcatctgaggtcaggagttcaagaccagcctggccaacatggtgaaacttcatctctacaaaaatacaaaaattagccaggcatgatggtgggtgcctgtaatcccagctactcgggaggctgaggcaggagaatcgcttgcacctgagaggtggaggttgcagtgagccgagatctatctgcaccattgcacttcagcctgggagacagagtaagactccatctcaaaaaaaaaaaaaaaaaaaaagaacttactctcaaaataaatacgtgtggctgactccacatatggtagggccaactgtataactagaagttctccaaataacttctgtggagaaaaaaaagtttattaaaggttaacttttttaaagtgctaactagaaccttactaacactgagatcgcaccaattgtttataacttagacagggccgggtgcagtggctcatgcctataatcccaacactttgggaggccgaggcaggtggatcacttgatgtcaggagttcgagaccagcctaaccaacatgatgaaaccccatctctactaaaaatacaaaaattagccaggcacggtggtacacgcctgtaatcccagctactggggagggtgaggcaggagaatctcttgaacccaggaggcggagattgcagtgggccaagatcgcaccattgcactctagccccagcaacaagagtgaaactctgtttcaaacaaacaaacaaaaaaaaaaacctcttggaccaggaaaatattttttaagggaggagtattttatcactggcattgtttaggattgcaggcacatgatgctaatgaaaagcagactaactattagttggttttattactgtttttgaactctctctctcccttttttttttttttgagacagagtctctctctctgtcacccaggctggaatgcagtgactgcagtctcagctcactacatcctctgcctcctcagttcaagtgattctcgtgcctcagcctcccgagtagctgggattacagggcaccacaccaggctaagtttttgtatttttagtagaggcagggtttcaccatgttgcccaggctggtctcaaactcctggcctcaagcgatctgcccatcttgacctcccaaagtgttgggattacaggcgtgagccaccgtgcctagccctgtttttgaactctctagagacagtccagccccttattacttgtcctgaggcagctgctcccttcacctggccccccgcattgtgttccggacccttgtcctggtggtgctaaagaatatctctgtcgatcctttggggactggggaaactgaggcccagtgccacgcgatgccatttgttcagggaagattaggtcatctgctaggtccccagtcacttgaccttcttcccagacaggaagaagctgctctgggtctctcagtgctccacgtgtctttgcacattgaaatgttttctgattttttttttttttttttgctgttacatttacttttaaaaaataacaagcaataaaatgttacatttgagaaggttgaaatgagaattgatttgagttaaattctagcagatttttcttagaagaatgatatcatcatctccagctacctgcaattgatctactctgaattaagaaagagacttccatttgttgtttatattttgcactcttgatgtgtttctttaaattatggtcatgggccaggtgtaggagctcacacctgtaatcccagcaccttgggactctgaggagggaggatcactggaggccaggagttcaagacctcgtctgtacagtaaattttaaaaattagccaggcatggtagcattcacctgtagtcttagctacttgggaggctgagatgggaggattgcttgagccagaactttgaggctacagtgagttattttcacgccactgccctctagcctggctgacagagcaagacctgcctcaaaaaaataagtaaaaaataaattaaatttcaatcattagcagtcattaggatatttaaatacagtatgttgaatcaaagttacgcatgtgtgtatttttttttccagagagttgtttatcatgtgggttttaatttaactttaaaaaaatgttggctggacagttgcccaaatggtatcatcagccatttggttgagaacgtatgtcctgcgggctcctctgtcactggagttttgctagctgacagccactggctagttagagactgcagtcagcacagatgcaggcgtggacttgcgcacgtaaccatgtcaatgcaaagccatcacttcttaaaaattctgaaccctgctgtctgagatggtggtgcagcggatagaactctgctctaagaggcagtagctaattccatgtcttctttgcccttgactagctgagtgactttgcacatggggcttgcctctctgttgccttgtctgcaaagtggaatcatcttttccttgctagacagaaggtggaccctggacctatggcctttttgagtttcccccccgcttcttagaaggacctctgatcctactgagtttaatacccacgggttaataattgggaaaagcaaaggaagcgcttctgtttaggtaattatatgcatgtttttgtctttttctggctggaaagatatccaagccactgggaaggtccgtggctacccagggtagccctctctggggagggctgctatatccaagagcccctcatgagaatttgaaaatcgaccatggtagggcctgctgacttttgacagctaatggtgtgctgagaattgtccctccaaagatgcctttccattccctcgggagagtctgggcagcccctactgggggctgggatgctggctcttccctcagcctccaccccaactgctctcttccctcctcccctccccagccccctaatttctctcacaaggctttgttctgcagcaacctttcctaatgcagtcctggcctcttcgcagcttcattacataaccttccgtggactcctggtccaaggatcaccccagaaagccagtcagaggtaggcacgcagctggggtccatttacttaccttccccaccccctcggaactcagaggtggtgcaggaatttggactccaagaattaacagctccaccaccatcaccagagccaaaactcaggatgcatgtgcttcatctgctgcttatttccagctgagagccagtggtgccatggttccttagggagccggtcccctgatgccggctcctggccccaaatctctctgatccgggctcttccagaatgtcttgtctccaccatcgcctttgaccaatggtgtccctttgcctggtaatgtcccctttgcctgatgatggccctgtcactcctctctttagcacagaggaggctgtttcatcccttcaagcctgccctcccttcaagtcttagctcaagttcaccttctccgcagagccttctccaatcttcttgactacgtctcctctcagctccagcaacctctgtctctggcactgattccttacttagctaagagaatcacagacacttggggctcaggacaatctgctttctctcttcttacccatggccttggactgtgtgtacctctttgtctccactcccaaacccaacccccagagggcagagagcatgttgtctgtccctttgctcagcatgaagccatgcgtgtggtagatcggcagagttccataacttgtgttgaccgaggggtcactttgctctgaaattacccctgtgtccttcagtatttgcacagatagcttcctggccagaccgaatatatccaagggcatggcccacctctgctcctgtttccaggtccctggtgggggttagttcatgccttcctcataatctgcccactggcctggtcctcaaggtcttcccaactgctcagccagagttgagaaaatgggtcgctccatcctgtttgtgtcgttctctccttcctggcccactctcctgcccacaggtatccaggggctgcctgtagcattagaggacatacatgcacatgcgtgggcatgggacactcacgtagcctccaagcacagcatcaataatgcattctgtgctttatagcatggaaagctgctctaaactttattacacagtggacatgtctgaagcagctcccaaatccacccctgagtgtgttggaattggcaagcctatcacttgggagtctagtttttttgttcgttaataatagatgcttcctgtggccccagcttggcaattttgatttaaagtgatcttaactgaagagactaatggacgggtctgaatttgtgccttttaagcacaaagtattgctcttaattaactggattctatcctttgagcaggcagaggccttcccccaagggcgtcattaacgatccacatctggacatcttccaaagccttcttctgtttcaggccaaccgcaggtgtgttcctgaacacccaggaggctatgagagccacatatgcctcccaaatacacacagtgtgcatgcccagggacatagagcagtgtgcaaagtcccattccatctctctccacctgggagaggatggctcttctgtctgattcatggctcaaagtggtaaaggagctccccactccccgtcccacgcctactcagagtctgcaaatatgtatgcgatatgagagctcgtcagttagctgtcttcagtgtggcgcacatttgaggagtctgactcccctccagcacaggccaatgtgcactgctctcctatctttgtacccccactgttgcactgtgcagaggttggagccatagaagtaccagagctgtgaaaggagaggccccctctcacctctgccctggtctccatccccactttctctaggaagctagtaggtgctgacaggggagagaagggaggggaggggtccagaaacagtggctcatgcctgcaatcctagcactttgggaggctgaggcaggaggatcatttgaggtcaggagtttgagaccagcctgggcaatgtagcaagaccctatctctacaaaaagaaaaaatgtaattagctgggtgtggtggtgggcacctgtagtcctagctacttgggaggatgaggtgggaggattgcttgagcccaagagtttgaggttacagtaagctgtgattgcaccactgcactccagcctgggcaacagagctgagaccctatctcaaaaaaagaaaaaaaaaaagaaaggagagagagagaaagaaaagaaaagaaaaaaaaaaaagaagggaagggaaagcccagaagagtgtggggagaggaggcggccgtcattctggggccctcagtgtgcacaaccagataacacatgctctgtgggcttttgtaccattttgcttgagcataaagaaaggaaggctgcccctaaatagaaagcactctggaggcaaacaaatctgactccaatcctggccctgccactttcccagctgaggacttagacaagcaccctagcctcttggacattctcagagccatctgctgcaagtgggtgctgccatacccaccttactgggcaggcttgggggaccaagggtggtaaatggctcagtctttcatgatgcggccacacagcaggtgcgccatccaggtccatttctttccttcctttcccccaaatcaagttgtcattaaagtactagtccacattaatgaaatcaactgtattaattttctatttgctgctataataaatcatcagaaatttagtggcttaaaccaacacaaatgtattaccttacagttctggaggccagaagccctccataggtgtcactgggctgaaatcaaggttttggcaaggttgcggtcctttctggagggtccaggggagaatccattttcttcctttttccagcttctaaaggtttcatgcattccttggctcatgatcttctatagctatagtcagaaaaattttccatcaatcatcttcaaagccagcaatggcaggatgagtcctcacatcaccttgctctgacaccagttctctgcctccctcttccacatgtcaggaccctcatgattactttgggctcactctgataatctgggatgatctctctattttagagtcagctgactgggaaccttaattccatctacaaccccaattcctctttgccatgtacagtgacatattcacaggttctggggattaggacgagcctgtctctgaaaggctactttacatgaaaattcatttttttaattaagatttttttttcctcttgagacaaggtctcactctatggttcaggctggagtgcagtggtatgatcacagctcactgcagcctcgacgtctctgggctcaggtgatcctcccacctcagcttccctagtagctggaactacaggggtgagcccccatgcccagctaatttttttttttttttttttttgagacagagtctcactcagtcacccaggctggtgtgcagtggtgcaatctcagctcacagcaacctccgcctcctgggttcaagtgattcttgtgcctcagcctcccaaggagctgggactacaggtgtgcaccaccacgcccgactaatttttgtatttttagtaaagatggggtttcaccatgttggccaggctggtctcaaactcctgatctcaagtgatccaccaacctcagcctctcaaagtgctgggattacaggtgtaagccaacatgcccggccccagctaatttttaaatattttttttgtagagatggggttttaccattttgtctaggctggtcttgaactcctgggctcaagcaaacctcccaccttggtctcccaaagtgctgggattacagcatgagccactgcactcggccttaagagaagatttaataattaatactttacaacaagatctggaagaggtgggatgagtaactaaatgaggatacaagtaacccgggtcatatttgctaatacccttggtcacattgaacttgatatcttatcagattttcctaatcagctcctttagcagcagtgttgcagcatcttatctcattttgttttttgtttttttgcctagcacatgcctgtaaatcactggattgaggtgtttagatgtttgttgtcctttggatgcttcttataaatccatatttcatggctccctggaaagtgctatgcaaatgataagctgcaaggatggaaaggaaattgcagtgctcctgaattgtaaatgggcttttacgaggaggtttctaattactcgctctttctcttgaactgaggagttgaagtgtaggtggcagatccataacagataatcatgtgtgtgatgtgacttcagcctgagcgtcgaggaccaagtcacagagcaggaacagccactctccagtgtccttggggctacgtctgaggagaacctgggatttcatatatgacctgcactggctggggggctctcttgacgtaacgtgttccctctgagcatgttacagattctgacattcttatgttccttctgtggagagacatgtacttagtgacctaactcactttagcatatttttgctcatcgtttgtgtagcttaaaggaatcagataattaccccctccccactactttcggaagcacaaatgcaatgccctagaattgtactggggactcaaaaagaaaagagagtagtaaaatctattaaaggggacaaagacagcctatatactacaagctttctatttttatggcagagaatgccattttctaagtaaacagagaactgcatttgacctgcaatatcaaatgcatggatttgatgctttggaaagcaactgttttctgcgttaatctgggtgtcttccgtgaaatgtcctcctgcctttggcttaaacactagctttgtctacagccattccatcctgaacctgcccaatcttgtctgaatcctggtttcaccactgacaagctgtgtgtccttgggcaagttacttcacctgtctgtgcttcagagtcctcatctgtgagttggggaatctggacagaatctaccccatagggcgtagtgaggatgtgttgaattatcccaagtggctacacagagtaagcactcaaatgatgtcatcgttgtcatgattgctgttaccagagcctagagttcattctgatactcgagtctgtggcccatccagcccaggtaaggaatagttggaggagttgggcatgttcagcttgaagaggagacgacaggggatatgggatagttgaatctgtgaagggccccctgggatgaagaactggcatgttctgtgtggctccagggcactgagcaggacccatttgccaaagtctcagggacacagtttctagctatagacagaaaaattttctgtcactcagaggatgaaaatagaatgagcccccttaagaggtaatgagctccctgtcattggaaggattccagaagagctaggtaaccactttaggtgctatcaaggggcttttttctttaaagtcctttccaaaagcttctgagattgcataaacaataggaagccatcttggtgctttaacacaaactctccccagtgatgagggttgagccaaagccagattggcaagcagagaggagacttgtgtacaaggagttcctcgagtcaattgctttttccttgttctagccagccagagggctcctgttggaaaacaggagaccggagaggctgaggcctgaccaaaccagcttctgcaggccagctgggaggccacaactcctacctacgggaaaactgaagggcatctctatttttagattagcaaaagaaaataaatttaagtttgagtctcctttgcaacttttaaaagacatctttattgagatgatcattcacattctataaaattcccccactttgagttacaattcagtggttttagtcttccttgatgattttgatggtcttttcttaaggctcttggaagacccagaagcctctcagacacaggtgggtgtggagggcgtagcacagaggcagacttctcatttcctgggtctcccctttaatgactctcagagacccctccttccccctgcccctggcttctaccccaggggtgtagagttttgccattttccaagcagaacttcatttcctcttctgtgtctacactctttgtgcttctttcttgccagctttttctcctttgcccgcccttccttccttccttccctccctccccccttccctccttccctctttccctccttccccccttccacccttccccccttccccccttccctccttccttccttccttcctgcctgccttccttccttccttccttccttccttccttccttccttccttccttcctggtatgtgactaatttctgtttcaggacataaatgttgtccaggctgttctttggtctttctgttggataatggacatttggcattgagagaggctgctttttctgaaatcatgttcttggggcccagaacctaggtgtgtgcttctgactttgttttcttcctgatccaaattctgatatgtccatttaaattgatctagacccacagggcactgtgggacagatcctcagtggaacatgactctgtaacgagagcattttgttttgtcaaaatgagaacatattattgcctttcatctgattgtaaacataatacatgtttataaaacagtataatgagacaaaaatgtagacactaataagggaaaatctccctaattgtatttctcttcacagagaaagcccctgttgggcatatatactctagtttgtttatttgtttgactacacatatatgtattcttttcttatgtataaaaattctgaacatgcacatttctgcaactactgttttcacttgatgatgcatggacctctctagagtgtacgtttcttcttccttacaaagcagttggcttcgcccagggtgcaccaggacacggttttggctctgtccccagggtgtcacgggaccaggggatgatctcacagggtctgccatctgccctgcctggccggaggctgcatcgagagggccaaggggcaccacgtgtcgtgggtactgtcaaacaagagccttcagagccttccacagtctttcttttgcttcccagcattgcttccccgctggtggactctgaatctagaactagctccaggcgcctctccaaattcagacgggagctggggcactattataatgcaaatctaggcaaagccctcccaataccaggatccagaatggggtggggccctttgccctgaaaagctgtttagtttgaaaatacaaacaggagacagaaaagtttggctaaattaatggataaagttttaacgatggtaaccatagtagggttcatcgacagccagcgatggttctgaacacttgacatgtattaactcacctaatccccacattttacagacaatgcaaaggaggctctgggaggttgagtgacttgccccaaagtcgcacagctcctaagtgaaggattcggagtggactccaggcagcctggtctgactccctgcactgcgctgtgcttatctctggccccaatgccgccatgcagaagtgtctgggggcactttgtctctgtcagacagaattcggagatgtgtatgcttgccctggtatggcacttctctttttttgagacagaatctcactctgtcaccctggctggagtgcagtggcatgatctcagctcactgcaacctccgcctcccaggttcaagcaattcttgtgcctcagcctcccaagtagctgggattatagatgtgcaccatcgtgcctagctaaatttttgtacttttagtaaagatgttgttttgctgtgttggccaagctgatctcgaacttttggcctcaagtgatctgcctacctcagcctcccaaagtgctgggattacaggcatgagccaccatgcctggcagtgtggcacttcttacgtgtgttcagcggacactgtttatcttctgtccctccaagacggtgctgagctcaggtcgttcattactggcagacaactgctgatttccaacagaattgccatcctcttctcccctgcgactttcagagtgtgacctcagactcaaaaattagaagtgaaaacatcttaaaaactatcaccttttcttcctaatcctcctctcccctccctgtcttccttgttgtccccatctaatgaactatcatggcaaaaagagcccatttctggtcattttctgtggcctttcaaactcccacctaccccactgctcctgggtgcattacccgaaagctgagacttcagtgcagaaagtgccaggccctctgtccccccagatcgccttccttgtcttccctgtgcttgcctgtcacattgtgtgggttccagcgctggaaggaatgaggaacagattctctggttctccttttgaagtttaccttcgctccaccacttctgagaccttcccggaagttgccccttgtttctctcctctccagggctgccccagagctgcctctcacctcttcctgctgtcaccccaccaccatcagggcagaagttgggacaaagcctctcctactggctcctgcttttctcccttaggtccagcctcctcttctccatcttcaggagtctccttctccactcacacgtcatgacttcagcacctcgcatcagtccagaatatgactgcttgttcaagtgccacctttctcatgcatttttttctagtgacaatcacagccaccctgtggggcaggagtgtcatcatccccatgtttcaaatgaagaattgcagttcagagagggcaagtgactggcccagcctcaacagctagccagtggaccccaccagggcttctgactccagtccgggttccctttccacccaaatccatggagggagctgagccgagaacaggtgtccttcaggaagacgtgaagccaaagcctccacctccaaactcaggggcccagggagtccaggcacccatccactcacaaggctggatatggtgcattccaggagaggggttgggggcgagtggcctctctgtgtacccgtggggatagatgcgcaagtggcatcgccacatcgtgagtcctggcttcatgggtgagctccaggtccaacgagaagccaagcagggggcccttcaagctcagctttgggcccgggtcggggtacagggtagagcgggcctccccagcccctgccatgaggccaaggcagtgcatcgttcgcagcgtacattcagaaaccaaagcctaggagctggttatcattccggtttacagctgatggaagagcaggtgcttccgagaacccacagtgctctttggccagtgacccaagggtgcctctgagaggcctcgcagcacccggaggtgctgctgaggcaacgccctgactgtaagaaggaccattcatcctcagagagtggccgtgatgctgctgcgacagtcccaccatccctcccgactctcactcccaacagacttcccactgtaaagctgaactctccagcaaatcacctctcgccagactctctcctcactctctctgggtccactagaggttcctcagcctctctttgccttggttttcccagctgtaaaatggagcaaagagggcctatgtacccacaaaggtgtggttggagcgactcctcctacattagggcctcgagtggggcttcatgattggttggtggaggtctccaaacccacccagtgccaccgaaggctgagactgcagatgcaatgccacaggtgtccttcctcagcctgggcagctgaacatcatgtgtaaaacggggataataagataataacagccccttgcacctatgtggctgtgaggattaaacaagataaatgtgtaacagtgcctggctatagaaatatttactcttgttattaagggaagaatatgtgtggctaaaaagggatcgaagatgtaaaagccaatccctccccctctagcatatttaagggtaatgttgagttggtttgtggaccatttgctgcctgttagagctggaaggtagggaccccctctcaacagcgatgctacaaattatacccattggaggtcaaccaaaagacaaagcttattggctggacatggtggctcacacctgtaatcctagcactttgggaggccaaggcaggcggatcacttgagatcaggagttcgagaccagcctggccaacatggtgaaaccccatccctactaaaaatacaaaaattagctgggcgtggtggtgcacacctgtaatcccagctactcaggaggctgaggcaggagaatcactagaacccaggaggtgaaggttgcagtgagccgagatcgcaccactgtactcaaaccgaggcaacagagggagacgcaatctcaaaaaaaaagaaaaaaagacaaagcttgttaataccagcatattgttaagggaataaagtaggctgcagaacaactggtgtaatatggtgccatgtagggaaaattacatgtgtgcataggagaggggtctgcaaggttgtgccctaagatgttagagtggttcctttgcttttctcttttataattttgtatttgacttttaaataaggaccataaatcacttttataaaatacattctctccagcccctactactcctttaaagaataagagtggtttgcccaagaaagacagttttttttgctctggttttcttgattctgacatcagaggaaactgcttctcatccacttggggctctgggttcaggggattcatttcaggcagattaaagtggtgaccaggggcattcgtggacacagggagggacaggagcaccatcagtttgtctcacacaaccactgtcatcctcactgaaggctgttgcctgatcaaaaacagtattgggccaggcacggtggctcacacctgtaataccaccactttgggaggctgaggtgagtggatcacttgaggtcaggagttcgagatcaacctggccaacatggtgaaaccttgtctctactaaaagttcaaaaattagccaggcgtggtgggtgcctgtagtcccagctacttgggaggctgaggcaggagaattgcttgaacccgagaggtagaggttgcagtgagccgagatggcaccaccacactccagcctgggcgaccgagggggactctgtcttaaaaaaaaaaaaaaaaaatatatatatatatatatatatatgtcaaaaatggggtagtttttagatctatagtagttctaaaaacaaaggccatccaagcatgacagatttacaagcactattggctattccagtagttacaatggaggagagaagcttttagttaaaacaaacaaacaacacaacaaacccagaaaccttaggtcaaaaccaaaattgtcctctcagacacaatctgggaattttctcatgacagtgggcattagccaactgacatcagcagcaaccatccgtgtgcacacagtggcaccacctcctcccaaaaagcagccttcatctatgccctcatacaatcgttgattattctctttggattgaggcccggaattatttaagtttcttcttgccagcatgagtctttcctttctgtatgctccttatcttctctctttaatttggcagttctgcttgaaatctgggtctttcattagtagtagttcaatttggttccagaacattctgtggtgtgatgcaatgtgaccagagctcacacttcagagctcttcaagggccagtcttactgagcacctcccagtggctgcctgtgtgctgggcgccacttgtggtgggcaggagagaggaggggacacaaaaggagacacagctccttcttagaagctcaaagttggggaccagctgccacagaagagtatgtttagcatctgagacaccaagatccagcgtcacaagggtgtttattaagcctcctcatctctttctttttctttttttttttttttttcctcaggcagtcttactctgtcacccaggctggagtgcagtggcatgatctcggctcactgcatgcaaccaccacctcccgggtttaagcaattctcctgcctcagcctccccagtagctgggattacaggtgcccaccaccacacccagctaatttttgtgtttttagtagagacagggtttcaccatgttggtcaggctggtctcgaactcctgacctcagatgattcacccacctcggcctcccagtgtgctgggattacaggtgtgagccaccgcgcctggccttgctgttgattcatctatagtatgtttgacttgatgacctccagttaccttagacagaggttctcatctaagctccaactttccatttcctttgtcctcgtctttccccttaacccctccacatttctctcaaaatcaccccacttctaaaaaatactgtttatttttcttttaaatttcaaattatctatactcattgaaataaatcaaaatagcatggaataagcgaaaaaaatggatcccacccttccccactcccattccctagggctaaccatagttaaccatttaatgactaggtttttttgttgttgttattttttatttatttattttgagacagagtcttactctgtcacccaggctggagtgcagtggtgtgatctcggctcactgcaacctctgcctcccaggttcaagcattctcctgcctctgcctcctgagtagctgggattacaggtgcctgccaccacacctggctaatttttgtacttttggtagagacagggtttctcaatgttagccaggctggtctcgaactcctggcctcaagtgatctgcccaccttggccttccaaaatactgggattaaggtatgagccaccgcacccagccctcctgggctcttttcctttagttgcactcgctccccgctcctggagtagagggatttccgagagactgtgggctccagccttcacctaggcccaggactaggatgcctgccctaacatttatctttataccttaaagcaaaacagctggaccataagcattcaagaacaaactgtgaataaggagaaagttctcccaggaaacaagagctttagttctgttgggccagcccttatattccttagctgttaccagtcactgcttgatttaatctcggctatcacttggcctgacaggtctgctgctggtgccaggatgtctgggttttgaagcctggctccattacatacttcctgtgtgaccttgggcaacttactcaacctgtctgttcctcagtttccccagctgtattatgtcagcataatagtttgttgtgtgaattaaatgaggtaataactggaaatgcttcaaacatggttcctatcatgagaaatcctgctttccgcctaaatgtgctggaaaattcctggtggtgcagaacaggagaccagagcaaaggaaagacagggtgcagaagccaaaaattaccttggagaacaaagcgcatgttaaggttatttttggattctaggtttatctctgcttggtcttcagttacctgcaagagatccatttaggggatttttgtttgtttttaacgatagctttattgagatataattcatatgccataaaagtcactcttttaaaatgtttccggtatattcacaaggctgtgcagccttccctgtccttgattccagtctgagtttttaactgaagggataaggaggaccacgctttccccagaccagaaccgcgggccagggggcgattctgctgagtcaccgcgggcgcctggtgcgcggcggcggagcccgggaccttccttggctgccccctagcgagggccgcagcgcagcctgagacacccgccggggccgctccacggccgtcggatttagactggaagctcggtccaggtccccagcttgatgcgcccgcggtgtaggagaccagcccgactcgggcttcccctgagcccctggactcttgactccagcagggcctgggtaatgaacgtcagctcccctttcccaaaggggttgctctgttgggaaggcacccgtttgatacagtagcatagagatgggttttagcatcaaaatatcagaattcaagccttgctctctgcttactagctgtgtgaccctaaaaaggtttctgaacgtctctgagcttcagtttcctcatcattccttctcacggggtggttgtgagcattacagagatcctctctgtgaagcccctgtgagtggctcatcctgagggctgaaataaacatgttattaataatccaaaactggcaagggatgttgactggtccccctcccttgcccaaggagctttctagaacctgagttatcattaccaaactgtactgccttgagtaagaaagttagaaggaatgggaaggatggtggcaggtggaggaaggcggattggtcatcacctccttgcagcaagaaacagccccagatcgtgggaaacctacagacctgctagacagactaggagcaaaagctggggctttaagaatccccagggaggttctcctgagagagtagccagttggattttgtaagcagagatttgtttggggaggaggtgacaacgtagggagcagaggggcaaagctgtcgggaatcctgccttgagggcagggatgtgtgttggggggagttgggtcactggggctcggtggccttgggcaagtttctacctctcaggtcctttacccacctagggtcgccatcctgcccacctcacaggttacagtgagcctggatgcactgtcatgggcaggtgcccaggaaaatggcagacatgttccaaacagcacgcagcattccccagtgatgcccagggtcaccttggaggtgggcgagatgcctggggtttctcgtccaccccacaacacctcaggggacagccaaagctgtcccttcaggtaagctgcacagaagatgtgaactctgctgcaaagactctattctttgggagcaaaagggacccagggtctcacctgcacatccctgtccctgagggcctaggggttcttggaggccccagccttggcaaaatgaggaagaaggtgaaggttgtctgggcccctgccaggctccttcctcggccacgcactccccttcctgcacacacacccttctccctccaccccatctccattgttgtcagaaaagtcacaataaaaaggtccatattgtctagttcccatacttttaatttttaaaattttatttatttatttatttatgtattttttgagacagagtcttaacccaggctggagttcagtggcatgatctaggctcactgcaacctctccctcctgggttcaagtgattctcatgcctcagcctcccgagtagctgagattacagatatgtgccactatgcccagctaatttttgtatttttagtagagacggggtttcaccatgttggccaggctggtctcgaactcctggcctcaagtgatctgcctgcctgagcctccggaagtgctgggatttcaggtgtgagccaccgcactcggctccacacttttcacttattaaaagactgtggtgtccatcaatggatgaatgaataaaccaatgtggactatccctcccattacccaaggaatgaagcacggagccgtgccaagatctggattcacagtgaaagaagccagtcaccaaaagccacgtgctgtgtgacttcccttatacgaaatatccagaagagatacatccatggtgacagaaagtagatgagcagctggggactggcgaaggggagaagggggagcagctgtctatgaggtccagcctttcttctgggtttggtgagaatgttttggaactagatagaggtgatagttgtacaacattgtgaatgtactaaatgccactgaatcattcattttaaatcgttctttacgttgcatgaattttaagtcaatcaaaaacagttgtttgaaaagagaaaagcctatgggtagcggcagcagtgattggatttatgattcgattccatggctcatccctcccctgcctcaccccctcgccctccgacgtcttcttcttttactctgaactgttatctttgttctcatctctctctctctctctcaaccctgcagacacttttccctttctttgtctgcccccaccctccagatttccgtgtctccagtgtctccctacgaggcatgaattgagactgggagggtgtgattctgaagaaggcaccaacagtgactcagctagccccttcccccaccccgccccccgggcctcaatttagctaaaaaaccacagggacggactcaggaggcaatacctttccaagggtccctaaaaaatgtcccattttagtgtccaggtttcactcaactttagtgcctcccctaaaatgtgttccttacctcccaccccactgcatctaagtcactgcctgagaaaacaggattgaggaaaggagaaaggaagagagagagagaggaggagagagagagagagggaggaaggctgatggatttagaaaagaagaaaacaagtggtctgaggaaaacagccttggtgtgtttattttcctgtctgtgtatcgcttctcggccttttggctaagatcaagtgtattttcctgtctgtgtgtctcgcttagattacagggatctgtgggtgatgacacgtctggtccaggctgcgtagtcacctcaagggcatgcttattgatgtgtttttcaattcactatctttgcatgggagtcccaggccaagaggcacagctgcgccatttgtctgttggtttagatatcctttatccagttcttccagagaaatcatcctgcccttctggaggaggtgggcagcaggggtcagagatgggagggaaaggaaggagccaggtccttggctaggatgccagggtcccctgcctctcacctggcctgggctggaggcctcctgctgtcctgtcactgatcactaccccgccccagcctcctgagttagaagacacaggctaaagtagagtatttcttcattgaaaaacccatacaaaataaaggttcataaaaaataaaaatttagactgggtgctgtggctcacacctgtgatcccagcactttgggaggccaaggcaggtggatcgcttgagccctggggttcatgaccagcctgggcaacatagtgaaaccccatctctacaaaaaatacaaaaaattagccaggcatggtggtgcatacctgtggtcccagcttctcagcctatggacccacatagaatacaatgtcagcataagaagggagccctggggtcaccaaatggtttgggcggcaaagaacctgaaggttgagagaagtggcttggttacccagctgttggatgtgagacctggccactgcttcttccataccctagacctgcaccctgacatctcaagtaaaaagttgggggatgttttatggtccaggatgaaggaagggcagtgaggggcagcggagcatcactttgcatttctgtctgcctcttactggctgtgtgacctggggcaggtaacttcccagactcctgggaatcataacacctatgatgatgatgatgatgatgatgatgatgacacctacctcaaggattgccctgaagggtcacagagatgcctgcaaggcacctgcatggagcaagcgccccttctctggcaggtgctgggtgagcactacctgctgccaggccctggggctatggcactgcgtgaccctgcaagtcctacctggcgaagctgtcgttcttgtgctcagtcagtgttggttgtaagactgagaagagtcacttcattttgctctccagggacatctttctgggtcctattttctgcctatgtcaagtagcgcctcaaggatgctcctgaaaatgggcttgtctttcttaacatggcaggtaggtcccaaagcattagcatggggcagctgacctagcccagccaatgcagtgcagtgactcttgcaaccgagtctaatcagaaggtccatgaacctacgagcatttcctgtcccaggatcagggtggaggctgagcctccctgcttagagattcttcccatgcattccacttttttccccaaaagaaaatattgacccttgagaggcacacagtttatttattttgcatagtaaatagtagcctgtattttaaggatgagttgatttctgcatcagcccctgtaggtcatcagccttctattggtgcatctgactctctctagccctgcagggatggtggagggggaggggaaggagggatctttattggaaaccaggacagtgagactcattgccctgtcatctgctctgtggtgctgaatgaggcagcccaacagagaaataccctgagcgagcatccccagcctccaaaacagtggcgcattgccctgagtcctgggaatgacctttgattctcctgctcctgacttggaacccatggaaacctctagaagcagctgaggaaaacccaacatgaaaagcagaactccacactgagaatataggaggtgatcggaacatacaatgattcttgctaagaccgattcacagtttttcttttttttcgatcgaagaaatactggagaagcctaaagaaggagtctaaaaactctggcacgtgggccaaaactgtccttgagctaagaatgattttcacatttttaagtggttgaaaaatgaaataaaataagatgatgttttgtgacacatgaaagctatgggaaattcaaattctaatatctataaatagtgttttatcagaacacagtcatgctcatttatttatgctcgatggctgctttcccgctacaattacgttgagcagttacaacagagaccacgtggcccacaaagccttacaatatttactatctggccctttccagaaaaaaatgtgccgactcttgaccttaacctcagcaatttgggaggccgaggcaggcggatcgcttgagctctggagttcatgaccagcctgggcaacatagtaagactccatctctacaaaaaatacaaaacattagccaggcatggtggtgcacacctgtggtcctagccactcgggagactgaggtgggaggatcgcctgagcccaggaagtcgaggctgcagtgagctgtgatggcaccactgcacctcagcctgggcgacagagcaagaccttgtctccaaataaataaataatgcaaagtaaaataaataaaaccatataaaaaggaatcaatttaaaattataatgaaagctggccgggcatggtggctcacgcctgtaatcccagcactttgggaggctgaggtgggtggatcacgaggccaggagatcgagaccatcttggctaacacggtgaaaccccgtctctactaaaaatacaaaaaaaaaattagccgggcacagtggcgggcgcctgtagtcccagctactcgggaggctgaggcaggagaatgtcttgaacccgggaggtggagcttgcagtgagccgagatcgtgccacttgcagtccagcctgggcgaaagagcgagactccgtctcaaaaacaaaaacaaaaacaaaaacaaaaaaaaattataatgaaagccaaggggcatagtagaacaaattttctagagctcattaagtcaaatgagtcaccagttagtaaaacgcagtcacggggaagagagggcaggattctttgaagcagcggctctcctaaaaacaacccacccttgtccagctgccttccctcctgagggtgttccctttgactgtgtgacccccatcccctatttcccaaccgtccaagcccacctctagcataatacgagcttttaatccctctccctgaccccaacccgattttgaagcccagtctagtattttctcaaatacacttcttggctccattccttcctttccatcacctctgccttttcactgcatgcttggaccactgcagtcagctccctatgaacagttgctctctacccatccaatcggccccgcctgctgctgccaaattcaccgagggcacctctgtggtgctgcctgtggacaaagtccaagccagccacctcacccacctacaggtgagtggggagcagccagcgtgtccagtggtttaccccatcgccacagacttggtgatgtgtcgatgtgcagagaaggggtgttggcagccacaacacaagcaaccccgccccatgtgagatctaagatgggcgtgctgggagccacctctgagaatccaacagaaggcagaggggagaacggctcacacggcacaaacactccttccttttttttttttctttttcctttttgaaaggagtctcactctattgcccaggcaggagtgcagtggtgcaatctcagctcactgcaacctccgcctcctaggttcaagcgattctccagcctcagcttcccaagtagctgggattacaggtacactccaccatgcccggctaatttttgtgtttttagtagagacggggtttccctatgttggccaggctggtcttgagctcctgacctcaggtgatctgcctgccttggcctcccaaagtgctgggattacaggtgtgagccatggggcctagcctccttccatttaaatgtatgcctaatttgcccattgagaacggctgagacgcattttaagtggccagggtctacttagagttagtgctcatgaccaggcccaggtcaagcctggctggccagatggtgcctttgacctgctctgtctctgtgcaaaggaatgagctgaaggatgggggtgcagtgtgtgggcagtgggctggggctggcaggactcagtgactaagggaagagaactttcctcactaccagcctgtcttttcagggcaccgcggggggctttgggacttggtgatgaacacagcacagagagctgtccagcatgcgggtccctggcttctcacacttcccaggctccttcagaggctctctccaaagggagctgctctctctagaacccatgaatttggaatataggcaaccactgcattggggaccactgacctcaaacatagagaccagagcaaatggggctcatcacgtgaaactcatctggaactctagcaggttcttttatatatatatatatatatatatatatatatatatatatatatatatatatattttttattattatactttaagttctagggtacatgtgcacaacatgcaggtttgttacatatgtatacatgtgccatgttggtgtgctgcacccattaattcatcatttacattaggtatatctcctaatgctatccctccccactccccccaccccacaacaggccccagtgtgtgatgttccccttcctgtgtccaagtgttctcattgttcaattcccacctacgagtgagaacatgctgtgtttggtttttttgtccttgcgatagtttgctgagaatgatggtttccagcttcatccatgtccctacaaaggacatgaactcatcattttttatggctgcatagtattccatggtgtatatgtgccacattttcttaatccagtctatcattgttggacatttgggttggttccaagtctttgctattgtgaatagtgccgcaataaacatacgtgtgcatgtgtctttataacagcatgatttatattcctttggttatatacccagtaatgagatggctgggtcaaatggtatttctagttctagatccctgaggaatcgccacactgtcttccacaatggttgaactagtttacagtcctaccaacagtgtaaaagtgttcctatttctccacatcctctccagcagctgttgtttcctgactttttaatgatcgccattctaactggtgtgagatgttatctcatggtggttttgatttgcatttctctgatggccagtgatgatgagcattttttcacgtgtctgttggcgaactctagcagcttcttttcacaagttcatggagagaggtttcccactgagggaatcacatctgtctgatcaaaagaggcttgggaaatggctctcctgttcattccctgaaaacctctgatggaaccactgccactgtggcagccccagcactggcaccccagccatgattggtgccccagccacatctctgctgtgagccccagagccctggttaattaatcatccacgtgttgatggggagaggcccattcacaaaagcgacataaagcccagggagacgtggccgtggcaagaagggtgtgggactacattccgcccccaactgagagattcagaaaccagaaaaaaatggaaaaacatactgtgctcttgggtgggaaaactaaatatcatgaagggagcaatttttatagttttggcctataatacaattccagccgaaatcccagtggaactttgagaatttgcaggaaaaaaaaaaatgtctaaagtacatctggaagacaaacttacaagaaggtcaaataattttgaaaaagaaaatgatatctaagcccacctagagaataagacttgagatccaaagctaaatcaggaggctctagcaaaattgacagataagcaggacagagtgcatggtgcattcacctggggaagagggcagattggtctacaaataggcctgggtccactgactttagctgttatatttggggagaaacttttcaacctcactccatcttaaacctaaaaatattccagatgaattaataaatataaaaaattagaccactaaaaatgtagaagaaaatggatgatctttctataccatagagcaatggaataaatcacaaaggaaaacagatttgactatataaaacttaaaccctgcccatcaaaaaccatcagaaaccaaaataaaaggcaaccaactggagaagatagttgccacaaatatgatcaagggttaatgttattcataaattaagagcccacacaagtcattagaataagcactgagacctgaacagacaagcaaaaagaatgagagtgggtcggcgcggcggctcatgcctgtaatcccagcactttggaaggctgaagcaggcggatcacttgatcccaggagttccaacaccagcctgagcaacatggtgaaaccctgcctctacaaaagtcataaatattagccgggtgtgatggcacacgcctgtagtcccagctactcaggaggctgaggtgggtggatcacttgagcccgggaggtagagtctgcagtgagccaagatcacaccgctgcactccagctggagcaacagagtgagaccctgacttaaaagaaaaaaaaaaaaaaagaggagaaaaatgctgatctcactagtaattaaaacatcaggccaggcgcagtggctcacacctttaatcccagcactctgggaggctgaggcaggcagatcacttgagatcaggagttctagaccagcttggccaacatggtgaaatcccgtctctacaaaaaatacaaaaattcgccaagcgtggtggcacatgcctgtgatcccagctactcgggaggctgagacaggagaattgcttgaacacgggaggcagaggttgcagtaagctgagatcgtaccattccagtccagcctgggctacagagcgagactctgtcccagaaaaaattaaaacatcacatatttaaacaactctaggatatcatttaaaaaaacattaatagactgttttttagagcacttttaggttcacagtgaaactgagtggaaggtacagagacttcccgtatgttccctgccctccacgtacagcctcccccactgccaacgtcctgcaccagagtggtacacttgttacaaccaatgaatcctcattaacatatcattatcacccaagttcatagtttacattagtaaaacatcatctttcatctataagcacaaaaattttttggcatttatttaggtgtatgattaactcagtgttgacaagactcacacttcatacccacttgcactgcatctgagaagcaattggtgtctacagccgctacaccctcaacaagcccgatcttgtttgaaaagcaattggtgatgcttctcaaaattctatggacaaagtcagccgggcatggtggctcatgcctgtaatccctaaactttgggaggccgaggcaggcagatcacctgaggtctggtgaaaccctgtctctactaaaaatgcaaaaattacccaggcatggtggctggggcctgtaatcccagctactcgggaggctgaggcaggagaatcgcttgaagcaaggaggcggaggtttcagtgagccaagattgcaccactgcactccagcctgggtgacaagagtgaaactccatctaaaaaaaaaaaattatggacaaagtttttcaaaaagatatttaatgcaactttatttgtaatattggaacatctgaggccatttcagtgctaactattaggggatggttaggaaaatatggtacatatgtggaaaggaacatttggtagttagtgcccctgatgtttacaaaggcttttagtgaccaacaaatgctcatgctataatcttatgtgaaaaaagcaagtagcataattgcaactatatttttaatgcatagaataaaaggctagaaggaaatatcacagatccttgacatacattcccaaacctttgtaaatccgcggattcatgaaaacagacacatttgcacaagtgcctgatcttttctgttatacattcattagaagtcaagccctggtgccacaaagtatctgccttttcaaatgtgatcagaatgttctcttttgcttcaaggccatttttcacgaagcagtggcatttttgcctcttcatcagagtcaccgtgtgccctggaggactgagaacagcagagccgttttaggatgggacagggcagccaggaggattgggctcactccctactgagtgcctcactcccgtacagcccccatagaggaagaggggttcaaatttattcctcagccagatggcatgtgccgcctgtcctggaatttcacatcacttatgatggaccaaaattccaaaagctgaatccatgattgtcaaagtctggtatggcaggatgtcaacagtaatcgtttctgggcagagggatgattttctcttcccatcttgctttgtataaatacattttctataataaggttgtattacttttctcatcaagaaatagcaaagtactgttttactcaaaatatgaatagagccaggcatggtggcagcttatgcctgtaatcccaacactttgagaggcggatatgggaggatcactttagcccaggagtttgagaccagcctgggcaacatagtgagacccccgtccccactcccccaaagaaaacccacaaagcatttatcctggattattcacaggggccaaaaaaaaaaaaaaaaattcaggcctcctatagccatgagctacgaatatgaaaatatgcaaatgtgtaagaaaagccagcacatccgatttttacttttactttcacacctctgtccaccatgttccaagagaagaaacttggtcattgaaaggaatagatcaaatccaaagaacaaaaccactgtgctcattaaacttcttagtgttcacaaagctttagctgcaggttgaatggggcaacccgaattggctggctcacctgggctgcagggagcagagatcgcgacactgcactccagcctgggcaacaaagcgagactctatctcaaaaaaaaaaaagttcataaattcaaagttatgaattatttttaaaataataataatttacaataaagatgaggacaaagtgtgagtaaatggtggtttctatccagctctgttgagctgaagtggcatctccctgctggggcttttggggaagaagggtgtgtgttgctcttcagatcccaagcctcatgcccctactgggccctgtggggtgcttctcagcccaccaggagagccaccgttggaacacacacgtgggggacctggtgggtgccggtgtggtgaatgggggccacagcctgactccaggaagccagcaaactcggagctggaggagtcaggacacccccgatgagtcaagagttggttttgctgccagttgacatctgattgaaccatctcttcacttctccgtgcctcactttccttaccagacaggctctgctgatgctgtccctctcctgttcagtcgtgccctcaccgttaaagagaaagagcaaactgctgggcagcagcattgatttttttaatgaagtggaaagagagctgggaataacaagtcgggcccacctcacctgcctcacctggtgggtttatttgttttgtttttttttttttgttttgagacagagtttcaccctgtcacccaggctggagtgcagtggtgtaatctcagctcactgcaacctccacctgccaggttcaattgattctcctgcctcagcctccccagtagctgggattacaggcacctgccacatgcctggctaattattgtatttttagtagagatggggttttaccatgttggccaggctggtctcgatcccctgacctcaggtgatccacccacctcggcctcccaaagtgctgagatcacaggcgtgagccaccatgcctggccgtcacctggtggtgttgaatatgaactgctgcggtgttggtaaattaagcaagcagatagatgtaaataacgcttgggcaggaatatggagcacgggatgaggatgggcggccaactgttagagagggtagcagggaggctgagatctgcctgccatgaactgggaggagaggctcctctctctcttcacccccactctgccccccaacactcctcagaacttatcctctcctcttctttccccaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaaggttagtggacagccatgcacagcaggcccagatcactgcaagccaaggggtggcgggaacagtttgcatccagaattgcaaagaaattttaaatacattattgtcttagactgtcagtaaagtaaagcctcattaatttgagtgggccaagataactcaagcagtgagataatggccagacacggtggctcacgcctgtaatcccagcactttggaaggcccaggcaggaggatcccttgaggccaggaatttgagaccggcctgggcaacatagcaagaccccgtctctaaaataatttaaaaattagccaggtgttgtggtgcatgtctatagtcctagctactcaggatgctgaggcagaaggatcacttgagcccaggagttcaaggttgcagtaagctgtgattataaaactgcactccagcctgagcaacagagcaagaccctgtcaaaaaaaaaagaaaagaaaaaagaaagaaagaaatttaccttgagttacccacatgagtgaatgtagggacagagattttagggccttaacaatctctcaaatacagggtactttttgaggcattagccacacctgttagcttataaatcagtggtattgattagcatgtaaaatatgtgactttaaacattgctttttatctcttacttagatcaggcctgagtggcctctctttagcaagagttggttagccctgggattcttactgtagccacattaataaacaacatcgacttctaaacattctataataccatcttttggccaaattgacttcgcctcttcctctctctttccaaatgaaatgtgtttcatttcactgtcagaccacatggttggggaccccacagagcacacagccctccctctgccttcccatgctggcccttcacccactgctggagtgccaggttggtccaagggttggaccaagttgtctgaggttgtctcaaggttggtcgaggctgtctccgcgctgggttgtgctacaaggagcccttctttccatgggtgtggctggcagtgagtgctcacagcaacagcccacagtgcagcccgagggcaggatggactcagtccctgcctccatacccatttctaaggaggcaaaatggcaaacactctacttttctcttttaatgctaaaaataagaaaacaccttgcagcccagggtatgggtagtgcatggaagccgtggagttgtgaggtgggaagtgacctctgctggatatgtctattcaggaagattgctggagtgggtggggtctctgggaggtcccctgagtgtgggaagctgggaccaccagctttctcgcacagggagtggccatcccagcttggagaggttccaggactggttgggaggcacgtttcagatttctatctgttgaatcagcgaagatattggattatgaggaatttgggaattaggaaagtgggtgcaggtgggttgggggtaggtgaaggaagacatgggcgtattgggggagcaggggctgctcagaggtgttccagaagctctgggtgaggaggtgagagggaccggggaatgcagctcggcccagcctccctgcctgaggtcagccatcacgtggtgatggcaagatggaaatgtgctttctgactgctccagccagtgctgccagattcagctccccagggagggcacctgagaggctccaagccaggagatctgttttctcctttgttttgtttttttttgttttgttttgttttattatactttaagttctagggtacatgtgcacaacgtgcaggtttgttacatatgtatacatgtgccatgttggtgtgctgcacccatcaacttgtcatttacattaggtatatctcctaatgctatccctcccccctccccccaccccctgttttctcctttgaatccttcttagaggccgggcgcggtggctcacgcctgtaatcccagcactttgggaggctgcggcaggaggattgcttgagcccaggagttccagaccagcctgggcaacatagtgagacctcgtctctacagataataattttaaaaattatccgggcatagtggcatgcacctatagtcccagctactcaagaggcagaggcaggaggatcacttgagcccaggaggcggaggttgccgtgagccaagatcccaccactgcactccagcctgggcgacagagacccccatgtcaaataataataataataaataaatccttctcagtcccttcctcactgtgtccccctccactgaatttttccacctcctctcccacttcccccactcccgctttccctctccttctctccccactccatctttttctttctctgctgtttctcgtccctccctcctctccatcccacaacactgcctaccctgtccctgccccaccctggtgctcaggatgtgtgaagtgaggggtggtagcccccaagacctcaaccccgaaggttagcctgttgaaaccactttctcccagctgcccccctggcagttggtgctgctgggggaaactgggattgggggccagattttgcctcttttcctgacaaagagagatgaagagttctctcaccaggtgcctgggactggggtgtgggtgtcccagcctatcccagcgcatctgttctgcatcatgattaatagtgctgctttcagccgggcgcggtggctcacacctgtaatcccagcactttgggaggctaaggtgggcagatcacaaggtcaggagttcgagaccagcctggccaacatggtgaaacctcgtctctactaaaaatacaaaaattaaccaggtgtggtggtgggtgcctgtagtcccagctacttgggaggctgaggcaggagaatcacttgaatctgggaagcagaggttgcagtgagccaagatcgtgccactgcactccagcctgggtgacagagtgagactccgtcctaaaaaaaaaggagttttgctctgtcgcccaggctggagtgtagtggcgccatctcggctcaccgcaacctgcgcctcccgggtgcaagcgattctcctgcctcagcctcccaagtagctaggattacaggcgcctaccaccacgcctggccagttcttgtatttttagaagagacggggtttcaccctgttggccaggctcgtctgggactcctgacctcaggtaatccgcccacctcagcctcccaaagtgctgggattgcaggcatgagccaccgtgcccagtcaactccttctcaaaaaaaaaaaaatagtgctgctttctctttcaagtgtcctgatttgggtgatagtaaatgccactctacttataagggatctacctcagaatgctaattgggacatttttgtagcactctactgttggcagcaggtgatgctcacaacagcccgtgagggtggatgacgtccgcttcacagatgacaaaggagcctcatgctcagaccgtgggctgccagagcaggtccatggctgcagccccacatggaccatatttcccccttgtcactctttccaccaagctcccttggaacttcagttattaagctctcttgggtggaatccaagttagaatcacaacatgtgcctcatatggattgtgccagtgaaaaatgacattctatttagaggcagggcagcctggcttagagtcagtttaaaatatgtattatgctgcaacaaatgtaccatgatcctgtaagatgttcacaacaagggaactggatgtggggtatactgtctgtactaacttcacaagttttctgtaaatctaaaactgttccaaaataacaagttcgtttaaaattaactccaggagaccaggtacggtagctaatgcctataatcccagcacttcggaaggctgaggcaggtggattgcttgagcccaggagtttgagacaagcctgggcaacatggtgaaatcctgtctctaaaaaaaatcacaaaaattagccaggtgtggtggcgcattcctgtagtcccagctacttgcggggctgaggtgggagaatcatctgagcccaggagtttgaggctgcagtgagctgtgattgtaccactgcactccaacctgggcaacagagcaagaccctgtctcaaaaaacaaaaatgaaataaagtccaggaaagaagtaggttttaccactcttattttctgaagagaaaactaaatttaatgtgtaaagtgaggacaagttcaccaagttagtgtttgagttgcctaaaatatgtttgctaaaactattcaaagctttcacataaaacatgatcagaagttctatgccaaaacatatgtgtgtgtatatatatatgcactatatatactgtatataaaaatgcaaaatctaaattgccaaccttttagaaattgctctgaaaggaaagcatttcaagataatttgcttacccaaagaatatactttccaagaaagcaagtaatacttaaggtgttcataatcctcatcaaattaattcttgctactgaaagcttacaaggagctgttttgatgtcgggtgtgacaggtttgacttggcagaaggtgtcactttactaacaacattttaaataagtgacagaagacaagaaactacacgttaaatgccagaacaaagagtgtctaagtggatgctaagagttgaaatatggctggatacctgcccaagagagctgaaaagtagatgaaagttggttacctataaactagtgcaccctaatgaattaaaaggtgttgatgagttaacttgttatgccttccagataagacatgcaaatggggcttcttcctccttcactacttccaagggatttaacaaggagaccaatgcaaatgataaggactgtagggctcaagctggggacagattggggaaagggggaccatcatgcccatatagatgtccctgtgccctggcagtcaaggctgctgaaaaataacaaaacccagaagtctgcgtgatgctgcctctccatttgtccaaagccttcttgcggcagtttgcaggcttttgcaaaagctccaggaccaaggagctatgttcatgctggaagcttgttcaggattagctgttctttgtgggatgggtgcagccagggccaggtgtccagggacagtgttttaacaaagggcatgaggtgtctgatctcacagtggaactccacttgcctttttttcatcttctcattctgcttcatgcacagaaccagccccatcctgaaactgactctaaattactcccgccccaggtggagtgcctttctcggagttcaacagagccttcctgtcgcccaagggacaactccactgaatgcccaagccacacccaaaacctaacaagtaaaaaccaaattctgtgctcccccatcctgggccattcctggtttctctactgctgttggtgataccaccatcagcttgtccatcatgaccctggccagttcctcccacaaccctccacagcacccagggacctcacctccattccatccgacacagatctcctcaccacaaaccttggttttgcaacagcagccatgagacctttacaccctccgcccttcatcctgtcccccactgaggccccagagccattccttaaagcagcgcgccacaaactataacccacaagccaattctggtacccagcctgttttgcacagccagtgaactgacaatgatcttttcatacagccagaaaaacaaaacaaaacaaaaaacaacaaaaaaaaaccccaccattctgagcatgtgacttccatgttcaagatgtctcatgttcagaaaggcccctggaaaaggaggaaggggagctgggcacaaagggagaccctctcagctgagctcctcccatccagacattttcctggacttcctatccaatgacttcccttagcttcttatcagccacccctgtctgcccaggaggctggaagatgtggccttttaactgggcacagctctgtcctctatcatatcagggctctgttcccaaggagggtagagagaatggacaccaggtggaccctcagcagtctgtgccacagagggagtgtttgcaatttccagactaaaagtccccatgtgcttgacggggtatgtgactacaacgtgatgcttgacttttcctcatatgaccagagccactttgtccatctggtacaatgtcagctatctgctaggggccctccaggattcccagtcaattccatatctgcatcaccaccattggcactaaataaaataaaatactcaagttcctgctggtgagcatgagcagtgctacactgggcccttcaaccaaggtgacatgataatgactgaaaataatcactgccacttattggggacgtctcatctgccaggcatggtacaaagtgctttaaataagcattcaacaatttcatgctgacagaagccctgtgagccagtggagctactactatgcccattatacaggggagaaaactgaggcagagagaggttaggtaattcgctcagcctcacacaaccaataggtggtggagccaggatttgggccccatctgcctgactctctagaggctctatcttccagtcttccagagttgagtctaagccatgaataggacaattagacagcagaggaaacccattcagccaccatgtgcatgaagagtaaggaatttctgtcatacagaggggagtgaattcactgagctgagagctgaggaaccattgatctgatggctgagacaccactgggaagactggagaggcttttctgggcatgcagtgccaggcacaggaggagctgagggaagatgactaagaggtactggcaaagaattcagaaattctgatggaagctttacatgttaccatcacatccatccatctatccacccatccatccacccatatcttcctccctccacccaatcatgcatacatccagtcatctatacaccacccacccacccatccatccatccatccatcccttcatccatcccatcatccatccaattatacatacatccaatcatatatctgtacataatccattcttccctcggttcatccatccatccattcatccatccatccacccatcccttccttcatccttcctatcatccatccaatcatatatctgtacataatccattcttccctcggttcatccatccatccattcatccatccatccacccatcccttccttcatccttcctatcatccatccaatcatacatatatccaatcatacatctgcacatcaccagctcatccatctatccatttatccatccatccttccttccatccatcattcatccatcatacatacatctaaccatacatctctacatcattcattcttccatcgattcatccaattatccatcattccttcctccatccatcccattatccatttgatcatacatatatcatctatacatcatccattcatccatccatccatccatccacccatatcttcatccaatcaatcatacatacatcgaatcatctacacatcacccatccatccatccatccattcatctatccacccatccatccatccatccatccattcatctatccacccatccatccatccatccatccatccatccatgtaaccatccagtcatatatccaattacacatccatccagttatacattcatacatgcatctaatcattcaattatacatacacacatccatataattctacatccaattatacctccatccaattacacattcatacacccacctaataaattattaattcatatatccatccatataattatacatcaattatacatccatctaatcattcagtaattcacccaccatccagtcatctatccaataatacattcatccaatcatccatccatccatccacccattcatccatccatccgtccgtccacccatcatggtatgagccatgatttaccacgatggtcccctgtggacagcccaggtggggcagaactgaagggaagcccagggctgcccccataaacatttgcctcctttacatggatgagaactagatccacatgtataaatcctcatgatttgaaggtgcttttaccaacattcactcatgggattctcccaggagctctaggaggaggcaggtagagttgaggtcatctcacgcattttacagatgaggaaacggaggccctgagaggcaggtccaaggccacctgaccagaaagaagtggaactgggacttgaacccagccatcttgccccttggtcccatgctctctagcctgtaactcctgcttcctggtggggcatctccaggaggaccctatcggctggccatgggcctgccctggagtcttttgctctgtgtggccatccttcctccctcaggagagtgtgtgctcccagagcacaggctgtatcttctgagcattttgtcccttcccagtacctagcactcagctctgtatacattgggctctcaagaattctcaaccttccagagtgtaaggccttgacctgctcagccctggatactgcatgatgcattgataagcccataaaataaccagggcagattgactcccagtggccaaagtgccacagggaagggacaattcagcccttctaggaggaggaggaggtagttttctcatttctattaaggcaacaaaagctgccttactaaggacattcttggtggagggcgtgactgtcaaccactgtgatcatttgggcctctcttgcccaggcttcccattctgaaaggacagttttattgtaggtacacatggctgccatttcaaatgtaactcacagcttgtccatcagtccttggaggtctttctatgaaaggagcttggtggcgtccaaacaccacccaatgtccacttagaagtaagcaccgtgtctgccctgagctgactccttttccaaggaaggggttggatcgctgagtgtttttccaggtgtctacttgttgttaattaatagcaatgacaaagcagaaggttcatgcgtagctcggctttctggtatttgctgcccgttgaccaatggaagataaacctttgcctcaggtggcaccactagctggttaagaggcactttgtcctttcacccaggagcaaacgcacatcacctgtgtcctcatctgatggccctggtgtggggcacagtcgtgttggcagggagggaggtggggttggtcccctttgtgggtttgttgcgaggccgtgttccagctgtttccacagggagcgattttcagctccacaggacactgctccccagttcctcctgagaacaaaagggggcgctggggagaggccaccgttctgagggctcactgtatgtgttccagaatctcccctgcagacccccactgaggacggatctgaggaaccgggctctgaaacctctgatgctaagagcactccaacagcggaaggtgggccccccttcagacgccccctccatgcctccagcctgtgcttagccgtgctttgagcctccctcctggctgcatctgctgctccccctggctgagagatgtgctcactccttcggtgctttgcaggacagcgtggtgggagctgagccttgcgtcgatgccttgcttgctggtgctgagtgtgggcaccttcatcccgtgtgtgctctggaggcagccacccttggacagtcccgcgcacagctccacaaagccccgctccatacgattgtcctcccacacccccttcaaaagccccctcctctctctttcttcaggggccagtaggtcccagagcagccatttggctgagggaaggggcaggtcagtggacatctgatcttggtttagtatccttcattttgggggctctgggtgtggcctgggcctctggactttggccacggtgtttgttccagcccttctcctaacctgtcctttccagacactcggcatctaggttattagcacctcgcatactttctgacatgctcctcagtcctgattttgaccatcttctcttgcttcccatctgtgtcagtcaagactgcatttggctgtaagaaacagaaaccccaactaactgtggcatttacatgaagaggtttacttttctcacataatcagatgtctagacttggccagcacctcaagggtcattgatgctctcctgtctttattttctgtcatctttagtggttggattgttgcctcatggttacaaagtggctgctgcacttccaggcatcacatctgcctttgaagcaggaacaagttgcaaagtaaagtggccaaaagggccctgaaactaaatgtgtccccttaggaaagcaggagttttcttgcaagtggcaatcttctgcttatgtctcattggccagagctgggtcttacggccaccccttgctgcgagcaaggctgggacattgagcattttgccgtccaacctctttagcagaataaaccaagggggaagaacgttaatagtggcttttgagtcactagttggcagtatctgcccctctatctttccatcctccccatggagtttcaaggttcctttctcagtacttcttcaggctctgcacgttcatttggatcttgtgtcttggggtgaaaaactggcccaagtgtctccccaagcatccacctttggattaatttggaaaatggctgtcaagtgcccgcctcttgcttggtataatgctacagctttagaggacgcagcaggcatgggccttgccgctgaggttcttagcctcatgagaatatccagatcagattctcttggctccttcttagagccagtgatgcaagacacttcctgctcatcttgtcgggacggttttacaagttgcctgccatcctgagaaagtctacaaaacgatgccagacctcatgccagcttcccaagccttgactctcagtgctccctcaacaggattctggaagaatctcccaaacaagtcgcaatgccctctggaccctgtgcaggcatgagactcaagagcattggctcccacccctggtggagggaacactgctggggctgggatcttgcctggttgctccgcctgcacccaagacaaccataattaaaatgtccttcattgaacttggaaagccttcaaagctgacaactccttatgtgtacccggaaaggcctgggagtgtgccagggcattgctcgggagggacgctgatttggaagcatttacctgatgagagactgacagcagctcctggtagccgagctttccctcctgcctctgctgtgaaggtggacccatccaacagtcaaatgcctgactctggacaggagcggacctatttattgccatgcaagggactctgcacttttgaattgtgggtcatgggcttggatttaggggttagagctgggagaagtcttggaagtcacctagagatgacactgccattttgcagatgaggaaaccgtccaatcaaaatggaccaaggacttgcccaaagcctcacagcaaaaccataggcccccgcactaaccccagagtccctgtgctgtcttaaggatcatatagttgtaagcaatcatctggttttcagtatttcttcttttaaaatgcctggggccatgcccagcagtctgtttcactgcagcgtttacacagggctgccgggctttcctggtggatgagctgggcggttcatgagccagaaccactcagcagcatgtcagtgtgcttcctggggagctggtagcaggggctccgggccctacttcagggctgctttctggcatatggctgatcccctcctcactcctcctccctgcattgctcctgcgcaagaagcaaaggtgaggggctgggtatggctcgtcctggcccctctaaggtggatctcggtggtttctagatgtgacagcacccttagtggatgagggagctcccggcaagcaggctgccgcgcagccccacacggagatcccagaaggaaccacaggtgagggtaagccccagagacccccaggcagtcaaggccctgctgggtgccccagctgacctgtgacagaagtgagggagctttgcgtgtttatcctcctgtggggcaggaacatgggtggattctggctcctgggaatcttgggttgtgagtagctcgatgccttggtgctcagttacctccctggctgcctgccagcctctcagagcatttagggccttctggacttctagatgctcctcatcttgcctcagtcagcgcgtcagttccagagacttctctgcagggttttctggggcaggtggtggcagacccgtgccttcttgacacctgaggtcagtccaccctcctgctcagactgcccagcacagggtcacctcccaaggggtggaccccaagatcacctgagcgcacagagggtgcagatgactggaccacaccttttggtgatcttaatgaggtggtcccagaggagctcagacatgcaatctagcatccagttctgggactctgtctccttttcaaacgtattcatgtagaacaggcatgacgagaatgccttgtcaacatgggtgatggggaatcaatcagacagggcgccgggctcaaggctgcagtcacccaagagtggctcagcccaccaggccctaggaaacgcctgcacagcctggagctcctggagtcatttccttcatgtcttcttcactgcacttacgtaaagatgccagccattggtttggtgatttggagggtgcccagttgcccaacaagaaatgcagaagaggcctagccaggatttcaccagcagtggagagtagagaagatgtggccagaaaagagtttcctttccctcctaaagatggtactccctgcagctactggggaagcctgcagcattctctagggctctgtgtgttgagagcagccccaccctggccccttctgagtgcatttctgctttgtgacttgatccgtgaagtcccctgagatgggcagaggggatgtcctcgaagctggggcagagcctcatccttgaacgtgaaggacgtttgaagactgtggcatgatcacaggatgagatcacagggaacttgagtttctctcctcctctcccttcacagttatttcactgagggaaatccctcccctgcccagaatgaaaactctagccaactcttgacttttccatcactccaaagtagttgaaagtacattagtctccacagtggcaaaacagtgtgcaaaagctaaataattagaacagccagtcccatgtgacagtcaaagcttctaactccattcaaagttgcagccattcccctcgagggctggcagggaggggaggggtaagagaaacaggaaggttcttactgagttggtcctggtgtgagctgcgtcacactccctgcagaggtttcaaggagactctctctctctctgtctccatggggaccttatttgaattcttctactcttaccccagcctgccatctccagctatcctcccctgaagagcccttctgctgcgctggattctggtggccatgtcatctcctcggccccgtgggagtctgaagatctggctgcagcctcacctctgaggtcctgctagttgccacctcttaaacatgatctgaggctcccatgcactctgacctgtgcccacatggggcccacgggaaacacgctggcaagcaaactgtgggtgtgcagacggttctcagggctgcagcacctgtcctttgctctgcccccaaagcaaggccagcccatcttccatcctctagtgttccttggtggggccctgaccacagtccaccaggtccctaaccagaggggacacacaccaggtgtcctcaatgtattgccttgaaacagttgtgctgggactgtgatggggggtggccatgtagccacccccaccacccccaagccactctctccaaggaaatcctcctaaagatccctttacatcctccatgtggtggggaggttctagagttgggtgcatgtgtcttcagctactgacaatgcagaccttagttggcacctcgctctggcctatcctgtttgctgttcttggcgctccagtgaaactccccatgggccatccagttggggtgcagtgtggccacccccttgcaggttcctgccttgctggagagcacagggccctcctggctcttgtaaaacactccccatggtacagagaggccagcagtgatgtgaggcccaacctccctccatggtgttcccaagcagctccctttctggggtcaaggggtggcaaagacagtgcagcgtccaatttctgactcaagccgggcctggctatcgcagctctgcactgtgtgtgacagcaaggcaactcacccagtgccgtggcagtgaccgtgtccgaggaagcctcctcacaccctctgtctcaaggactctggcatttagctggacttgctgtagctctgagcctttctgccattgccatcaccttgtcagaaactcaggccgaatctgcactcagagttgtgcccaggcagttgagccaacacttgctcagcgatattgtcacatgacaaggcactgtcaccactgggcatcgtgggtagcgcagtgtcggctggatggacccggagggtgtctgtgtcatgctagtgctagtgatgggagccccgtgagcccattgcccgccctcccatgccctcagcagctgcctggggacagccaatggcctgggtgtttctgaggctaccacatggcttccaggaaactcgagaacctttctctcccttgcctacactcttcacacaggcctgtgctggccagcggtggggatccggcattcctatcttaggtgcagagagtgactgactcattgcaggcctgggagataagactgatggcccagccagcaagatgtatggatttctcagaggcagtggcctctgtcattgtcctcaggaaatgctggtgattctggtggcctgaggtcaatgcatgtcaacgtggccaacttgccttataaactttttttctggacaattgcgtgcactgtcctgtaacagtgtcctgttgtttatgatgcagaaataggtgtttttaaagcctattgattttggtactattaatgtggtcaggaactttctcagtctttcttgtttggggtgagctgtggcttcctaaacaggaacccaagacacccccaaaagctgctcaccagcactgccagcctccctcttaccaagtagcacccgttcaggacattctgcgaaaggcatttgcccagaagttgggaggaaggaaatgtaacattttggggcacctaccatatgccaggcaccaggctaaacgtgttcacacaaattctcttactaaccctcaccatccttctacaagacaaactagtatcttcatcttggggttcaagatgaggaaatggaggctcagagaggttgaatgaatgccggtgcctggatatgaaccccatctgcctgactccgcaacccaggcaaagtctttccttgaacttcccagcagccactgcttagacacagcctccacaaccatggctcagcagcaaattgcttctctgacctcactcagcctgtgtgtccttgttgagtgaggcattcaggaccctggtcccaaagtggagaaagtctttcctactaggtcatagctacacctgcatgtgggtgctgtgccttttgtttagtgaacttttatcaccagcatcctcagcaatgacatttgcagagaagccagagctgaggcaccttggtattcttgggatgtgactttcctgaatgtttaagggaaaatgcccgaaggtacagagagcttggtttctagtaaacaataactgtcttgcttttaccccccttcatttgctgacacatacaccagctgaagaagcaggcattggagacacccccagcctggaagacgaagctgctggtcacgtgacccaaggtcagtgaactggaattgcctgccatgacttgggggttggggggagggacatggggtgggctctgccctgaaaagatcatttggacctgagctctaattcacaagtccaggagattttagggagttggttcttatcaaaggttggctactcagatatagaaagagccctagtggtttttttctaataccatttctgggtaattcctaaggcatttagtgttctgaaagatgctagccttgtccagcctgggagttgagaatgaatgtctaacagaaactctaggccgggcctggtggctcacgcctctaatcccagcactatgggagacccaggtgggcagatcacctgaggtcaggagtttgagaccagcctggccaacatgtgaaatcctgtctcactacaaataaaaaaattagccgggtgtggtggtaggtgcctataatcccagctactcaggaggctgaggcaggacaatcgctcgaacccaggaggtggacgttgcagtgagccgagatcgcatcattgcactccagcctgggcaacaaaagcaaaactccgtctcaaaaaaaaaaaagaaactcaaatatgtgtgacaggcgattctcactgcaggctgccctgtggctgatccaggagcaaggccttaaccatgtcatccccaagcgattgcttgtaaactttcttctgtgcagccttcaacccttattatgattttcttctcaggaaccaaactgctgtattcaagaaaggcagctttgtgtaatcatttatcataaatatcttaagaaaaatcctagagattcctaattttaggaaatgggagacctatggtactgatataatgtgggctgggcttgttttctgtcatttgctagataaatgaacttgagagcctactgtaaaatgtggaagcttctagattgcagaagggctggaaagacactgttcttttctcccgagtgatgggatctgtccagtatttagagctgcctctgaggccatctgattctaggagactctgcctcgttgaggatattttgaggcctaactacacattcctgcccccagagaggtcacagcctatagcaggctgatgtttctcatgtcacatggcacagaaaggcacattttcgttctcaggctaacaaagagcttcaaaaactattagaagggacagtggctataagagaagaacctcagtcaatgtgtgaaattaactaggaacctggctcctgtttcttttaggtcatgtttttcagcttaggtaaaactagaggctttgataaagcatgacctctagaaatcattgcttttcataaatggaagtgggtttgagttttttctactgattgttagtgcaggtgatgtctacatgcccccagaacatattccatgcaacaaaaaaagcccaggtcaccgtctttgctgggaacttgacttttgtgctcactgaattttaagctttctgacagcagcctggaatcatggagggataaagtacctattagtaagatggaaaaaggtgtttcaggttggagctgcagtctgttgagagtaagctatgggaaggcctgtatacgaggggtggacttttcttctgtaagtgtccagagaccaggcctcctgaagagggcatgggggcttaacttacctggactactgtgtttacaatactcatttatcttgaactcctcctaacccctgagaattgctacatttagtatttgctgagtacttcctagcatcctagggaatcaatagaacattctcccaaccaggctgggtgcggtggctcatgtctgtaatcccagcactttgggaggccaaggtaggcagatcccttgaggccaggagtgcaagactagcctggctgacatggtgaaaccccgtctttactaaaaatacaaaagttagccaggcatggtggtacacacctgtaatcccagctacatgggaggagtaggaggcaggagaattgcttgaacctgggaggtggaggttgctgtgagccgagatcatgccactgcactccagcctgggcgacagagtgagtgagactctgtttaaaaaaaaaaaaaaaaagaacattctcctaacctggcttcttcctccaggggtgtaattaatcatgtcagtttcctcattgatacacacacacacacactacaatcctgtatccattacttttcaaggtacatttactatttacgtttggggtccttgtctcttttttaatagtgtttcttaaagtcttgtattatatcagagtacagtaacatcccagtcaagagcactctagtaagctctaggaggaaagcgacttccggaaggcagtggagacctgtcctgttggggcagcataggggcagcccctgcctctggtcagttctggcgctcaggctcagggttgcctctgggctgttcttcccagagactgacaaagggctcccataaggcacctgcagagcctgtgagaagctgaagtcaatgttttcctgacaccagttgatctgtgcaggatccattgatttaaccacctgctgtgtggcatgcactgtggtcgatgccaggaacaggaattggaggggcccatgagcatggccagtatcacaggctggaggtgctgctgcgctctgaccgggcctcttggggatgagcccatgtcaaccaccttgcctccgatggggtcgggcccacaggttacctttgtgtgtccatgaccacaccttcctccccgacctcatccaaatctctttcttttccaagcccctgaatccttcagggctgcaggttttgtttaaagcagagctggtgagttgcataggttgttgcattgggactagatggggtgttcaaagagttgggagttaaaaaacataaagggtatttattaggagaaccaaggagtgtaattctcctgttcttaatatgcggccaggttaatgaatgtcacgtgaatgaaccagaaaaaaatgaagtgtgcccttgatcagctgggttggtgtgcagcaagctgtgtgaccaggggacagcagtggtcctgagggccgtcactgtctgccgtgcagagcccttcctcccacgggggcctacctcacctgtgccaagggcttgtctgtggtcagtgacctggatagatctgaatggggcttctttttcgaggagtcttatggcaggtctctcagtaaagactccattcttgatgatcacacattttggattttccaaatctgtcagagaatgggcttgaggcggggtttgtgggcactagtttcactggtttcatttaccaaaaaggggagcagaagtcaagtatggtggctcatccctgtaatcccagaggcaagagaattgcttgagcccaggagttcgagaccagcctgagcaacataaggagaccccgtctccacaaaaatgaaaaataacattttagtcagacgtggtggcatgcatctgtggtcccagctgcttgggagggtgagatgggagggttgtttgagccctggagttaaagttgcaatgagctgtgattgcaccactgcactctagcctgggtgacagaacgagaccctgtctcaaaaaaaaaaaaaaagaaagaaagaaaggaaaaaaaaaactcatgcctgtaatcccagcactttggggaccggggtgggcagatcacgaggtcaggagatcaagactatcctggccaacatggtgaaaccccgtttctactaaaaatacaaaaattagccaggtgtggtggcacgtgcctgtaatcccagttactcgggaggctgaggcaggagaatcgcttgaaccagggagtcagaggttgcagtgagctgagatcgtgccactgtactccagcctgggcgacagagtgagactctgtctcaaaccaaaaaaaaggggtggggggcgggggcaggagaacagtgagaggtagggagaggaaaggggattctcgctacacccaaaccagataccatctagaggctagaatctttgggaggctcaaattccctagaaagcaggagaagcttctgtagccctcccgctttcccagtagattaagcccagggcggctccagatgtgtgacatgctctgtgcccaaccagagcccatcataggcagaggaataacacccacaccagaagggccctcggaggtcaccacgtccaagaaccctctttacagatgaggaaactgaggcccagagaggggagagccacctagcgagctggtggcggctagaccaggagagctgtcattccaagcaagcaaaggcaacgagacgagcccagagctgtgctcccatctctttgttagggggcctgggatgccctctcagtgtcattttgtccaggatgatgctccctctcttaagcgattaatgcgcccttgctaaccttttgctatcgctgcctcttcaaaccagaggagttgagagttccgggccggcagaggaaggcgcctgaaaggcccctggccaatgagattagcgcccacgtccagcctggaccctgcggagaggcctctggggtctctgggccgtgcctcggggagaaagagccagaagctcccgtcccgctgaccgcgagccttcctcagcaccgtcccgtttgcccagcgcctcctccaacaggaggccctcaggagccctccctggagtggggacaaaaaggcggggactgggccgagaagggtccggcctttccgaagcccgccaccactgcgtatctccacacagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaaggtctgtgtcttgagcttcttcgctccttccctggggacctcccaggcctcccaggctgcgggcactgccactgagcttccaggcctcccgactcctgctgcttctgacgttcctaggacgccactaaatcgacacctgggtgcagctgctccactccctcggcctcctcccgtgctcaggctgtggccgcacgcgcccctcacgcttgcccgccactctgcatgtcaccagcacccccgctccgtgctacccaccttgtttgactctctggccacttgatttgtccacaacggcccatcagcccacaggaggtttggtgggtgccttccaccgacaggatgacgggtgccctcatggtgtctagaactctccaaccctcccatgtaggcataagcagccccactttgcagatgaggaaacggaggctcagagaagtacagtaacttgccgaaggccaatgagtagtaagtgacagagccaggtttgggatccaggtaggttgtctctgaaagacacgcctgtcctgcatcccacaacgcctcccaggaggtgctggagtgtggacgcctaacacagagatgtgcagggcacacacagcaggtgacacacacagcatccagaggtggcccagagctcatgctgtgcctttggcccagtgccctgcccccacccactctgccttgtggcaggaagacaaggagcagacacaagatctccctggtccacatgccaccacctccctctgcagaggacaaggggatcctcatgctggcattggagggggttgagcagggcccaccttgagccctcaggagcacgaccacagcagccctgcagggagggattggtgggaggagagtcccaagtatcagggagaggagagttggtgtcccacaggagacctcagagccacaaggcgagcttgttcataaatttgggacccttagcatttcacagttatttgcagagcccagaaatggatgttactgaagctcacagttgcaagcatctgttaaatttttattagattttacttttagggaaaactttgaaatgctataaagaagcctgtgtttaaaagttaagacagaggctgggggcgatggctcacgcctgtaatctcagcactttgggaggccaaggcaggtggatcatttgaggttaggagttcgagaccagcctggccaacatggtgagaccctgtctctactaaaattacaaaaaattagctgggcgtggtggcgggcacctgtagtcccagctactggggaggctgaagcaggataagtgcttgaacccaggaggcagaggttacagtgagccaagatcacaccactgtaccctaagcctgggcgacagagtgagactctgtctcaaaaaataaaataaaataaagttaagagagaaaaaaatatatcctatatcctttgttaaattccaaaacagtaggggacaaataactgacttgacaggttactacaatatttcctgaaatgatgttttcttgaatactggcctactagaggttcataggtgtgtttggattaaaaaagagttccatggcccagtgactgggggaaaaaaataaaagactaaagtaagttaaacaggcttttctgctgcaggacttgtcagagcctttaatgtactaatggccattgtgaccctctgagaaggtcacagagtgggtttcccaaacttacttgattctacctgctaacatttcctggaggaagtttgggaaatgccgatttagcagattcttttgttgtgccgtggatggtgctggttgatgtgggcaaaacaaagaacacgtgagtcagatccgcctggggctcttactaaagtgcaggttcccaggtgccactttaggcttacagacccagttgtggggtaagcctgggagtcttttagcaggtgattctgccacatagtatagttggaaaacctctgggcatactcattgctggtccctctagaaatccaggtgacaatagccaatgagaagctccaagagacccagttgtccatggggtagagggaatgtgatattgaaaccaaagaagaaaatctatgatcagttttcagcagtgactgtcaagagaaggagaagggtgagttagcgctgatgctggctgacaggtcagcgggttggtttcaccaaggagtgtgatgaaggctgatgttgtctgtgggaatgtatgatggtaactggtttgtagctaatttggggaagcagtgagaattcgtgccctttgaagaccagtaagtggcaagaaacccaccaggcctggctcagggctgggctgggcttggctcgtctcagagcagctggggctggtggccaaagccaccattagtgaggggcaggccctgggggtacaaccagcaactaggggacaaagacaaccctgccagcctctcctattctggaggcgtgtgaccagaaatggagatgggttggtcagcataagatggccaggaaggtggaaatcaggactgctggcaatctagccacatgggcaggggagccgggtggttccaggcagtttccaaggccaagagggtgagcaggcacctcacagggaatcagggccaagcctggctgcagtgtggagacaatgcacccacccccatccttggatcttgcaggaggctgggtcctcactgagctaccaacatccatggccctgaggcttttaaaacacccatccatggagtggggctggtcccagtggggtgaggctgaccctggcagaaacagggcaggagcctgtgggttagggagactgcaccttccttagatagcctccatgccatcatgtccccgtgacagtttctgctgcgtcccctctgcatggtcccaccctcggccagcctgctgccccctcttgccaggttgcgctaatcagtgaccccagtgtgctgtgttgatactaacaatgcgaggcctagcagattcaagggaaaagagaaccaactgggtttccaccagacccaactaaacaaacatggacctatcccagagaaatccagcttcaccacagctggctttctgtgaacagtgaaaatggagtgtgacaagcattcttattttatattttatcagctcgcatggtcagtaaaagcaaagacgggactggaagcgatgacaaaaaagccaaggtaagctgacgatgccacggagctctgcagctggtcaagtttacagagaagctgtgctttatgtctgattcattctcatatataatgtggggagtatttgtcactaaagtacagctgtcatttaaagtgctttgtattttggggcaggcttttaaaaagtccagcatttattagttttgatacttaccccagggaagagcagttggcaggttcatgaagtcatgctcctaattccagctttcttagtgtactttcagtgagaccctgacagtaaatgaaggtgtgtttgaaaaccaaacccaggacagtaaatgaaggtgtgtttgaaaaccagccctaggacagtaaatgaagccatcttctcactgcataaactgcacccagatcttcgcccatccttctcagtatttcacttcacccattgtttactgtctcaatgactggggaaatgtctggggaaatgctcccgtaattgcacagtggcgtttttcctggaaaatcccaccatggctctagataagacctatttttcttaaaggtatctaaaatttccagcataaattctgtctgaaacacctgaattttaatcagtactggagcccggagggcatctccagttgccacatagctctgagcattcagtggtgtgttgagggctgctcccggaagtgcctgcagagtcagggctccccagcctcatctagtgaggcagtggaagggcctgtggggatttggagagctggcctgggtctctgaagtgatagtgacagctgcttgtcaatcacggtgcacatttagtgctgggggcagggggcagggaataccagcctcatgcatgcatgcattcatttgttccttccttcattcattcattcagtacacatgggtacaacatccctgccctggagttgcccagagtctagggaggggaaagatctattaccctgggcctcggccagctggggagtgctgctggtggagaggggccgtgtgcagcgagggaaggaggagtcgtcaatacccccaccccagctttgctttcttgtcatcagccccagggccccagcctgtgtccctcctctcccattgctacttcatctcctgggtcctccttaccaagcctgaccacacagagggccttggccgcttccatggggaattggaaagcaataagatagcatcccctagaagcccagtgaagtctgggacaggacccttctctgagctctgacttgctcttggaaacacttcgaggcttagcctccccactttgtttcccgagagtgtgacctgttcccctccaaacacccccttctcctccagggccatgcccacccgtcaaaatcccccacgggcaggacgaactgtgggtgtcagtcaccatctatcctgcatcctggttccagggccccccccagccccgcctccatagggacaggcgtgcagacacccgtccctggctgcttcctcttgtggaatgggttcaaaagtaagcagtgttgtttacactgacaaactgaaaaaaaaagaaaaagagataacattggaggcttggcacagtggctcatgcctgtaatcccagcactttgggaggctaaggtgggaggatgtccccagcccaagagttctagaccagcctgggcaacatagcaagaccccatctcaaaaaaaaaatttaattggccaggcagaggtgggaggatcacttgaacccaaagggtggaggctgcagtgagccgtgatggcaccactgcactccagccagggcaacagagggagaccctgtctctaaaacaaacaaacaaacaaacaaacaaaagagttaacattggccagattaggattcaccagatagtgttaatattagtttgatttgagactttaatcagaaagcacatgtgtggtgggggtgggtgtaacctaagtcaggtagaatctttccaacttggggggggcacactcctgattgtagccatatgagtctgtcagtgtggtggaagaggccatgggttaatgggcaggtaaaaaagcaccttgcctggaattgagtagaaagtaaggcccttcagaccccgtgacacacttggggacattttcttgagtaacatcctaagattcatgtaccttgatgatctccatcaacttactcatgtgaagcacctttaaaccagtcgtctccaaattcaggggcacagtaacatccaacaggctggagaaagaacgtactagaacttccattcctttttcatgtcctcttctaaaagctttgtcagggccaggcgcggtggctcacgcctgtaatcccagcactttgggaggccgagacgggtggatcacgaggtcaggagatcgagaccatcctggctaacacagtgaaaccccatctctactaaaaatacaaaaaaacgagccgggcgtggtggtgggcgcctgtagtcccagctactcgggaggctgaggcaggagaatggcgtgaacccaggaggcagagcttgcagtgagccgagattgcaccactgcagtccagcctgggcgacagagcgagactccgtctcaaaaaagaaaaagaaaaagaaaaagaactgtgattggggaggacggtcactttcctgttcttactgatcagaagggatattaagggtacctgattcaaacagcctggagatcactgctttcaaccattacctgccttatttatttttagttactgtccttttttcagtttgtttccctcctccatgtgctgacttttattttgattttatttatgtttatgtttaagacatccacacgttcctctgctaaaaccttgaaaaataggccttgccttagccccaaacaccccactcctggtagctcagaccctctgatccaaccctccagccctgctgtgtgcccagagccaccttcctctcctaaatacgtctcttctgtcacttcccgaactggcagttctggagcaaaggagatgaaactcaaggtaaggaaaccacctttgaaaagaaccaggctgctctgctgtggtttgcaaatgtggggtttgtttatttgttttttagcctcaaagacctttcttcaaatgagttctggcatagaagcaccgtgtaaaatagttagaattctgggcaaaggggaaaagagagctgggggccatccctctcagcaccccacaggctctcatagcagcagctcctaagacacctggtgggaccttggtttcgaaatcgctactctaaggctgggcacggtggctcacacctgtaatcccagctctttaggaggccgaggagggtggatcacctgagatcaggagttcgagaccagcctggctaacatggcaaaaccctgtctctactaaaaatacaaaaattagccgggcgtggtggtatgcgtggtggtaatcgcagctactcgggaggctgaggcacaaggattgcttgaaccccagaggcagaggttgtagttagctccagcttgggcgacagagcaagaccctgtcgcaaaaattgtttaaaaaacaaacccaaaattgctactctcattgggttcctttgcccattcctgattttggcaagagaaatgcttccagattgccctgatctgggtaggacagcatcacgccatagcaacactgccccgtgagctcactgccccctcaactagcttgtggtccttggttaatgtcagtttcttttttgagtttgtgttatgtctaagggtcatctgctgggtaacggaacccagggactgccctagtccctagactgtgccatgcccgactctgccagctttgtcagtgatgctggtgctcgcctcctcgggtgctcgcctggtctgagcacacccaaggagttcttgaggccttagggttgtttgcgagagaatgaaagaacacgacctagctctctttagcatccttggtcaggttcaacactgcccccaggggcctctggtggagccaaccaccatcagccaaataaatccataattagagtcagaaaatggatgtctgcatatgtgtagtgcactaatgtcctgccgatgattgacatggagtggagagtgacctgatcattgctgtgagctctgctggccttggcacaactcatgctgataactaatgcacacagttcctctgggaggaaatgtcctcagggaacttggagtttgggtggggatgtgggtttgtgtgcccagcaagcccttgtggttgtagcagacactagtggcatctaggaggcaaagggtcaccccagtcttagccacgttttgagtcaaggtggcggagtggggctggtgttgactcttggtggcagtaacttttcccaatggtgaaaaacccctctatcatgtttcatttacagggggctgatggtaaaacgaagatcgccacaccgcggggagcagcccctccaggccagaagggccaggccaacgccaccaggattccagcaaaaaccccgcccgctccaaagacaccacccagctctggtaagaagaacgttctcttgaatcttagaggaagctgaagctctcagaggtacagccttcattttaggaggccttaggccactgagaatgaataacccctggcagctggtcagcagcttgcagtttactaagcactggagtcttcattgccttctcagtccttttgatttctgaggcaaatgttgaatccctacctttttttttttttttcttttgagacagagtttcgcttttgttatccaggccggagtgcagtggtgtgatctcagctcactgcatcctccacctcccaggttcaagcgattctcctacctcagcctccctagtagctgggattacaggcacctgccactatgcccggctaattttttgtatttttagtagagacagggtttcaccatgttggccaggctggtctcgaacgcctgacctcaggtgatccacctgcctcggcctcccaaagtgctgggattacaggcatgagccaccactcccagcctgaatcctcactttttatcaatgaagaaattgaggctgattctgcagcatgataaaaaaaaatacagaaaaaggaaaaaaaagaaagaaatcgagcctctgagagtttgcttgactgagtctaaccagctcattttaaacccgaggaaaatgcagtcacatgactactaagtggcagctctcggagcctctctggccccaagtccagggttccatagaggcagccccagcatggcatgttttcagtccccaaatgagactctggagacaaatgtctctggagacagagcagcagcctggataagtcacaatgggtgacgtcactcagggctcaacccctgggcagcttaacttgctagggacgttaggagtctgctgcaaaacctgagggtcttagctgagcagtcacaggctgggcccgttgccctgggctcctgtgagtaaaacccagtcaattttgagtacccagtaaggcatccattgagttattttgcagccaggagtgctattaagaacagtcgcggctgggcgtggtggctcatgcctgtaatcccagcactttgggaggccaaggtgggcggatcacctgaggtcaggagttcgagaccagcttggccaacatggcaaaaccccgtctctaataaaaatacaaaataattagctgggcgtggtggcgggcgcctgtaatcccagcttctcaggagggtgaggaaggagaatcacttgaacccaggaggcagaggttgcagtgagctgagatcgcaccattgcactccagcctggatgacaaaagtgagattccttctcaaaaaaaaaaaaaaaaaaacagtcgtcctctttggggattagggacagcctgcctgcctgcccgagcacttctctcttccattgccccagtgaagtattccaggcccctgggtttagactctgcaccatgtaggggtgtctgacctgcacttgctccttggtggcacgggcagcctatggcacttgctgcgggctgtgaccaaagcctggcctggatcttggatcttggtgactctgcttctccctggcctgagggagctgcccagagcctgcccaccacctgctgcgtgtctttgcggtggcatttctcgcacacatgccgtgcagtggcacccccaaggatggccattcactaaggcccattgtttttgtcttttcgcttcgtgttttctggcctggtgtttttctcatatacatgtgatccagggataattcccagaattttgacaggattttaagtagcgtttggatcctgctgtttttttttcacttaacatcgggccagttgactcacactctgttttttgttgttgtttttttgagacggagtctcactgtgtcacccaggctgaagtgcagtggcacaatcttggcatactgcaacctctgcttcccaaattcaagcagttttcctgcctcagcctcctgagtagctgggactacaggcacaggccaccacgccctgctaatttttgtatttttagtaaagacagggtttcaccattttggccagcctagtctcgaactcctgacctcaagtgatccgcccacctcggcctcccaaagtgctgggattacaggggactcacactttgtaacaacctgaaacaacgtgatgcatttccctttgggtcttacctgctcttcggtggctgcctgcaggtggagagaccctcccccttgggcccctcgaccttgtttcagaatggggcccctgctgggccagctgtgggtgcctgccacgtgaaggactcattaaggccctgtttaagcctgatgataataaggctttcgtggatttttctctttaagcgactaagcaagtccagagaagaccaccccctgcagggcccagatctgagagaggtactcgggagcctacttcgctgggagcagcctccctttgcgtgtgtggccattcactggcttgtgtttctagagccgggaggacccttttctgcaatgcagggttcacacagggttcgcagcctgaagatggagcagtccgaattctcttccctgtgcagtttgcgcagctgtgtttgtctgatgggctttctaatcctgtgtgctctccttgacttcagggacaatggcattacaggcatgagccaccatgcctggctgtctccctatgtttcagatgaagacataggcttaaggaggtcaggtgacttgcccacgaccactctgtaaataagaggcatgaaaagtatttggagccaccaccaccaagcccactggtcaccctgggtctctgaagtcagggaggcaggaggatgggaggtctgaggaggcagagaggctgagcctggaggccctggaggccgaggccccatctgttgtttccttatgtggaaaataagaggcttcgtttgtcctattgccacagagcgtactacttcaggaacatccaagacatggaaatccgcagggcacggtggctcacgtctataatcccggcactttgggaggttgaggtgggagaatcgcttgaggccagaagttcaagaccagcctgagcaacatagtcagaccccgtctctataaaaaacattatttttaaaaaagacatggaagtcaaattctaaaaactggtgctggctgggtgcggtggctcatgcctataatcccagcactttgggaggccgaggcgggtggatcacctgaggtcaggagttcaagaccagcctggccaacatggtaaaacctctactaaagaaatctttactgaaaatacaaaaatccagtctctactaaaataagtctctactaaaaatacaaaaattagccaggcgtggtgctgcacacctgtaatatcagctactcgggaggctgaggcaggagactcgcttgatcccatgcagcggaggttgcagtgagccgagatcacgccattgcactccagcctgggcatcagaataagactccgtctcaaaaaaaaaaccacaaaaaaacaaaacaacaacaaaagaaaactagtgcttattcgtcactggccaagctgcccattggctacatgggtgcttcaaagagctgcccttctccaggtctggccagcaggtatgtgttacagcaaatgcctggggcagcggcaggggcattgctgcgggaagcttctggacttgcaggaaagctaagttctcagactgcaggggagctaagcacacctcggcacagggtgaggcctgcggttctcagacttcagtctttgtggagcttgagaaaaatgaggctttgcaggtcccacccctagagattctgctctatccactcttgaaggggatcgagaaatttgcattttgcaactcccactttcctccttgaaagctccggagattctgacgcagggttccgtgggccacactttggaaaatacagacccatgagatagaataccagactgttgaagtgtaacgggggcctgggaagtgcagtaacagaagcaagtttgagggtaaaggacacccagaggagggagggacagcatctgcatggagaggagaagagaccccccagcagcttccagggtgttggaagggtgcgctagtaactgctatgcatggcaggtggggaactgtacgtcagggcacagcagcatgaagcggtatggctcgtgtggacagctagggacaggcaggcgtggagcaggcatcctgttctgaaggccaaatcccacagaggagccagggtgctggcaggagccctgaactagccgaacagctgaacagctgaacattcaccctgtggggaaagggtcagaagcgtccaggcttgagggcacagctgggtctcgtcactgcatcacccttatttaggataaaggccctgaagaattgtattagaggttggcaaagcatatctaccacctcctggagccacgctggccgcagggattataattatttccattttcaaattaaggcctctgagctcagagaggggaagttacttgtctgaggccacacagcttgttggagcccatctcttgacccaaagactgtggagccgagttggccacctctctgggagcgggtattggatggtggttgatggttttccattgctttcctgggaaaggggtgtctctgtccctaagcaaaaaggcagggaggaagagatgcttccccagggcagccgtctgctgtagctgcgcttccaacctggcttccacctgcctaacccagtggtgagcctgggaatggacccacgggacaggcagcccccagggccttttctgaccccacccactcgagtcctggcttcactcccttccttccttcccaggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgagagtggctggctgcgcgtggaggtgtggggggctgcgcctggaggggtagggctgtgcctggaagggtagggctgcgcctggaggtgcgcggttgagcgtggagtcgtgggactgtgcatggaggtgtggggctccccgcacctgagcacccccgcataacaccccagtcccctctggaccctcttcaaggaagttcagttctttattgggctctccactacactgtgagtgccctcctcaggcgagagaacgttctggctcttctcttgccccttcagcccctgttaatcggacagagatggcagggctgtgtctccacggccggaggctctcatagtcagggcacccacagcggttccccacctgccttctgggcagaatacactgccacccataggtcagcatctccactcgtgggccatctgcttaggttgggttcctctggattctggggagattgggggttctgttttgatcagctgattcttctgggagcaagtgggtgctcgcgagctctccagcttcctaaaggtggagaagcacagacttcgggggcctggcctggatccctttccccattcctgtccctgtgcccctcgtctgggtgcgttagggctgacatacaaagcaccacagtgaaagaacagcagtatgcctcctcactagccaggtgtgggcgggtgggtttcttccaaggcctctctgtggccgtgggtagccacctctgtcctgcaccgctgcagtcttccctctgtgtgtgctcctggtagctctgcgcatgctcatcttcttataagaacaccatggcagctgggcgtagtggctcacgcctataatcccagcactttgggaggctgaggcaggcagatcacgaggtcaggagttcgagaccaacctgaccaacagggtgaaacctcgtctctactaaaaatacaaaaatacctgggcgtggtggtggtgcgcgcctataatcccagctactcaggaggctgaggcaggagaatcgcttgaacccaggaggcagaggttgcagtgagccgagatagtgccactgcactccagtttgagcaacagagcgagactctgtctcaaaacaaaataaaacaaaccaaaaaaacccaccatggcttagggcccagcctgatgacctcatttttcacttagtcacctctctaaaggccctgtctccaaatagagtcacattctaaggtacgggggtgttggggaggggggttagggcttcaacatgtgaatttgcggggaccacaattcagcccaggaccccgctcccgccacccagcactggggagctggggaagggtgaagaggaggctgggggtgagaaggaccacagctcactctgaggctgcagatgtgctgggccttctgggcactgggcctcggggagctagggggctttctggaaccctgggcctgcgtgtcagcttgcctcccccacgcaggcgctctccacaccattgaagttcttatcacttgggtctgagcctggggcatttggacggagggtggccaccagtgcacatgggcaccttgcctcaaaccctgccacctccccccacccaggatcccccctgcccccgaacaagcttgtgagtgcagtgtcacatcccatcgggatggaaatggacggtcgggttaaaagggacgcatgtgtagaccctgcctctgtgcatcaggcctcttttgagagtccctgcgtgccaggcggtgcacagaggtggagaagactcggctgtgccccagagcacctcctctcatcgaggaaaggacagacagtggctcccctgtggctgtggggacaagggcagagctccctggaacacaggagggagggaaggaagagaacatctcagaatctccctcctgatggcaaacgatccgggttaaattaaggtccggccttttcctgctcaggcatgtggagcttgtagtggaagaggctctctggaccctcatccaccacagtggcctggttagagaccttggggaaataactcacaggtgacccagggcctctgtcctgtaccgcagctgagggaaactgtcctgcgcttccactggggacaatgcgctccctcgtctccagactttccagtcctcattcggttctcgaaagtcgcctccagaagccccatcttgggaccaccgtgactttcattctccagggtgcctggccttggtgctgcccaagaccccagaggggccctcactggcctttcctgccttttctcccattgcccacccatgcacccccatcctgctccagcacccagactgccatccaggatctcctcaagtcacataacaagcagcacccacaaggtgctcccttccccctagcctgaatctgctgctccccgtctggggttccccgcccatgcacctctgggggcccctgggttctgccataccctgccctgtgtcccatggtggggaatgtccttctctccttatctcttcccttcccttaaatccaagttcagttgccatctcctccaggaagtcttcctggattcccctctctcttcttaaagcccctgtaaactctgaccacactgagcatgtgtctgctgctccctagtctgggccatgagtgagggtggaggccaagtctcatgcatttttgcagcccccacaagactgtgcaggtggccggccctcattgaatgcggggttaatttaactcagcctctgtgtgagtggatgattcaggttgccagagacagaaccctcagcttagcatgggaagtagcttccctgttgaccctgagttcatctgaggttggcttggaaggtgtgggcaccatttggcccagttcttacagctctgaagagagcagcaggaatggggctgagcagggaagacaactttccattgaaggcccctttcagggccagaactgtccctcccaccctgcagctgccctgcctctgcccatgaggggtgagagtcaggcgacctcatgccaagtgtagaaaggggcagatgggagccccaggttatgacgtcaccatgctgggtggaggcagcacgtccaaatctactaaagggttaaaggagaaagggtgacttgacttttcttgagatattttgggggacgaagtgtggaaaagtggcagaggacacagtcacagcctcccttaaatgccaggaaagcctagaaaaattgtctgaaactaaacctcagccataacaaagaccaacacatgaatctccaggaaaaaagaaaaagaaaaatgtcatacagggtccatgcacaagagcctttaaaatgacccgctgaagggtgtcaggcctcctcctcctggactggcctgaaggctccacgagcttttgctgagacctttgggtccctgtggcctcatgtagtacccagtatgcagtaagtgctcaataaatgtttggctacaaaagaggcaaagctggcggagtctgaagaatccctcaaccgtgccggaacagatgctaacaccaaagggaaaagagcaggagccaagtcacgtttgggaacctgcagaggctgaaaactgccgcagattgctgcaaatcattgggggaaaaacggaaaacgtctgttttcccctttgtgcttttctctgttttcttctttgtgcttttctctgttttcaggatttgctacagtgaacatagattgctttggggccccaaatggaattattttgaaaggaaaatgcagataatcaggtggccgcactggagcaccagctgggtaggggtagagattgcaggcaaggaggaggagctgggtggggtgccaggcaggaagagcccgtaggccccgccgatcttgtgggagtcgtgggtggcagtgttccctccagactgtaaaagggagcacctggcgggaagagggaattcttttaaacatcattccagtgcccgagcctcctggacctgttgtcatcttgaggtgggcctcccctgggtgactctagtgtgcagcctggctgagactcagtggccctgggttcttactgctgacacctaccctcaacctcaaccactgcggcctcctgtgcaccctgatccagtggctcattttccactttcagtcccagctctatccctatttgcagtttccaagtgcctggtcctcagtcagctcagacccagccaggccagcccctggttcccacatcccctttgccaagctcatccccgccctgtttggcctgcgggagtgggagtgtgtccagacacagagacaaaggaccagcttttaaaacattttgttggggccaggtgtggtggctcacacctaatcccaacacctggggaggccaaggcagaaggatcacttgagtccaggagttcaagaccagcctgggcaacatagggagaccctgtctctacaattttttttttaattagctgggcctgttggcactctcctgtagttccagctactctagaggctgaggtgggaggactgcttgagcctgggaggtcagggctgcaatgagccatgttcacaccactgaacgccagcctgggcgagaccctgtatcaaaaaagtaaagtaaaatgaatcctgtacgttatattaaggtgccccaaattgtacttagaaggatttcatagttttaaatacttttgttatttaaaaaattaaatgactgcagcatataaattaggttcttaatggaggggaaaaagagtacaagaaaagaaataagaatctagaaacaaagataagagcagaaataaaccagaaaacacaaccttgcactcctaacttaaaaaaaaaaatgaagaaaacacaaccagtaaaacaacatataacagcattaagagctggctcctggctgggcgcggtggcgcatgcctgtaatcccaacactttgggaggccgatgctggaggatcacttgagaccaggagttcaaggttgcagtgagctatgatcataccactacaccctagcctgggcaacacagtgagactgagactctattaaaaaaaaaatgctggttccttccttatttcattcctttattcattcattcagacaacatttatggggcacttctgagcaccaggctctgtgctaagagcttttgcccccagggtccaggccaggggacaggggcaggtgagcagagaaacagggccagtcacagcagcaggaggaatgtaggatggagagcttggccaggcaaggacatgcagggggagcagcctgcacaagtcagcaagccagagaagacaggcagacccttgtttgggacctgttcagtggcctttgaaaggacagcccccacccggagtgctgggtgcaggagctgaaggaggatagtggaacactgcaacgtggagctcttcagagcaaaagcaaaataaacaactggaggcagctggggcagcagagggtgtgtgttcagcactaaggggtgtgaagcttgagcgctaggagagttcacactggcagaagagaggttggggcagctgcaagcctctggacatcgcccgacaggacagagggtggtggacggtggccctgaagagaggctcagttcagctggcagtggccgtgggagtgctgaagcaggcaggctgtcggcatctgctggggacggttaagcaggggtgagggcccagcctcagcagcccttcttggggggtcgctgggaaacatagaggagaactgaagaagcagggagtcccagggtccatgcagggcgagagagaagttgctcatgtggggcccaggctgcaggatcaggagaactggggaccctgtgactgccagcggggagaagggggtgtgcaggatcatgcccagggaagggcccaggggcccaagcatgggggggcctggttggctctgagaagatggagctaaagtcactttctcggaggatgtccaggccaatagttgggatgtgaagacgtgaagcagcacagagcctggaagcccaggatggacagaaacctacctgagcagtggggctttgaaagccttggggcggggggtgcaatattcaagatggccacaagatggcaatagaatgctgtaactttcttggttctgggccgcagcctgggtggctgcttccttccctgtgtgtattgatttgtttctcttttttgagacagagtcttgctgggttgcccaggctggagtgcagtggtgcgatcatagctcactgcagccttgaagtcctgagctcaagagatccttccacctcagcctcctgagtagttgggaccacaggcttgcaccacagtgcccaactaatttcttatattttttgtagagatggggtttcactgtgtcgcccaggatggtcttgaactcctgggctcaagtgatcctcctgcctcagcctcgcaaattgctgggattacaggtgtgagccaccatgcccgaccttctctttttaagggcgtgtgtgtgtgtgtgtgtgtgtgggcgcactctcgtcttcaccttcccccagccttgctctgtctctacccagtcacctctgcccatctctccgatctgtttctctctccttttacccctctttcctccctcctcatacaccactgaccattatagagaactgagtattctaaaaatacattttatttatttattttgagacagagtctcactctgtcacccaggctggagtgcagtggtgcaatctcggctcactgcaacctccgcctcccaggttgaagcaactctcctgcctcagcctccctagtagctgggattacaagcacacaccaccatgcctagcaaatttttatatttttagtagaggaggggtgtcaccatgtttgccaagctggtctcaaactcctggcctcaggtgatctgcctaccttggtctcccaaagtgctgggattacaggtgtgagccaccacgcctgcccttaaaaatacattatatttaatagcaaagccccagttgtcactttaaaaagcatctatgtagaacatttatgtggaataaatacagtgaatttgtacgtggaatcgtttgcctctcctcaatcagggccagggatgcaggtgagcttgggctgagatgtcagaccccacagtaagtggggggcagagccaggctgggaccctcctctaggacagctctgtaactctgagaccctccaggcatcttttcctgtacctcagtgcttctgaaaaatctgtgtgaatcaaatcattttaaaggagcttgggttcatcactgtttaaaggacagtgtaaataattctgaaggtgactctaccctgttatttgatctcttctttggccagctgacttaacaggacatagacaggttttcctgtgtcagttcctaagctgatcaccttggacttgaagaggaggcttgtgtgggcatccagtgcccaccccgggttaaactcccagcagagtattgcactgggcttgctgagcctggtgaggcaaagcacagcacagcgagcaccaggcagtgctggagacaggccaagtctgggccagcctgggagccaactgtgaggcacggacggggctgtggggctgtggggctgcaggcttggggccagggagggagggctgggctctttggaacagccttgagagaactgaacccaaacaaaaccagatcaaggtctagtgagagcttagggctgctttgggtgctccaggaaattgattaaaccaagtggacacacacccccagccccacctcaccacagcctctccttcagggtcaaactctgaccacagacatttctcccctgactaggagttccctggatcaaaattgggagcttgcaacacatcgttctctcccttgatggtttttgtcagtgtctatccagagctgaagtgtaatatatatgttactgtagctgagaaattaaatttcaggattctgatttcataatgacaaccattcctcttttctctcccttctgtaaatctaagattctataaacggtgttgacttaatgtgacaattggcagtagttcaggtctgctttgtaaatacccttgtgtctattgtaaaatctcacaaaggcttgttgccttttttgtggggttagaacaagaaaaagccacatggaaaaaaaatttcttttttgtttttttgtttgcttgtttttttgagacagagtttcactctgtcgcccaggctggagtgcagtggtgcgatctccgcccactgcaagctccacctcccgggttcatgctattctcctgtctcagcctcccaagtagctgggactgcaggtgcccgccaccacacctggctaatttttttgtatttttagtagagacggggtttcaccgtgttagccaggatggtctcaatctcctgacctcgtcatctgcctgcctcggcctcccaaagtgctgagattacaggcgtgagccaccgtgcccggccagaaaaaaacatttctaagtatgtggcagatactgaattattgcttaatgtcctttgattcatttgtttaatttctttaatggattagtacagaaaacaaagttctcttccttgaaaaactggtaagttttctttgtcagataaggagagttaaataacccatgacatttccctttttgcctcggcttccaggaagctcaaagttaaatgtaatgatcactcttgtaattatcagtgttgatgcccttcccttcttctaatgttactctttacattttcctgctttattattgtgtgtgttttctaattctaagctgttcccactcctttctgaaagcaggcaaatcttctaagccttatccactgaaaagttatgaataaaaaatgatcgtcaagcctacaggtgctgaggctactccagaggctgaggccagaggaccacttgagcccaggaatttgagacctgggctgggcagcatagcaagactctatctccattaaaactatttttttttatttaaaaaataatccgcaaagaaggagtttatgtgggattccttaaaatcggagggtggcatgaattgattcaaagacttgtgcagagggcgacagtgactccttgagaagcagtgtgagaaagcctgtcccacctccttccgcagctccagcctgggctgaggcactgtcacagtgtctccttgctggcaggagagaatttcaacattcaccaaaaagtagtattgtttttattaggtttatgaggctgtagccttgaggacagcccaggacaactttgttgtcacatagatagcctgtggctacaaactctgagatctagattcttctgtggctgcttctgacctgagaaagttgcggaacctcagcgagcctcacatggcctccttgtccttaacgtggggacggtgggcaagaaaggtgatgtggcactagagatttatccatctctaaaggaggagtggattgtacattgaaacaccagagaaggaattacaaaggaagaatttgagtatctaaaaatgtaggtcaggcgctcctgtgttgattgcagggctattcacaatagccaagatttggaagcaacccaagtgtccatcaacagacaaatggataaagaaaatgtggtgcatatacacaatggaatactattcagccatgaaaaagaatgagaatctgtcatttgaaacaacatggatggaactggaggacattatgttaagtgaaataagccagacagaaggacagacttcacatgttctcacacatttgtgggagctaaaaattaaactcatggagatagagagtagaaggatggttaccagaggctgaggagggtggaggggagcagggagaaagtagggatggttaatgggtacaaaaacgtagttagcatgcatagatctagtattggatagcacagcagggtgacgacagccaacagtaatttatagtacatttaaaaacaactaaaagagtgtaattggactggctaacatggtgaaaccccgtctctactaaaaatacaaaaattagctgggcatggtggctcacgcctgtaatcccagcactttgggaggccgaggcgggccgatcacgaggtcaggagatcgagaccatcctagctaacatggtgaaaccccgtctctactacaaatacaaaaaaaagaaaaaattagccgggcatggtggtgggcgcctgtagtcccagctactcgggaggctgaggcaggagaatggcgtgaacccgggaggcggagcttgcagtgagccgagatcgcgccactgcactccagcctgggcgacaaggcaagattctatctcaaaaaaataaaaataaaataaaataaaataataaaataaaataaaataaaataaaataaataaaataaaataaaatgtataattggaatgtttataacacaagaaatgataaatgcttgaggtgatagataccccattcaccgtgatgtgattattgcacaatgtatgtctgtatctaaatatctcatgtaccccacaagtatatacacctactatgtacccatataaatttaaaattaaaaaattataaaacaaaaataaataagtaaattaaaatgtaggctggacaccgtggttcacgcctgtaatcccagtgctttgtgaggctgaggtgagagaatcacttgagcccaggagtttgagaccggcctgggtgacatagcgagaccccatcatcacaaagaatttttaaaaattagctgggcgtggtagcacataccggtagttccagctacttgggagaccgaggcaggaggattgcttgagcccaggagtttaaggctgcagtgagctacgatggcgccactgcattccagcctgggtgacagagtgagagcttgtctctattttaaaaataataaaaagaataaataaaaataaattaaaatgtaaatatgtgcatgttagaaaaaatacacccatcagcaaaaagggggtaaaggagcgatttcagtcataattggagagatgcagaataagccagcaatgcagtttcttttattttggtcaaaaaaaataagcaaaacaatgttgtaaacacccagtgctggcagcaatgtggtgaggctggctctctcaccagggctcacagggaaaactcatgcaacccttttagaaagccatgtggagagttgtaccgagaggttttagaatatttataactttgacccagaaattctattctaggactctgtgttatgaaaataacccatcatatggaaaaagctcctttcagaaagaggttcatgggaggctgtttgtattttttttttctttgcatcaaatccagctcctgcaggactgtttgtattattgaagtacaaagtggaatcaatacaaatgttggatagcaggggaacaatattcacaaaatggaatgggacatagtattaaacatagtgcttctgatgaccgtagaccatagacaatgcttaggatatgatatcacttcttttgttgttttttgtattttgagacgaagtctcattctgtcacccaggctggagttcagtggcgccatctcagctcactgcaacctccatctcccgggttcaagctattctccttcctcaacctcccgagtagctgggttgcgcaccaccatgcctggctaacttttgtatttttagtacagacggggtttcaccacgttggccaggctgctcttgaactcctgacgtcaggtgatccaccagccttgacctcccaaagtgctaggattacaggagccactgtacccagcctaggatatgatatcacttcttagagcaagatacaaaattgcatgtgcacaataattctaccaagtataggtatacaggggtagttatatataaatgagacttcaaggaaatacaacaaaatgcaatcgtgattgtgttagggtggtaagaaaacggtttttgctttgatgagctctgttttttaaaatcgttatattttctaataaaaatacatagtcttttgaaggaacataaaagattatgaagaaatgagttagatattgattcctattgaagattcagacaagtaaaattaaggggaaaaaaaacgggatgaaccagaagtcaggctggagttccaaccccagatccgacagcccaggctgatggggcctccagggcagtggtttccacccagcattctcaaaagagccactgaggtctcagtgccattttcaagatttcggaagcggcctgggcacggctggtccttcactgggatcaccacttggcaattatttacacctgagacgaataaaaaccagagtgctgagattacaggcatggtggcttacgcttgtaatcggctttgggaagccgaggtgggctgattgcttgagcccaggagtttcaaactatcctggacaacatagcatgacctcgtctctacaaaaaatacaaaaaatttgccaggtgtggtggcatgtgcctgtggtcccagctacttgggaggctgaagtaggagaatcccctgagccctgggaagtcgaggctgcactgagccgtgatggtgtcactgcactccagcctgggtgacaaagtgagaccctatctcacaaagaaaaaaaacaaaacaaaaaacccaaagcacactgtttccactgtttccagagttcctgagaggaaaggtcaccgggtgaggaagacgttctcactgatctggcagagaaaatgtccagtttttccaactccctaaaccatggttttctatttcatagttcttaggcaaattggtaaaaatcatttctcatcaaaacgctgatattttcacacctccctggtgtctgcagaaagaaccttccagaaatgcagtcgtgggagacccatccaggccacccctgcttatggaagagctgagaaaaagccccacgggagcatttgctcagcttccgttacgcacctagtggcattgtgggtgggagagggctggtgggtggatggaaggagaaggcacagcccccccttgcagggacagagccctcgtacagaagggacaccccacatttgtcttccccacaaagcggcctgtgtcctgcctacggggtcagggcttctcaaacctggctgtgtgtcagaatcaccaggggaacttttcaaaactagagagactgaagccagactcctagattctaattctaggtcagggctaggggctgagattgtaaaaatccacaggtgattctgatgcccggcaggcttgagaacagccgcagggagttctctgggaatgtgccggtgggtctagccaggtgtgagtggagatgccggggaacttcctattactcactcgtcagtgtggccgaacacatttttcacttgacctcaggctggtgaacgctcccctctggggttcaggcctcacgatgccatccttttgtgaagtgaggacctgcaatcccagcttcgtaaagcccgctggaaatcactcacacttctgggatgccttcagagcagccctctatcccttcagctcccctgggatgtgactcaacctcccgtcactccccagactgcctctgccaagtccgaaagtggaggcatccttgcgagcaagtaggcgggtccagggtggcgcatgtcactcatcgaaagtggaggcgtccttgcgagcaagcaggcgggtccagggtggcgtgtcactcatccttttttctggctaccaaaggtgcagataattaataagaagctggatcttagcaacgtccagtccaagtgtggctcaaaggataatatcaaacacgtcccgggaggcggcagtgtgagtaccttcacacgtcccatgcgccgtgctgtggcttgaattattaggaagtggtgtgagtgcgtacacttgcgagacactgcatagaataaatccttcttgggctctcaggatctggctgcgacctctgggtgaatgtagcccggctccccacattcccccacacggtccactgttcccagaagccccttcctcatattctaggagggggtgtcccagcatttctgggtcccccagcctgcgcaggctgtgtggacagaatagggcagatgacggaccctctctccggaccctgcctgggaagctgagaatacccatcaaagtctccttccactcatgcccagccctgtccccaggagccccatagcccattggaagttgggctgaaggtggtggcacctgagactgggctgccgcctcctcccccgacacctgggcaggttgacgttgagtggctccactgtggacaggtgacccgtttgttctgatgagcggacaccaaggtcttactgtcctgctcagctgctgctcctacacgttcaaggcaggagccgattcctaagcctccagcttatgcttagcctgcgccaccctctggcagagactccagatgcaaagagccaaaccaaagtgcgacaggtccctctgcccagcgttgaggtgtggcagagaaatgctgcttttggcccttttagatttggctgcctcttgccaggagtggtggctcgtgcctgtaattccagcactttgggagactaaggcgggaggttcgcttgagcccaggagttcaagaccagcctgggcaacaatgagacccctgtgtctacaaaaagaattaaaattagccaggtgtggtggcacgcacctgtagtcccagctacttgggaggctgaggtgggaggattgcctgagtccgggaggcggaagttgcaaggagccatgatcgcgccactgcacttcaacctaggcaacagagtgagactttgtctcaaaaaacaatcatataataattttaaaataaatagatttggcttcctctaaatgtccccggggactccgtgcatcttctgtggagtgtctccgtgagattcgggactcagatcctcaagtgcaactgacccacccgataagctgaggcttcatcatcccctggccggtctatgtcgactgggcacccgaggctcctctcccaccagctctcttggtcagctgaaagcaaactgttaacaccctggggagctggacgtatgagacccttggggtgggaggcgttgatttttgagagcaatcacctggccctggctggcagtaccgggacactgctgtggctccggggtgggctgtctccagaaaatgcctggcctgaggcagccacccgcatccagcccagagggtttattcttgcaatgtgctgctgcttcctgccctgagcacctggatcccggcttctgccctgaggccccttgagtcccacaggtagcaagcgcttgccctgcggctgctgcatggggctaactaacgcttcctcaccagtgtctgctaagtgtctcctctgtctcccacgccctgctctcctgtccccccagtttgtctgctgtgaggggacagaagaggtgtgtgccgcccccacccctgcccgggcccttgttcctgggattgctgttttcagctgtttgagctttgatcctggttctctggcttcctcaaagtgagctcggccagaggaggaaggccatgtgctttctggttgaagtcaagtctggtgccctggtggaggctgtgctgctgaggcggagctggggagagagtgcacacgggctgcgtggccaacccctctgggtagctgatgcccaaagacgctgcagtgcccaggacatctgggacctccctggggcccgcccgtgtgtcccgcgctgtgttcatctgcgggctagcctgtgacccgcgctgtgctcgtctgcgggctagcctgtgtcccgcgctctgcttgtctgcggtctagcctgtgacctggcagagagccaccagatgtcccgggctgagcactgccctctgagcaccttcacaggaagcccttctcctggtgagaagagatgccagcccctggcatctgggggcactggatccctggcctgagccctagcctctccccagcctgggggccccttcccagcaggctggccctgctccttctctacctgggacccttctgcctcctggctggaccctggaagctctgcagggcctgctgtccccctccctgccctccaggtatcctgaccaccggccctggctcccactgccatccactcctctcctttctggccgttccctggtccctgtcccagcccccctccccctctcacgagttacctcacccaggccagagggaagagggaaggaggccctggtcataccagcacgtcctcccacctccctcggccctggtccaccccctcagtgctggcctcagagcacagctctctccaagccaggccgcgcgccatccatcctccctgtcccccaacgtccttgccacagatcatgtccgccctgacacacatgggtctcagccatctctgccccagttaactccccatccataaagagcacatgccagctgacaccaaaataattcgggatggttccagtttagacctaagtggaaggagaaaccaccacctgccctgcaccttgttttttggtgaccttgataaaccatcttcagccatgaagccagctgtctcccaggaagctccagggcggtgcttcctcgggagctgactgataggtgggaggtggctgcccccttgcaccctcaggtgaccccacacaaggccactgctggaggccctggggactccaggaatgtcaatcagtgacctgccccccaggccccacacagccatggctgcatagaggcctgcctccaagggacctgtctgtctgccactgtggagtccctacagcgtgccccccacaggggagctggttctttgactgagatcagctggcagctcagggtcatcattcccagagggagcggtgccctggaggccacaggcctcctcatgtgtgtctgcgtccgctcgagcttactgagacactaaatctgttggtttctgctgtgccacctacccaccctgttggtgttgctttgttcctattgctaaagacaggaatgtccaggacactgagtgtgcaggtgcctgctggttctcacgtccgagctgctgaactccgctgggtcctgcttactgatggtctttgctctagtgctttccagggtccgtggaagcttttcctggaataaagcccacgcatcgaccctcacagcgcctcccctctttgaggcccagcagataccccactcctgcctttccagcaagatttttcagatgctgtgcatactcatcatattgatcacttttttcttcatgcctgattgtgatctgtcaatttcatgtcaggaaagggagtgacatttttacacttaagcgtttgctgagcaaatgtctgggtcttgcacaatgacaatgggtccctgtttttcccagaggctcttttgttctgcagggattgaagacactccagtcccacagtccccagctcccctggggcagggttggcagaatttcgacaacacatttttccaccctgactaggatgtgctcctcatggcagctgggaaccactgtccaataagggcctgggcttacacagctgcttctcattgagttacacccttaataaaataatcccattttatcctttttgtctctctgtcttcctctctctctgcctttcctcttctctctcctcctctctcatctccaggtgcaaatagtctacaaaccagttgacctgagcaaggtgacctccaagtgtggctcattaggcaacatccatcataaaccaggtagccctgtggaaggtgagggttgggacgggagggtgcagggggtggaggagtcctggtgaggctggaactgctccagacttcagaaggggctggaaaggatattttaggtagacctacatcaaggaaagtgttgagtgtgaaacttgcgggagcccaggaggcgtggtggctccagctcgctcctgcccaggccatgctgcccaagacaaggtgaggcgggagtgaagtgaaataaggcaggcacagaaagaaagcacatattctcggccgggcgctgtggctcacgcctgtaattccagcactttgggaggccaaggtgggtggatcatgaggtcaggagattgagaccatcctggctaacacagtgaaaccccgtctctactaaaaatacaaaaaattagccgggcgtggtggtgggcgcctgtagtcccagctactccggaggctgaggcaggaaaatggcgtgaacccggaaggcggagcttgcagtgagcggagtgagcagagatcgcgccactgcactccagcctgggcgacagagcgagactccgtctcaaaaaaaaaaagcacatgttctcgcttctttgtgggatccaggagatagagaatagaaggatggttaccagaggctgggaagggtagtgaggggatggtggggggatggtcaatgggtacaaaaaaaatagaataagacctagtatttgatagtgcaacagggtgactatagtcaataataatttaattgtacatttaaaaataactaaaagatagccgggtgcagtggcttacgtctgtaatcccagtactttgggaggctgaggtgggcgtttgagaccagcctggccaacatggtgaaaccccatctctactaaaaatacaaaaattagccaggcatggtggcgggcgcctgtaatcccagctactcgggaggctgaggcaggagaatcacttgaacctgggaggcagaggttgcagtgagccgagatcttgccactgcactccagcctgggtgacagtgaaactccgtctcaaaaataaaaataaaaatacagctgggcacggtggctcacgcctgtaatcccagcactttgggaggccgaggcgagcggatcacaaggtcaggagatatagaccatcctggctaacacggtgaaacccggtctctactaaaaatacaaaaaattagccaggcgtggtggcaggtgcctatagtcccagctactcacaaggctgaggcaggagaatggcatgaacctgggaggcggagcttgcagtgagccgagattgtgccactgcactccagcctgggcgagagagtgagactccgtctcaaaacaaaaacaaaaacaaaaacaaaaacaaacacacaacaaaaacctaaaagaatataaatggattgtttgtaacacaaaggacaaatgtttgaggggatggataccccattttccatgatgtgattattatacattgtgtgtctgtatcaaaacatctcatgagccccataaatatatacacctaactatgtacccacaaaaattaaaaaaatatattttttaaggtgaagagggaggcgagatgctggccttaacccctaacccgttgttctccctgcaagctgtccacagggcctctcagactcgaggttcagctatatggatgcatgagcttggtccccagccaacatgggagacacttcaccatcggcagcagctacagcacaggaaccctgggtcactgccatgtcccctctgtgactttgtttaaacagaaaatgatgctctgggccggctgtggtggcccacacctataatcccagcaccttgggaggcgggggtgggcagattgcctgaggtcaggagttggagatcagcctggccgacatggcgaaaccccatgtctactaaaaatacaaaaactagccaggcatggtggcacatgcctgtaatcccagctacttgggaggctgaagcaggagaatcacttgaacccaggaggcagaggctgagtgagccaagatcgtgccaatgcactccagcttgggtgagggagtgagactccgtctcaaaaaaaaaaaaaaagaaagaaaaagaaaagaaagtgatcctactggaaccatgcttactcccctccccacctcacactgtgtagaaattagtgctgtcggccaggcgcggtggctcatgcctgtaatcgcagcactttgggaggccaaggcaggcggatcacgaggtcaggagatcaagaccatcctggctaacacagtgaaaccctgtctctactaaaaatacaaaaaattagccgggcatggtggcaggcacctgtagtcccaactacttgggaggctgaggcaggagaatggcatgaacctgggaggcggagcttgcagtgagccaagatcgcgccactgcataccagcctaggtgacagagtgagactcagcaaaaaaagaaagaaagaaagaaagaaatcagtgctgtctatacttctttctgcagtgatggaaatattctgtatctgtgctgtccagtatagtagccactagctacatgtggcacttgaaacatggctggtacagttgaggaagagtggctgccatatcggacgacacagctatagattctgtcaccccaccccgagagtccagagcggggacttctgccttaggccctattcagggctgatttttacttgaacccttactgtgggaagagaaggccatgagaagttcagtctagaatgtgactccttattttctggctcccttggacactttgtgggatttagtctccctgtggaaagtattccacaagtggtgccaccaccccagctgtgagagcagctgggagctgcttttgtcatctttccctggaaagtcctgtgggctgtctcttcctcatgccttgtcccatgcttgggcatggtgtcaagcgtcaggagggagaaagggtccttatttatttatttagagagggacccttcttctgttcccaggctggagtgcagtggtgcgatctcggctcactgcaacctccgcctcctgggttcaagtgattctcctgcctcagcctcctgagtagctgagattacaggcacatgccaacatgcctggctaattttttttttttttttttttttttttttttgagatggagttgtactctcattgcccaggctggaatgtaatggcacaatctcggctcactgcaacctccacctcctggattcaagcaattctcctgtctcagcttcccaagtagctgggattacaggtgcccgccaccatgctcaactaatttttgtattttttttttagtagagacgaggtttcaccatgttggtcagactggtctcaaactcctgacctcaggtgatccacctgcctcggcctcccaaagtgctaggattacaggcatgagccaccacgcccggcctgaaagggttcttatttagtgtgcattttgacattcaatttaattccaaggtcttgtggggtcatggtttacaggatgttgatatagaaaagacttcacttaatgggccgggcgcagtggctcatgcctgtaatcccagcactttgggaggccgaggcaggcagatcaggaggtcaggagattgagaccatcctggctaacacagtgaaaccccatctctactgaaaatacaaaaaattagctgggcgtggtggcaggcacctgtagtcccagccactcggttggctgaggcaggagaatggcatgaacccgggaggcggagcttgcagtgagcagagaccatgccactgcactccagcctgggcgacagagcaagactctgtctcaagaaaaaaaaaaaaaaaacagactttacttactggaagccaaccaatgtatatttagagtaatttttcctgggctgagctgtcatttacttttgcagtatctcaagaagaagagtttacagtgtaaatatttgatgcacactttgattatatagatgaagcaaactattttcaagagctttgcaaggacttacttgtatccaaacaccattctaaaggagtcttacctacttctaaaggctggtctctacttggaaccacttgcttggccctggttcaagtcctgctgcaaacctggaagtcctgtcattgtcttcttccctccagagcagtggcacccaatctaatttttgctgtgccccagcagcccctggcactttgccctgtagactgcagacctcatgtaatgtatgttaagtccacagaaccacagaagatgatggcaagatgctcttgtgtgtgttgtgttctaggaggtggccaggtggaagtaaaatctgagaagcttgacttcaaggacagagtccagtcgaagattgggtccctggacaatatcacccacgtccctggcggaggaaataaaaaggtaaagggggtagggtgggttggatgctgcccttgggtatatgggcattaatcaagttgagtggacaaaggctggtccagttcccagaggaggaaaacagaggcttctgtgttgactggctggatgtgggccctcagcagcatccagtgggtctccactgcctgtctcaatcacctggagctttagcacgtttcacacctgggccccaacctggagaggctgaccaatgggtctcaggggcagctcggttgctggagtttttgtttttatttatttttatgtatttaaggcagggtctctgtattagtccattctcacactgctaataaagacatacccaagactgggtaatttataaaggaaagaggtttaatggactcacagttccacatggctggggaggcctcaaaatcatggcggaaggcaaaggagaagcaaaggcatttcttacatggcgacaggcaagagagcgtgtgcaggggaactcccatttataaaaccatcagacctcatgagatttattcactatcatgagaacagcatgggaaagacccgcccccatgattcagttacctcccactgggtccctcccatgacacatggaattatgggagctacaattcaagatgagatttgggtggggacacagccaaaccatatcagtctccctctgtcatccaggctggagtgcactggcatgatctcggctcactgcagcctctacctccctgggtcaggtgatcttcccacctcagcctcccaggtagctggaactacaggtacctgccactatgcctggctaaatattttgtatttcctgtggagacgaggttttgccacgttgcccaggctggtcttgaactcctgaggtcaagcaatatgcccacctcggcctcccaaggtgctgggattacaggtgtgagccacagtgctcggcctaagtcactgcagtttttaaagctcccaggtgattcttcagtgcagtcaaaagtgagaactggctgggtgcggtggctcatgcctgtaatcccagcaccttgggaggcgaaggtgggcagatggcttgaggtcaggagttcaagaccagcctggccaacatggtaaaaccccatctctactaaaaatacaaaagttagctgggtgtggtggtgcgtgcctgtaatcccagctacttgggaggctgaggcatgagaattgcttgaacccaggggacagaggttgtagtgagccgagatcgtgccactgcactccagcctgggcaacagagtgagattccatctcacaaaaaaaaaaaaaaaagcgagaaccactgtcctaggccctgatgtttgcaggcaactaaaaaaggaagtggacatccccagtcagctgtggcgcaccaagaacaagtcatgggaacataacctaattttctaaatgggttactaggcacttagagcaaaacaatgatgccgaaatcctgatttcagcaaagcctctgcctgcctgtcttggaagtatccacatgaggctgctggggccttggtgtccccagcagtttctagtctctaggtcttgctgtgggtgtctgtgcagtgagggtgtgtgtggcgctgggtgagctctgtctaggcctggcacaggatgcggtctggtagctgctgcttctcttctgcagaagcgcagccaagcaccctctggggtttcaggcccacacccagcctgaagttctgggagtggctcactttccaaccttcagggtctcccagcagctgactggggagtggtggagggaaaagggattgtattagtccgttttcacgccgctgatgaagacatacccgatactgggcagtctaaaagatagaggtctgatggactcacagttccacgtgactggggaggcctgacaatcatggtggaaggtgaaaggcttgtctcacacggtggcagacaagagaaaagagcttgtgcaggggaactcccctttataaaaccatcagatctcgggagacttattcactatcatgagaacagcacgggaaagaccctcctctatgattcaattacctcccaccaggtccctcccacaacatgtaggaattgtgggaactacaattcaagatgacatttgggtggggacacagccaaaccatatcagggcgtcccagaaagggtatagggtctgagacccaagtcagcatgagaaagtatgcttctcatggtggcccagttgggtggaagtggcagccgggccgtctttccaccaggccactcaagtagcagctgagagacccctgccctggccagtccccgccctcccctcttgccactgcctctggttctgaacagatgggcaccctcatcttgtatttgtgattaatgtctaacaatgtagttttgtgagaagggtttgctgatacagccttgctgcagatgctgcgaactgtggcctggggcagaccttacctccagacacgccctgaggcaggggagggcactggcccgtagctggccgagagctctcgggttgcgcgacagggatacttttcagcggctgggtcgctatccaaagtgagaaaacgaggagggaccaggaggctgtccgcctcaagagatgtgggggccaggtccagttatctggggaagcagtaagcttctctgctgtttctaaccccaggcctcccctggtctaaggcagggcctcccagcctcggggcactttaaagatatctgggcctggccccatccccacagtctgactgagtgggtctggatagggcctgagcattggtgatttcctgggtgaaaggaggcccctcacagtctctggaagcttctctgtgttaggaaaagctctgggcttgactctgctttgaaagtcaagatccgcaaatcctctcagcctcagtttctccttcagcaagatgaaatggaaatgctgtacctacgtcccggggtggttgtgagacccaaaaaagacaatgttctggaaggttcctggtgcgttgcagtcctctaagaacctgagttagagccacgctgagtctcagcttcttggctccttctgtttcaaactcgtccatgtgatagctcaggaagggtaggcagggccctgccccctactcagaaaacaccatcctggtcctggggatccccgcagcattagtcccctgttttcccagtgtattgagaaaaattgctaacaagcagtggggcacaccaccagcctcctgggttcctttcagtttggggatttttggacattcccaggaatgtcttaaaaaacacttcaaaaaacattaacataaatatttttatcaaagcctgtattaaatggtctttcaagaaaatacagtaacaggtcaggcatggtggctcatgcctgtaaccccagcactttgggaggccaaggcaggcagatcacctgaaatcaggagttcaagaccaacctggccaacacagccaaatcccatctctacaaaaaatacaaaaattagctgggtgtggtggcacacacctgtagtcccagctacttgggaggccgaggcaggagaattgcttgatcccggaggcggaggttgcagtgagctgagatcgtgccactgcactccagcgtgggtgacaaggtgaatctttgtctcaaaaaaaaaaaaaaaaaaaagataaaatacagtatacagtaatagagaacaatccttttttcaaagtagtgaccccaaatgaacaaaatatgcatctagcttaaatgcgaacctggttttctctacgcccattcaagcccctgcaataggggcccttcaccccgcatccatggactcctaaaattatatggaaaatggctgtgtgtgagtgtggatggacatgtgcacacatatttttggctttaccagatgctcaaagagcctaggacccaaaaagggctgagaatgaccgtgtcggccacttcagggtcatcaggaattgctgtgcactgctcacttctccagtgaacactttctgcttctgtgtttcctggtatcctttgggactcctggctaggtcatgtgtttctctactttcaaaagggcttcagccaggcacgatggcatgagcctgtagtcccagttgctctggaggttaaggtgggaagattgcttgagcccaggaatttgaggccagcctgggcaagtagataggtagatgattgatagatagatagatagataaatagatggatagataagtcgctagacagtcatccatccacccatccacacataaaaaggcctttgtcatgtcatgttttgtggcccacctgccagtgttgcccacagttgctgcccctccaaactcatcagtcactggcaaacaggaggaatgtgtggctcatgtctgggcatcagtggctgtgggagacatccttgatcttctccagcttctccttccacattttcctttgcaatctggcaatatctattaaaataaaatgtgcatgccttttgacctaagagcttcacttctaggacccacttacacgtgtgtgacatgatgttcatacgggtttatttatctgaggttgttcatacacaccattgcctgtaatcactaaaggcgggagcagcctacacatccatccacagaggagtagatgccttttggtacatccgtggcgacggaatactaagcagcctgtgtatctatacactcacacgtgtttgtttatgtgtggaatatctctggagggtacacaagaaacttaaaatgatcactgtctctggggagggtacctgggtgcctgggaggcaggtcagggaaggagtgggcacaggtattaccaattggaagacaataaaaacaacagctcctggccaggcgcagtggctcacgcctgtaatggcagcactctgagaggctgaggcgggcagattgcttgcgtccaggagttcaagaccagcctgggcaacatagcaaaaccccgtttctattaaaaatacaaaaaattagccaggtgtggtggcatgcacctgtaatcccagctactcgggaggctgaggtgggagaatcacctgagcctgggaggtcaaggctgcagtgaggtgagattgtgccaccgcactctagcctgggcgatagagcaagaccctgtctcaaaaacaaacaaaaaacagtccctggcactctgggccaggcctggcagggcagttggcagggctggtctttctctggcacttcatctcaccctccctcccttcctcttcttgcagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 3 - DNA, 6816 bp, Homo sapiens
  source 1..6816: mol_type = unassigned DNA
ggacggccgagcggcagggcgctcgcgcgcgcccactagtggccggaggagaaggctcccgcggaggccgcgctgcccgccccctcccctggggaggctcgcgttcccgctgctcgcgcctgcgccgcccgccggcctcaggaacgcgccctcttcgccggcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagacatccacacgttcctctgctaaaaccttgaaaaataggccttgccttagccccaaacaccccactcctggtagctcagaccctctgatccaaccctccagccctgctgtgtgcccagagccaccttcctctcctaaatacgtctcttctgtcacttcccgaactggcagttctggagcaaaggagatgaaactcaagggggctgatggtaaaacgaagatcgccacaccgcggggagcagcccctccaggccagaagggccaggccaacgccaccaggattccagcaaaaaccccgcccgctccaaagacaccacccagctctgcgactaagcaagtccagagaagaccaccccctgcagggcccagatctgagagaggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 4 - DNA, 18 bp, synthetic construct
  misc_feature 1..18: note = Synthetic construct
  source 1..18: mol_type = other DNA
cgtgtttgatattatcct

SEQ ID NO: 5 - DNA, 18 bp, synthetic construct
  misc_feature 1..18: note = Synthetic construct
  source 1..18: mol_type = other DNA
accttaagtattacttgc

SEQ ID NO: 6 - DNA, 19 bp, synthetic construct
  misc_feature 1..19: note = Synthetic construct
  source 1..19: mol_type = other DNA
tcaactcttagcatccact

SEQ ID NO: 7 - DNA, 18 bp, synthetic construct
  misc_feature 1..18: note = Synthetic construct
  source 1..18: mol_type = other DNA
cttatcatttgcattggt

SEQ ID NO: 8 - DNA, 19 bp, synthetic construct
  misc_feature 1..19: note = Synthetic construct
  source 1..19: mol_type = other DNA
ctgttagacattcattctc

SEQ ID NO: 9 - DNA, 17 bp, synthetic construct
  misc_feature 1..17: note = Synthetic construct
  source 1..17: mol_type = other DNA
cgtattatgctagaggt

SEQ ID NO: 10 - DNA, 12 bp, synthetic construct
  misc_feature 1..12: note = Synthetic construct
  source 1..12: mol_type = other DNA
gtttgatattat

SEQ ID NO: 11 - DNA, 12 bp, synthetic construct
  misc_feature 1..12: note = Synthetic construct
  source 1..12: mol_type = other DNA
ttaagtattact

SEQ ID NO: 12 - DNA, 13 bp, synthetic construct
  misc_feature 1..13: note = Synthetic construct
  source 1..13: mol_type = other DNA
actcttagcatcc

SEQ ID NO: 13 - DNA, 12 bp, synthetic construct
  source 1..12: mol_type = other DNA
atcatttgcatt

SEQ ID NO: 14 - DNA, 13 bp, synthetic construct
  source 1..13: mol_type = other DNA
ttagacattcatt

SEQ ID NO: 15 - DNA, 11 bp, synthetic construct
  source 1..11: mol_type = other DNA
attatgctaga

SEQ ID NO: 16 - DNA, 20 bp, synthetic construct
  source 1..20: mol_type = other DNA
atttccaaattcacttttac

SEQ ID NO: 17 - DNA, 19 bp, synthetic construct
  source 1..19: mol_type = other DNA
ctttaatttaatcactcat

SEQ ID NO: 18 - DNA, 16 bp, synthetic construct
  source 1..16: mol_type = other DNA
gcattctaatagcagc

SEQ ID NO: 19 - DNA, 16 bp, synthetic construct
  source 1..16: mol_type = other DNA
cgtgtttgatattatc

SEQ ID NO: 20 - DNA, 16 bp, synthetic construct
  source 1..16: mol_type = other DNA
gtgtttgatattatcc

SEQ ID NO: 21 - DNA, 16 bp, synthetic construct
  source 1..16: mol_type = other DNA
tgtttgatattatcct

SEQ ID NO: 22 - DNA, 16 bp, synthetic construct
  source 1..16: mol_type = other DNA
accttaagtattactt

SEQ ID NO: 23 - DNA, 16 bp, synthetic construct
  source 1..16: mol_type = other DNA
ccttaagtattacttg

SEQ ID NO: 24 - DNA, 16 bp, synthetic construct
  source 1..16: mol_type = other DNA
cttaagtattacttgc

SEQ ID NO: 25 - DNA, 16 bp, synthetic construct
  source 1..16: mol_type = other DNA
tcaactcttagcatcc

SEQ ID NO: 26 - DNA, 16 bp, synthetic construct
  source 1..16: mol_type = other DNA
caactcttagcatcca

SEQ ID NO: 27 - DNA, 16 bp, synthetic construct
  source 1..16: mol_type = other DNA
aactcttagcatccac

SEQ ID NO: 28 - DNA, 16 bp, synthetic construct
  source 1..16: mol_type = other DNA
actcttagcatccact

SEQ ID NO: 29 - DNA, 16 bp, synthetic construct
  source 1..16: mol_type = other DNA
cttatcatttgcattg

SEQ ID NO: 30 - DNA, 16 bp, synthetic construct
  source 1..16: mol_type = other DNA
ttatcatttgcattgg

SEQ ID NO: 31 - DNA, 16 bp, synthetic construct
  source 1..16: mol_type = other DNA
tatcatttgcattggt

SEQ ID NO: 32 - DNA, 16 bp, synthetic construct
  source 1..16: mol_type = other DNA
ctgttagacattcatt

SEQ ID NO: 33 - DNA, 16 bp, synthetic construct
  source 1..16: mol_type = other DNA
tgttagacattcattc

SEQ ID NO: 34 - DNA, 16 bp, synthetic construct
  source 1..16: mol_type = other DNA
gttagacattcattct

SEQ ID NO: 35 - DNA, 16 bp, synthetic construct
  source 1..16: mol_type = other DNA
ttagacattcattctc

SEQ ID NO: 36 - DNA, 16 bp, synthetic construct
  source 1..16: mol_type = other DNA
cgtattatgctagagg

SEQ ID NO: 37 - DNA, 16 bp, synthetic construct
  source 1..16: mol_type = other DNA
gtattatgctagaggt

SEQ ID NO: 38 - DNA, 10 bp, synthetic construct
  source 1..10: mol_type = other DNA
gtttgatatt

SEQ ID NO: 39 - DNA, 10 bp, synthetic construct
  source 1..10: mol_type = other DNA
tttgatatta

SEQ ID NO: 40 - DNA, 10 bp, synthetic construct
  source 1..10: mol_type = other DNA
ttgatattat

SEQ ID NO: 41 - DNA, 10 bp, synthetic construct
  source 1..10: mol_type = other DNA
ttaagtatta

SEQ ID NO: 42 - DNA, 10 bp, synthetic construct
  source 1..10: mol_type = other DNA
taagtattac

SEQ ID NO: 43 - DNA, 10 bp, synthetic construct
  source 1..10: mol_type = other DNA
aagtattact

SEQ ID NO: 44 - DNA, 10 bp, synthetic construct
  source 1..10: mol_type = other DNA
actcttagca

SEQ ID NO: 45 - DNA, 10 bp, synthetic construct
  source 1..10: mol_type = other DNA
ctcttagcat

SEQ ID NO: 46 - DNA, 10 bp, synthetic construct
  source 1..10: mol_type = other DNA
tcttagcatc

SEQ ID NO: 47 - DNA, 10 bp, synthetic construct
  source 1..10: mol_type = other DNA
cttagcatcc

SEQ ID NO: 48 - DNA, 10 bp, synthetic construct
  source 1..10: mol_type = other DNA
atcatttgca

SEQ ID NO: 49 - DNA, 10 bp, synthetic construct
  source 1..10: mol_type = other DNA
tcatttgcat

SEQ ID NO: 50 - DNA, 10 bp, synthetic construct
  source 1..10: mol_type = other DNA
catttgcatt

SEQ ID NO: 51 - DNA, 10 bp, synthetic construct
  source 1..10: mol_type = other DNA
ttagacattc

SEQ ID NO: 52 - DNA, 10 bp, synthetic construct
  source 1..10: mol_type = other DNA
tagacattca

SEQ ID NO: 53 - DNA, 10 bp, synthetic construct
  source 1..10: mol_type = other DNA
agacattcat

SEQ ID NO: 54 - DNA, 10 bp, synthetic construct
  source 1..10: mol_type = other DNA
gacattcatt

SEQ ID NO: 55 - DNA, 10 bp, synthetic construct
  source 1..10: mol_type = other DNA
attatgctag

SEQ ID NO: 56 - DNA, 10 bp, synthetic construct
  source 1..10: mol_type = other DNA
ttatgctaga

(19) United States
(12) Patent Application Publication
(10) Pub. No.: US 2026/0327615 A1
(43) Pub. Date: Oct. 22, 2026

(54) COMPOSITIONS AND METHODS FOR MODULATING TAU EXPRESSION

(71) Applicant: Denali Therapeutics Inc., South San Francisco, CA (US)

(72) Inventors: Scarlett BARKER, San Francisco, CA (US); Sarah L. DEVOS, San Francisco, CA (US); Mai B. THAYER, South San Francisco, CA (US); Richard KLAR, Munich (DE); Sven MICHEL, Bernried (DE); Frank JASCHINSKI, Puchheim (DE); Larissa ROTHKIRCH, Koenigsdorf (DE)

(21) Appl. No.: 19/485,779

(22) PCT Filed: Jun. 21, 2024
(86) PCT No.: PCT/US2024/034923

§ 371 Date: Nov. 19, 2025

Related U.S. Application Data
(60) Provisional application No. 63/509,696, filed on Jun. 22, 2023.

(51) Int. Cl. C12N 15/113; A61K 31/7115; A61K 31/712; A61K 31/7125

(52) U.S. Cl. CPC C12N 15/113; A61K 31/7115; A61K 31/712; A61K 31/7125; C12N 2310/11; C12N 2310/315; C12N 2310/3231; C12N 2310/3341; C12N 2310/341; C12N 2310/351

(57) ABSTRACT

Provided herein are MAPT antisense oligonucleotides that are capable of modulating the expression of a MAPT target nucleic acid. Also provided herein are methods of use thereof.

### CROSS-REFERENCE TO RELATED APPLICATIONS

This application claims the benefit of U.S. Provisional Application No. 63/509,696, filed Jun. 22, 2023, which is incorporated herein by reference

### SEQUENCE LISTING

The instant application contains a Sequence Listing which has been submitted electronically in XML format and is hereby incorporated by reference in its entirety. Said XML copy, created on Jun. 17, 2024, is named DNL-038-02-WO_SeqListing. XMP and is 328 kb in size.

### BACKGROUND

Alzheimer's disease is a progressive neurodegenerative disorder that is currently the seventh leading cause of death in the United States and is the most common cause of dementia among older adults. A prominent pathological feature in Alzheimer's disease and other neurodegenerative diseases is the aberrant aggregation and inclusion formation of microtubule-associated protein tau. Mutations in the microtubule associated protein tau (MAPT) gene that encodes tau have been linked to tauopathies that are linked to neurodegeneration.

### SUMMARY

Certain embodiments provide a MAPT antisense oligonucleotide (MAPT ASO) comprising a nucleic acid sequence that comprises at least 9 contiguous nucleobases of a sequence selected from any one of SEQ ID NOs: 4-9 and 19-37 and at least one modified internucleoside linkage and/or at least one modified sugar.

Certain embodiments provide MAPT ASOs 17-19 nucleotides in length and comprising the nucleobase sequence of any one of SEQ ID NOs: 4-9, 19-37, and 38-56, wherein the MAPT ASO comprises at least one modified internucleoside linkage and/or at least one modified sugar. In certain embodiments, the MAPT ASO comprises a nucleobase sequence consisting of the sequence of any one of SEQ ID NOs: 4-9. In certain embodiments, MAPT ASO of claim 1 or 2, wherein the MAPT ASO is a gapmer having a gap segment consisting of the nucleobase sequence of any one of SEQ ID NOs: 10-15.

Certain embodiments provide a MAPT ASO comprising the nucleobase sequence of any one of SEQ ID NOs: 10-15 and 38-56 and at least one modified internucleoside linkage and/or at least one modified sugar.

Certain embodiments provide a MAPT ASO comprising a nucleic acid sequence as set forth in any one of SEQ ID Nos: 4-9 and 19-37 that comprises at least one modified internucleoside linkage and/or at least one modified sugar.

Certain embodiments provide a MAPT ASO 16-20 nucleotides in length comprising the nucleobase sequence of any one of SEQ ID NOs: 10-15 and 38-56 and at least one modified internucleoside linkage and/or at least one modified sugar.

Certain embodiments provide a MAPT ASO 16 nucleotides in length comprising the nucleobase sequence of any one of SEQ ID NOs: 10-15 and 38-56 and at least one modified internucleoside linkage and/or at least one modified sugar.

Certain embodiments provide a MAPT ASO gapmer comprising a gap segment having the nucleobase sequence of any one of SEQ ID NOs: 10-15 and 38-56 and at least one modified internucleoside linkage and/or at least one modified sugar. In some embodiments, the MAPT ASO gapmer is 16 nucleotides in length wherein the 5′ and 3′ wing segments are each 3 nucleotides in length.

Certain embodiments, provide MAPT ASOs, wherein the MAPT ASO is a gapmer having a gap segment consisting of the nucleobase sequence of any one of SEQ ID NOs: 10-15, wherein the MAPT ASO comprises at least one modified internucleoside linkage and at least one modified sugar. In certain embodiments, the MAPT ASO is 15 to 25 linked nucleosides in length. In certain embodiments, the MAPT ASO is 16 to 19 linked nucleosides in length.

Certain embodiments, provide a MAPT antisense oligonucleotide (MAPT ASO), wherein the MAPT ASO is a gapmer 16 nucleotides in length and comprises a gap segment consisting of the nucleobase sequence of any one of SEQ ID NOs: 38-56 wherein the MAPT ASO comprises at least one modified internucleoside linkage and at least one modified sugar. In certain embodiments, the MAPT ASO comprises a nucleobase sequence consisting of the sequence of any one of SEQ ID NOs: 19-37.

Certain embodiments provide a MAPT ASO comprising at least 9 contiguous nucleobases complementary to a sequence present in nucleobases 66,750 to 66,786; 66,760 to 66,786; 66,750 to 66,776; 66,754 to 66,786; or 66,760 to 66,776 of SEQ ID NO: 1, wherein the nucleobase sequence of the MAPT ASO is at least 90% complementary to SEQ ID NO: 1, and wherein the MAPT ASO comprises at least one modified internucleoside linkage and/or at least one modified sugar.

Certain embodiments provide a MAPT ASO comprising at least 9 contiguous nucleobases complementary to a sequence present in nucleobases 78,431 to 78,468; 78,431 to 78,458; 78,441 to 78,468; or 78,441 to 78,458 of SEQ ID NO: 1, wherein the nucleobase sequence of the MAPT ASO is at least 90% complementary to SEQ ID NO: 1, and wherein the MAPT ASO comprises at least one modified internucleoside linkage and/or at least one modified sugar.

Certain embodiments provide a MAPT ASO comprising at least 9 contiguous nucleobases complementary to a sequence present in nucleobases 78,621 to 78,659; 78,621 to 78,649; 78,631 to 78,659; or 78,631 to 78,649 of SEQ ID NO: 1, wherein the nucleobase sequence of the MAPT ASO is at least 90% complementary to SEQ ID NO: 1, and wherein the MAPT ASO comprises at least one modified internucleoside linkage and/or at least one modified sugar.

Certain embodiments provide a MAPT ASO comprising at least 9 contiguous nucleobases complementary to a sequence present in nucleobases 78,818 to 78,855; 78,828 to 78,855; 78,818 to 78,845; or 78,828 to 78,845 of SEQ ID NO: 1, wherein the nucleobase sequence of the MAPT ASO is at least 90% complementary to SEQ ID NO. 1, and wherein the MAPT ASO comprises at least one modified internucleoside linkage and/or at least one modified sugar.

Certain embodiments provide a MAPT ASO comprising at least 9 contiguous nucleobases complementary to a sequence present in nucleobases 78,431 to 78,659; 78,431 to 78,649; 78,441 to 78,659; or 78,441 to 78,649 of SEQ ID NO: 1, wherein the nucleobase sequence of the MAPT ASO is at least 90% complementary to SEQ ID NO: 1, and wherein the MAPT ASO comprises at least one modified internucleoside linkage and/or at least one modified sugar.

Certain embodiments provide a MAPT ASO comprising at least 9 contiguous nucleobases complementary to a sequence present in nucleobases 78,641 to 78,855; 78,641 to 78,845:78,631 to 78,855; or 78,631 to 78,845 of SEQ ID NO: 1, wherein the nucleobase sequence of the MAPT ASO is at least 90% complementary to SEQ ID NO: 1, and wherein the MAPT ASO comprises at least one modified internucleoside linkage and/or at least one modified sugar.

Certain embodiments provide a MAPT ASO comprising at least 9 contiguous nucleobases complementary to a sequence present in nucleobases 78,431 to 78,855; 78,441 to 78,855; 78,431 to 78,845; 78,441 to 78,845; 78441 to 78,498; 78,517 to 78,678; or 78,697 to 78,845 of SEQ ID NO: 1, wherein the nucleobase sequence of the MAPT ASO is at least 90% complementary to SEQ ID NO: 1, and wherein the MAPT ASO comprises at least one modified internucleoside linkage and/or at least one modified sugar.

Certain embodiments provide a MAPT ASO comprising at least 9 contiguous nucleobases complementary to a sequence present in nucleobases 90,205 to 90,243; 90,205 to 90,233:90,215 to 90,243; 90,208 to 90,243; or 90,215 to 90,233, wherein the nucleobase sequence of the MAPT ASO is at least 90% complementary to SEQ ID NO: 1, and wherein the MAPT ASO comprises at least one modified internucleoside linkage and/or at least one modified sugar.

In some embodiments, the MAPT ASO is conjugated to a targeting ligand or delivery vehicle to form a conjugate. The targeting ligand can be, but is not limited to, a molecule that specifically binds to the transferrin receptor (TfR) or a molecule expressed on the luminal surface of the blood brain barrier. The molecule can be, but is not limited to, an anti-TfR antibody or a TfR binding fragment thereof, or a Fc polypeptide modified to bind the TfR (as described in WO2023279099, incorporated herein by reference).

Certain embodiments provide a pharmaceutical composition comprising a MAPT ASO as described herein or a conjugate comprising a MAPT ASO as described herein and a pharmaceutically acceptable carrier or diluent.

Certain embodiments provide a method of generating a neuronal cell with decreased Tau expression, the method comprising delivering to the neuron cell a MAPT ASO as described herein or a conjugate comprising a MAPT ASO as described herein, wherein the MAPT ASO decreases the expression level of an endogenous MAPT gene. The neuronal cell can be, but is not limited to, a brain cell, a deep brain cell, or a spinal cord cell.

Certain embodiments provide a method of modifying a neuronal cell to decrease Tau expression, the method comprising delivering to the neuron cell a MAPT ASO as described herein or a conjugate comprising a MAPT ASO as described herein, wherein the MAPT ASO decreases the expression level of an endogenous MAPT gene. The neuronal cell can be, but is not limited to, a brain cell, a deep brain cell, or a spinal cord cell.

Certain embodiments provide a method of modifying a neuronal cell to decrease Tau expression, the method comprising delivering to the neuron cell a MAPT ASO as described herein or a conjugate comprising a MAPT ASO as described herein, wherein the MAPT ASO specifically reduces the expression level of a MAPT transcript in the cell. The neuronal cell can be, but is not limited to, a brain cell, a deep brain cell, or a spinal cord cell.

Certain embodiments provide a method of delivering a MAPT ASO to the CNS of a human subject in need thereof, comprising administering to the subject a pharmaceutical composition comprising a MAPT ASO as described herein, wherein said MAPT ASO decreases the expression level of an endogenous MAPT gene.

Delivery of a described MAPT ASO to a neuronal cell can be used to treat a neurodegenerative disorder. Delivery of a described MAPT ASO to a neuronal cell or the can be used to treat a tau-associated neurodegenerative disorder. The neurodegenerative disorder can be, but is not limited to, Alzheimer's disease.

Certain embodiments provide a method of delivering a MAPT ASO to cells of the CNS of a human subject, comprising administering to the subject a pharmaceutical composition comprising a MAPT ASO as described herein, wherein said MAPT ASO is administered by intrathecal injection.

Certain embodiments provide a method of delivering a MAPT ASO to cells of the CNS of a human subject, comprising administering to the subject a pharmaceutical composition comprising a MAPT ASO as described herein, wherein said MAPT ASO is conjugated to a molecule (e.g., targeting ligand) or delivery vehicle that facilitates transport of the MAPT ASO across the blood brain barrier. The MAPT ASO can be linked to any molecule or delivery vehicle know in the art that facilitates transport across the blood brain barrier. Such molecules and delivery vehicles include, but are not limited to, brain shuttles (e.g., as disclosed in WO2018210898, WO2015101588, and WO2014033074, each is which is incorporated herein by reference).

Certain embodiments provide a method of treating a tau associated neurodegenerative disorder in a human subject in need thereof, the method comprising administering to the human subject a MAPT ASO as described herein or a composition (e.g., a pharmaceutical composition) comprising a MAPT ASO as described herein.

Certain embodiments provide a method of treating Alzheimer's disease, the method comprising administering to a human subject in need thereof, a MAPT ASO as described herein or a composition (e.g., a pharmaceutical composition) comprising a MAPT ASO as described herein.

Certain embodiments provide a method of reducing MAPT messenger ribonucleic acid (mRNA) expression in a human subject in need thereof, the method comprising administering to the human subject a MAPT ASO as described herein or a composition (e.g., a pharmaceutical composition) comprising a MAPT ASO as described herein.

Certain embodiments provide a MAPT ASO as described herein or a composition (e.g., a pharmaceutical composition) comprising a MAPT ASO as described herein for use in treating a tau associated neurodegenerative disorder in a human subject in need thereof.

Certain embodiments provide a MAPT ASO as described herein or a composition (e.g., a pharmaceutical composition) comprising a MAPT ASO as described herein for use in treating Alzheimer's disease in a human subject in need thereof.

Certain embodiments provide a a MAPT ASO as described herein or composition (e.g., a pharmaceutical composition) comprising a MAPT ASO as described herein for use in reducing MAPT mRNA expression in a human subject in need thereof.

Certain embodiments provide the use of a MAPT ASO in the preparation of a medicament for reducing MAPT mRNA expression in a human subject in need thereof.

### BRIEF DESCRIPTION OF THE FIGURES

FIG. 1 illustrates human tau knockdown in the cortex 2 weeks post in vivo ICV bolus.

FIG. 2 illustrates the effect of 5-methyl cytosine (5meC) addition on liver toxicity profile.

FIG. 3 illustrates ED50 curve in brain.

FIG. 4 illustrates ED50 curve in spinal cord.

FIG. 5 illustrates knockdown duration of action in brain and knockdown at 5 week and 9 week timepoints.

FIG. 6 illustrates knockdown duration of action in spinal cord and knockdown at 5 week and 9 week timepoints.

FIG. 7A-B illustrate results from rat renal toxicity assessments for (A) serum blood urea nitrogen (BUN); (B) creatinine.

FIG. 7C illustrate results from rat renal toxicity assessments for urine KIM-1: Creatinine ratio.

### DETAILED DESCRIPTION

Antisense oligonucleotides are single-stranded small synthetic nucleic acid polymers that can be used to modulate gene expression. They can target pre-mRNA, mRNA, or non-coding RNA to induce degradation, modulate splicing events, or interfere with protein translation. Described herein are antisense oligonucleotides that target MAPT RNA transcripts (e.g., a MAPT mRNA, such as a pre-mRNA or mature mRNA) and reduce the expression level of the MAPT gene.

The MAPT gene encodes the microtubule-associated protein tau (also referred herein as tau) whose transcript undergoes complex, regulated alternative splicing, giving rise to several mRNA species. MAPT transcripts are differentially expressed in the nervous system, depending on stage of neuronal maturation and neuron type. MAPT gene mutations have been associated with several neurodegenerative disorders, such as Alzheimer's disease, Pick's disease, frontotemporal dementia, cortico-basal degeneration and progressive supranuclear palsy. In some embodiments, a human MAPT gene has the sequence identified in GENBANK Accession No. NT_010783.15 (see, e.g., SEQ ID NO: 1 comprising nucleotides 9,240,000 to 9,381,000). In some embodiments, a MAPT pre-mRNA has the sequence of SEQ ID NO: 2. An embodiment of a mRNA transcript is provided as SEQ ID NO: 3.

The MAPT ASOs described herein can be used to treat Tau-associated disorders, e.g., Alzheimer's disease.

As used herein, the singular forms “a,” “an,” and “the” include plural referents unless the content clearly dictates otherwise.

As used herein, the terms “about” and “approximately,” when used to modify an amount specified in a numeric value or range, indicate that the numeric value as well as reasonable deviations from the value known to the skilled person in the art, for example ±20%, ±10%, or ±5%, are within the intended meaning of the recited value.

The term “antisense oligonucleotide (ASO)” refers to single strands of DNA-like or RNA-like molecules (e.g., comprising a modified internucleoside linkage(s), a modified nucleobase(s), and/or a modified sugar(s), such as those described herein) that are complementary or partially complementary to a chosen target polynucleotide sequence, e.g., an mRNA. By binding to a complementary target sequence ASOs can alter or modulate gene expression through a number of mechanisms, including, e.g., by altering splicing (exon exclusion or exon inclusion); by recruiting RNase H leading to target degradation; through translation inhibition; and by small RNA inhibition.

As used herein, a “MAPT antisense oligonucleotide” or “MAPT ASO” or “MAPT targeting antisense oligonucleotide” refers to an ASO that is capable of binding (hybridizing) to a MAPT target nucleic acid (e.g., an RNA transcript, such a MAPT mRNA (e.g., a MAPT pre-mRNA or a MAPT mature mRNA) or cDNA) in a sequence specific manner, resulting the reduction of MAPT gene expression.

A “transferrin receptor” or “TfR” as used herein refers to transferrin receptor protein 1. Transferrin receptor protein 1 sequences from several species are known (e.g., human, accession number NP_001121620.1 (also: NP_001300894.1, NP_001300895.1, NP_003225.2; chimpanzee, accession number XP_003310238.1; rhesus monkey, NP_001244232.1; dog, NP_001003111.1; cattle, NP_001193506.1; mouse, NP_035768.1; rat, NP_073203.1; and chicken, NP_990587.1). The term “transferrin receptor” also encompasses allelic variants of exemplary reference sequences, e.g., human sequences, that are encoded by a gene at a transferrin receptor protein 1 chromosomal locus. Full length transferrin receptor protein includes a short N-terminal intracellular region, a transmembrane region, and a large extracellular domain. The extracellular domain is characterized by three domains: a protease-like domain, a helical domain, and an apical domain.

The terms “identical” or percent “identity,” in the context of two or more polypeptide sequences, refer to two or more sequences or subsequences that are the same or have a specified percentage of amino acid residues, e.g., at least 60% identity, at least 65%, at least 70%, at least 75%, at least 80%, at least 85%, at least 90%, or at least 95% or greater, that are identical over a specified region when compared and aligned for maximum correspondence over a comparison window, or designated region as measured using one a sequence comparison algorithm or by manual alignment and visual inspection.

For sequence comparison of polypeptides, typically one amino acid sequence acts as a reference sequence, to which a candidate sequence is compared. Alignment can be performed using various methods available to one of skill in the art, e.g., visual alignment or using publicly available software using known algorithms to achieve maximal alignment. Such programs include the BLAST programs, ALIGN, ALIGN-2 (Genentech, South San Francisco, Calif.) or Megalign (DNASTAR). The parameters employed for an alignment to achieve maximal alignment can be determined by one of skill in the art. For sequence comparison of polypeptide sequences for purposes of this application, the BLASTP algorithm standard protein BLAST for aligning two proteins sequence with the default parameters is used.

The terms “corresponding to,” “determined with reference to,” or “numbered with reference to” when used in the context of the identification of a given nucleotide residue in a nucleic acid sequence, refers to the position of the residue of a specified reference sequence when the given nucleotide sequence is maximally aligned and compared to the reference sequence. The nucleic acid sequence that is aligned to the reference sequence need not be the same length as the reference sequence.

As used herein, the terms “nucleic acid” and “polynucleotide” refer to deoxyribonucleotides or ribonucleotides and polymers thereof in either single- or double-stranded form, composed of monomers (nucleotides) containing a sugar moiety, phosphate and a nucleobase. Unless specifically limited, the term encompasses both modified and unmodified nucleic acids.

As used herein, the term “nucleobase” refers to nitrogen-containing compounds that can be linked to a sugar moiety to form nucleosides, which in turn are components of nucleotides. The ability of nucleobases to form base pairs and to stack one upon another leads directly to long-chain helical structures such as ribonucleic acid (RNA) and deoxyribonucleic acid (DNA). Nucleobases may be naturally occurring (i.e., adenine (A), cytosine (C), guanine (G), thymine (T), and uracil (U)) or modified (e.g., 5-methyl cytosine). Thus, the nucleobase moieties may be shown by the corresponding letter code for each nucleobase, e.g., A, T, G, C or U, wherein each letter may optionally include modified nucleobases that are functionally equivalent (e.g., based on Watson Crick base pairing ability).

As used herein, the term “nucleoside” refers to a compound comprising a nucleobase and sugar moiety (e.g., deoxyribose or ribose, or a modified variant thereof). The term nucleoside includes both modified and unmodified nucleosides.

As used herein, the term “nucleotide” refers to a compound comprising a nucleobase, a sugar moiety, and one or more phosphate groups. The term nucleotide includes both modified and unmodified nucleotides.

As used herein, the term “internucleoside linkage” means the covalent linkages between two nucleosides in an oligonucleotide. Nucleosides may be linked via natural (i.e., a phophodiester (PO) linkage) or modified linkages.

The terms “chemical modification”, “modification” or “modified” may refer to a chemical change in a compound when compared to its naturally occurring counterpart. For example, a nucleobase, a sugar moiety or an internucleoside linkage may be chemically modified.

The terms “nucleotide sequence” and “nucleic acid sequence” and “nucleic acid strand” and “nucleobase sequence” refer to a sequence of bases (purines and/or pyrimidines, or synthetic derivatives thereof) in a polymer of DNA or RNA, or combinations thereof, which can be single-stranded or double-stranded, optionally containing synthetic, non-natural or altered nucleotides capable of incorporation into DNA or RNA polymers, and/or backbone modifications (e.g., a modified oligomer).

The terms “oligo”, “oligonucleotide” and “oligomer” may be used interchangeably and refer to such sequences of purines and/or pyrimidines. For example, the oligonucleotide may comprise chemically modified or unmodified nucleic acid molecules (RNA or DNA) having a length of less than about, e.g., about 200 nucleotides (for example, less than about 100 or 50 nucleotides). The oligonucleotide can, e.g., be single stranded DNA or RNA (e.g., an ASO); double stranded DNA or RNA (e.g., small interfering RNA (siRNA)), including double stranded DNA or RNA having a hairpin loop; or DNA/RNA hybrids. In some embodiments, the oligonucleotide has a length ranging from about 5 to about 60 nucleotides, or about 10 to about 50 nucleotides. In some embodiments, the oligonucleotide has a length ranging from about 5 to about 30 nucleotides or from about 15 to about 30 nucleotides. In some embodiments, the oligonucleotide has a length ranging from about 18 to about 24 nucleotides.

The terms “modified oligos”, “modified oligonucleotides” or “modified oligomers” may be similarly used interchangeably, and refer to such sequences that contain synthetic, non-natural or altered bases, sugars and/or backbone modifications. Reference herein to oligonucleotides, nucleic acids, polynucleotides, includes modified oligonucleotides, modified nucleic acids, and modified polynucleotides (i.e., oligonucleotides, nucleic acids, polynucleotides having one or more modified bases, sugars, or internucleoside linkages)

A “modified nucleotide” is a nucleotide other than a ribonucleotide (2′-hydroxyl nucleotide) or a deoxyribonucleotide (2′-H nucleotide). A modified nucleotide can comprise one or more of: a modified nucleobase, a modified ribose (sugar) moiety, and a modified internucleoside linkage to another nucleoside. Modified nucleotides include nucleotide mimics. Modified nucleosides include abasic nucleosides, which lack a nucleobase, and nucleosides in which the ribose is substituted for a non-sugar moiety (e.g., a sugar surrogate, e.g., a morpholino or as in a peptide nucleic acid).

A “2′-deoxynucleoside” is a nucleoside comprising a 2′-deoxyribose sugar moiety. In naturally occurring DNA, 2′-deoxynucleosides comprise a ribose having a β-D ribosyl configuration.

A “2′-substitution modified nucleoside” or “2′ substituted nucleoside” or “2′ modified nucleoside” is a nucleoside comprising a 2′-substitution (e.g., a group other than hydrogen or hydroxyl) at the 2′-OH group of a ribosyl sugar moiety. A 2′ substituted nucleoside comprises at least one 2′-substituent group other than H or OH at the 2′ carbon of the nucleoside ribose.

“2′-MOE modified nucleoside” or “2′-MOE nucleoside” is a nucleoside comprising a 2′-OCH2CH2OCH3 (O-methoxyethyl) substitution at the 2′-OH group of a ribosyl sugar moiety.

A “2′-NMA modified nucleoside” or “2′-NMA nucleoside” is a nucleoside comprising 2′-O-CH2-C(═O)—NH—CH3 (O—N-methyl acetamide) substitution at the 2′~OH group of a ribosyl sugar moiety.

A “2′-OMe modified nucleoside” or “2′-OMe nucleoside” is a nucleoside comprising a 2′-OCH3 substitution at the 2′-OH group of a ribosyl sugar moiety.

A “2′-F modified nucleoside” or “2′-F nucleoside” is a nucleoside comprising a 2′-fluoro substitution in place of the 2′-OH group of a ribosyl sugar moiety.

A “bicyclic nucleoside” (also termed bridged nucleoside) is a nucleoside comprising a bicyclic sugar moiety. A “bicyclic sugar” or “bicyclic sugar moiety” is a modified sugar moiety comprising two rings, wherein the second ring is formed via a bridge connecting two of the atoms in the first ring thereby forming a bicyclic structure. In some embodiments, the first ring of the bicyclic sugar moiety is a furanosyl moiety, such as a ribosyl sugar moiety of a nucleoside.

A “non-bicyclic modified sugar moiety” is a modified sugar moiety that comprises a modification, such as a substituent, that does not form a bridge between two atoms of the sugar to form a second ring.

A “constrained ethyl” or “cEt” or “cEt modified sugar moiety” or “cEt sugar moiety” is a β-D ribosyl bicyclic sugar moiety wherein the second ring of the bicyclic sugar is formed via a bridge connecting the 4′-carbon and the 2′-carbon of the β-D ribosyl sugar moiety, wherein the bridge has the formula 4′-CH(CH3)-O-2′, and wherein the methyl group of the bridge is in the S configuration. A “cEt modified nucleoside” or “cEt nucleoside” is a nucleoside comprising a cEt modified sugar moiety.

A “locked nucleic acid” or “LNA nucleoside” or “LNA” is a bicyclic nucleoside having a 4′-CH2-O-2′ bridge between the 4′ and the 2′ furanosyl ring atoms.

A “sugar surrogate” is a moiety having other than a ribosyl moiety of a modified nucleoside. Oligonucleotides comprising one or more suitable sugar surrogates retain the ability to hybridize to complementary target nucleobase or nucleic acid sequences.

An “internucleoside linkage” is the covalent linkage between adjacent nucleosides in an oligonucleotide. As used herein “modified internucleoside linkage” means any internucleoside linkage other than a phosphodiester internucleoside linkage.

A “phosphorothioate internucleoside linkage” is a modified internucleoside linkage in which one of the non-bridging oxygen atoms of a phosphodiester internucleoside linkage is replaced with a sulfur atom.

A “5-methylcytosine” comprises a cytosine modified with a methyl group attached to the 5 position. A 5-methylcytosine is a modified nucleobase.

An “abasic nucleoside” comprises a nucleoside lacking a nucleobase.

A “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In some embodiments, the molecules are modified oligonucleotides.

A “stabilized phosphate group” refers to a 5′-chemical moiety that results in stabilization of a S′-phosphate moiety of the S′-terminal nucleoside of an oligonucleotide, relative to the stability of an unmodified S′-phosphate of an unmodified nucleoside under biologic conditions. Stabilized phosphate groups include, but are not limited to, 5′-vinyl phosphonates and S′-cyclopropyl phosphonate.

The MAPT ASOs described herein may be synthesized using standard solid or solution phase synthesis techniques that are known in the art. In certain embodiments, the MAPT ASOs are synthesized using solid-phase phosphoramidite chemistry (U.S. Pat. No. 6,773,885) with automated synthesizers. Chemical synthesis of nucleic acids allows for the production of various forms of the nucleic acids with modified linkages, chimeric compositions, and nonstandard bases or modifying groups attached in chosen places through the nucleic acid's entire length.

The term “complementary” as used herein refers to the broad concept of complementary base pairing between two nucleic acids aligned in an antisense position in relation to each other. When a nucleotide position in both of the molecules is occupied by nucleotides normally capable of base pairing with each other, then the nucleic acids are considered to be complementary to each other at this position. Thus, two nucleic acids are substantially complementary to each other when at least about 50%, at least about 60%, or at least about 80% of corresponding positions in each of the molecules are occupied by nucleotides which normally base pair with each other (e.g., A:T (A:U for RNA) and G:C nucleotide pairs).

The term percent “complementary,” in the context of two or more nucleotide sequences, refer to two or more sequences or subsequences that are the complementary or have a specified percentage of nucleotides, e.g., at least 60% identity, at least 65%, at least 70%, at least 75%, at least 80%, at least 85%, at least 90%, or at least 95% or greater, that are complementary over a specified region when compared and aligned for maximum correspondence over a comparison window, or designated region as measured using one a sequence comparison algorithm or by manual alignment and visual inspection.

The terms “identical” or percent “identity,” in the context of two or more nucleotide sequences, refer to two or more sequences or subsequences that are the same or have a specified percentage of nucleotides, e.g., at least 60% identity, at least 65%, at least 70%, at least 75%, at least 80%, at least 85%, at least 90%, or at least 95% or greater, that are identical over a specified region when compared and aligned for maximum correspondence over a comparison window, or designated region as measured using one a sequence comparison algorithm or by manual alignment and visual inspection.

It will be understood that in determining percent complementarity or percent identity, chemical modifications are disregarded if the functional capacity of the nucleobase to form Watson Crick base pairing is retained (e.g., S-methyl cytosine is considered identical to a cytosine for the purpose of calculating % identity).

For sequence comparison of oligonucleotides (e.g., to determine identity or complementarity), typically one nucleotide sequence acts as a reference sequence, to which a candidate sequence is compared. Alignment can be performed using various methods available to one of skill in the art, e.g., visual alignment or using publicly available software using known algorithms to achieve maximal alignment. Such programs include the BLAST programs, ALIGN, ALIGN-2 (Genentech, South San Francisco, Calif.) or Megalign (DNASTAR). The parameters employed for an alignment to achieve maximal alignment can be determined by one of skill in the art.

As used herein, “hybridize” or “hybridization” means the pairing of complementary nucleotide sequences (e.g., an antisense compound and its target nucleic acid; or between antisense and sense strands). As used herein, “specifically hybridizes” means the ability of a reference nucleic acid to hybridize to one nucleic acid molecule with greater affinity than it hybridizes to another.

“Expression” refers to the transcription and/or translation of an endogenous gene, heterologous gene or nucleic acid segment, or a transgene in cells. For example, expression may refer to the transcription and stable accumulation of sense (mRNA) or functional RNA. Expression may also refer to the production of protein.

The term “gene” refers to a nucleic acid (e.g., DNA or RNA) sequence that comprises coding sequences necessary for the production of a polypeptide or precursor.

The phrase “modulating the expression of a target gene or sequence” means a change (e.g., an increase or decrease) in expression of the target gene or sequence (e.g., via degradation of the target or translation inhibition). For example, it includes inhibiting, reducing or decreasing the expression of a target gene or sequence. This also includes a change in alternative splicing, which may result in a change in the absolute or relative amount of a particular splice variant.

The term “subject,” “individual,” and “patient,” as used interchangeably herein, refer to a mammal, including but not limited to humans, non-human primates, rodents (e.g., rats, mice, and guinea pigs), rabbits, cows, pigs, horses, and other mammalian species. In some embodiments, the patient is a human.

The terms “treatment,” “treating,” and the like are used herein to generally mean obtaining a desired pharmacologic and/or physiologic effect. The terms “treating,” “treatment,” and the like, include the methods or steps taken to provide relief from, or amelioration or alleviation of the number, severity, adverse effect, and/or frequency of one or more symptoms or pathological consequences of a disease, disorder, or condition in a subject. Treating the disease, disorder, or condition can include ameliorating at least one symptom of the particular disease, disorder, or condition, even if the underlying pathophysiology is not affected. “Treating” or “treatment” may refer to any indicia of success in the treatment or amelioration of an injury, disease, or condition, including any objective or subjective parameter such as abatement, remission, improvement in patient survival, increase in survival time or rate, diminishing of symptoms or making the injury, disease, or condition more tolerable to the patient, slowing in the rate of degeneration or decline, or improving a patient's physical or mental well-being. Treatment can be therapeutic in terms of a partial or complete cure of a disease, condition, symptom or adverse effect attributed to the disease, disorder, or condition. Treatment can be prophylactic in terms of preventing or partially preventing a disease, or a symptom or condition of the disease. Preventing includes providing prophylaxis with respect to the occurrence or recurrence of a disease in a subject that may be predisposed to the disease but has not yet been diagnosed with the disease. Preventing also includes providing prophylaxis with respect to the occurrence or recurrence of a symptom or pathological consequence of a disease in a subject that may be predisposed to the symptom or pathological consequence of the disease but has not yet been diagnosed with the symptom or pathological consequence the disease. Treatment can also be prophylactic in terms of delaying onset of a disease, or a symptom or condition of the disease. Delaying development of a disease or symptom or pathological consequence of the disease indicates deferring, hindering, slowing, retarding, stabilizing, suppressing, and/or postponing development of the disease or symptom or pathological consequence of the disease. The delay can be of varying lengths of time, depending on the history of the disease and/or individual being treated. Treating can refer to both therapeutic treatment alone, prophylactic treatment alone, or both therapeutic and prophylactic treatment. Those in need of treatment (subjects in need thereof) can include those previously diagnosed with a disease, disorder, or condition, or those identified as being at risk of developing a disease, disorder, or condition. Additionally, “treating” or “treatment” may refer to the modulation of the target gene expression such as gene knockdown or gene knockout. For instance, the expression of the target gene or sequence is inhibited or reduced, e.g., by at least about 10%, 20%, 30%, 40%, 50%, 60%, 70%, 80%, 90%, 95%, 99% or 100%, as compared to the expression in a control. The treatment or amelioration of symptoms can be based on objective or subjective parameters. The effect of treatment can be compared to an individual or pool of individuals not receiving the treatment, or to the same patient prior to treatment or at a different time during treatment.

As used herein, “ameliorate” refers to the use of an effective amount of a MAPT ASO and to conjugates comprising the same for improving at least one symptom (e.g., as compared to the same symptom in the absence of the treatment). In certain embodiments, amelioration is the reduction in the severity or frequency of a symptom or the delayed onset or slowing of progression in the severity or frequency of a symptom.

The terms “prevent” or “prevention” refer to the use of an effective amount of a MAPT ASO and to conjugates comprising the same for reducing or eliminating the occurrence or recurrence of a disease or a disease symptom in a subject, such as in a subject prone to developing or re-developing the disease.

The term “pharmaceutically acceptable excipient” refers to a non-active pharmaceutical ingredient that is biologically or pharmacologically compatible for use in humans or animals, such as but not limited to a buffer, carrier, or preservative.

As used herein, a “therapeutic amount” or “therapeutically effective amount” of an agent is an amount of the agent that treats, alleviates, abates, or reduces the severity of symptoms of a disease in a subject. A “therapeutic amount” or “therapeutically effective amount” of an agent may improve patient survival, increase survival time or rate, diminish symptoms, make an injury, disease, or condition more tolerable, slow the rate of degeneration or decline, or improve a patient's physical or mental well-being. The therapeutically effective amount may vary according to factors such as the disease state, age, sex, and weight of the subject, and the populations of cells administered.

A “dose,” “unit dose.” or “dosage” refers to physically discrete units suitable for use in a subject, each unit containing a predetermined quantity of active pharmaceutical ingredient and/or a pharmaceutical composition.

The term “administer” refers to a method of delivering agents, compounds, or compositions to the desired site of biological action. These methods include, but are not limited to, topical delivery, parenteral delivery, intravenous delivery, intradermal delivery, intramuscular delivery, intrathecal delivery, colonic delivery, rectal delivery, or intraperitoneal delivery. In some embodiments, the proteins described herein are administered intravenously.

The term “control” or “control value” refers to a reference value or baseline value. Appropriate controls can be determined by one skilled in the art. In some instances, control values can be determined relative to a baseline within the same subject or experiment, e.g., a measurement of MAPT gene expression taken prior to treatment with a MAPT ASO as described herein or a conjugate or composition thereof can be a control value for a post-treatment measurement of MAPT levels in the same subject. In other instances, the control value can be determined relative to a control subject (e.g., a healthy control or a disease control) or an average value in a population of control subjects (e.g., healthy controls or disease controls, e.g., a population of 10, 20, 50, 100, 200, 500, 1000 control subjects or more), e.g., a measurement of a subject's level of MAPT gene expression either at baseline or after treatment can be compared to a healthy control value.

### I. MAPT Targeting Oligonucleotides

Disclosed herein are MAPT antisense oligonucleotides (MAPT ASOs) complementary to the human MAPT gene. The MAPT ASOs as described herein are at least 90% complementary to a sequence in the human MAPT gene. In some embodiments, the MAPT ASOs as described herein are at least 90% complementary to a sequence in SEQ ID NO: 1. In some embodiments, a MAPT ASO as described herein comprises or consists of a nucleic acid sequence that is at least 70%, at least 75%, at least 80%, at least 85%, at least 90%, at least 93%, at least 95%, at least 96%, at least 97%, at least 98%, at least 99%, or 100% complementary to a sequence present in SEQ ID NO: 1. In some embodiments, a MAPT ASO as described herein comprises or consists of a nucleic acid sequence 10 to 25 nucleotides in length that is at least 70%, at least 75%, at least 80%, at least 85%, at least 90%, at least 93%, at least 95%, at least 96%, at least 97%, at least 98%, at least 99%, or 100% complementary to a sequence in SEQ ID NO: 1. In some embodiments, a MAPT ASO as described herein comprises or consists of a nucleic acid sequence 10 to 25 nucleotides in length that is at least 90% or at least 95% complementary to a sequence in SEQ ID NO: 1. In some embodiments, a MAPT ASO as described herein comprises or consists of a nucleic acid sequence 10 to 25 nucleotides in length that is 100% complementary to a sequence in SEQ ID NO: 1.

For example, in some embodiments, disclosed herein are MAPT ASOs comprising at least 9, at least 10, at least 11, at least 12, at least 13, at least 14, at least 15, at least 16, at least 17, at least 18, or at least 19 contiguous nucleobases that are complementary to a sequence present in nucleobases 66,750 to 66,786; 66,760 to 66,786; 66,750 to 66,776; 66,754 to 66,786; or 66,760 to 66,776 of SEQ ID NO: 1.

In some embodiments, disclosed herein are MAPT ASOs comprising at least 9, at least 10, at least 11, at least 12, at least 13, at least 14, at least 15, at least 16, at least 17, at least 18, or at least 19 contiguous nucleobases that are complementary to a sequence present in nucleobases 78,431 to 78,468; 78,431 to 78,458; 78,441 to 78,468; or 78,441 to 78,458 of SEQ ID NO: 1.

In some embodiments, disclosed herein are MAPT ASOs comprising at least 9, at least 10, at least 11, at least 12, at least 13, at least 14, at least 15, at least 16, at least 17, at least 18, or at least 19 contiguous nucleobases that are complementary to a sequence present in nucleobases 78,621 to 78,659; 78,621 to 78,649; 78,631 to 78,659; or 78,631 to 78,649 of SEQ ID NO: 1.

In some embodiments, disclosed herein are MAPT ASOs comprising at least 9, at least 10, at least 11, at least 12, at least 13, at least 14, at least 15, at least 16, at least 17, at least 18, or at least 19 contiguous nucleobases that are complementary to a sequence present in nucleobases of 78,818 to 78,855; 78,828 to 78,855; 78,818 to 78,845; or 78,828 to 78,845 of SEQ ID NO: 1.

In some embodiments, disclosed herein are MAPT ASOs comprising at least 9, at least 10, at least 11, at least 12, at least 13, at least 14, at least 15, at least 16, at least 17, at least 18, or at least 19 contiguous nucleobases that are complementary to a sequence present in nucleobases of 78,431 to 78,659; 78,431 to 78,649; 78,441 to 78,659; or 78,441 to 78,649 of SEQ ID NO: 1.

In some embodiments, disclosed herein are MAPT ASOs comprising at least 9, at least 10, at least 11, at least 12, at least 13, at least 14, at least 15, at least 16, at least 17, at least 18, or at least 19 contiguous nucleobases that are complementary to a sequence present in nucleobases of 78,641 to 78,855; 78,641 to 78,845; 78,631 to 78,855; or 78,631 to 78,845 of SEQ ID NO: 1.

In some embodiments, disclosed herein are MAPT ASOs comprising at least 9, at least 10, at least 11, at least 12, at least 13, at least 14, at least 15, at least 16, at least 17, at least 18, or at least 19 contiguous nucleobases that are complementary to a sequence present in nucleobases of 78,431 to 78,855; 78,441 to 78,855; 78,431 to 78,845; 78,441 to 78,845; 78441 to 78,498; 78,517 to 78,678; or 78,697 to 78,845 of SEQ ID NO: 1.

In some embodiments, disclosed herein are MAPT ASOs comprising at least 9, at least 10, at least 11, at least 12, at least 13, at least 14, at least 15, at least 16, at least 17, at least 18, or at least 19 contiguous nucleobases that are complementary to a sequence present in nucleobases of 90,205 to 90,243; 90,205 to 90,233; 90,215 to 90,243; 90,208 to 90,243; or 90,215 to 90,233 of SEQ ID NO: 1.

ASOs range from about 10 to 30 base pairs (bp) in length, but may be longer or shorter. For example, in certain embodiments, the ASO is about 10 to about 60 nucleotides in length (i.e., about 10 to about 60 linked nucleosides in length), or about 10 to about 50 nucleotides in length, or about 10 to about 40 nucleotides in length. In certain embodiments, the ASO is about 10 to 30 nucleotides in length, or about 12 to 30 nucleotides in length, or about 14 to about 30 nucleotides in length, or about 15 to about 30 nucleotides in length, or about 16 to about 30 nucleotides in length, or about 17 to about 30 nucleotides in length, or about 18 to about 30 nucleotides in length, or about 18 to about 28 nucleotides in length or about 18 to 26 nucleotides in length, or about 18 to about 24 nucleotides in length, or about 15 to about 25 nucleotides in length, or about 16 to about 20 nucleotides in length, or about 16 to about 19 nucleotides in length, or about 17 to about 19 nucleotides in length. In certain embodiments, the ASO is about 15, 16, 17, 18, 19, 20, 21, 22, 23, 24, 25, 26, 27, 28, 29 or 30 nucleotides in length. In certain embodiments, the ASO is 16, 17, 18, or 19 nucleotides in length.

While the length of the MAPT ASO may vary, in certain embodiments, the MAPT ASO is from about 10 to about 60 nucleotides in length, or from about 10 to about 30 nucleotides in length, or from about 18 to about 30 nucleotides in length or from about 15 to about 25 nucleotides in length, or from about 16 to about 20 nucleotides in length. In some embodiments, the MAPT ASO is about 16 to about 20 nucleotides in length. In some embodiments, the MAPT ASO is about 16 to about 19 nucleotides in length. In some embodiments, the ASO is about 17 to about 19 nucleotides in length. In some embodiments, the MAPT ASO is 16, 17, 18, 19, or 20 nucleotides in length. In some embodiments, the MAPT ASO is 16 nucleotides in length. In some embodiments, the MAPT ASO is 17 nucleotides in length. In some embodiments, the MAPT ASO is 18 nucleotides in length. In some embodiments, the MAPT ASO is 19 nucleotides in length. In some embodiments, the MAPT ASO is 20 nucleotides in length.

In some embodiments, a MAPT ASO disclosed herein comprises or consists of a nucleic acid sequence comprising or consisting of at least 9, at least 10, at least 11, at least 12, at least 13, at least 14, at least 15, at least 16, at least 17, at least 18, or at least 19 contiguous nucleobases of a sequence selected from any one of SEQ ID NOs: 4-9 and 19-37 (Table 1) and at least one modified internucleoside linkage and/or at least one modified sugar. In some embodiments, a MAPT ASO disclosed herein comprises a nucleic acid sequence selected from any one of SEQ ID NOs: 4-9 and 19-37 and further comprises at least one modified internucleoside linkage and/or at least one modified sugar. In some embodiments, a MAPT ASO disclosed herein consists of a nucleic acid sequence selected from any one of SEQ ID NOs: 4-9 and 19-37 and further comprises at least one modified internucleoside linkage and/or at least one modified sugar. In some embodiments, the MAPT ASO disclosed herein targets an exon of the MAPT gene and comprises or consists of a nucleic acid sequence of any one of SEQ ID NOs: 4 and 19-21 and further comprises at least one modified internucleoside linkage and/or at least one modified sugar. In some embodiments, the MAPT ASO disclosed herein targets an intron of the MAPT gene and comprises or consists of a nucleic acid sequence selected from any one of SEQ ID NOs: 5-9 and 22-37 and further comprises at least one modified internucleoside linkage and/or at least one modified sugar. In some embodiments, the MAPT ASOs disclosed herein comprise 15 to 25 linked nucleosides. In some embodiments, the MAPT ASOs disclosed herein comprise 16 to 20 linked nucleosides. In some embodiments, the MAPT ASOs disclosed herein comprise 16 to 19 linked nucleosides. In some embodiments, the MAPT ASOs disclosed herein comprise 17 to 19 linked nucleosides. In some embodiments, the MAPT ASO disclosed herein (e.g., 15 to 25 or 17 to 19 nucleotides in length) are at least 70%, at least 75%, at least 80%, at least 85%, at least 90%, at least 93%, at least 95%, at least 96%, at least 97%, at least 98%, at least 99%, or 100% complementary to SEQ ID NO: 1.

In some embodiments, a MAPT ASO disclosed herein is 16-20 nucleotides in length, and comprises the sequence of any one of SEQ ID NOs: 10-15 and 38-56 (Table 2), wherein the MAPT ASO further comprises at least one modified internucleoside linkage and/or at least one modified sugar. In some embodiments, a MAPT ASO disclosed herein is 17-19 nucleotides in length, and comprises the sequence of any one of SEQ ID NOs: 10-15 and 38-56, wherein the MAPT ASO further comprises at least one modified internucleoside linkage and/or at least one modified sugar. In some embodiments, a MAPT ASO disclosed herein comprises a gapmer ASO having a gap segment comprising the sequence of any one of SEQ ID NOs: 10-15 and 38-56. In some embodiments, a MAPT ASO disclosed herein comprises a gapmer ASO having a gap segment consisting of the sequence of any one of SEQ ID NOs: 10-15 and 38-56. In some embodiments, a MAPT ASO comprises a sequence comprising 100% identity to any of the SEQ ID NOs: 10-15 and 38-56 (Table 2) and at least 85%, at least 90%, at least 95% or 100% identity to any of SEQ ID NOs: 4-9 and 19-37 (Table 1).

TABLE 1

| SEQ ID NO. | Sequence (5′→3′) |
|---|---|
| 4 | cgtgtttgatattatcct |
| 5 | accttaagtattacttgc |
| 6 | tcaactcttagcatccact |
| 7 | cttatcatttgcattggt |
| 8 | ctgttagacattcattctc |
| 9 | cgtattatgctagaggt |
| 19 | cgtgtttgatattatc |
| 20 | gtgtttgatattatcc |
| 21 | tgtttgatattatcct |
| 22 | accttaagtattactt |
| 23 | ccttaagtattacttg |
| 24 | cttaagtattacttgc |
| 25 | tcaactcttagcatcc |
| 26 | caactcttagcatcca |
| 27 | aactcttagcatccac |
| 28 | actcttagcatccact |
| 29 | cttatcatttgcattg |
| 30 | ttatcatttgcattgg |
| 31 | tatcatttgcattggt |
| 32 | ctgttagacattcatt |
| 33 | tgttagacattcattc |
| 34 | gttagacattcattct |
| 35 | ttagacattcattctc |
| 36 | cgtattatgctagagg |
| 37 | gtattatgctagaggt |

TABLE 2

| SEQ ID NO. | Sequence (5′→3′) |
|---|---|
| 10 | gtttgatattat |
| 11 | ttaagtattact |
| 12 | actcttagcatcc |
| 13 | atcatttgcatt |
| 14 | ttagacattcatt |
| 15 | attatgctaga |
| 38 | gtttgatatt |
| 39 | tttgatatta |
| 40 | ttgatattat |
| 41 | ttaagtatta |
| 42 | taagtattac |
| 43 | aagtattact |
| 44 | actcttagca |
| 45 | ctcttagcat |
| 46 | tcttagcatc |
| 47 | cttagcatcc |
| 48 | atcatttgca |
| 49 | tcatttgcat |
| 50 | catttgcatt |
| 51 | ttagacattc |
| 52 | tagacattca |
| 53 | agacattcat |
| 54 | gacattcatt |
| 55 | attatgctag |
| 56 | ttatgctaga |

### Illustrative Oligonucleotide Modifications

In certain embodiments, a MAPT ASO described herein may comprise at least one nucleic acid modification, such as those selected from the group consisting of a modified internucleoside linkage, a modified nucleobase, a modified sugar, and combinations thereof (e.g., comprises at least one modified internucleoside linkage and/or at least one modified sugar). Such modifications may be used to alter pharmacokinetics (improved nuclease resistance resulting in a longer half-life), pharmacodynamics (superior affinity for the target RNA), or endocytic uptake. However, many modifications preclude cleavage by RNase H, which is the desired mechanism of action for many ASOs. Thus, certain RNase H ASOs may be designed as chimeras, where different bases are a mix of different chemistries, or as gapmers, where some modifications are placed on the “wings” and not the central bases.

Accordingly, a MAPT ASO described herein may comprise one or more nucleic acid modifications. In certain embodiments, A MAPT ASO comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 40, or more modifications. In certain embodiments, a MAPT ASO described herein comprises one or more nucleotide modifications (e.g., to the nucleobase or sugar moiety). In certain embodiments, 25% or more of the nucleotides present in the MAPT ASO are modified. In certain embodiments, 50% or more of the nucleotides present in the MAPT ASO are modified. In certain embodiments, 75% or more of the nucleotides present in the MAPT ASO are modified. In certain embodiments, 100% of the nucleotides present in the MAPT ASO are modified.

### Modified Sugar Moiety, Nucleosides/Nucleotides

In certain embodiments, the MAPT ASO comprises one or more nucleobase modifications. In certain embodiments, the MAPT ASO comprises one or more modifications to one or more sugar moieties (e.g., furanosyls comprising substitutions at the 2′-position, the 3′-position, the 4′-position and/or the 5′-position). In certain embodiments, substituted sugar moieties include bicyclic sugar moieties. In some embodiments, the bicyclic sugar moieties comprise a chemical bridge between the 4′ and 2′ positions of the sugar, wherein each chemical bridge is independently selected from: 4′-CH(R)—O-2′ and 4′-(CH₂) 2-O-2′, wherein each R is independently selected from H, C₁-C₆ alkyl and C₁-C₆ alkoxy. In some embodiments, the bicyclic sugar moieties comprise a chemical bridge between the 4′ and 2′ positions of the sugar, wherein each chemical bridge is 4′-CH(R)—O-2′ and wherein each R is independently H.

Modified nucleosides/nucleotides include, but are not limited to, 2′-O methyl (2′OMe) residues, 2′ O-methoxyethyl (MOE) residues, constrained nucleic acid residues (e.g., S-cEt, R-cEt, S-cMOE, and R-cMOE), peptide nucleic acid (PNA) residues, locked nucleic acid (LNA) residues, and 5-methylcytidine residues (methylated cytosine residues) (see, also, Scoles, et al., Neurol Genet April 2019, 5 (2) e323). In certain embodiments, the MAPT ASO comprises one or more 2′-MOE residues. In certain embodiments, the MAPT ASO comprises one or more OMe residues or F residues (e.g., 2′-F or 2′OMe). In certain embodiments, the MAPT ASO comprises one or more constrained (e.g., S-cEt, R-cEt, S-cMOE, and R-cMOE) and/or LNA residues. Nucleic acids are considered “locked” when they have a methylene bridge connection made between 2′-oxygen and the 4′-carbon of the ribose sugar molecule. In certain embodiments, the MAPT ASO is a morpholino (i.e., comprises certain modifications to the sugar moiety).

In certain embodiments, a MAPT ASO described herein comprises one or more LNA residues. In certain embodiments, a MAPT ASO described herein comprises one or more 5-methylcytidine residues. In certain embodiments, a MAPT ASO described herein comprises one or more LNA residues and one or more 5-methylcytidine residues. In some embodiments, every cytosine in a MAPT ASO is a 5-methylcytidine.

### Modified Internucleoside Linkage

In certain embodiments, the MAPT ASO comprises one or more modifications to one or more internucleosides linkages (i.e., the natural phosphodiester (PO) linkage is modified). In certain embodiments, such modifications are made to, e.g., reduce nuclease activity. Thus, in certain embodiments, a MAPT ASO comprises 1, 2, 3, 4, 5, 6, 7, 8, 9, 10, 11, 12, 13, 14, 15, 16, 17, 18, 19, 20, 21, 22, 23, 24, 25 or more modified internucleoside linkages. In certain embodiments, 25% or more of the internucleoside linkages are modified. In certain embodiments, 50% or more of the internucleoside linkages are modified. In certain embodiments, 75% or more of the internucleoside linkages are modified. In certain embodiments, 100% of the internucleoside linkages present in the MAPT ASO are modified.

Backbone modifications are known in the art and include, but are not limited, to, phosphorothioate (PS) linkages, chiral phosphorothioate linkages, phosphorodiamidate linkages, phosphorodithioate linkages, aminoalkylphosphotriester linkages, phosphotriester linkages, thiophosphate linkages, phosphonate linkages, methyl phosphonate linkages, alkyl phosphonate linkages, 3′ alkylene phosphonate linkages, chiral phosphonate linkages, 3′-amino phosphoramidate linkages, aminoalkylphosphoramidate linkages, phosphinate linkages, thionoalkylphosphonate linkages, thionophosphoramidate linkages, thionoalkyl-phosphotriester linkages, borano-phosphate linkages, morpholino linkages and peptide nucleic acid (PNA) linkages. For example, in certain embodiments, one or more (e.g., 1, 2, 3, 4, 5, 6, 7, 8, 9, 10, 11, 12, 13, 14, 15, 16, 17, 18, 19, 20, 21, 22, 23, 24, 25 or more) of the internucleoside linkages in the MAPT ASO are replaced with phosphorothioate (PS) linkages. In certain embodiments, the MAPT ASO comprises a mix of modified and unmodified linkages. The modification at one internucleoside linkage can be independent of the modification at another internucleoside linkage. In certain embodiments, every internucleoside linkage in a MAPT ASO is a modified linkage. In certain embodiments, every internucleoside linkage in a MAPT ASO is a PS linkage. In some embodiments, every internucleoside linkage in an LPA ASO is a phosphorothioate linkage. In certain other embodiments, one or more (e.g., 1, 2, 3, 4, 5, 6, 7, 8, 9, 10, 11, 12, 13, 14, 15, 16, 17, 18, 19, 20, 21, 22, 23, 24, or 25 or more) of the internucleoside linkages in the MAPT ASO are replaced with a phosphorodiamidate linkages. In certain embodiments, the MAPT ASO is a phosphorodiamidate morpholino (PMO).

In certain embodiments, the internucleoside linkages are stereorandom with regard to the chiral centers (Rp and Sp). In certain other embodiments, the Rp and Sp configurations in the MAPT ASO are optimized in particular configurations.

### Antisense Oligonucleotide Motifs

In certain embodiments, the nucleic acid modifications with the MAPT ASO are included in a pattern. In certain embodiments, the MAPT ASO is a gapmer. The modification pattern of a gapmer MAPT ASO generally has the formula 5′-Xₐ—Yₐ—Zₐ-3′, with Xₐ and Zₐ as flanking regions around a gap region Yₐ. In certain embodiments, the Yₐ region (gap region) is a contiguous stretch of nucleotides (i.e., linked nucleosides), e.g., a region of at least 6 DNA nucleotides, which are capable of recruiting an RNAse, such as RNAse H. In certain embodiments, the Yₐ, region is at least 8 DNA nucleotides. In certain embodiments, the Yₐ region is about 9 to about 15 DNA nucleotides. In certain embodiments, the Yₐ region is about 11 to about 13 DNA nucleotides. In certain embodiments, the Yₐ region is 10, 11, 12, or 13 DNA nucleotides. In certain embodiments, the gapmer binds to the target nucleic acid, at which point an RNAse is recruited and can then cleave the target nucleic acid. In certain embodiments, the Yₐ region is flanked both 5′ and 3′ by regions Xₐ and Zₐ, which each comprise modified nucleotides, e.g., one to six modified nucleotides in each of Xₐ and Zₐ. In certain embodiments, the Yₐ region is flanked both 5′ and 3′ by regions Xₐ and Zₐ, wherein Xₐ and Zₐ comprise modified nucleotides having modified sugars. In certain embodiments, each nucleotide in Xₐ and each nucleotide in Zₐ comprises a modified nucleotide having a sugar modification. Each modified nucleotide can independently be, but is not limited to, a 2-MOE modified nucleotide, a bicyclic nucleotide, a LNA nucleotide, or a cET modified nucleotide. In certain embodiments, the modified nucleotides are present in the 5′ and 3′ regions of the MAPT ASO, while certain modified nucleotides and/or modified linkages may or may not be present in the central (gap (i.e., Yₐ)) portion of the molecule. In certain embodiments, the modified nucleotides are present in the 5′ and 3′ regions of the MAPT ASO and certain modified nucleotides are not present in the central portion of the molecule (e.g., LNA residues are not present in the central portion; however, the central region may contain modified linkages, such as PS linkages). In certain embodiments, Xₐ and Zₐ are each independently about 3 to about 6 nucleotides in length. In certain embodiments, Xₐ and Zₐ are each independently 3, 4, or 5 nucleotides in length. In certain embodiments, Xₐ and Zₐ each comprise 3 modified nucleotides (e.g., comprising a modified sugar). In certain embodiments, Xₐ and Zₐ each comprise 3 modified nucleotides wherein each comprises a modified sugar. In certain embodiments, Xₐ and Zₐ each comprise 3 modified nucleotides wherein each comprises a modified sugar and a modified internucleoside linkage. In certain embodiments, the 3 modified nucleotides are arranged in tandem in each of Xₐ and Zₐ.

In certain embodiments, the MAPT ASO is a gapmer comprising LNA and PS modifications. For example, in certain embodiments, the MAPT ASO is a gapmer having a modification pattern of the formula 5′-Xₐ—Yₐ—Zₐ-3′, with Xₐ and Zₐ as flanking regions around a gap region Yₐ, wherein Xₐ and Zₐ each comprise 3 LNA modified nucleotides (e.g., 3 consecutive LNA modified nucleotides), and wherein the gap region Yₐ comprises PS linkages. In some embodiments, every internucleotide linkage in the MAPT ASO comprises a PS linkage. In some embodiments, the MAPT ASO contains a mixture of PS linkages and another modified internucleoside linkage. In certain embodiments, the MAPT ASOfurther comprises one or more 5-methylcytidine residues. In certain embodiments, the gap region Yₐ does not comprise LNA residues.

In certain embodiments, and MAPT ASO comprises from 5′ to 3′: a 5′ wing segment having from 1 to 6 nucleosides, wherein each nucleoside of the 5′ wing segment comprises a modified sugar; a gap segment having from 8 to 15 nucleosides, wherein each nucleoside of the gap segment is a deoxynucleoside; and a 3′ wing segment having from 1 to 6 nucleosides, wherein each nucleoside of the 3′ wing segment comprises a modified sugar. In some embodiments, every internucleotide linkage in the MAPT ASO comprises a modified internucleoside linkage. In some embodiments, every internucleotide linkage in the MAPT ASO comprises a PS linkage. In some embodiments, the MAPT ASO contains a mixture of PS linkages and another modified internucleoside linkage. In some embodiments, every nucleoside in 5′ and 3′ wing segments comprises a 2-MOE modified nucleoside, a bicyclic nucleoside, a LNA nucleoside, or a cET modified nucleoside.

In certain embodiments, the MAPT ASO comprises, from 5′ to 3′: a 5′ wing segment having 3 nucleosides, wherein each nucleoside of the 5′ wing segment comprises a modified sugar; a gap segment having from 11 to 13 nucleosides, wherein each nucleoside of the gap segment is a deoxynucleoside; and a 3′ wing segment having from 3 nucleosides, wherein each nucleoside of the 3′ wing segment comprises a modified sugar. In some embodiments, every internucleotide linkage in the MAP ASO comprises a modified internucleoside linkage. In some embodiments, every internucleotide linkage in the MAP ASO comprises a PS linkage. In some embodiments, every nucleoside in 5′ and 3′ wing segments comprises a 2-MOE modified nucleotide, a bicyclic nucleotide, a LNA nucleotide, or a cET modified nucleotide.

In some embodiments, provided herein is a MAPT ASO comprising or consisting of a modified nucleic acid sequence having at least about 70%, at least 75%, at least 80%, at least 85%, at least 90%, at least 93%, at least 95%, at least 96%, at least 97%, at least 98%, at least 99%, or 100% identity to a modified sequence as shown in Table 7. In certain embodiments, the MAPT ASO comprises a modified nucleic acid sequence as shown in Table 7. In certain embodiments, the MAPT ASO comprises a nucleobase sequence consisting of a modified nucleic acid sequence as shown in Table 7. In certain embodiments, the MAPT ASO comprises a nucleobase sequence consisting of a modified nucleic acid sequence as shown in Table 7; wherein the MAPT AOS is conjugated to a targeting ligand or delivery vehicle (e.g., a vehicle that facilitates transport across the blood brain barrier). The MAPT ASOs in Table 7 are gapmers having wing segments having LNA modified nucleosides. Similar MAPT ASO gapmers can be made having wing segments having other ribose-modified nucleosides, including, but not limited to, 2′-MOE nucleotides and/or cEt nucleosides.

In some embodiments, provided herein are modified oligonucleotides (MAPT ASOs) according to any of the following chemical structures:

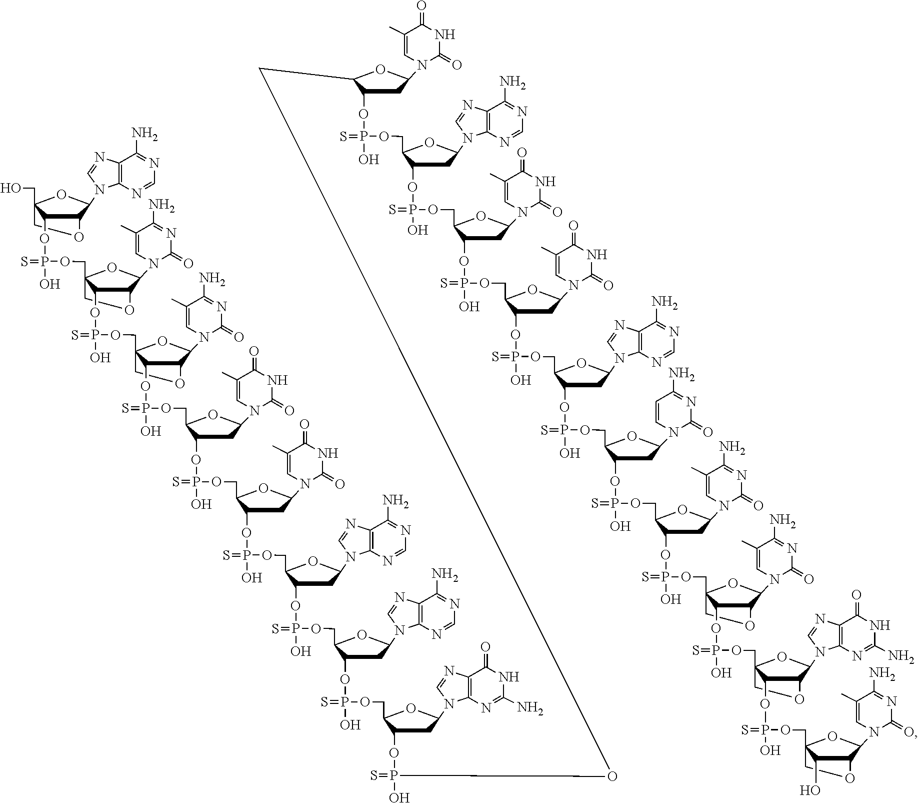

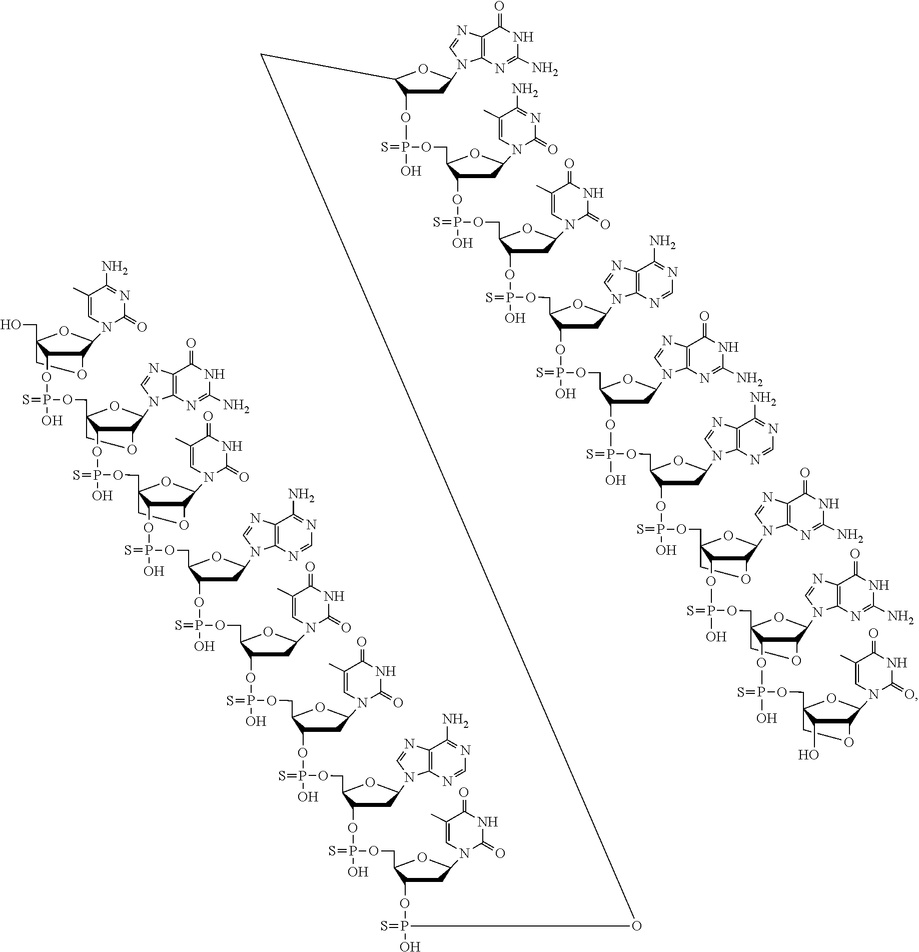

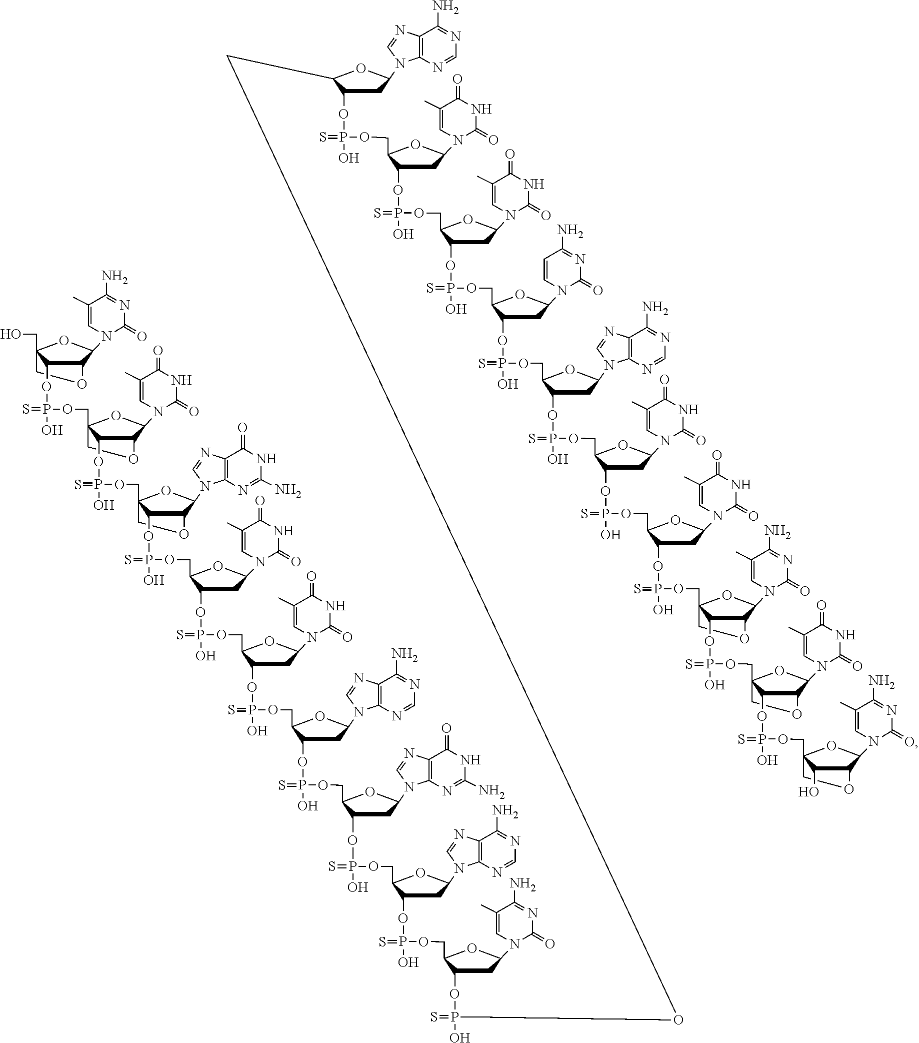

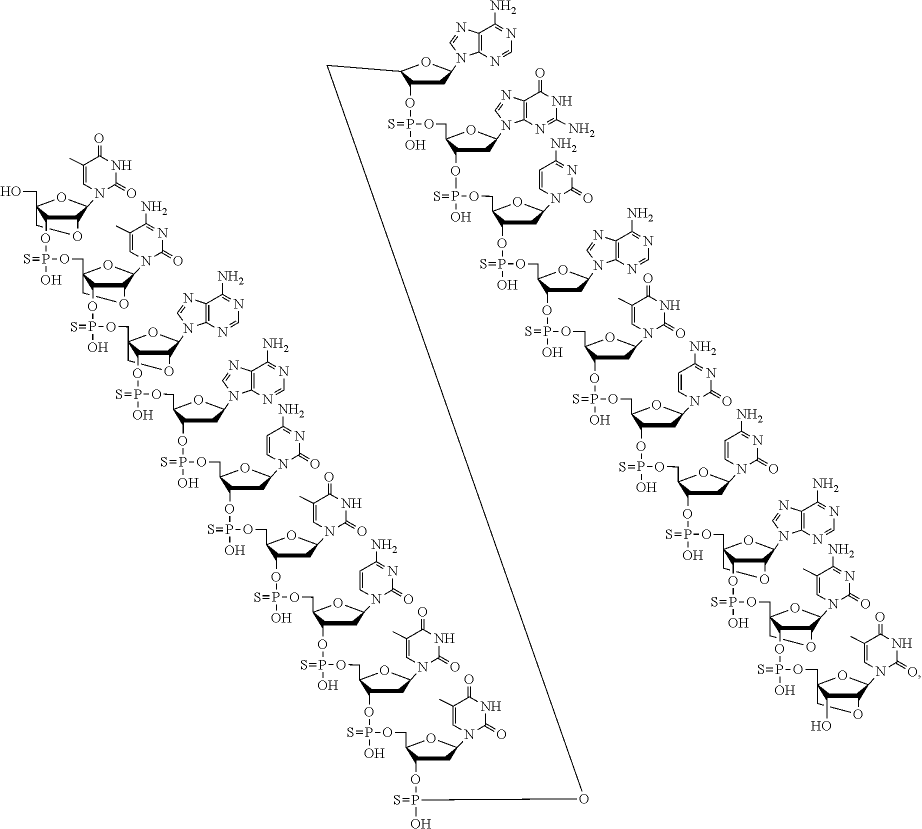

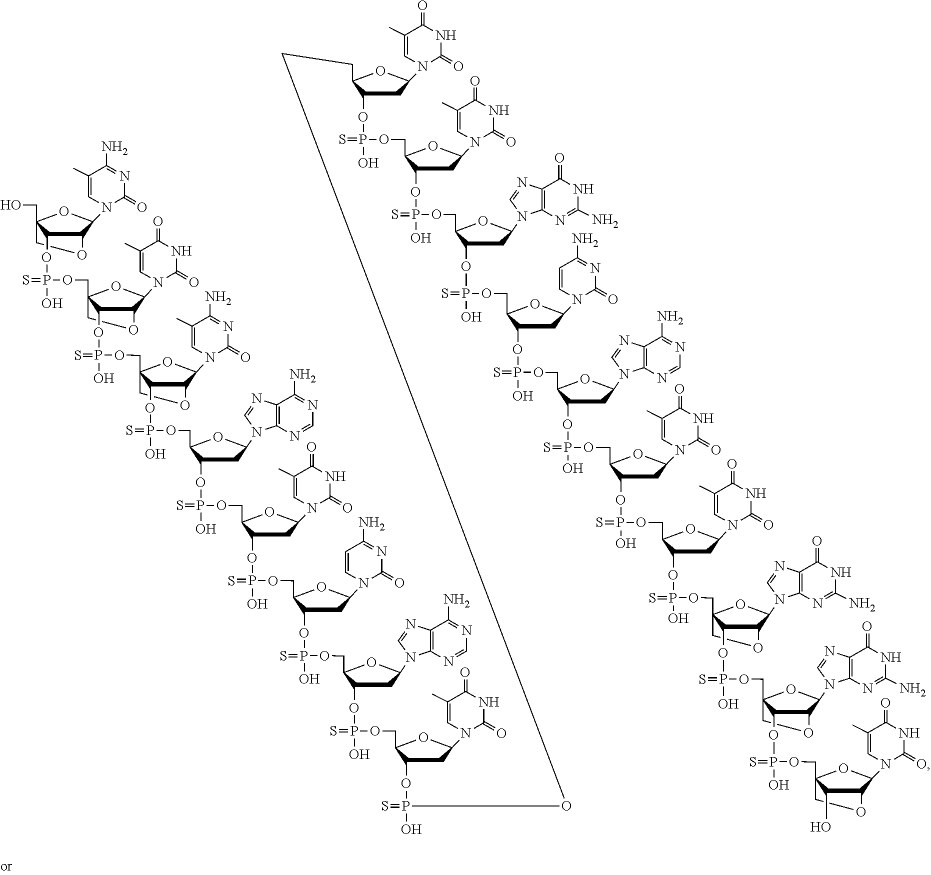

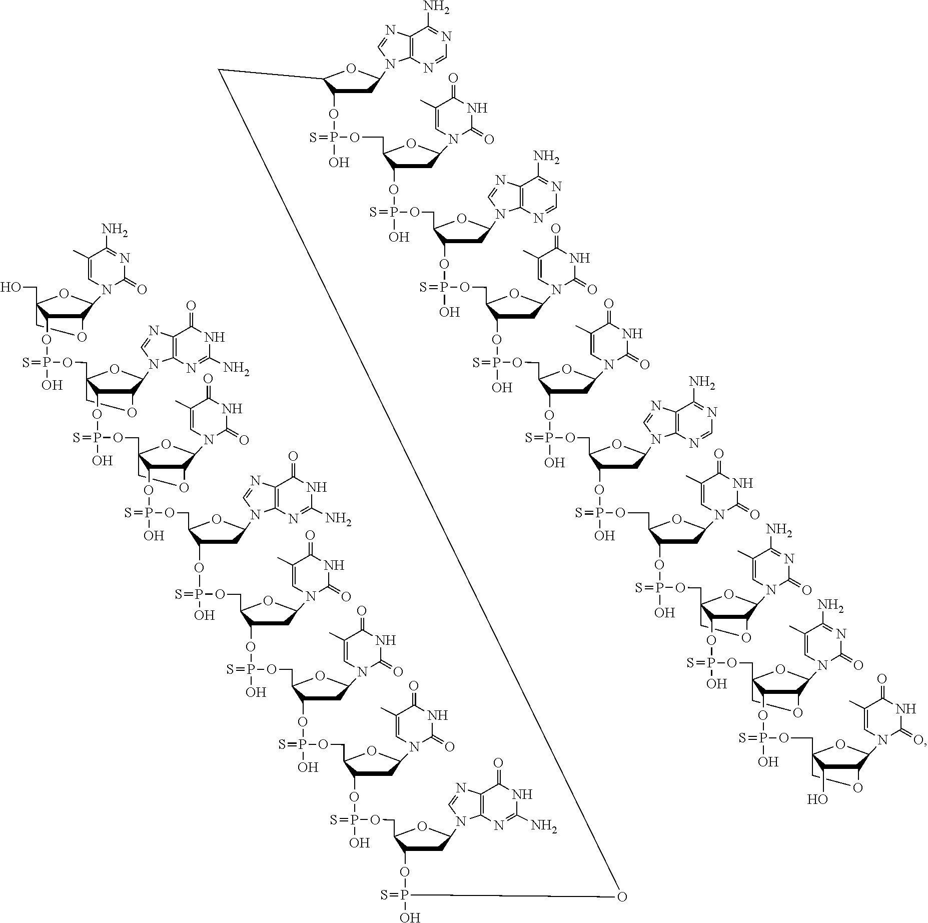

or salts thereof.

### ASO Terminal Clipping

In certain embodiments, MAPT ASOs targeted to a Tau nucleic acid may be shortened or truncated. In some embodiments, the MAPT ASOs may be shortened or truncated by endonuclease activity. For example, a single subunit (e.g., a nucleotide or a portion thereof) may be deleted from the 5′ end (5′ truncation), or alternatively from the 3′ end (3′ truncation). A shortened or truncated antisense compound targeted to a Tau nucleic acid may have two or more subunits deleted from the 5′ end, or alternatively may have two or more subunits deleted from the 3′ end, of the MAPT ASO.

### II. MAPT ASO Conjugates

Any of the described MAPT ASOs can be linked to a targeting group or a delivery vehicle. The MAPT ASO can be linked to the targeting group or a delivery vehicle directly or indirectly. The MAPT ASO can be linked to the targeting group or a delivery vehicle at the 5′ end of the ASO, and the 3′ end of the ASO or to any nucleotide in the ASO. The targeting group or delivery vehicle can be attached (linked) to a sugar moitety, a base moiety, an internucleoside linkage, or a terminus (5′ or 3′) of the MAPT ASO. Any targeting group or delivery vehicle known in the art and suitable for facilitating or increasing delivery of a MAPT ASO to a target cell or tissue in a subject can be conjugated to the MAPT ASO. A targeting group can be, but is not limited to, a molecule that specifically binds to the transferrin receptor (TfR) or a molecule expressed on the luminal surface of the blood brain barrier. The molecule can be, but is not limited to, an anti-TfR antibody or a TfR binding fragment thereof, or a Fc polypeptide modified to bind the TfR. A delivery vehicle can be, but is not limited to, a molecule, a brain shuttle, a protein, a liposome, a lipoplex, or a lipid nanoparticle.

In some embodiments, a MAPT ASO is linked to a targeting group or delivery vehicle via a linker. Any linker known in the art that is suitable for conjugating a MAPT ASO to another molecule may be used.

### III. Methods of Use

The MAPT ASOs may be used for a variety of purposes, including therapeutic indications.

In some embodiments, methods of reducing the expression of a MAPT gene in a subject are described, the methods comprising administering an effective amount of a MAPT ASO, conjugate, or composition as described herein to the subject. In some embodiments, MAPT ASOs, conjugates, or compositions thereof for use in reducing the expression of a MAPT gene in a cell of subject are provided. In certain embodiments, administration of a described MAPT ASO (or a conjugate or composition comprising the MAPT ASO) to a cell or subject reduces expression of the MAPT gene in the cell or subject. Expression can be reduced by more than 10%, more than 20%, more than 30%, more than 40%, more than 50%, more than 60%, more than 70%, more than 80%, more than 90%, or more than 95%, compared to the expression in a control (e.g., a cell or subject that was not administered the MAPT ASO, conjugate or composition as described herein) or compared to the level of expression of MAPT in the cell or subject prior to administration of the MAPT ASO.

Certain embodiments provide a method of reducing MAPT mRNA expression in a subject in need thereof, the method comprising administering to the subject a MAPT ASO, conjugate or composition as described herein.

Certain embodiments also provide a method of reducing Tau expression in a subject in need thereof, the method comprising administering to the subject a MAPT ASO, conjugate or composition as described herein.

In certain embodiments, a MAPT ASO as described herein may be used to reduce MAPT expression in a brain cell. The brain cell can be, but is not limited to a neuron, a deep brain cell, a cell of the hippocampus, or a cell of the entorhinal cortex. Thus, certain embodiments provide a method of generating a neuron cell with decreased Tau expression, the method comprising delivering to the neuron cell a MAPT ASO, a conjugate or a composition as described herein, wherein the MAPT ASO decreases the expression level of a MAPT gene (e.g., an endogenous MAPT gene). Certain embodiments also provide a method of modifying a neuron cell to decrease Tau expression, the method comprising delivering to the neuron cell a MAPT ASO, a conjugate or a composition as described herein, wherein the MAPT ASO decreases the expression level of a MAPT gene (e.g., an endogenous MAPT gene). In certain embodiments, the MAPT ASO binds to a MAPT transcript and reduces the expression level of a MAPT transcript in the cell. In certain embodiments, the MAPT ASO binds to a MAPT transcript and recruits RNase H, which degrades the transcript.

In certain embodiments, a MAPT ASO as described herein may be used to reduce MAPT expression in a spinal cord or a cell of the spinal cord.

In certain embodiments, a MAPT ASO as described herein is delivered to a cell within the brain of a subject, such as a neuron, a deep brain cell, a cell of the hippocampus, or a cell of the entorhinal cortex.

Certain embodiments provide a method of delivering a MAPT ASO to the CNS of a subject in need thereof, comprising administering to the subject a MAPT ASO, a conjugate or a composition as described herein, wherein said MAPT ASO decreases the expression level of a MAPT gene (e.g., an endogenous MAPT gene).

In certain embodiments, the MAPT ASO decreases the expression level of an endogenous MAPT gene or reduces the level of a MAPT transcript by at least about 10%, about 20%, about 30%, about 40%, about 50%, about 60%, about 70%, about 80%, about 90%, or about 95% as compared to the level MAPT transcript in the absence of administering the MAPT ASO. In certain embodiments, the expression of an endogenous MAPT gene or level of a MAPT transcript is reduced by at least about 50% relative to the level of expression prior to administration of the MAPT ASO. In certain embodiments, the expression of an endogenous MAPT gene or level of MAPT transcript is reduced by at least about 70% relative to the level of expression prior to administration of the MAPT ASO.

The MAPT ASOs, conjugates, and compositions (e.g., pharmaceutical compositions) described herein may be used to treat, prevent, or ameliorate diseases, disorders, and conditions associated with Tau. Thus, in some embodiments, provided herein are methods of treatment, prevention, or amelioration of diseases, disorders, and conditions associated with Tau in a subject in need thereof. In certain embodiments, tau associated disease is a tau associated neurodegenerative disorder. In certain embodiments, tau associated diseases include Tauopathies, Alzheimer's Disease, Fronto-temporal Dementia (FTD), FTDP-17, Progressive Supranuclear Palsy (PSP), Chronic Traumatic Encephalopathy (CTE), Corticobasal Ganglionic Degeneration (CBD), Epilepsy, and Dravet's Syndrome.

Accordingly, certain embodiments provide methods of treating a tau-associated neurodegenerative disorder in a subject in need thereof, the method comprising administering to the subject a MAPT ASO, a conjugate, or a composition as described herein.

In certain embodiments, the tau-associated neurodegenerative syndrome is Alzheimer's Disease. Thus, certain embodiments provide methods of treating Alzheimer's disease, the methods comprising administering to a subject in need thereof, a MAPT ASO, a conjugate, or a composition as described herein. The subject may be diagnosed with Alzheimer's disease, diagnosed with one or more symptoms of Alzheimer's disease, or be at risk of developing Alzheimer's disease or one or more symptoms associated with Alzheimer's disease.

In certain embodiments, the subject is a human subject.

A MAPT ASO, MAPT ASO conjugate, or a composition comprising a MAPT ASO or conjugate as described herein may be administered to a subject at a therapeutically effective amount or dose. The dosages, however, may be varied according to several factors, including the chosen route of administration, the formulation of the composition, patient response, the severity of the condition, the subject's weight, and the judgment of the prescribing physician. The dosage can be increased or decreased over time, as required by an individual patient.

In various embodiments, a MAPT ASO, MAPT ASO conjugate, or composition comprising a MAPT ASO or conjugate as described herein is administered parenterally. In some embodiments, the MAPT ASO, conjugate, or composition is administered intravenously. Intravenous administration can be by infusion, e.g., over a period of from about 10 to about 30 minutes, or over a period of at least 1 hour, 2 hours, or 3 hours. In some embodiments, the MAPT ASO or conjugate is administered as an intravenous bolus. Combinations of infusion and bolus administration may also be used.

In some parenteral embodiments, a MAPT ASO, conjugate, or composition is administered intraperitoneally, subcutaneously, intradermally, or intramuscularly. In some embodiments, the MAPT ASO, conjugate, or composition is administered intradermally or intramuscularly. In some embodiments, the MAPT ASO, conjugate, or composition is administered intrathecally, such as by epidural administration, or intracerebroventricularly.

In other embodiments, a MAPT ASO, conjugate, or composition as described herein may be administered orally, by pulmonary administration, intranasal administration, intraocular administration, or by topical administration. Pulmonary administration can also be employed, e.g., by use of an inhaler or nebulizer, and formulation with an aerosolizing agent.

### IV. Pharmaceutical Compositions and Kits

In another aspect, pharmaceutical compositions and kits comprising a MAPT ASO or conjugate as described herein are provided.

### Pharmaceutical Compositions

Guidance for preparing formulations for use as described herein can be found in any number of handbooks for pharmaceutical preparation and formulation that are known to those of skill in the art.

In some embodiments, a pharmaceutical composition comprises a MAPT ASO or MAPT ASO conjugate as described herein and further comprises one or more pharmaceutically acceptable carriers, diluents, and/or excipients.

As used herein, the term pharmaceutically acceptable carrier includes any solvents, dispersion media, or coatings that are physiologically compatible and that preferably does not interfere with or otherwise inhibit the activity of the active agent. Various pharmaceutically acceptable excipients are well-known. In some embodiments, the carrier is suitable for intravenous, intrathecal, intracerebroventricular, intramuscular, oral, intraperitoneal, transdermal, topical, or subcutaneous administration. Pharmaceutically acceptable carriers can contain one or more physiologically acceptable compounds that act, for example, to stabilize the composition or to increase or decrease the absorption of the MAPT ASO or conjugate. Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers. Other pharmaceutically acceptable carriers and their formulations are also available in the art.

The pharmaceutical compositions described herein can be manufactured in a manner that is known to those of skill in the art, e.g., by means of conventional mixing, dissolving, granulating, dragee-making, emulsifying, encapsulating, entrapping, or lyophilizing processes. The following methods and excipients are merely exemplary and are in no way limiting.

For oral administration, a MAPT ASO or conjugate as described herein can be formulated by combining it with pharmaceutically acceptable carriers that are well-known in the art. Such carriers enable the compounds to be formulated as tablets, pills, dragees, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the MAPT ASOs or conjugates with a solid excipient, optionally grinding a resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone. If desired, disintegrating agents can be added, such as a cross-linked polyvinyl pyrrolidone, agar, or alginic acid or a salt thereof such as sodium alginate.

As disclosed above, a MAPT ASO or conjugate as described herein can be formulated for parenteral administration by injection, e.g., by bolus injection or continuous infusion. For injection, the MAPT ASOs or conjugates can be formulated into preparations by dissolving, suspending, or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers, and preservatives. In some embodiments, MAPT ASOs or conjugates can be formulated in aqueous solutions, preferably in physiologically compatible buffers such as Hanks's solution, Ringer's solution, or physiological saline buffer. Formulations for injection can be presented in unit dosage form, e.g., in ampules or in multi-dose containers, with an added preservative. The compositions can take such forms as suspensions, solutions, or emulsions in oily or aqueous vehicles, and can contain formulatory agents such as suspending, stabilizing, and/or dispersing agents.

Typically, a pharmaceutical composition for use in in vivo administration is sterile. Sterilization can be accomplished according to methods known in the art, e.g., heat sterilization, steam sterilization, sterile filtration, or irradiation.

Dosages and desired drug concentration of pharmaceutical compositions as described herein may vary depending on the particular use envisioned.

### Kits

In some embodiments, kits comprising a MAPT ASO or a MAPT ASO conjugate as described herein are provided. In some embodiments, the kits are for use in reducing the expression of a MAPT gene

The described MAPT ASOs (including MAPT ASO conjugates) and pharmaceutical compositions comprising MAPT ASOs disclosed herein may be packaged or included in a kit, container, pack, or dispenser. The MAPT ASOs and pharmaceutical compositions comprising said MAPT ASOs may be packaged in pre-filled syringes or vials. Any of the MAPT ASOs or pharmaceutical compositions containing a MAPT ASO identified herein can be formulated or packaged in single-dose or multi-dose format. Any of the MAPT ASOs or pharmaceutical compositions containing a MAPT ASO identified herein can be formulated for repeat dosing.

In some embodiments, the kit further comprises instructional materials containing directions (i.e., protocols) for the practice of the methods described herein (e.g., instructions for using the kit for administering a composition across the blood-brain barrier). While the instructional materials typically comprise written or printed materials, they are not limited to such. Any medium capable of storing such instructions and communicating them to an end user is contemplated herein. Such media include, but are not limited to, electronic storage media (e.g., magnetic discs, tapes, cartridges, chips), optical media (e.g., CD-ROM), and the like. Such media may include addresses to internet sites that provide such instructional materials.

### EXAMPLE

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results. Efforts have been made to ensure accuracy with respect to numbers used (e.g., amounts, temperatures, etc.), but some experimental error and deviation may be present. The practice of the present invention will employ, unless otherwise indicated, conventional methods of protein chemistry, biochemistry, recombinant DNA techniques and pharmacology, within the skill of the art. Such techniques are explained fully in the literature.

### Example 1: In Vitro Screening of ASOs to the MAPT Gene

Selection of ASO sequences specific for a given target sequence is based upon analysis of the chosen target sequence and determination of a number of factors, including in vitro and in vivo potency, and liver toxicity profile.

### Initial MAPT ASO Screening

Initial identification of 393 MAPT ASO sequences 100% complementary to the MAPT pre-mRNA (Ensembl ENST0000034429) SEQ ID NO: 2 and or MAPT mRNA (refseq NM_001123066) SEQ ID NO: 3 were made based on computational predictions of target binding, specificity, efficacy and safety.

Knockdown efficacy of the MAPT-specific ASOs in human MiaPaCa-2 and HDLM2 cells was tested. The cells were treated with MAPT-specific ASO or control oligonucleotide at a concentration of 5 μM without the use of a transfection reagent. After three days treatment, cells were lysed. MAPT and HPRT1 mRNA expression was analyzed using the QuantiGene Singleplex assay (ThermoFisher). MAPT expression values were normalized to HPRT1 values and the degree of knockdown in comparison to mock-treated cells was determined.

### Two-Concentration Screen

55 MAPT-specific ASOs with a knockdown efficacy in both tested cell lines of >99.9% (at 5 μM), were tested in a two-concentration screen (100 nM, 1000 nM) in MiaPaCa-2 cells. After three days treatment, cells were lysed. MAPT and HPRT1 mRNA expression was analyzed using the QuantiGene Singleplex assay (ThermoFisher). MAPT expression values were normalized to HPRT1 values and the degree of knockdown in comparison to mock-treated cells was determined. 22 ASOs were selected for investigation of the TLR9 activating capacity and concentration response relations ship (IC50 determination).

### Investigation of the TLR9 Activating Capacity

The 22 ASOs were tested for their TLR9-dependent proinflammatory potential. A reporter cell line (HEK-Blue-hTLR9 cells, Invivogen) was treated with MAPT-specific ASOs, a positive and a negative control without the use of transfection reagent at a concentration of 5 μM. After 20 h, QUANTI-Blue™ Solution (Invivogen) was added to the cells. Optical density was measured 2 hours later in order to determine the TLR9 activating capacity of the tested ASOs.

### In Vitro IC50 Determination

Investigation of concentration-response relationship and determination of half maximal inhibitory concentration (IC50) values was performed in MiaPaCa-2 cells for the 22 selected MAPT-specific ASOs. Cells were treated with the MAPT-specific ASOs at the following concentrations: 5000, 1667, 556, 185, 62, 21, 7 nM without the addition of a transfection reagent. After three days treatment, cells were lysed. MAPT and HPRT1 mRNA expression was analyzed by the QuantiGene Singleplex assay (Thermo Fisher). MAPT expression values were normalized to HPRT1 values and the degree of knockdown in comparison to mock-treated cells was determined. Results are shown in Table 3.

TABLE 3

| | IC₅₀ [nM] | R² |
|---|---|---|
| Control ASO B | 65 | 0.98 |
| ASO1 | 129 | 0.98 |
| ASO2 | 46 | 0.97 |
| ASO3 | 90 | 0.98 |
| ASO4 | 132 | 0.98 |
| ASO5 | 92 | 0.99 |
| ASO6 | 29 | 0.99 |
| ASO7 | 102 | 0.95 |
| ASO8 | 112 | 0.99 |
| ASO9 | 114 | 0.95 |
| ASO10 | 118 | 0.98 |
| ASO11 | 129 | 0.97 |
| ASO12 | 95 | 0.99 |
| ASO13 | 58 | 0.99 |
| ASO14 | 132 | 0.98 |
| ASO15 | 134 | 0.98 |
| ASO16 | 46 | 0.99 |
| ASO17 | 67 | 0.97 |
| ASO18 | 62 | 0.96 |
| ASO19 | 120 | 0.98 |
| ASO20 | 155 | 0.98 |
| ASO21 | 65 | 0.99 |
| ASO22 | 98 | 0.98 |

15 sequences were selected based on the combination of in vitro efficacy, potency (Table 3 above) and safety results from further in vivo safety and potency analyses. The 15 sequences selected were: ASO2, ASO3, ASO5, ASO6, ASO7, ASO8, ASO9, ASO10, ASO12, ASO13, ASO16, ASO17, ASO18, ASO21, and ASO22.

### Example 2: In Vivo Knockdown Screening

The 15 selected ASOs noted above were synthesized according to the procedures described below for in vivo dosing. The ASOs were diluted in sterile saline and administered to hTau mice (mTau−/−) via intracerebroventricular (ICV) injection at a single dose of 50 μg or 100 μg in 10 μL total volume per mouse. The 50 μg or 100 μg dose was determined based on in vivo tolerability of each ASO. ASO sequences that induced acute neurotoxicity when administered via ICV bolus were administered at a 50 μg dose.

Two weeks post dose, tissues (brain and spinal cord) were harvested and terminal blood was collected. MAPT expression was measured in the brain and spinal cord as follows. A piece of the frontal lobe and cervical spinal cord was homogenized with a bead homogenizer in Trizol for bulk RNA isolation. Briefly, homogenized tissues were incubated with chloroform for 3-5 minutes to allow for phase separation after centrifugation. The aqueous phase was then incubated with isopropanol for 10 minutes to allow for RNA precipitation followed by a 75% ethanol wash and resuspension in nuclease-free water. MAPT expression was then measured by qPCR using the Express One-Step Superscript Kit and normalized to expression of the housekeeping gene Gapdh. A panel of glial activation markers (Aifl, Gfap, Tlr9, Itgax) were also quantified by qPCR using the Express One-Step Superscript Kit to determine if any ASO triggered a chronic neuroinflammatory response.

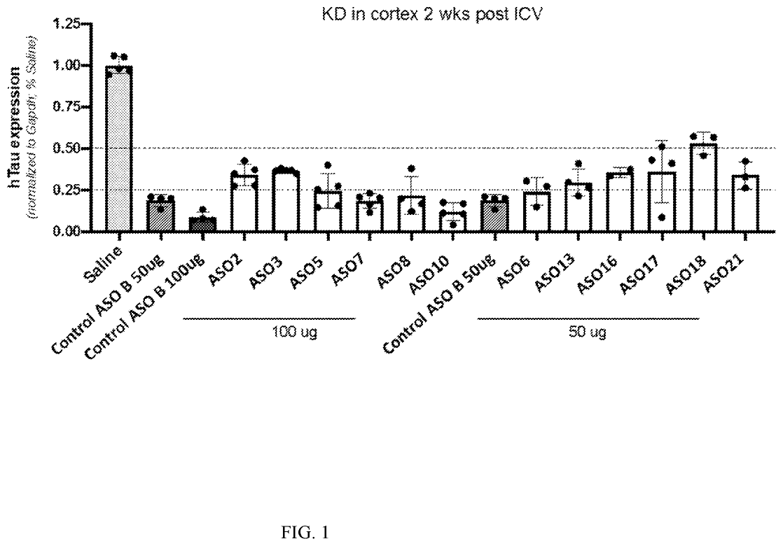

Human tau knockdown from each of the ASOs (except for ASO9, ASO12, and ASO22) are shown in FIG. 1. Mice dosed with ASO9, ASO12, and ASO22 exhibited acute neurotoxicity and were sacrificed. All ASOs were shown to achieve at least 50% knockdown in vivo. ASO10 and ASO7 demonstrated the highest level of human tau knockdown at 100 ug (88% and 82%, respectively). ASO6 and ASO13 demonstrated the highest level of human tau knockdown at 50 ug (76% and 70%, respectively). ASO10 was eliminated from further analysis due to extreme glial activation (i.e., significant upregulation of the entire panel of glial activation markers) persisting two weeks post dose.

### Example 3: In Vivo Liver Toxicity Screening

Liver safety was assessed for each ASO. Alanine transaminase (ALT), aspartate aminotransferase (AST), and lactate dehydrogenase (LDH) are enzymes found in high quantities in liver. When liver cells are damaged, they release these enzymes into the bloodstream. Therefore, serum ALT, AST, and LDH levels were measured to determine if the ASOs induced liver damage.

The ASOs in Table 4 below were diluted in sterile saline and administered to wildtype mice subcutaneously at a dose of 20mpk daily for five consecutive days. The mice were weighed daily for 12 days, and blood was drawn on Day 5, Day 8, and Day 12. Serum was prepared by allowing the blood samples to clot followed by centrifugation. ALT, AST, and LDH levels were then measured in the serum sample. The summary of results is shown below in Table 4.

TABLE 4

| | Bodyweight | | | | |
|---|---|---|---|---|---|
| | change | ALT | AST | LDH | Survival |
| Control | Low | Mid | Mid | Low | |
| ASO 2 | Low | Mid | Mid | Low | |
| ASO 3 | Low | Mid | Mid | High | |
| ASO 5 | High | N/A* | N/A* | N/A* | All mice dead Day 10 |
| ASO 6 | Low | Low | Low | Low | |
| ASO 7 | Mid | Mid | Mid | Mid | |
| ASO 8 | Mid | Mid | Mid | Mid | |
| ASO 9 | Low | Low | Mid | Low | |
| ASO 12 | Mid | Mid | Mid | Mid | |
| ASO 13 | Low | Low | Low | Low | |
| ASO 16 | High | N/A* | N/A* | N/A* | 1 mouse dead at Day 11; |
| | | | | | 1 mouse data at Day 12 |
| ASO 17 | High | N/A* | N/A* | N/A* | All mice dead at Day 8 |
| ASO 18 | Low | Low | Low | Low | |
| ASO 21 | High | N/A* | N/A* | N/A* | 2 mice dead at Day 7; |
| | | | | | 3 mice dead at Day 8 |
| ASO 22 | Low | Low | Low | Low | |

The compilation of survival data, body weight change data, and liver enzyme data were used to select 9 ASOs (ASO2, ASO6, ASO7, ASO8, ASO9, ASO12, ASO13, ASO18, and ASO22) for follow-up potency studies.

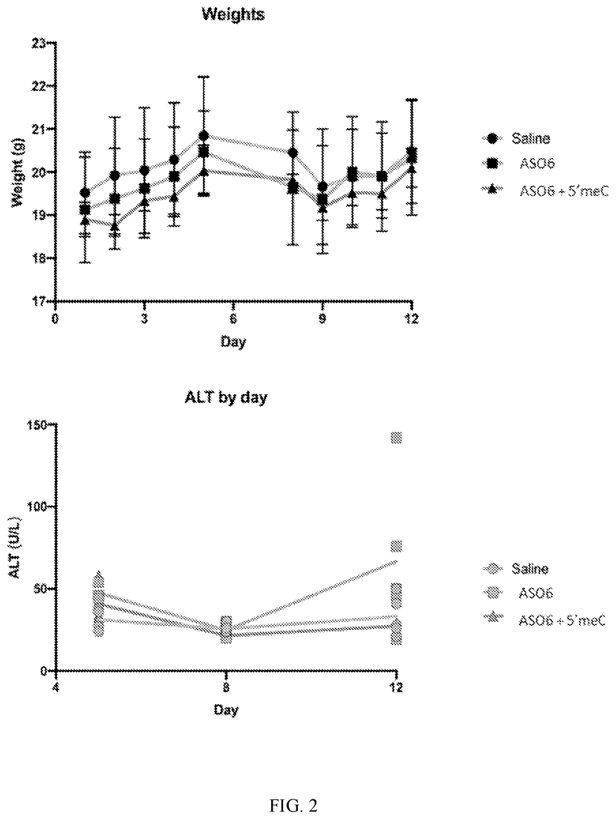

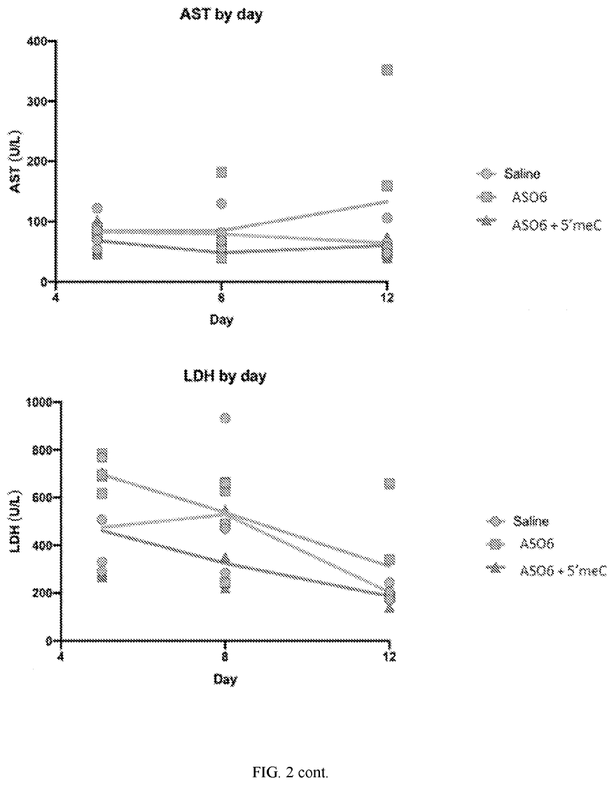

Furthermore, to determine if internal cytosine methylation, i.e., methylating the cytosines in the gap region of the ASO, has an impact on the liver safety profile of an ASO, the internal cytosines in ASO 6 were replaced with 5-methyl cytosines (See Table 7; base sequence SEQ ID NO: 5, wherein all cytosines are modified to 5-methyl cytosine). Liver safety was analyzed using the same protocols described above. The results from this liver toxicity study are shown in FIG. 2. The addition of 5-methyl cytosine had only a minor beneficial effect on the liver safety profile of ASO6, i.e., the minor increase in liver enzyme levels were ameliorated by cytosine methylation.

### Example 4: In Vivo ED50 Screening

The nine ASOs selected above (based on lack of chronic neuroinflammation and lack of significant liver toxicity) were diluted in sterile saline and administered to hTau (mTau−/−) mice via ICV injection at several doses ranging from 5-50 μg according to Table 5 below. All ASOs were dosed at a final volume of 10 uL. ASO12 was subsequently eliminated from the study due to acute neurotoxicity in the form of seizures and delayed recovery time after ICV injection. ASO9 and ASO22 were not tested in the initial high dose screen due to acute neurotoxicity related to the route of administration.

TABLE 5

| | 100 | 50 | 25 | 15 | 5 | 0 | All doses for |
|---|---|---|---|---|---|---|---|
| | μg | μg | μg | μg | μg | μg | ED50 curves |
| Control | e.d. ^{A} | e.d. ^{A} | | e.d. | e.d. | e.d. ^{A} | 100, 50, 15, 5, 0 |
| ASO2 | e.d. ^{A} | e.d. | | e.d. | | e.d. ^{A} | 100, 50, 15, 0 |
| ASO6 | | e.d. ^{A} | | e.d. | e.d. | e.d. ^{A} | 50, 15, 5, 0 |
| ASO7 | e.d. ^{A} | e.d. | | e.d. | | e.d. ^{A} | 100, 50, 15, 0 |
| ASO8 | e.d. ^{A} | e.d. | | e.d. | | e.d. ^{A} | 100, 50, 15, 0 |
| ASO9 | | | e.d. | e.d. | e.d. | e.d. ^{A} | 25, 15, 5, 0 |
| ASO13 | | e.d. ^{A} | | e.d. | e.d. | e.d. ^{A} | 50, 15, 5, 0 |
| ASO18 | | e.d. ^{A} | | e.d. | e.d. | e.d. ^{A} | 50, 15, 5, 0 |
| ASO22 | | | e.d. | e.d. | e.d. | e.d. ^{A} | 25, 15, 5, 0 |

^{A} = data available from previous study, not dosed in this present study;
e.d. = evaluated dose.

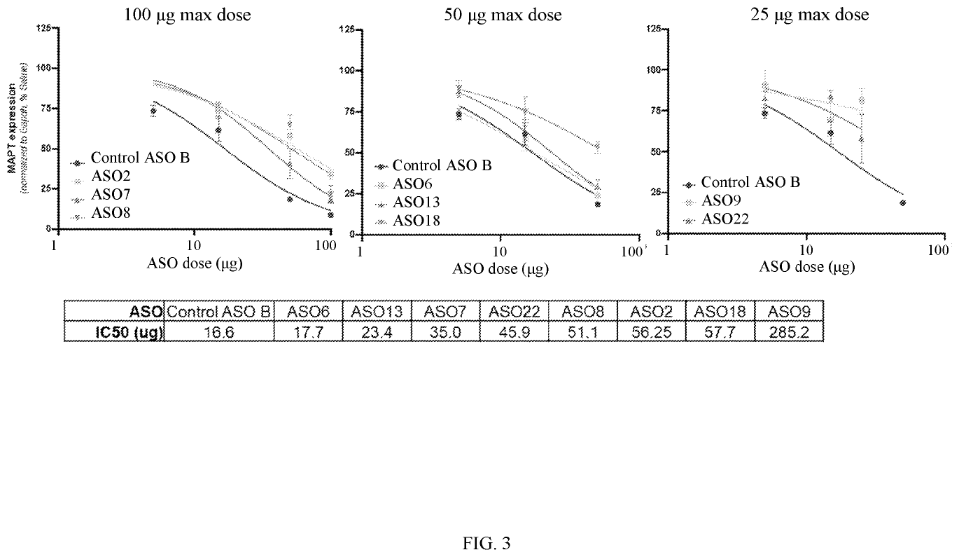

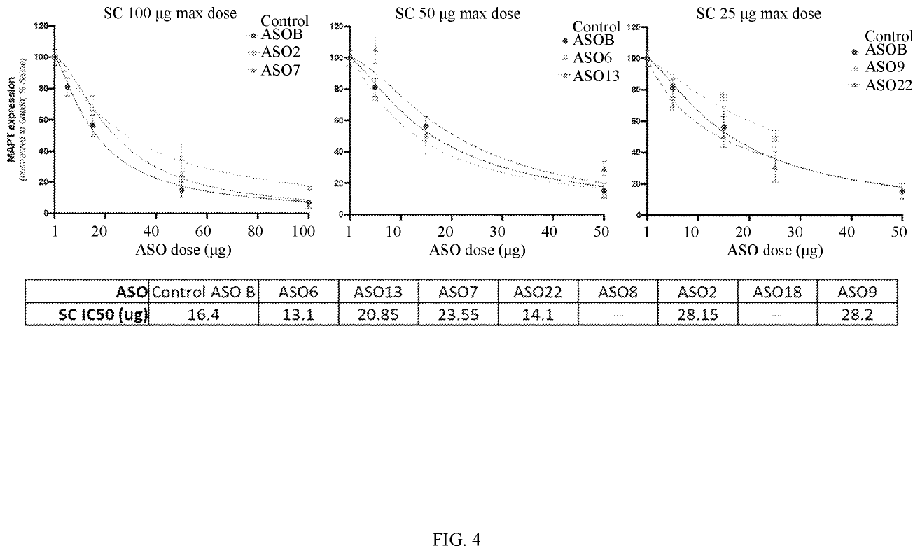

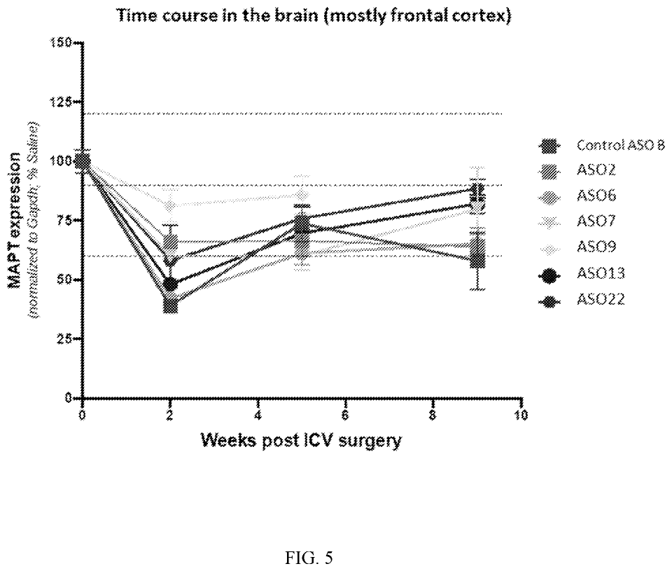

Two weeks post dose, tissues were harvested and MAPT expression was measured in the brain and spinal cord via bulk RNA isolation followed by qPCR of MAPT and Gapdh according to the protocol described above. MAPT knockdown in cortex and spinal cord are shown in FIG. 3 and FIG. 4 respectively. ASO2, ASO6, ASO7, AS09, ASO13, and ASO22 were selected for further study (see, Table 7 for corresponding modified sequences).

### Example 5: In Vivo ASO Half Life Determination

Five ASOs (ASO2, ASO6, ASO7, ASO13, and ASO22) were diluted in sterile saline and administered to hTau (mTau−/−) mice via ICV injection at a moderate dose of 25 μg in 10 μL total volume. These ASOs were selected on the basis of low liver toxicity and high knockdown potency, with the exception of ASO2, which is not as potent but is mouse cross-reactive. Tissues were harvested at 5 and 9 weeks post-dose to determine the duration of tau knockdown. ASO9 was administered to hTau (mTau−/−) mice but tissues were harvested at 5 weeks only. MAPT expression was measured in the brain and spinal cord via bulk RNA isolation followed by qPCR of MAPT and Gapdh according to the protocol described above.

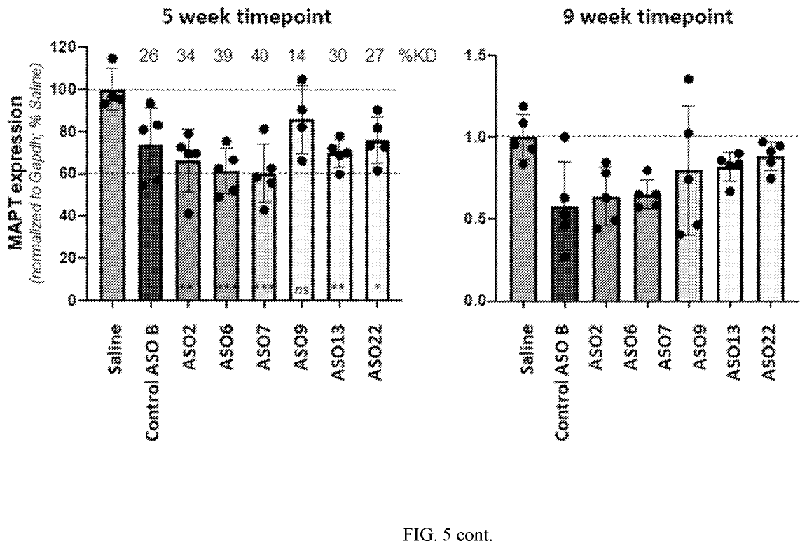

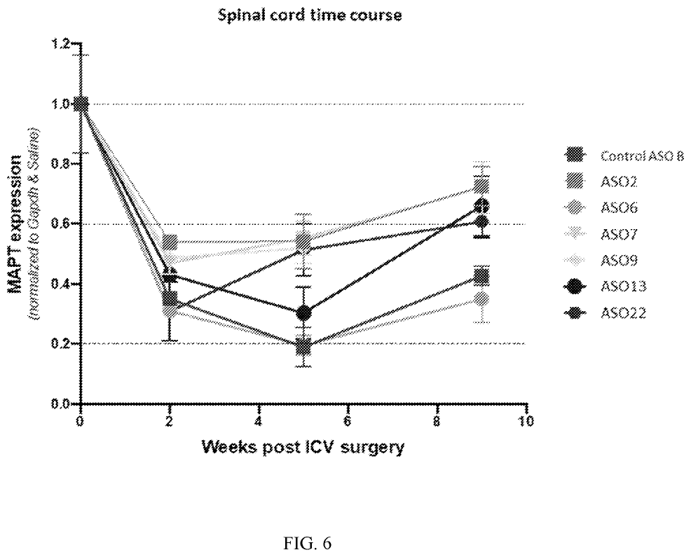

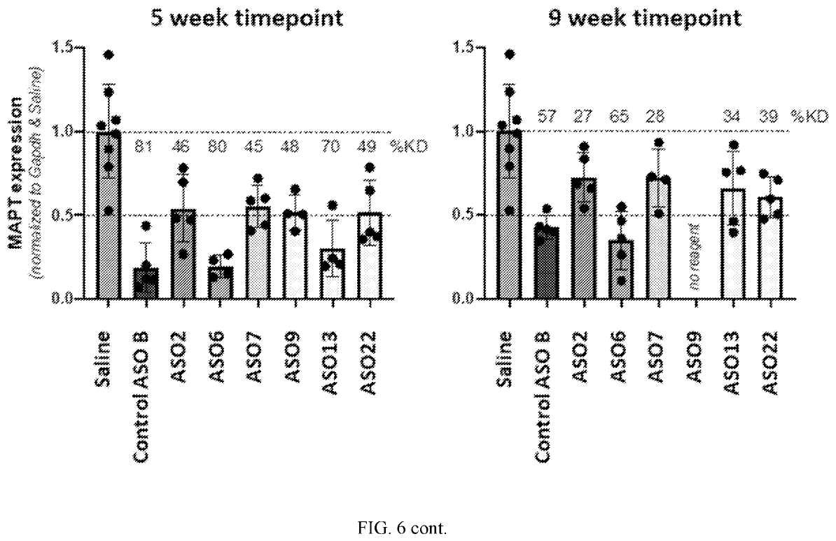

Results for knockdown duration of action in brain and spinal cord are shown in FIGS. 5 and 6 respectively and Table 6. MAPT knockdown is shown to persist for many weeks after ICV injection, with ASO6 sustaining robust tau knockdown for the longest period of time.

TABLE 6

| | Average MAPT expression, normalized to Gapdh and saline-treated cohort | | | | | | | | |
|---|---|---|---|---|---|---|---|---|---|
| | Timepoint | | Control | | | | | | |
| | post dose | Saline | ASO B | ASO2 | ASO6 | ASO7 | ASO9 | ASO13 | ASO22 |
| Brain | 5 weeks | 1.0000 | 0.7379 | 0.6640 | 0.6119 | 0.6022 | 0.8583 | 0.6963 | 0.7590 |
| | 9 weeks | 1.0000 | 0.5794 | 0.6396 | 0.6521 | 0.7979 | | 0.8200 | 0.8850 |
| Spinal | 5 weeks | 1.0000 | 0.1889 | 0.5415 | 0.1954 | 0.5523 | 0.5198 | 0.3031 | 0.5130 |
| Cord | 9 weeks | 1.0000 | 0.4268 | 0.7253 | 0.3496 | 0.7211 | | 0.6598 | 0.6076 |

### Example 6: In Vivo Rat Renal Toxicity Determination

The rat is considered the most sensitive species for detecting ASO-mediated kidney toxicity and are the preferred species to use for risk assessment because they can overpredict susceptibility of renal toxicity in humans. The objective of this study was to determine the effects, if any, on kidney function and morphology (gross and histologic evaluation) following repeat dose administration of five ASOs (ASO2, ASO6, ASO9, ASO13, and ASO22) in a 2-week rat study.

Male Wistar Hannover rats (7-8 weeks old) were divided into 7 groups of five animals each. Each group was administered two 40 mg/kg subcutaneous injections of ASO (Control ASO A, ASO2, ASO6, ASO9, ASO13, or ASO22) or saline (vehicle control) on Days 1 and 8; animals were euthanized on Day 15. Body weight measurements were performed prior to dosing on Days 1 and 8, and on Days, 5, 10, and 15. Clinical observations were performed following dosing on Days 1 and 8. Blood samples were collected for measurement of blood urea nitrogen (BUN) and creatinine as markers of kidney function on Days 5, 10, and 15. On Day 15, kidneys were collected for microscopic evaluation, and urine was collected for measurement of KIM-1:creatinine ratio as an index for renal injury.

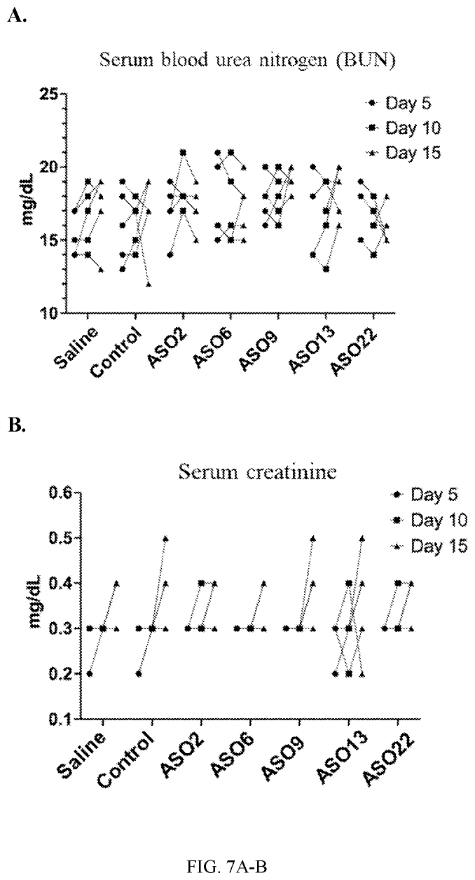

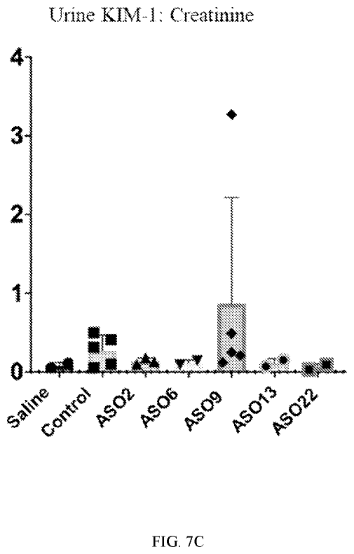

All ASOs were well tolerated in Wistar Hannover rat with no changes in clinical signs or body weight throughout the treatment period. Serum BUN and creatinine (FIG. 7A-B) levels were within normal historical range in all treatment groups and comparable to vehicle controls. On Day 15, changes in urine KIM-1:creatinine ratio were considered below critical limit and of a magnitude of change commonly observed in rats under similar study conditions (FIG. 7C). One animal from test group ASO9 was above the normal range for Wistar Hannover rats (KIM-1:Creatinine ratio: ~0.1-2); however, this was considered of questionable relevance based on lack of similar magnitude of change observed in other rats in the same group.

Microscopic minimal to mild proximal tubular epithelial degeneration associated with minimal regeneration and minimal to mild interstitial, perivascular, and/or periglomerular mononuclear cellular infiltrates in the kidney were observed in every ASO-treated group. These microscopic changes were considered non-adverse, based on minimal to mild severity and the absence of correlating functional changes in clinical signs, body weight, and serum and urinary kidney biomarkers (BUN, creatinine, and KIM-1). In conclusion, no evidence of adverse effects on kidney function or histopathology was detected in Wistar Han rats administered two 40 mg/kg doses of six selected ASOs (Control ASO A, ASO2, ASO6, AS09, ASO13, or ASO22) in this study.

TABLE 7

| | SEQ | |
|---|---|---|
| | ID | Corresponding |
| Name | NO | Modified Sequence** |
| ASO 2 with LNAs and PS | 4 | +C*+G*+T*G*T*T*T*G* |
| linkages | | A*T*A*T*T*A*T*+C*+ |
| | | C*+T |
| ASO 6 with LNAs and PS | 5 | +A*+C*+C*T*T*A*A*G* |
| linkages | | T*A*T*T*A*C*T*+T*+ |
| | | G*+C |
| ASO 6 with LNAs, PS | 5 | +A*+C*+C*T*T*A*A*G* |
| linkages, and internal | | T*A*T*T*A*%C*T*+T*+ |
| 5-methyl C | | G*+C |
| ASO 7 with LNAs and PS | 6 | +T*+C*+A*A*C*T*C*T* |
| linkages | | T*A*G*C*A*T*C*C*+ |
| | | A*+C*+T |
| ASO 9 with LNAs and PS | 7 | +C*+T*+T*A*T*C*A*T* |
| linkages | | T*T*G*C*A*T*T*+G*+ |
| | | G*+T |
| ASO 13 with LNAs and PS | 8 | +C*+T*+G*T*T*A*G*A* |
| linkages | | C*A*T*T*C*A*T*T*+ |
| | | C*+T*+C |
| ASO 22 with LNAs and PS | 9 | +C*+G*+T*A*T*T*A*T* |
| linkages | | G*C*T*A*G*A*+G*+G*+ |
| | | T |
| ASO 2.1 with LNAs and | 19 | +C*+G*+T*G*T*T*T*G* |
| PS linkages | | A*T*A*T*T*+A*+T*+C |
| ASO 2.2 with LNAs and | 20 | +G*+T*+G*T*T*T*G*A* |
| PS linkages | | T*A*T*T*A*+T*+C*+C |
| ASO 2.3 with LNAs and | 21 | +T*+G*+T*T*T*G*A*T* |
| PS linkages | | A*T*T*A*T*+C*+C*+T |
| ASO 6.1 with LNAs and | 22 | +A*+C++C*T*T*A*A*G* |
| PS linkages | | T*A*T*T*A*+C*+T*+T |
| ASO 6.2 with LNAs and | 23 | +C*+C*+T*T*A*A*G*T* |
| PS linkages | | A*T*T*A*C*+T*+T*+G |
| ASO 6.3 with LNAs and | 24 | +C*+T*+T*A*A*G*T*A* |
| PS linkages | | T*T*A*C*T*+T*+G*+C |
| ASO 7.1 with LNAs and | 25 | +T*+C*+A*A*C*T*C*T* |
| PS linkages | | T*A*G*C*A*+T*+C*+C |
| ASO 7.2 with LNAs and | 26 | +C*+A*+A*C*T*C*T*T* |
| PS linkages | | A*G*C*A*T*+C*+C*+A |
| ASO 7.3 with LNAs and | 27 | +A*+A*+C*T*C*T*T*A* |
| PS linkages | | G*C*A*T*C*+C*+A*+C |
| ASO 7.4 with LNAs and | 28 | +A*+C*+T*C*T*T*A*G* |
| PS linkages | | C*A*T*C*C*+A*+C*+T |
| ASO 9.3 with LNAs and | 29 | +C*+T*+T*A*T*C*A*T* |
| PS linkages | | T*T*G*C*A*+T*+T*+G |
| ASO 9.3 with LNAs and | 30 | +T*+T*+A*T*C*A*T*T* |
| PS linkages | | T*G*C*A*T*+T*+G*+G |
| ASO 9.3 with LNAs and | 31 | +T*+A*+T*C*A*T*T*T* |
| PS linkages | | G*C*A*T*T*+G*+G*+T |
| ASO 13.1 with LNAs and | 32 | +C*+T*+G*T*T*A*G*A* |
| PS linkages | | C*A*T*T*C*+A*+T*+T |
| ASO 13.2 with LNAs and | 33 | +T*+G*+T*T*A*G*A*C* |
| PS linkages | | A*T*T*C*A*+T*+T*+C |
| ASO 13.3 with LNAs and | 34 | +G*+T*+T*A*G*A*C*A* |
| PS linkages | | T*T*C*A*T*+T*+C*+T |
| ASO 13.4 with LNAs and | 35 | +T*+T*+A*G*A*C*A*T* |
| PS linkages | | T*C*A*T*T*+C*+T*+C |
| ASO 22.1 with LNAs and | 36 | +C*+G*+T*A*T*T*A*T* |
| PS linkages | | G*C*T*A*G*+A*+G*+G |
| ASO 22.2 with LNAs and | 37 | +G*+T*+A*T*T*A*T*G* |
| PS linkages | | C*T*A*G*A*+G*+G*+T |

**wherein recited base sequence is modified as follows: “+” indicates LNA nucleosides, all others are DNA nucleosides, all LNA C are 5-methyl cytosine, all internucleoside linkages are phosphorothioate internucleoside linkages indicated by “*”, %C = internal 5-methyl cytosines

TABLE 8
Control ASOs.

| | SEQ | |
|---|---|---|
| | ID | Corresponding |
| Name | NO | Modified Sequence** |
| Control ASO A with LNAs | 16 | +A*T*+T*+T*+C*C*A* |
| and PS linkages | | A*A*T*T*C*A*C*T*+ |
| | | T*+T*T*+A*+C |
| Control ASO B with LNAs | 17 | +C*+T*+T*+T*A*A*T* |
| and PS linkages | | T*T*A*A*T*%C*A*%C*+ |
| | | T*+C*+A*+T |
| Control ASO C with | 18 | +G*+C*+A*T*T*C*T*A* |
| LNAs and PS linkages | | A*T*A*G*C*+A*+G*+C |

**wherein recited base sequence is modified as follows: “+” indicates LNA nucleosides, all others are DNA nucleosides, all LNA C are 5-methyl cytosine, all internucleoside linkages are phosphorothioate internucleoside linkages indicated by “*”, %C = internal 5-methyl cytosines

1. A MAPT antisense oligonucleotide (MAPT ASO) 17-19 nucleotides in length and comprising the nucleobase sequence of any one of SEQ ID NOs: 4-9, 19-37, and 38-56, wherein the MAPT ASO comprises at least one modified internucleoside linkage and/or at least one modified sugar.

2. The MAPT ASO of claim 1, wherein the MAPT ASO comprises a nucleobase sequence consisting of the sequence of any one of SEQ ID NOs: 4-9.

3. The MAPT ASO of claim 1 or 2, wherein the MAPT ASO is a gapmer having a gap segment consisting of the nucleobase sequence of any one of SEQ ID NOs: 10-15.

4. A MAPT antisense oligonucleotide (MAPT ASO), wherein the MAPT ASO is a gapmer having a gap segment consisting of the nucleobase sequence of any one of SEQ ID NOs: 10-15, wherein the MAPT ASO comprises at least one modified internucleoside linkage and at least one modified sugar.

5. The MAPT ASO of claim 4, wherein the MAPT ASO is 15 to 25 linked nucleosides in length.

6. The MAPT ASO of claim 5, wherein the MAPT ASO is 16-19 nucleotides in length.

7. A MAPT antisense oligonucleotide (MAPT ASO), wherein the MAPT ASO is a gapmer 16 nucleotides in length and comprises a gap segment consisting of the nucleobase sequence of any one of SEQ ID NOs: 38-56 wherein the MAPT ASO comprises at least one modified internucleoside linkage and at least one modified sugar.

8. The MAPT ASO of claim 7, wherein the MAPT ASO comprises a nucleobase sequence consisting of the sequence of any one of SEQ ID NOs: 19-37.

9. The MAPT ASO of any one of claims 1-8, wherein the at least one modified internucleoside linkage is a phosphorothioate internucleoside linkage.

10. The MAPT ASO of any one of claims 1-9, wherein the MAPT ASO contains at least one modified sugar comprising a bicyclic sugar.

11. The MAPT ASO of claim 10, wherein the MAPT ASO comprises 2-6 modified sugars each comprising a bicyclic sugar.

12. The MAPT ASO of claim 11, wherein each bicyclic sugar comprises a chemical bridge between the 4′ and 2′ positions of the sugar, wherein each chemical bridge is independently selected from the group consisting of: 4′-CH(R)—O-2′ and 4′-(CH₂)₂—O-2′, wherein each R is independently selected from H, C₁-C₆ alkyl and C₁-C₆ alkoxy.

13. The MAPT ASO of claim 12, wherein each bicyclic sugar comprises a chemical bridge between the 4′ and 2′ positions of the sugar, wherein each chemical bridge is 4′-CH(R)—O-2′, and wherein each R is independently H.

14. The MAPT ASO of any one of claims 1-13, further comprising a at least one modified nucleobase.

15. The MAPT ASO of claim 14, wherein the modified nucleobase comprises a 5-methyl cytosine.

16. The MAPT ASO of any one of claims 1-15, wherein the MAPT ASO comprises, from 5′ to 3′:
a 5′ wing segment having from 3 nucleosides, wherein each nucleoside of the 5′ wing segment comprises a modified sugar;
a gap segment having from 10-13 nucleosides, wherein each nucleoside of the gap segment is a deoxynucleoside; and
a 3′ wing segment having from 3 nucleosides, wherein each nucleoside of the 3′ wing segment comprises a modified sugar.

17. A MAPT ASO consisting of:
(a) 17 contiguous nucleobases complementary to nucleobases 66,760 to 66,776 of SEQ ID NO: 1;
(b) 18 contiguous nucleobases complementary to nucleobases 78,441 to 78,458 of SEQ ID NO: 1;
(c) 19 contiguous nucleobases complementary to nucleobases 78,631 to 78,649 of SEQ ID NO: 1;
(d) 18 contiguous nucleobases complementary to nucleobases 78,828 to 78,845 of SEQ ID NO: 1; or
(e) 19 contiguous nucleobases complementary to nucleobases 90,215 to 90,233,
wherein the MAPT ASO comprises at least one modified internucleoside linkage and/or at least one modified sugar.

18. A MAPT ASO according to the following chemical structure: 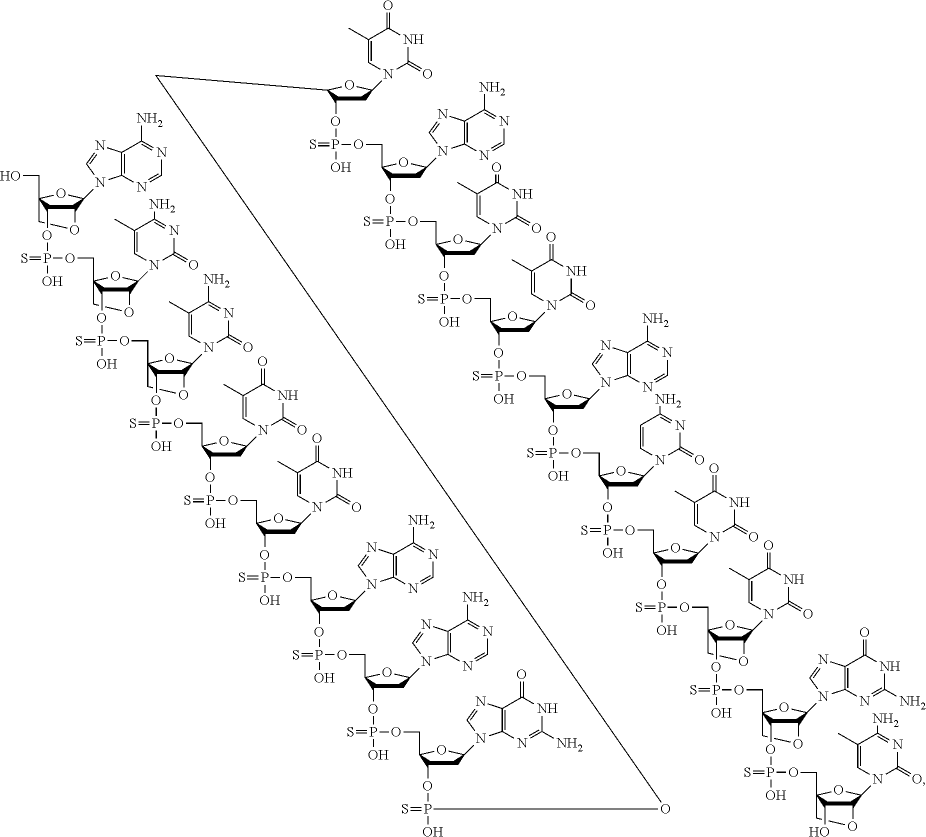
or a salt thereof.

19. A MAPT ASO according to the following chemical structure: 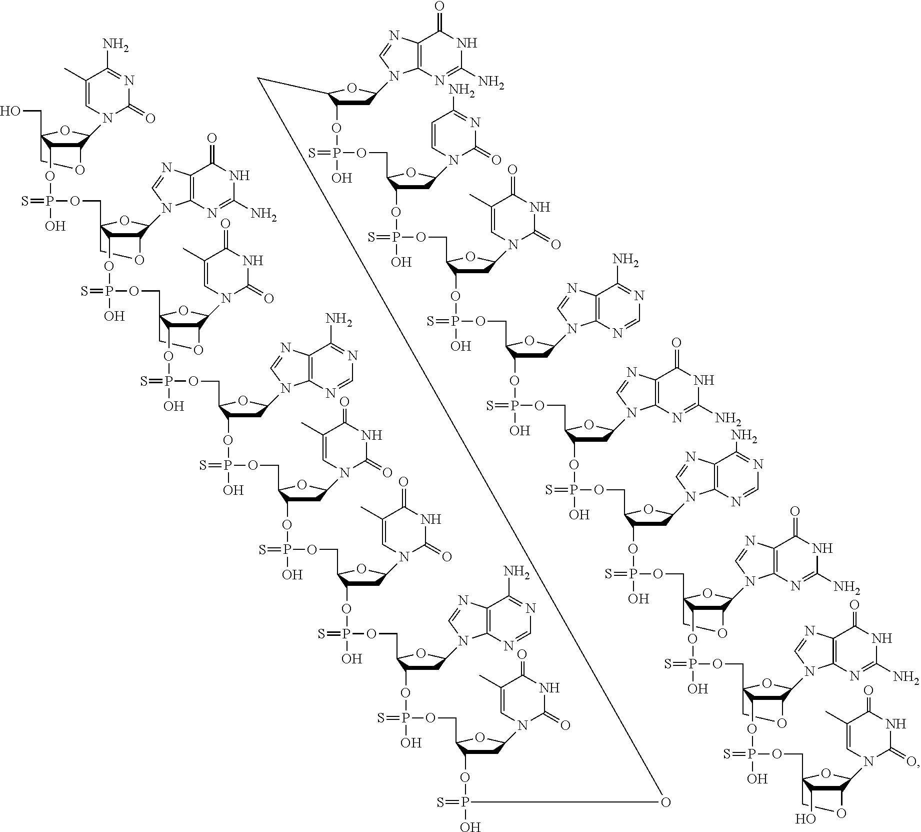
or a salt thereof.

20. A MAPT ASO according to the following chemical structure: 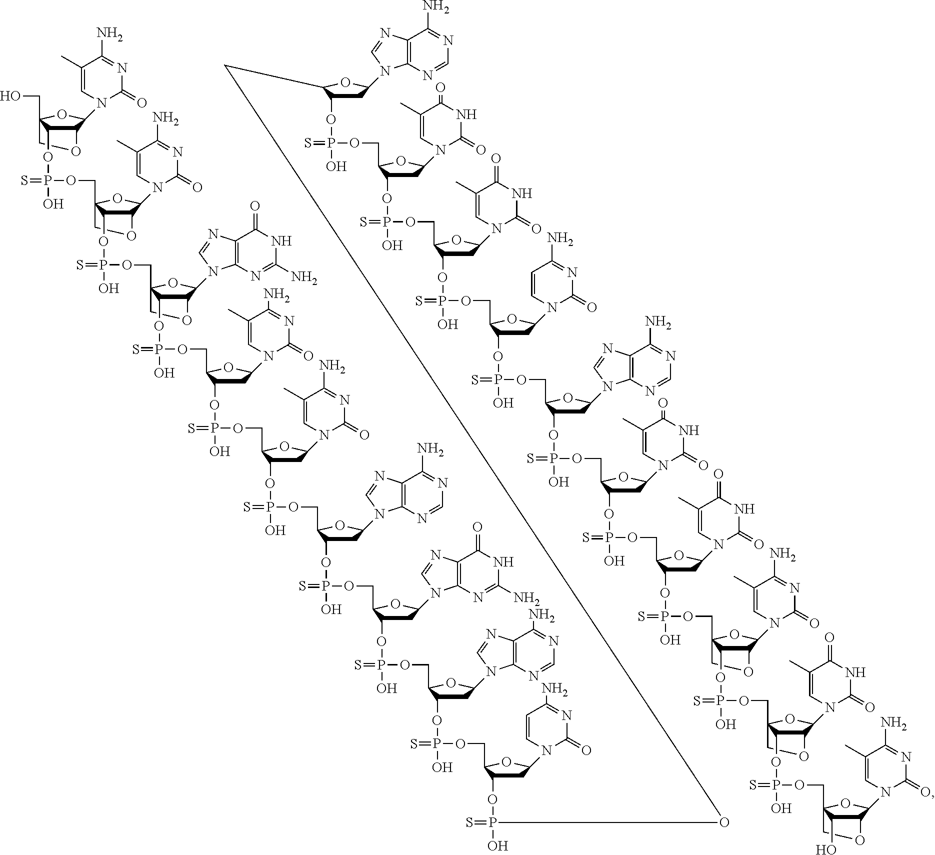
or a salt thereof.

21. A MAPT ASO according to the following chemical structure: 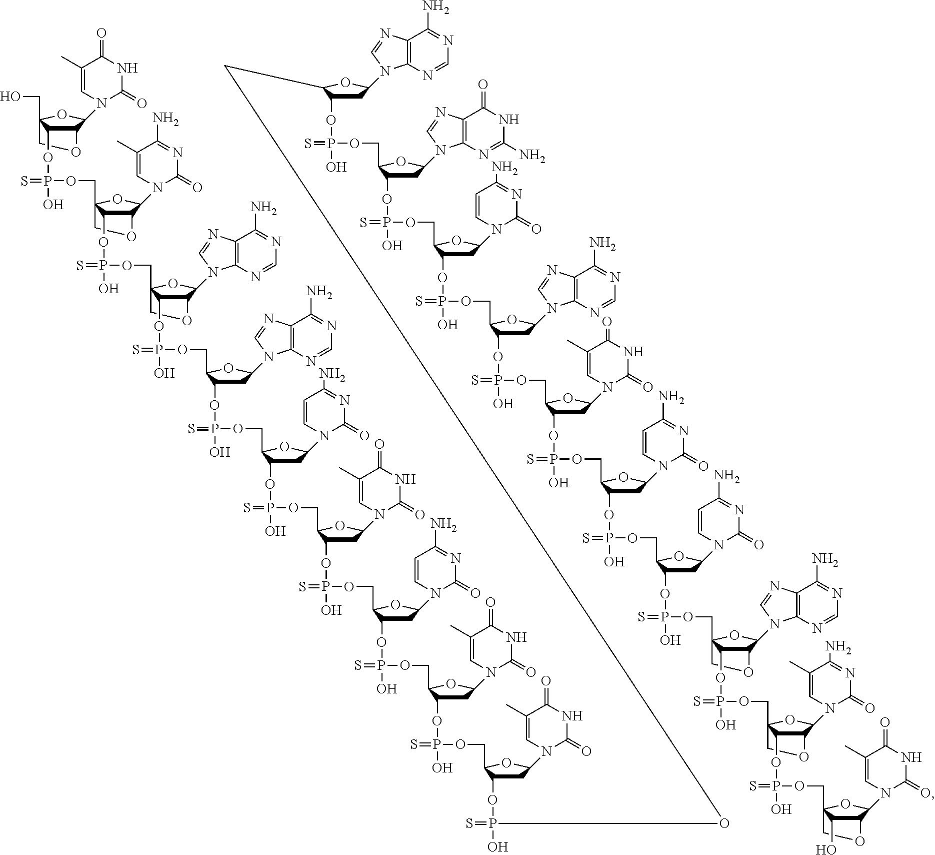
or a salt thereof.

22. A MAPT ASO according to the following chemical structure: 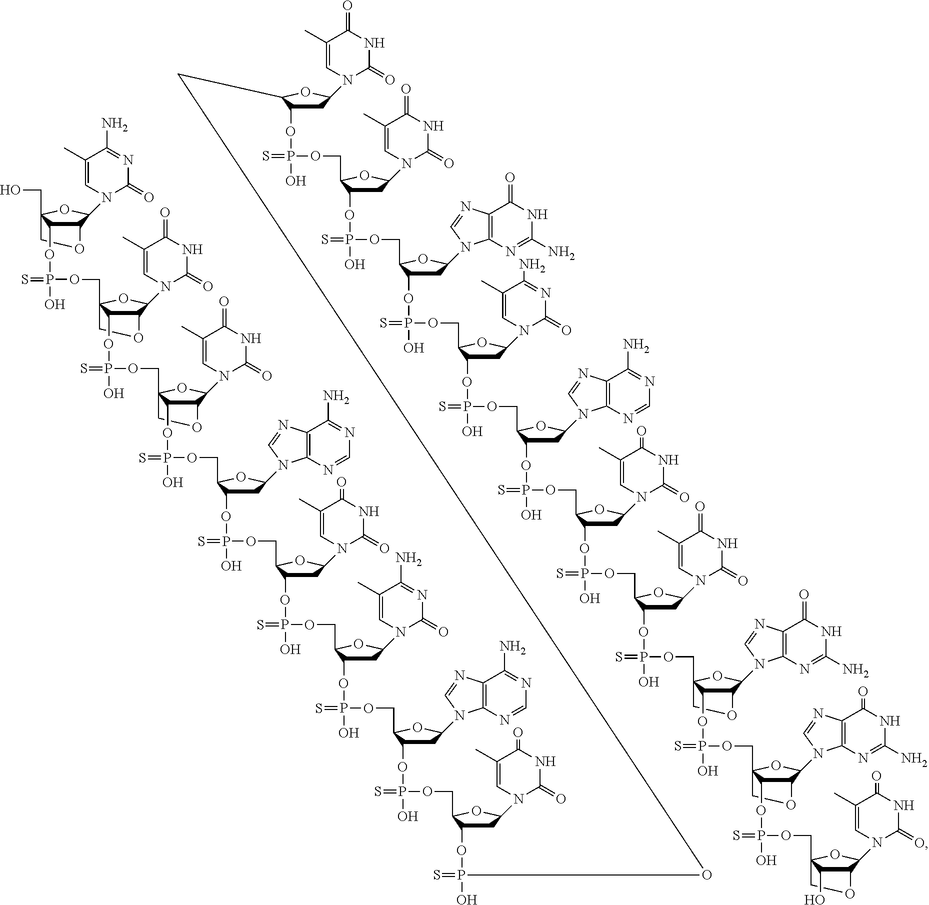
or a salt thereof.

23. A MAPT ASO according to the following chemical structure: 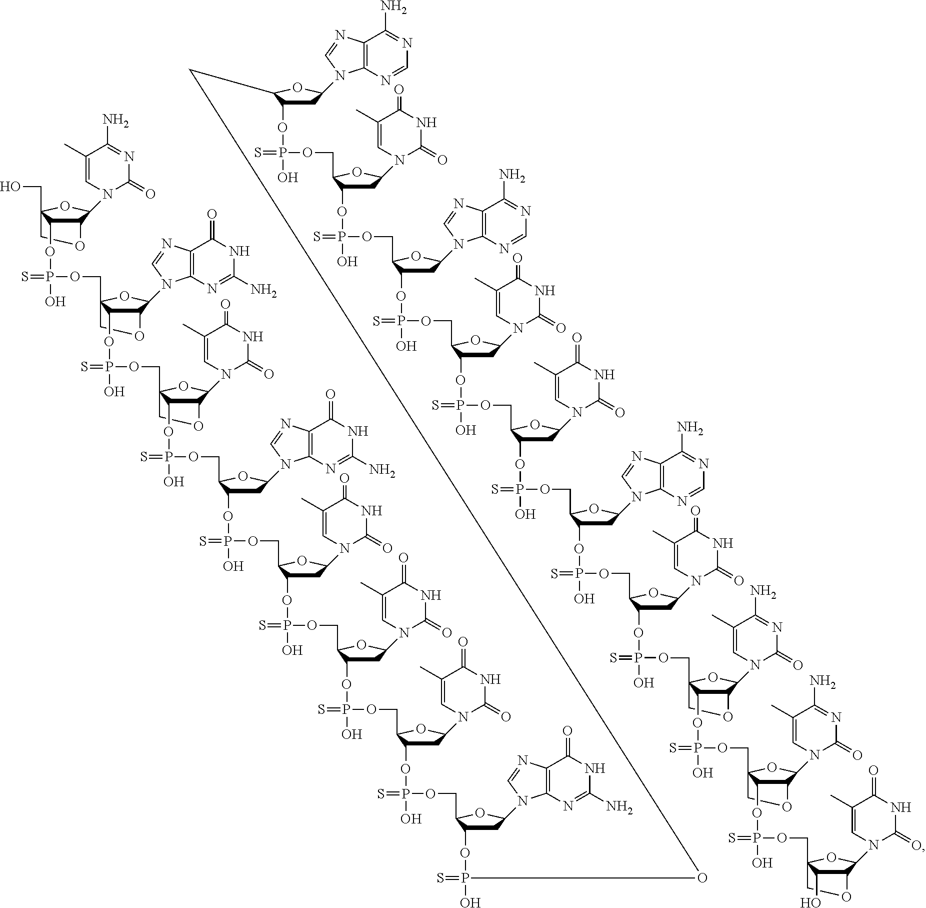
or a salt thereof.

24. The MAPT ASO of any one of claims 1-23, wherein the MAPT ASO is conjugated to a targeting ligand or a delivery vehicle.

25. The MAPT ASO of claim 24, wherein the targeting ligand specifically binds to the transferrin receptor (TfR) or a molecule expressed on the luminal surface of the blood brain barrier.

26. A pharmaceutical composition comprising a MAPT ASO of any one of claims 1-25 and a pharmaceutically acceptable carrier or diluent.

27. A method of generating a neuron cell with decreased tau expression, the method comprising delivering to the neuron cell a MAPT ASO of any one of claims 1-25, wherein the MAPT ASO decreases the expression level of an endogenous MAPT gene.

28. A method of modifying a neuron cell to decrease tau expression, the method comprising delivering to the neuron cell a MAPT ASO of any one of claims 1-25, wherein the MAPT ASO decreases the expression level of an endogenous MAPT gene.

29. A method of modifying a neuron cell to decrease tau expression, the method comprising delivering to the neuron cell a MAPT ASO of any one of claims 1-25, wherein the MAPT ASO specifically reduces the expression level of a MAPT transcript in the cell.

30. A method of reducing expression of tau in a cell of the spinal cord of a subject comprising administering the MAPT ASO of any one of claims 1-25 or the pharmaceutical composition of claim 26 by intrathecal administration.

31. A method of reducing tau expression in a subject comprising administering the MAPT ASO of any one of claims 1-25 or the pharmaceutical composition of claim 26 to the subject.

32. The method of claim 31, wherein tau expression is reduced in the CNS of the subject.

33. The method of claim 31, wherein the MAPT ASO is administered to the subject by intrathecal administration.

34. The method of any one of claims 27-33 wherein the MAPT ASO decreases the expression level of an endogenous MAPT gene or reduces the level of a MAPT transcript by at least about 10%, at least 20%, at least 30%, at least 40%, at least 50%, at least 60%, at least 70%, at least 80%, at least 90%, or at least 95% as compared to the level without administering the MAPT ASO.

35. The method of any one of claims 31-34, wherein the MAPT ASO is administered to the subject intravenously.

36. A method of treating a tau-associated neurodegenerative disorder in a human subject in need thereof, the method comprising administering to the human subject the MAPT ASO of any one of claims 1-25 or the pharmaceutical composition of claim 26.

37. The method of claim 36, wherein the tau-associated neurodegenerative syndrome is Alzheimer's Disease.

38. A method of treating Alzheimer's disease, the method comprising administering to a human subject in need thereof, the pharmaceutical composition of claim 26.

39. A method of reducing MAPT messenger ribonucleic acid (mRNA) expression in a human subject in need thereof, the method comprising administering to the human subject the MAPT ASO of any one of claims 1-25 or the pharmaceutical composition of claim 26.

40. The pharmaceutical composition as described in claim 26, for use in delivering a MAPT ASO to the CNS of a human subject in need thereof, wherein said MAPT ASO decreases the expression level of an endogenous MAPT gene.

41. The pharmaceutical composition as described in claim 26 for use in treating a tau-associated neurodegenerative disorder in a human subject in need thereof.

42. A pharmaceutical composition as claim 41, wherein the tau-associated neurodegenerative disorder is Alzheimer's.

43. The pharmaceutical composition as described in claim 26 for use in reducing MAPT mRNA expression in a human subject in need thereof.
